(19) United States
(12) Patent Application Publication
(10) Pub. No.: US 2023/0165846 A1
(43) Pub. Date: Jun. 1, 2023

(54) HETEROCYCLIC GLP-1 AGONISTS

(71) Applicant: Gasherbrum Bio, Inc., South San Francisco, CA (US)

(72) Inventors: Qinghua MENG, Shanghai (CN); Xichen LIN, Shanghai (CN); Andrew JENNINGS, San Francisco, CA (US)

(21) Appl. No.: 17/799,609

(22) PCT Filed: Feb. 9, 2021
(86) PCT No.: PCT/CN2021/076260

§ 371 Date: Aug. 12, 2022

(30) Foreign Application Priority Data
Feb. 13, 2020 (CN) PCTCN2020075103
Feb. 13, 2020 (CN) PCTCN2020075105

(51) Int. Cl. A61K 31/444; A61K 38/26; A61K 38/28; A61K 31/137; A61K 31/155; A61K 39/395; A61K 38/17; A61K 31/423; A61K 31/506; A61K 31/497; C07D 471/04

(52) U.S. Cl. CPC A61K 31/444; A61K 31/137; A61K 31/155; A61K 31/423; A61K 31/497; A61K 31/506; A61K 38/26; A61K 38/28; A61K 38/1709; A61K 39/3955; C07D 471/04

(57) ABSTRACT

Provided are GLP-1 agonists of Formula (I) or (II), including pharmaceutically acceptable salts and solvates thereof, pharmaceutical compositions, and methods of using the same.

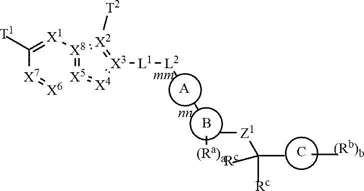

### CROSS REFERENCE TO RELATED APPLICATIONS

The application claims the benefit of International Patent Application No. PCT/CN2020/075105, filed on Feb. 13, 2020; and International Patent Application No. PCT/CN2020/075103, filed on Feb. 13, 2020, each of which is incorporated herein by reference in its entirety.

### TECHNICAL FIELD

This disclosure relates to GLP-1 agonists, pharmaceutical compositions, and methods of use thereof.

### BACKGROUND

Incretin metabolic hormones, including glucagon-like peptide-1 (GLP-1) and glucose-dependent insulinotropic polypeptide (GIP), are important in the regulation of glucose homeostasis. Medicaments targeting this family of intestinal peptides, such as GLP-1 agonists, have been shown to suppress glucagon production, decrease gastric motility, and increase satiety.

Diabetes mellitus refers to a group of metabolic disorders characterized by persistent hyperglycemia. The most common form, type 2 diabetes mellitus (T2DM) is an acquired condition that accounts for more than 90% of diabetes cases. Typical onset occurs in obese or otherwise sedentary adults and begins with insulin resistance. Though lifestyle changes can be useful in management of this disorder, patients with T2DM may be required to take antidiabetic medications, including dipeptidyl peptidase-4 inhibitors, SGLT2 inhibitors, and sulfonylureas, among others.

In healthy individuals, the incretin hormones glucose-dependent insulinotropic polypeptide (GIP) and glucagon-like peptide 1 (GLP-1) provide tandem modulation of insulin secretory response to glucose ingestion. While this incretin effect is significantly diminished (if at all present) in cases of T2DM, GLP-1 retains insulinotropic properties, even as endocrine pancreatic response to GIP is effectively halted. As such, incretin mimetics and other GLP-1-based therapies can help stimulate insulin production in T2DM patients.

### SUMMARY

The present application describes heterocyclic GLP-1 agonists, as well as pharmaceutical compositions comprising the compounds disclosed herein. Also provided are methods for treating GLP-1-associated diseases, disorders, and conditions.

Accordingly, provided herein are compounds of Formula I:

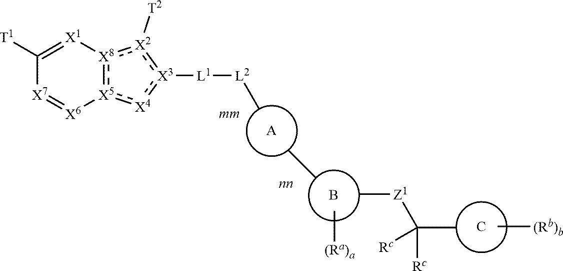

or a pharmaceutically acceptable salt or solvate thereof, wherein:

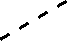

indicates an optional single or double bond, as allowed by valence;
each of X¹, X², X³, X⁴, X⁵, X⁶, X⁷, and X⁸ is independently selected from the group consisting of C, CH, and N, provided that at least two and no more than four of X¹, X², X³, X⁴, X⁵, X⁶, X⁷, and X⁸ are N;
T¹ is C(═O)OH or a carboxylic acid bioisostere;
T² is a (C₁-C₆)alkyl optionally substituted with (C₃-C₆)cycloalkyl, 3- to 6-membered heterocycloalkyl, phenyl, 5- to 6-membered heteroaryl, (C₁-C₆)alkoxy, CN, or (C₂-C₄)alkynyl, wherein each of the (C₃-C₆)cycloalkyl, 3- to 6-membered heterocycloalkyl, phenyl, or 5- to 6-membered heteroaryl is optionally substituted with 1-4 R^{x};
each R^{x} is independently selected from the group consisting of OH, SH, CN, NO₂, halogen, (C1-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino;
L¹ is (C₁-C₃)alkylene, which is optionally substituted with 1-3 R^{L};
L² is a bond, —O—, -S(O)₀₋₂-, or —NH—;
each R^{L} is independently selected from the group consisting of: halogen, (C₁-C₃)alkyl, and (C₁-C₃)haloalkyl; or
a pair of R^{L} on the same or on adjacent carbon atoms, taken together with the atom(s) to which each is attached, forms a (C₃-C₆)cycloalkyl ring;

Ring A is selected from the group consisting of:
partially unsaturated monocyclic (C₅-C₈)cycloalkylene optionally substituted with 1-4 substituents each independently selected from the group consisting of: halogen, (C₁-C₃)alkyl, (C₁-C₃)haloalkyl, (C₁-C₃)alkoxy, and (C₁-C₃)haloalkoxy; and
partially unsaturated monocyclic 5- to 8-membered heterocycloalkylene optionally substituted with 1-4 substituents each independently selected from the group consisting of: halogen, (C_{I}-C₃)alkyl, (C₁-C₃)haloalkyl, (C₁-C₃)alkoxy, and (C₁-C₃)haloalkoxy;
wherein mm represents the point of attachment to L², and nn represents the point of attachment to Ring B;

Ring B is selected from the group consisting of:

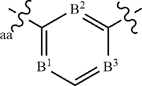

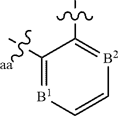

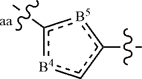

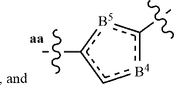

wherein aa represents the point of attachment to Ring A;
each of B¹, B², and B³ is independently selected from the group consisting of CR¹ and N;
each of B⁴ and B⁵ is independently selected from the group consisting of N, NR¹, C, CR¹, O, and S, provided that the ring containing B⁴ and B⁵ is heteroaryl;
R¹ is selected from the group consisting of H, halogen, and (C₁-C₆)alkyl;
each R^{a} is independently selected from the group consisting of (C₁-C₆)alkyl, (C₁-C₃)alkyl(C₃-C₆)cycloalkyl, (C₁-C₃)alkyl(3- to 5-membered heterocycloalkyl), -C(O)NR²R³, and (C₁-C₆)fluoroalkyl;
each R² and R³ is independently selected from the group consisting of H and (C₁-C₆)alkyl;
a is an integer selected from 0-3;
Z¹ is —O— or —NH—;
each R^{c} is independently selected from the group consisting of H, (C₁-C₆)alkyl, and (C₁-C₃)haloalkyl;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)alkoxy, halogen, (C₃-C₆)cycloalkyl, and CN; and
b is an integer selected from 0-3.

Also provided herein are compounds of Formula II:

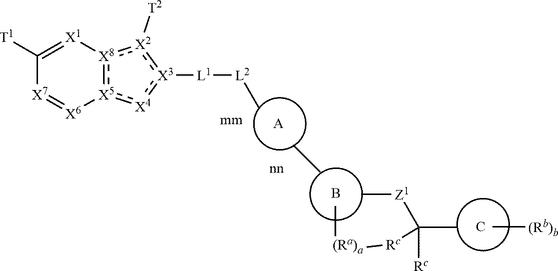

or a pharmaceutically acceptable salt or solvate thereof, wherein:

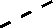

indicates an optional single or double bond, as allowed by valence;
each of X¹, X², X³, X⁴, X⁵, X⁶, X⁷, and X⁸ is independently selected from the group consisting of C, CH, and N, provided that at least two and no more than four of X¹, X², X³, X⁴, X⁵, X⁶, X⁷, and X⁸ are N;
T¹ is C(═O)OH or a carboxylic acid bioisostere;
T² is a (C₁-C₆)alkyl optionally substituted with (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{x};
each R^{x} is independently selected from the group consisting of OH, SH, CN, NO₂, halogen, (C1-C₆)alkyl, (C₂-C₆)alkenyl, (C₂-C₆)alkynyl, (C₁-C₆)haloalkyl, (C₁-C₆)cyanoalkyl, (C_{I}-C₆)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino;
L¹ is (C₁-C₃)alkylene, which is optionally substituted with 1-3 R^{L};
L² is a bond, —O—, -S(0)₀₋₂-, or —NH—;
each R^{L} is independently selected from the group consisting of: halogen, (C₁-C₃)alkyl, and (C₁-C₃)haloalkyl; or
a pair of R^{L} on the same or on adjacent carbon atoms, taken together with the atom(s) to which each is attached, forms a (C₃-C₆)cycloalkyl ring;

Ring A is selected from the group consisting of:
phenylene optionally substituted with 1-4 R^{Y};
5- to 6-membered heteroarylene optionally substituted with 1-3 R^{Y};
wherein mm represents the point of attachment to L², and nn represents the point of attachment to Ring B; and
each R^{Y} is independently selected from the group consisting of halogen, cyano, —OH, oxo, (C_{I}-C₃)alkyl, (C_{I}-C₃)haloalkyl, (C₁-C₃)alkoxy, and (C₁-C₃)haloalkoxy;

Ring B is selected from the group consisting of:

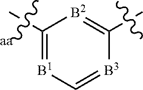

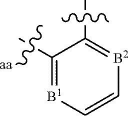

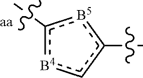

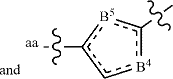

wherein aa represents the point of attachment to Ring A;
each of B¹, B², and B³ is independently selected from the group consisting of CR¹ and N;
each of B⁴ and B⁵ is independently selected from the group consisting of N, NR¹, C, CR¹, O, and S, provided that the ring containing B⁴ and B⁵ is heteroaryl;
R¹ is selected from the group consisting of H, halogen, and (C₁-C₆)alkyl;
each R^{a} is independently selected from the group consisting of (C1-C₆)alkyl, (C₁-C₃)alkyl(C₃-C₆)cycloalkyl, (C1-C₃)alkyl(3- to 5-membered heterocycloalkyl), —C(O)NR²R³, and (Cᵢ-C₆)fluoroalkyl;
each R² and R³ is independently selected from the group consisting of H and (C₁-C₆)alkyl;
a is an integer selected from 0-3;
Z¹ is —O— or —NH—;
each R^{c} is independently selected from the group consisting of H, (C₁-C₆)alkyl, and (C₁-C₃)haloalkyl;
Ring C is selected from the group consisting of phenyl, 5- to 6-membered heteroaryl, (C₃-C₆)cycloalkyl, (C₅-Cᵢₒ)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)alkoxy, halogen, (C₃-C₆)cycloalkyl, and CN; and
b is an integer selected from 0-3.

Also provided herein are pharmaceutical compositions comprising a compound of Formula I or II, or a pharmaceutically acceptable salt or solvate thereof, and a pharmaceutically acceptable excipient.

Also provided herein are methods for treating type 2 diabetes mellitus in a patient in need thereof, the methods comprising administering to the patient a therapeutically effective amount of a compound of Formula I or II, or a pharmaceutically acceptable salt or solvate thereof, or a pharmaceutical composition thereof.

Also provided herein are methods for treating type 2 diabetes mellitus in a patient, the methods comprising administering to a patient identified or diagnosed as having type 2 diabetes mellitus a therapeutically effective amount of a compound of Formula I or II, or a pharmaceutically acceptable salt or solvate thereof, or a pharmaceutical composition thereof.

Also provided herein are methods for treating diabetes mellitus in a patient, the methods comprising determining that the patient has type 2 diabetes mellitus; and administering to the patient a therapeutically effective amount of a compound of Formula I or II, or a pharmaceutically acceptable salt or solvate thereof, or a pharmaceutical composition thereof. In some embodiments, the step of determining that the patient has type 2 diabetes mellitus includes performing an assay to determine the level of an analyte in a sample from the patient, wherein the analyte is selected from the group consisting of hemoglobin A1c (HbA1c), fasting plasma glucose, non-fasting plasma glucose, or any combination thereof. In some embodiments, the level of HbA1c is greater than or about 6.5%. In some embodiments, the level of fasting plasma glucose is greater than or about 126 mg/dL. In some embodiments, the level of non-fasting plasma glucose is greater than or about 200 mg/dL.

In some embodiments, the methods further comprise obtaining a sample from the patient. In some embodiments, the sample is a body fluid sample. In some embodiments, the patient is about 40 to about 70 years old and is overweight or obese. In some embodiments, the patient has a body mass index (BMI) greater than or about 22 kg/m². In some embodiments, the patient has a BMI greater than or about 30 kg/m².

In some embodiments, the methods for the treatment of type 2 diabetes mellitus comprise a reduction in fasting plasma glucose levels. In some embodiments, the fasting plasma glucose levels are reduced to about or below 100 mg/dL.

In some embodiments, the methods for the treatment of type 2 diabetes mellitus comprise a reduction in HbAlc levels. In some embodiments, the HbAlc levels are reduced to about or below 5.7 %.

In some embodiments, the methods for the treatment of type 2 diabetes mellitus comprise a reduction in glucagon levels.

In some embodiments, the methods for the treatment of type 2 diabetes mellitus comprise an increase in insulin levels.

In some embodiments, the methods for the treatment of type 2 diabetes mellitus comprise a decrease in BMI. In some embodiments, the BMI is decreased to about or below 25 kg/m².

In some embodiments, the compound of Formula I or II, or a pharmaceutically acceptable salt or solvate thereof, or a pharmaceutical composition thereof, is administered orally.

In some embodiments, the methods of treatment for type 2 diabetes mellitus further comprise administering an additional therapy or therapeutic agent to the patient. In some embodiments, the additional therapy or therapeutic agent is selected from the group consisting of an antidiabetic agent, an anti-obesity agent, a GLP-1 receptor agonist, an agent to treat non-alcoholic steatohepatitis (NASH), gastric electrical stimulation, dietary monitoring, physical activity, or any combinations thereof. In some embodiments, the antidiabetic agent is selected from the group consisting of a biguanide, a sulfonylurea, a glitazar, a thiazolidinedione, a dipeptidyl peptidase 4 (DPP-4) inhibitor, a meglitinide, a sodium-glucose linked transporter 2 (SGLT2) inhibitor, a glitazone, a GRP40 agonist, a glucose-dependent insulinotropic peptide (GIP), an insulin or insulin analogue, an alpha glucosidase inhibitor, a sodium-glucose linked transporter 1 (SGLT1) inhibitor, or any combinations thereof. In some embodiments, the biguanide is metformin. In some embodiments, the anti-obesity agent is selected from the group consisting of neuropeptide Y receptor type 2 (NPYR2) agonist, a NPYR1 or NPYR5 antagonist, a human proislet peptide (HIP), a cannabinoid receptor type 1 (CB1R) antagonist, a lipase inhibitor, a melanocortin receptor 4 agonist, a farnesoid X receptor (FXR) agonist, phentermine, zonisamide, a norepinephrine/dopamine reuptake inhibitor, a GDF-15 analog, an opioid receptor antagonist, a cholecystokinin agonist, a serotonergic agent, a methionine aminopeptidase 2 (MetAP2) inhibitor, diethylpropion, phendimetrazine, benzphetamine, a fibroblast growth factor receptor (FGFR) modulator, an AMP-activated protein kinase (AMPK) activator, or any combinations thereof. In some embodiments, the GLP-1 receptor agonist is selected from the group consisting of liraglutide, exenatide, dulaglutide, albiglutide, taspoglutide, lixisenatide, semaglutide, or any combinations thereof. In some embodiments, the agent to treat NASH is selected from the group consisting of an FXR agonist, PF-05221304, a synthetic fatty acid-bile conjugate, an anti-lysyl oxidase homologue 2 (LOXL2) monoclonal antibody, a caspase inhibitor, a MAPK5 inhibitor, a galectin 3 inhibitor, a fibroblast growth factor 21 (FGF21) agonist, a niacin analogue, a leukotriene D4 (LTD4) receptor antagonist, an acetyl-CoA carboxylase (ACC) inhibitor, a ketohexokinase (KHK) inhibitor, an ileal bile acid transporter (IBAT) inhibitor, an apoptosis signal-regulating kinase 1 (ASK1) inhibitor, or any combinations thereof. In some embodiments, the compound of Formula I or II, or a pharmaceutically acceptable salt or solvate thereof, or a pharmaceutical composition thereof, and the additional therapeutic agent are administered as separate dosages sequentially in any order.

Also provided herein are methods for modulating insulin levels in a patient in need of such modulating, the method comprising administering to the patient an effective amount of a compound of Formula I or II, or a pharmaceutically acceptable salt or solvate thereof, or a pharmaceutical composition thereof. In some embodiments, the modulation results in an increase of insulin levels.

Also provided herein are methods for modulating glucose levels in a patient in need of such modulating, the method comprising administering to the patient an effective amount of a compound of Formula I or II, or a pharmaceutically acceptable salt or solvate thereof, or a pharmaceutical composition thereof. In some embodiments, the modulation results in a decrease of glucose levels.

Also provided herein are methods for treating a GLP-1 associated disease, disorder, or condition, the method comprising administering to a patient in need thereof an effective amount of a compound of Formula I or II, or a pharmaceutically acceptable salt or solvate thereof, or a pharmaceutical composition thereof. In some embodiments, the disease, disorder, or condition is selected from the group consisting of type 1 diabetes mellitus, type 2 diabetes mellitus, early onset type 2 diabetes mellitus, idiopathic type 1 diabetes mellitus (Type lb), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn’s disease, short bowel syndrome, Parkinson’s, Alzheimer’s disease, impaired cognition, schizophrenia, Polycystic Ovary Syndrome (PCOS), or any combination thereof. In some embodiments, the disease, disorder, or condition is selected from the group consisting of type 2 diabetes mellitus, early onset type 2 diabetes mellitus, obesity, weight gain from use of other agents, gout, excessive sugar craving, hypertriglyceridemia, dyslipidemia, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson’s disease, Polycystic Ovary Syndrome (PCOS), or any combination thereof. In some embodiments, the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

Other features and advantages of the invention will be apparent from the following detailed description and figures, and from the claims.

### DETAILED DESCRIPTION

Provided herein are heterocyclic GLP-1 agonists for use in the management of T2DM and other conditions where activation of GLP-1 activity is useful.

### Definitions

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

As used herein, the term “halo” or “halogen” means —F (sometimes referred to herein as “fluoro” or “fluoros”), —C1 (sometimes referred to herein as “chloro” or “chloros”), -_Br (sometimes referred to herein as “bromo” or “bromos”), and -I (sometimes referred to herein as “iodo” or “iodos”).

As used herein, the term “alkyl” refers to saturated linear or branched-chain monovalent hydrocarbon radicals, containing the indicated number of carbon atoms. For example, “(C₁-C₆)alkyl” refers to saturated linear or branched-chain monovalent hydrocarbon radicals of one to six carbon atoms. Non-limiting examples of alkyl include methyl, ethyl, 1-propyl, isopropyl, 1-butyl, isobutyl, sec-butyl, tert-butyl, 2-methyl-2-propyl, pentyl, neopentyl, and hexyl.

As used herein, the term “alkylene” refers to a divalent alkyl containing the indicated number of carbon atoms. For example, “(C_{I}-C₃)alkylene” refers to a divalent alkyl having one to three carbon atoms (e.g., —CH₂—, —CH(CH₃)—, —CH₂CH₂—, or —CH₂CH₂CH₂—). Similarly, the terms “cycloalkylene”, “heterocycloalkylene”, “arylene”, and “heteroarylene” mean divalent cycloalkyl, heterocycloalkyl, aryl, and heteroaryl, respectively.

As used herein, the term “alkenyl” refers to a linear or branched mono-unsaturated hydrocarbon chain, containing the indicated number of carbon atoms. For example, “(C₂-C₆)alkenyl” refers a linear or branched mono unsaturated hydrocarbon chain of two to six carbon atoms. Non-limiting examples of alkenyl include ethenyl, propenyl, butenyl, or pentenyl.

As used herein, the term “alkynyl” refers to a linear or branched di-unsaturated hydrocarbon chain, containing the indicated number of carbon atoms. For example, “(C₂-C₆)alkynyl” refers to a linear or branched di-unsaturated hydrocarbon chain having two to six carbon atoms. Non-limiting examples of alkynyl include ethynyl, propynyl, butynyl, or pentynyl.

As used herein, the term “cycloalkyl” refers to a saturated or partially unsaturated cyclic hydrocarbon, containing the indicated number of carbon atoms. For example, “(C₃-C₆)cycloalkyl” refers to a saturated or partially unsaturated cyclic hydrocarbon having three to six ring carbon atoms. Non-limiting examples of cycloalkyl include cyclopropyl, cyclobutyl, cyclopentyl, and cyclohexyl. Cycloalkyl may be partially unsaturated. Non-limiting examples of partially unsaturated cycloalkyl include cyclohexenyl, cyclopentenyl, cycloheptenyl, cyclooctenyl, and the like. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

As used herein, the term “heterocycloalkyl” refers to a mon-, bi-, tri-, or polycyclic nonaromatic ring system containing indicated number of ring atoms (e.g., 3-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocycloalkyl groups include piperazinyl, pyrrolidinyl, dioxanyl, morpholinyl, tetrahydrofuranyl, and the like. Heterocycloalkyl groups may be partially unsaturated. Non-limiting examples of partially unsaturated heterocycloalkyl include dihydropyrrolyl, dihydropyridinyl, tetrahydropyridinyl, dihydrofuranyl, dihydropyranyl, and the like. Heterocycloalkyl may include multiple fused and bridged rings. Non-limiting examples of fused/bridged heteorocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocycloalkyl also includes spirocyclic rings (e.g., spirocyclic bicycle wherein two rings are connected through just one atom). Non-limiting examples of spirocyclic heterocycloalkyl include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

As used herein, the term “aryl” refers to a mono-, bi-, tri- or polycyclic hydrocarbon group containing the indicated numbers of carbon atoms, wherein at least one ring in the system is aromatic (e.g., C₆ monocyclic, C₁₀ bicyclic, or C₁₄ tricyclic aromatic ring system). Examples of aryl groups include phenyl, naphthyl, tetrahydronaphthyl, and the like.

As used herein, the term “heteroaryl” refers to a mono-, bi-, tri- or polycyclic group having indicated numbers of ring atoms (e.g., 5-6 ring atoms; e.g., 5, 6, 9, 10, or 14 ring atoms); and having 6, 10, or 14 pi electrons shared in a cyclic array; wherein at least one ring in the system is aromatic (but does not have to be a ring which contains a heteroatom, e.g. tetrahydroisoquinolinyl, e.g., tetrahydroquinolinyl), and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others.

As used herein, the term “haloalkyl” refers to an alkyl radical as defined herein, wherein one or more hydrogen atoms is replaced with one or more halogen atoms. Non-limiting examples include fluoromethyl, difluoromethyl, trifluoromethyl, 2-fluoroethyl, 2,2-difluoroethyl, 2,2,2-trifluoroethyl, chloromethyl, dichloromethyl, chloroethyl, trichloroethyl, bromomethyl, and iodomethyl.

As used herein, the term “alkoxy” refers to an —O—alkyl radical, wherein the radical is on the oxygen atom. For example, “C₁₋₆ alkoxy” refers to an -O-(C₁₋₆ alkyl) radical, wherein the radical is on the oxygen atom. Examples of alkoxy include methoxy, ethoxy, propoxy, isopropoxy, butoxy and tert-butoxy. Accordingly, as used herein, the term “haloalkoxy” refers to an —O—haloalkyl radical, wherein the radical is on the oxygen atom.

As used herein, “ [symbol] ” indicates an optional single or double bond, as allowed by valence. As used herein, “ [symbol] ” indicates the point of attachment to the parent molecule.

As used herein, the term “compound,” is meant to include all stereoisomers, geometric isomers, tautomers, and isotopes of the structures depicted. Compounds herein identified by name or structure as one particular tautomeric form are intended to include other tautomeric forms unless otherwise specified.

As used herein, when a ring is described as being “aromatic”, it means the ring has a continuous, delocalized π-electron system. Typically, the number of out of plane π-electrons corresponds to the Hückel rule (4n+2). Examples of such rings include: benzene, pyridine, pyrimidine, pyrazine, pyridazine, pyridone, pyrrole, pyrazole, oxazole, thioazole, isoxazole, isothiazole, and the like. When a ring system comprising at least two rings is described as “aromatic”, it means said ring system comprises one or more aromatic ring(s). Accordingly, when a ring system comprising at least two rings is described as “non-aromatic”, none of the constituent rings of said ring system is aromatic.

As used herein, when a ring is described as being “partially unsaturated”, it means the ring has one or more additional degrees of unsaturation (in addition to the degree of unsaturation attributed to the ring itself; e.g., one or more double bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like. When a ring system comprising at least two rings is described as “partially unsaturated”, it means the ring system comprises one or more partially unsaturated ring(s), provided that none of the constituent rings of the ring system is aromatic.

As used herein, the term “carboxylic acid bioisostere” means a group which has chemical and physical similarities producing broadly similar biological properties to a carboxylic acid (see Lipinski, Annual Reports in Medicinal Chemistry, 1986,21,p283 “Bioisosterism In Drug Design”; Yun, Hwahak Sekye, 1993, 33, pages 576-579 “Application Of Bioisosterism To New Drug Design”; Zhao, Huaxue Tongbao, 1995, pages 34-38 25 “Bioisosteric Replacement And Development Of Lead Compounds In Drug Design”; Graham, Theochem, 1995, 343, pages 105-109 “Theoretical Studies Applied To Drug Design:ab initio Electronic Distributions In Bioisosteres”). Examples of suitable carboxylic acid bioisostere include: sulfo, phosphono, alkylsulfonylcarbamoyl, tetrazolyl, arylsulfonylcarbamoyl, heteroarylsulfonylcarbamoyl, N-methoxycarbamoyl, 3-hydroxy-3-cyclobutene-1,2-dione, 3,5-dioxo-1,2,4-oxadiazolidinyl or heterocyclic phenols such as 3-hydroxyisoxazolyl and 3-hydoxy-1-methylpyrazolyl.

The term “tautomer” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the invention, and the naming of the compounds does not exclude any tautomer.

The term “GLP-1R” or “GLP-1 receptor” as used herein is meant to include, without limitation, nucleic acids, polynucleotides, oligonucleotides, sense and antisense polynucleotide strands, complementary sequences, peptides, polypeptides, proteins, homologous, and/or orthologous GLP-1R molecules, isoforms, precursors, mutants, variants, derivatives, splice variants, alleles, different species, and active fragments thereof.

The term “GLP-1 associated disease” as used herein is meant to include, without limitation, all those diseases, disorders, or conditions in which modulating glucagon-like peptide-1 (GLP-1) receptor signaling can alter the pathology and/or symptoms and/or progression of the disease, disorder, or condition.

The term “GLP-1 agonist” or “GLP-1 RA” as used herein refers to an agonist of the glucagon-like peptide-1 (GLP-1) receptor. GLP-1 RAs enhance glucose-dependent insulin secretion; suppress inappropriately elevated glucagon levels, both in fasting and postprandial states; and slow gastric emptying. Karla et al., Glucagon-like peptide-1 receptor agonists in the treatment of type 2 diabetes: Past, present, and future, Indian J Endocrinol Metab. 2016 Mar-Apr; 20(2): 254-267. GLP-1 RAs have been shown to treat type 2 diabetes. Examples of GLP-1 RAs include, but are not limited to, albiglutide (TANZEUM®), dulaglutide (LY2189265, TRULICITY®), efpeglenatide, exenatide (BYETTA®, BYDUREON®, Exendin-4), liraglutide (VICTOZA®, NN2211), lixisenatide (LYXUMIA®), semaglutide (OZEMPIC®), tirzepatide, ZP2929, NNC0113-0987, BPI-3016, and TT401. See, also, for example, additional GLP-1 recceptor agonists described in U.S. Pat. Nos. 10,370,426; 10,308,700; 10, 259,823; 10,208,019; 9,920,106; 9,839,664; 8,129,343; 8,536,122; 7,919,598; 6,414,126; 6,628,343; and RE45313; and International Publication Nos. WO 2019/239319; WO 2019/239371; WO 2020/103815; WO 2020/207474; WO20202/34726; WO2020/044266; WO2020117987; and WO2020263695.

The term “pharmaceutically acceptable” as used herein indicates that the compound, or salt or composition thereof is compatible chemically and/or toxicologically with the other ingredients comprising a formulation and/or the patient being treated therewith.

The term “therapeutic compound” as used herein is meant to include, without limitation, all compounds of Formula I or II, or pharmaceutically acceptable salts or solvates thereof (e.g., a compound of any one of Formulas IA and IB, or any one of Formulas IIA, IIB, and IIC, or a pharmaceutically acceptable salt or solvate thereof), and all compositions (e.g., pharmaceutical compositions) wherein a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof) is a component of the composition.

The term “administration” or “administering” refers to a method of giving a dosage of a compound or pharmaceutical composition to a vertebrate or invertebrate, including a mammal, a bird, a fish, or an amphibian. The method of administration can vary depending on various factors, e.g., the components of the pharmaceutical composition, the site of the disease, and the severity of the disease.

The terms “effective amount” or “effective dosage” or “pharmaceutically effective amount” or “therapeutically effective amount,” as used herein, refer to a sufficient amount of a chemical entity (e.g., a compound of Formula I or II, or a pharmaceutically acceptable salt or solvate thereof (e.g., a compound of any one of Formulas IA and IB, or or any one of Formulas IIA, IIB, and IIC, or a pharmaceutically acceptable salt or solvate thereof)) being administered which will relieve to some extent one or more of the symptoms of the disease or condition being treated, and can include curing the disease. “Curing” means that the symptoms of active disease are eliminated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study. In some embodiments, a “therapeutically effective amount” of a compound as provided herein refers to an amount of the compound that is effective as a monotherapy or combination therapy.

The term “excipient” or “pharmaceutically acceptable excipient” means a pharmaceutically-acceptable material, composition, or vehicle, such as a liquid or solid filler, diluent, carrier, solvent, or encapsulating material. In some embodiments, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutical composition” refers to a mixture of a compound of Formula I or II, or a pharmaceutically acceptable salt or solvate thereof (e.g., a compound of any one of Formulas IA and IB or or any one of Formulas IIA, IIB, and IIC, or a pharmaceutically acceptable salt or solvate thereof) as described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to, rectal, oral, intravenous, aerosol, parenteral, ophthalmic, pulmonary, and topical administration.

The terms “treat,” “treating,” and “treatment,” in the context of treating a disease, disorder, or condition, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The term “preventing”, as used herein, is the prevention of the onset, recurrence or spread, in whole or in part, of the disease or condition as described herein, or a symptom thereof.

The terms “subject”, “patient” or “individual”, as used herein, are used interchangeably and refers to any animal, including mammals such as mice, rats, other rodents, rabbits, dogs, cats, swine, cattle, sheep, horses, primates, and humans. In some embodiments, the term refers to a subject, particularly a mammalian subject, for whom diagnosis, prognosis, or therapy is desired or needed. In some embodiments, the patient is a human. In some embodiments, the subject has experienced and/or exhibited at least one symptom of the disease, disorder, or condition to be treated and/or prevented.

The terms “treatment regimen” and “dosing regimen” are used interchangeably to refer to the dose and timing of administration of each therapeutic agent in a combination of the invention.

The term “pharmaceutical combination”, as used herein, refers to a pharmaceutical treatment resulting from the mixing or combining of more than one active ingredient and includes both fixed and non-fixed combinations of the active ingredients.

The term “combination therapy” as used herein refers to a dosing regimen of two different therapeutically active agents (i.e., the components or combination partners of the combination), wherein the therapeutically active agents are administered together or separately in a manner prescribed by a medical care taker or according to a regulatory agency as defined herein.

The term “modulation”, as used herein, refers to a regulation or an adjustment (e.g., increase or decrease) and can include, for example agonism, partial agonism or antagonism.

### Compounds

In one aspect, provided herein are compounds of Formula I:

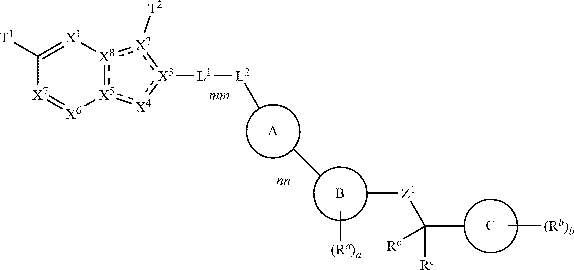

or a pharmaceutically acceptable salt or solvate thereof, wherein:

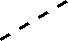

indicates an optional single or double bond, as allowed by valence;
each of X¹, X², X³, X⁴, X⁵, X⁶, X⁷, and X⁸ is independently selected from the group consisting of C, CH, and N, provided that at least two and no more than four of X¹, X², X³, X⁴, X⁵, X⁶, X⁷, and X⁸ are N;
T¹ is C(═O)OH or a carboxylic acid bioisostere;
T² is a (C₁-C₆)alkyl optionally substituted with (C₃-C₆)cycloalkyl, 3- to 6-membered heterocycloalkyl, phenyl, 5- to 6-membered heteroaryl, (C₁-C₆)alkoxy, CN, or (C₂-C₄)alkynyl, wherein each of the (C₃-C₆)cycloalkyl, 3- to 6-membered heterocycloalkyl, phenyl, or 5- to 6-membered heteroaryl is optionally substituted with 1-4 R^{x};
each R^{x} is independently selected from the group consisting of OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino;
L¹ is (C₁-C₃)alkylene, which is optionally substituted with 1-3 R^{L};
L² is a bond, —O—, -S(O)₀₋₂-, or —NH—;
each R^{L} is independently selected from the group consisting of: halogen, (C₁-C₃)alkyl, and (C₁-C₃)haloalkyl; or
a pair of R^{L} on the same or on adjacent carbon atoms, taken together with the atom(s) to which each is attached, forms a (C₃-C₆)cycloalkyl ring;

Ring A is selected from the group consisting of:
partially unsaturated monocyclic (C₅-C₈(cycloalkylene optionally substituted with 1-4 substituents each independently selected from the group consisting of: halogen, (C₁-C₃)alkyl, (C₁-C₃)haloalkyl, (C₁-C₃)alkoxy, and (C₁-C₃)haloalkoxy; and
partially unsaturated monocyclic 5- to 8-membered heterocycloalkylene optionally substituted with 1-4 substituents each independently selected from the group consisting of: halogen, (C₁-C₃)alkyl, (C₁-C₃)haloalkyl, (C₁-C₃)alkoxy, and (C₁-C₃)haloalkoxy;
wherein mm represents the point of attachment to L², and nn represents the point of attachment to Ring B;

Ring B is selected from the group consisting of:

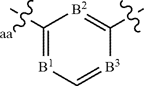

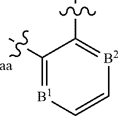

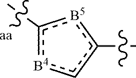

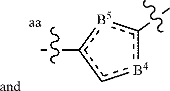

wherein aa represents the point of attachment to Ring A;
each of B¹, B², and B³ is independently selected from the group consisting of CR¹ and N;
each of B⁴ and B⁵ is independently selected from the group consisting of N, NR¹, C, CR¹, O, and S, provided that the ring containing B⁴ and B⁵ is heteroaryl;
R¹ is selected from the group consisting of H, halogen, and (C₁-C₆)alkyl;
each R^{a} is independently selected from the group consisting of (C₁-C₆)alkyl, (C₁-C₃)alkyl(C₃-C₆)cycloalkyl, (C₁-C₃)alkyl(3- to 5-membered heterocycloalkyl), —C(O)NR²R³, and (C₁-C₆)fluoroalkyl;
each R² and R³ is independently selected from the group consisting of H and (C₁-C₆)alkyl;
a is an integer selected from 0-3;
Z¹ is —O— or —NH—;
each R^{c} is independently selected from the group consisting of H, (C₁-C₆)alkyl, and (C₁-C₃)haloalkyl;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)alkoxy, halogen, (C₃-C₆)cycloalkyl, and CN; and
b is an integer selected from 0-3.

In some embodiments, provided herein are compounds of Formula I:

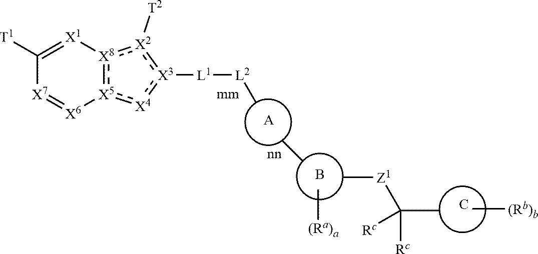

or a pharmaceutically acceptable salt or solvate thereof, wherein:

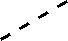

indicates an optional single or double bond, as allowed by valence;
each of X¹, X², X³, X⁴, X⁵, X⁶, X⁷, and X⁸ is independently selected from the group consisting of C, CH, and N, provided that at least two and no more than four of X¹, X², X³, X⁴, X⁵, X⁶, X⁷, and X⁸ are N;
T¹ is C(═O)OH or a carboxylic acid bioisostere;
T² is a (C₁-C₆)alkyl optionally substituted with (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{x};
each R^{x} is independently selected from the group consisting of OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino;
L¹ is (C₁-C₃)alkylene, which is optionally substituted with 1-3 R^{L};
L² is a bond, —O—, -S(O)₀₋₂-, or —NH—;
each R^{L} is independently selected from the group consisting of: halogen, (C₁-C₃)alkyl, and (C₁-C₃)haloalkyl; or
a pair of R^{L} on the same or on adjacent carbon atoms, taken together with the atom(s) to which each is attached, forms a (C₃-C₆)cycloalkyl ring;

Ring A is selected from the group consisting of:
partially unsaturated monocyclic (C₅-C₈)cycloalkylene optionally substituted with 1-4 substituents each independently selected from the group consisting of: halogen, (C₁-C₃)alkyl, (C₁-C₃)haloalkyl, (C₁-C₃)alkoxy, and (C₁-C₃)haloalkoxy; and
partially unsaturated monocyclic 5- to 8-membered heterocycloalkylene optionally substituted with 1-4 substituents each independently selected from the group consisting of: halogen, (C₁-C₃)alkyl, (C₁-C₃)haloalkyl, (C₁-C₃)alkoxy, and C₁-C₃)haloalkoxy;
wherein mm represents the point of attachment to L², and nn represents the point of attachment to Ring B;

Ring B is selected from the group consisting of:

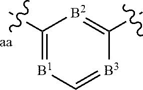

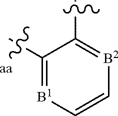

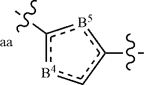

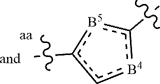

wherein aa represents the point of attachment to Ring A;
each of B¹, B², and B³ is independently selected from the group consisting of CR¹ and N;
each of B⁴ and B⁵ is independently selected from the group consisting of N, NR¹, C, CR¹, O, and S, provided that the ring containing B⁴ and B⁵ is heteroaryl;
R¹ is selected from the group consisting of H, halogen, and (C₁-C₆)alkyl;
each R^{a} is independently selected from the group consisting of (C₁-C₆)alkyl, (C₁-C₃)alkyl(C₃-C₆)cycloalkyl, (C₁-C₃)alkyl(3- to 5-membered heterocycloalkyl), —C(O)NR²R³, and (C₁-C₆)fluoroalkyl;
each R² and R³ is independently selected from the group consisting of H and (C₁-C₆)alkyl;
a is an integer selected from 0-3;
Z¹ is —O— or —NH—;
each R^{c} is independently selected from the group consisting of H, (C₁-C₆)alkyl, and (C₁-C₃)haloalkyl;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)alkoxy, halogen, (C₃-C₆)cycloalkyl, and CN; and
b is an integer selected from 0-3.

Embodiments of Formula I can include any one or more of the features delineated below and/or in the claims.

In some embodiments, X⁸ is C; and X⁵ is C.

In some embodiments, X³ is C.

In some embodiments, X² is N.

In some embodiments, X⁴ is N.

In some embodiments, X³ is C; X² is N; and X⁴ is N.

In some embodiments, X⁷ is CH.

In some embodiments, X⁸ is C; X⁵ is C; and X⁷ is CH.

In some embodiments, X⁸, X⁵, and X³ are C; X² and X⁴ are N; X⁷ is CH; and X¹ and X⁶ are independently CH or N. For example, X¹ and X⁶ are CH. As another non-limiting example, X¹ is N; and X⁶ is CH. As yet another non-limiting example, X¹ is CH; and X⁶ is N.

In some embodiments, X⁸, X⁵, and X³ are C; X⁷ and X⁶ are CH; X¹ is N; and X² and X⁴ is N.

In some embodiments, the

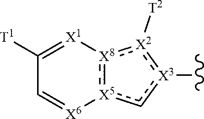

moiety has the formula:

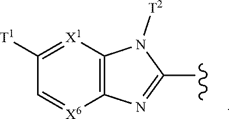

In some embodiments, the

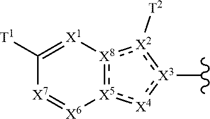

moiety has the formula:

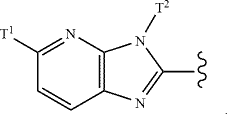

In some embodiments, the

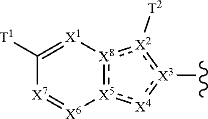

moiety has the formula:

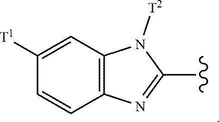

In some embodiments, the

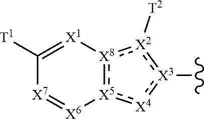

moiety has the formula:

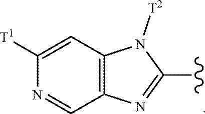

In some embodiments, T¹ is C(═O)OH.

In some embodiments, T¹ is a carboxylic acid bioisostere.

In some embodiments (when T¹ is a carboxylic acid bioisostere) , T¹ is a 5-membered heteroaryl including from 2-4 heteroatoms each independently selected from the group consisting of N, O, and S, wherein the heteroaryl is optionally substituted with from 1-4 substituents each independently selected from the group consisting of hydroxy, (C₁-C₆)alkyl, (C₁-C₆)haloalkyl, and halogen.

In some embodiments, T¹ is tetrazolyl, which is optionally substituted with from 1-2 substituents each independently selected from the group consisting of hydroxy, (C₁-C₆)alkyl, (C₁-C₆)haloalkyl, and halogen. For example, T¹ is selected from the group consisting of:

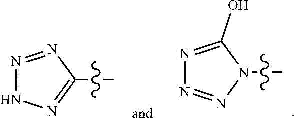

In some embodiments, T¹ is triazolyl or oxadiazolyl, which is optionally substituted with from 1-2 substituents each independently selected from (C₁-C₆)alkyl and hydroxy. For example, T¹ is

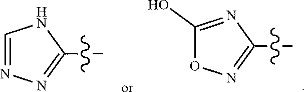

In some embodiments, T¹ is a ring (e.g., a 4-6 membered ring, e.g., a 5-membered ring) including from 0-3 heteroatoms each independently selected from the group consisting of N, O, and S, wherein the ring is substituted with from 1-2 oxo and further optionally substituted from 1-2 substituent each independently selected from the group consisting of hydroxy, (C₁-C₆)alkyl, (C₁-C₆)haloalkyl, and halogen. For example, T¹ is

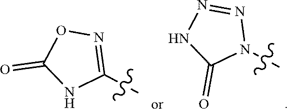

In some embodiments, T¹ is (C₁-C₆)alkyl which is substituted with from 1-3 hydroxy and further optionally substituted with from 1-10 fluoro. In certain of these embodiments, T¹ is (C₁-C₆)alkyl which is substituted with from 1-3 hydroxy and further substituted with from 1-10 fluoro. For example, T¹ is

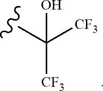

In some embodiments, T¹ is C(=O)NHS(O)₂(C₁-C₄)alkyl. For example, T¹ is C(=O)NHS(O)₂Me.

In some embodiments, T¹ is selected from the group consisting of the following:

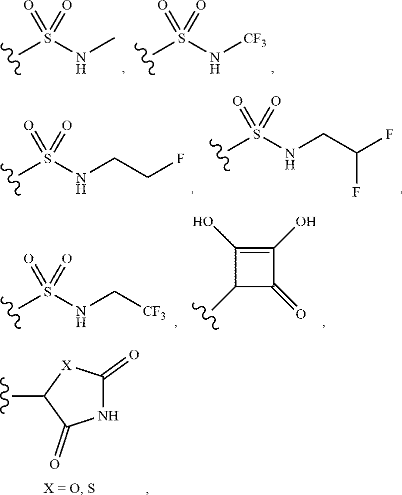

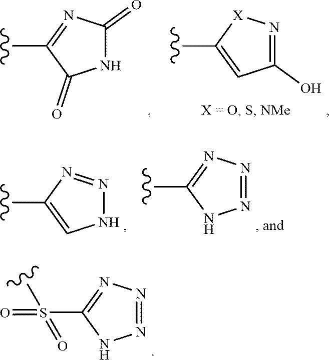

In some embodiments, T² is (C₁-C₃)alkyl which is substituted with (C₃-C₆)cycloalkyl, 3- to 6-membered heterocycloalkyl, phenyl, or 5- to 6-membered heteroaryl.

In some embodiments, T² is (C₁-C₃)alkyl which is substituted with (C₃-C₆)cycloalkyl or 3- to 6-membered heterocycloalkyl.

In some embodiments, T² is (C₁-C₃)alkyl which is substituted with 3- to 6-membered heterocycloalkyl.

In some embodiments, T² is (C₁-C₃)alkyl which is substituted with 4- to 6-membered heterocycloalkyl.

In some embodiments, T² is (C₁-C₃)alkyl which is substituted with oxetanyl.

In some embodiments, T² is

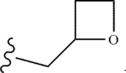

In some embodiments, T² is

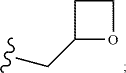

and the stereogenic center of T² has (S)-configuration.

In some embodiments, T² is (C₁-C₃)alkyl which is substituted with (C₃-C₆)cycloalkyl, wherein the (C₃-C₆)cycloalkyl is optionally substituted with 1-4 R^{x}. In some embodiments, T² is (C₁-C₃)alkyl which is substituted with cyclopropyl, wherein the cyclopropyl is optionally substituted with CN (e.g., cyclopropyl substituted with CN). For example, T² can be

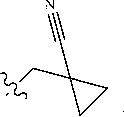

As another non-limiting example, T² can be

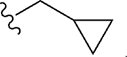

In some embodiments, T² is (C₁-C₃)alkyl which is substituted with (C₁-C₆)alkoxy. In some embodiments, T² is (C₁-C₃)alkyl which is substituted with methoxy. For example, T² can be —CH₂CH₂OCH₃.

In some embodiments, T² is (C₁-C₆)alkyl which is substituted with CN. In some embodiments, T² is branched (C₃-C₆)alkyl which is substituted with CN. For example, T² can be

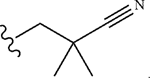

In some embodiments, T² is (C₁-C₃)alkyl which is substituted with (C₂-C₄)alkynyl. For example, T² can be

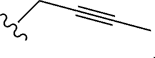

As a non-limiting example, the

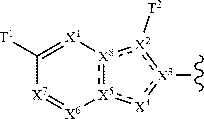

moiety can be:

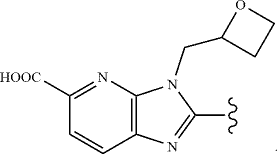

In some embodiments, L² is a bond.

In some embodiments, L² is —O—.

In some embodiments, L¹ is (C₁-C₂)alkylene, which is optionally substituted with 1-3 R^{L}.

In some embodiments, L¹ is CH₂.

In some embodiments, L¹ is CH₂CH₂.

In some embodiments, L¹ is CH₂CH₂, which is substituted with 1-3 R^{L}.

In some embodiments, L² is a bond; and L¹ is C₁₋₃ (e.g., C₁, C₂, or C₃) alkylene, which is optionally substituted with 1-3 R^{L}.

In some embodiments, L² is a bond; and L¹ is CH₂.

In some embodiments, L² is a bond; and L¹ is CH₂CH₂.

In some embodiments, L² is -0-; and L¹ is C₁₋₂ alkylene, which is optionally substituted with 1-3 R^{L}. As a non-limiting example, L² is —O—; and L¹ is CH₂.

In some embodiments, mm is para to nn.

In some embodiments, Ring A is partially unsaturated monocylic (C₅-C₈)cycloalkylene optionally substituted with 1-4 substituents each independently selected from the group consisting of: halogen, (C₁-C₃)alkyl, (C₁-C₃)haloalkyl, (C₁-C₃)alkoxy, and (C₁-C₃)haloalkoxy.

In some embodiments, Ring A is partially unsaturated monocylic C₆ cycloalkylene optionally substituted with from 1-4 substituents each independently selected from the group consisting of: halogen, (C₁-C₃)alkyl, (C₁-C₃)haloalkyl, (C₁-C₃)alkoxy, and (C₁-C₃)haloalkoxy.

In some embodiments, Ring A is cyclohexenylene optionally substituted with from 1-4 substituents each independently selected from the group consisting of: halogen, (C₁-C₃)alkyl, (C₁-C₃)haloalkyl, (C₁-C₃)alkoxy, and (C₁-C₃)haloalkoxy.

In some embodiments, Ring A is unsubstituted cyclohexenylene.

In some embodiments, Ring A is

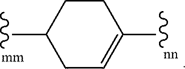

In some embodiments, Ring A is partially unsaturated monocyclic 5- to 8-membered heterocycloalkylene optionally substituted with from 1-4 substituents each independently selected from the group consisting of: halogen, (C₁-C₃)alkyl, (C₁-C₃)haloalkyl, (C₁-C₃)alkoxy, and (C₁-C₃)haloalkoxy.

In some embodiments, Ring A is partially unsaturated monocyclic 5- to 6-membered heterocycloalkylene optionally substituted with from 1-4 substituents each independently selected from the group consisting of: halogen, (C₁-C₃)alkyl, (C₁-C₃)haloalkyl, (C₁-C₃)alkoxy, and (C₁-C₃)haloalkoxy.

In some embodiments, Ring A is tetrahydropyridinylene which is optionally substituted with from 1-4 substituents each independently selected from the group consisting of: halogen, (C₁-C₃)alkyl, (C₁-C₃)haloalkyl, (C₁-C₃)alkoxy, and (C₁-C₃)haloalkoxy.

In some embodiments, Ring A is unsubstituted tetrahydropyridinylene.

In some embodiments, Ring A is

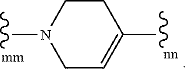

In some embodiments, Ring B is

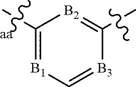

In some embodiments of (B—I), B² is N.

In some embodiments of (B—I), B¹ and B³ are independently CR¹.

In some embodiments of (B—I), one of B¹ and B³ is N; and the other one of B¹ and B³ is CR¹. For example, B¹ is N; and B³ is CR¹. As another non-limiting example, B¹ is CR¹; and B³ is N.

In some embodiments, Ring B is

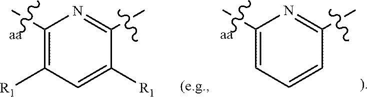

In some embodiments, Ring B is

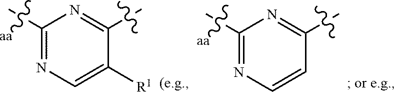

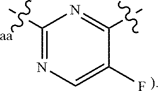

In some embodiments, Ring B is

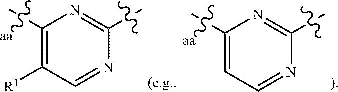

In some embodiments, Ring B is

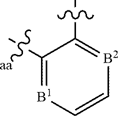

In some embodiments of (B—II), B² is CR¹.

In some embodiments of (B—II), B² is N.

In some embodiments of (B—II), B¹ is N.

In some embodiments of (B—II), B¹ is CR¹.

In some embodiments, Ring B is

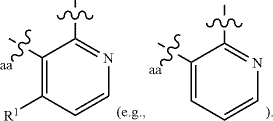

In some embodiments, Ring B is

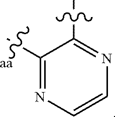

In some embodiments, Ring B is

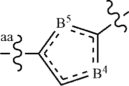

In some embodiments of (B—IV), B⁵ is N.

In some embodiments of (B—IV), B⁴ is selected from the group consisting of NR¹, S, and O. For example, B⁴ can be S.

In some embodiments, Ring B is

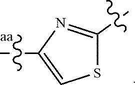

In some embodiments, each R¹ is independently H or halogen.

In some embodiments, each R¹ is H.

In some embodiments, a is 0.

In some embodiments, Z¹ is —O—.

In some embodiments, Z¹ is —NH—.

In some embodiments, each R^{c} is H.

In some embodiments, each R^{c} is an independently selected (C₁-C₆)alkyl or (C₁-C₃)haloalkyl.

In some embodiments, Z¹ is O; and each R^{c} is H.

In some embodiments, Ring C is selected from the group consisting of: phenyl, 5-to 6-membered heteroaryl, and 5- to 10-membered bicycloheteroaryl.

In some embodiments, Ring C is phenyl.

In some embodiments, Ring C is pyridyl.

In some embodiments, b is 1-3.

In some embodiments, b is 2.

In some embodiments, Ring C is phenyl; and b is 2.

In some embodiments, the

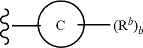

moiety is

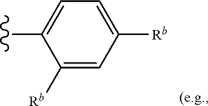

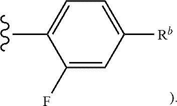

In some embodiments, Ring C is pyridyl (e.g., 2-pyridyl); and b is 1.

In some embodiments, the

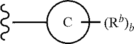

moiety is

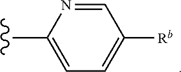

In some embodiments, each occurrence of R^{b} is independently selected from the group consisting of: (C₁-C₆)alkyl, (C₁-C₆)alkoxy, halogen, and CN.

In some embodiments, each occurrence of R^{b} is independently halogen or CN. For example, b is 2; one occurrence of R^{b} is halogen (e.g., —F or -Cl); and the second occurrence of R^{b} is —CN. As another non-limiting example, b is 2; and each occurrence of R^{b} is an independently selected halogen (e.g., each R^{b} is independently —Cl or -F).

In some embodiments, each occurrence of R^{b} is independently selected from the group consisting of —F, —Cl, and CN. As non-limiting examples, the

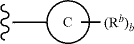

moiety can be

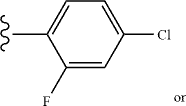

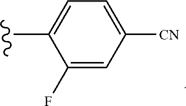

In some embodiments, the compound of Formula I is a compound of Formula IA:

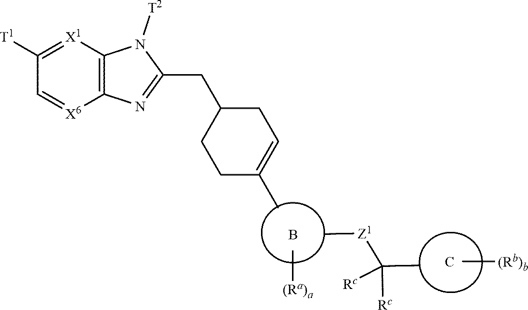

or a pharmaceutically acceptable salt or solvate thereof.

In some embodiments, the compound of Formula I is a compound of Formula IB:

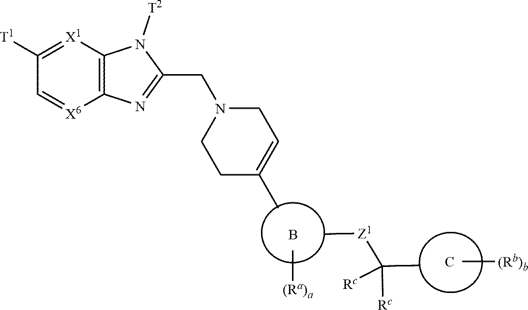

or a pharmaceutically acceptable salt or solvate thereof.

In some embodiments of Formula IA or IB, X¹ is N.

In some embodiments of Formula IA or IB, X⁶ is CH.

In some embodiments of Formula IA or IB, X¹ is N; and X⁶ is CH.

In some embodiments of Formula IA or IB, T¹ is C(═O)OH.

In some embodiments of Formula IA or IB, T² is (C₁-C₃)alkyl which is substituted with 3- to 6-membered heterocycloalkyl.

In some embodiments of Formula IA or IB, T² is (C₁-C₃)alkyl which is substituted with oxetanyl.

In some embodiments of Formula IA or IB, T² is is

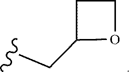

In some embodiments of Formula IA or IB, T² is (C₁-C₃)alkyl which is substituted with (C₃-C₆)cycloalkyl, wherein the (C₃-C₆)cycloalkyl is optionally substituted with CN (e.g., cyclopropyl substituted with CN; or unsubstituted cyclopropyl). For example, T² can be

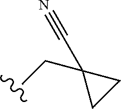

As another non-limiting example, T² can be

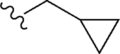

In some embodiments of Formula IA or IB, T² is (C₁-C₃)alkyl which is substituted with (C₁-C₆)alkoxy. In some embodiments, T² is (C₁-C₃)alkyl which is substituted with methoxy. For example, T² can be —CH₂CH₂OCH₃.

In some embodiments of Formula IA or IB, T² is (C₁-C₆)alkyl which is substituted with CN. In some embodiments, T² is branched (C₃-C₆)alkyl which is substituted with CN. For example, T² can be

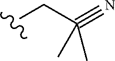

In some embodiments of Formula IA or IB, T² is (C₁-C₃)alkyl which is substituted with (C₂-C₄)alkynyl. For example, T² can be

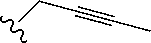

In some embodiments of Formula IA or IB, Ring B is

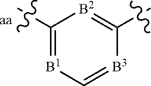

In some embodiments of Formula IA or IB, Ring B is

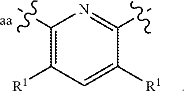

In some embodiments of Formula IA or IB, Ring B is selected from the group consisting of:

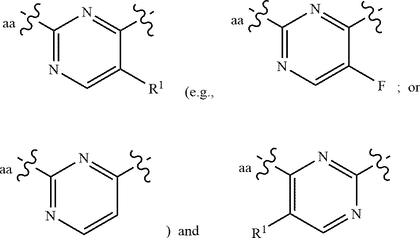

For example, Ring B can be

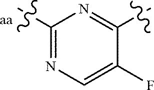

In some embodiments of Formula IA or IB, Ring B is

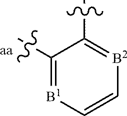

In some embodiments of Formula IA or IB, Ring B is

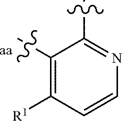

In some embodiments of Formula IA or IB, Ring B is

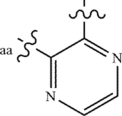

In some embodiments of Formula IA or IB, Ring B is

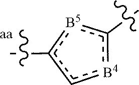

In some embodiments of Formula IA or IB, Ring B is

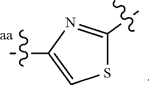

In some embodiments of Formula IA or IB, each R¹ is independently H or halogen.

In some embodiments of Formula IA or IB, each R¹ is H.

In some embodiments of Formula IA or IB, a is 0.

In some embodiments of Formula IA or IB, Z¹ is —O—.

In some embodiments of Formula IA or IB, each R^{c} is H.

In some embodiments of Formula IA or IB, Ring C is phenyl.

In some embodiments of Formula IA or IB, b is 1-3.

In some embodiments of Formula IA or IB, b is 2.

In some embodiments of Formula IA or IB, Ring C is phenyl; and b is 2.

In some embodiments of Formula IA or IB

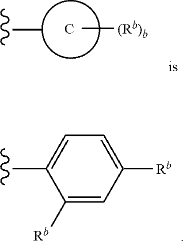

In some embodiments of Formula IA or IB, each occurrence of R^{b} is independently selected from the group consisting of: (C₁-C₆)alkyl, (C₁-C₆)alkoxy, halogen, and CN.

In some embodiments of Formula IA or IB, each occurrence of R^{b} is independently selected from the group consisting of —F, —Cl, and CN.

In some embodiments, a compound of Formula I is selected from the group consisting of the compounds in Table C1 or a pharmaceutically acceptable salt or solvate thereof.

TABLE C1

| Compound # | Structure |
|---|---|
| 101 | 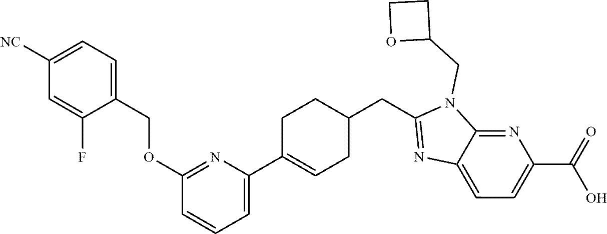 [SMILES: N#Cc1ccc(COc2cccc(C3=CCC(Cc4nc5ccc(C(=O)O)nc5n4CC4CCO4)CC3)n2)c(F)c1] |
| 102 | 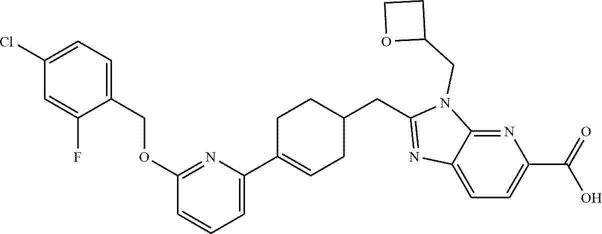 [SMILES: O=C(O)c1ccc2nc(CC3CC=C(c4cccc(OCc5ccc(Cl)cc5F)n4)CC3)n(CC3CCO3)c2n1] |
| 103 | 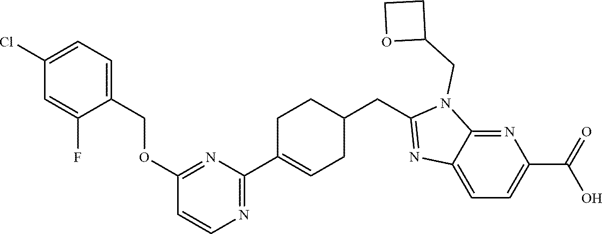 [SMILES: O=C(O)c1ccc2nc(CC3CC=C(c4nccc(OCc5ccc(Cl)cc5F)n4)CC3)n(CC3CCO3)c2n1] |
| 104 | 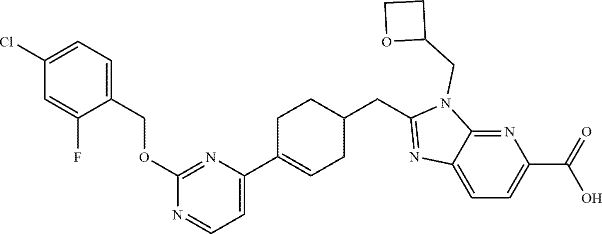 [SMILES: O=C(O)c1ccc2nc(CC3CC=C(c4ccnc(OCc5ccc(Cl)cc5F)n4)CC3)n(CC3CCO3)c2n1] |
| 105 | 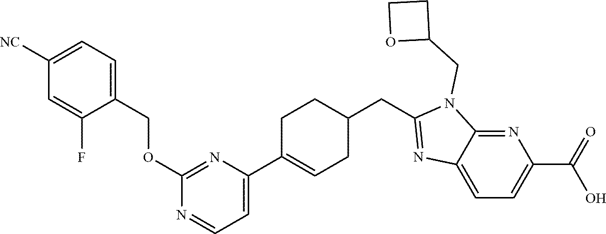 [SMILES: N#Cc1ccc(COc2nccc(C3=CCC(Cc4nc5ccc(C(=O)O)nc5n4CC4CCO4)CC3)n2)c(F)c1] |
| 106 | 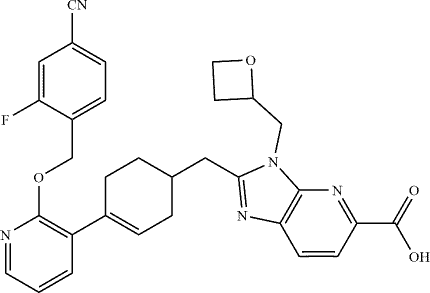 [SMILES: N#Cc1ccc(COc2ncccc2C2=CCC(Cc3nc4ccc(C(=O)O)nc4n3CC3CCO3)CC2)c(F)c1] |
| 107 | 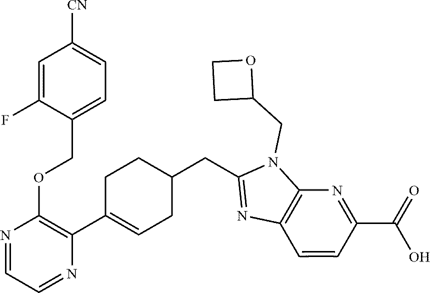 [SMILES: N#Cc1ccc(COc2nccnc2C2=CCC(Cc3nc4ccc(C(=O)O)nc4n3CC3CCO3)CC2)c(F)c1] |
| 108 | 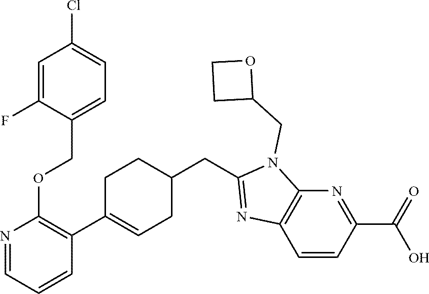 [SMILES: O=C(O)c1ccc2nc(CC3CC=C(c4cccnc4OCc4ccc(Cl)cc4F)CC3)n(CC3CCO3)c2n1] |
| 109 | 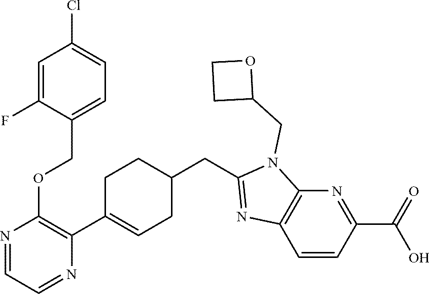 [SMILES: O=C(O)c1ccc2nc(CC3CC=C(c4nccnc4OCc4ccc(Cl)cc4F)CC3)n(CC3CCO3)c2n1] |
| 110 | 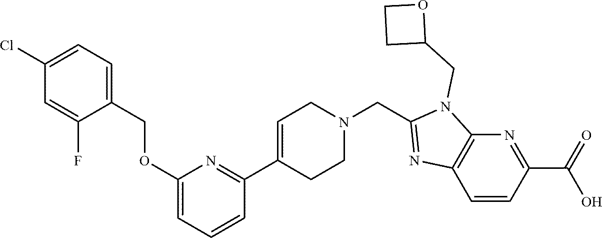 [SMILES: O=C(O)c1ccc2nc(CN3CC=C(c4cccc(OCc5ccc(Cl)cc5F)n4)CC3)n(CC3CCO3)c2n1] |
| 111 | 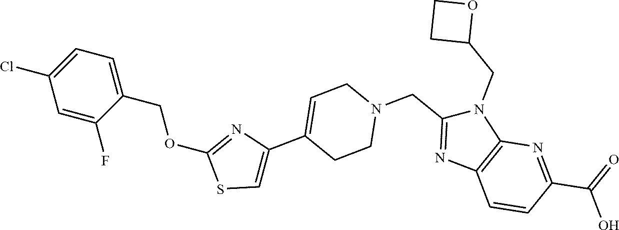 [SMILES: O=C(O)c1ccc2nc(CN3CC=C(c4csc(OCc5ccc(Cl)cc5F)n4)CC3)n(CC3CCO3)c2n1] |
| 112 | 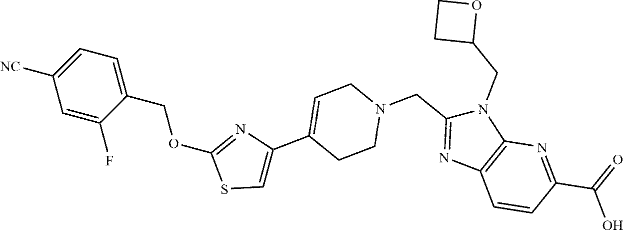 |
| 113 | 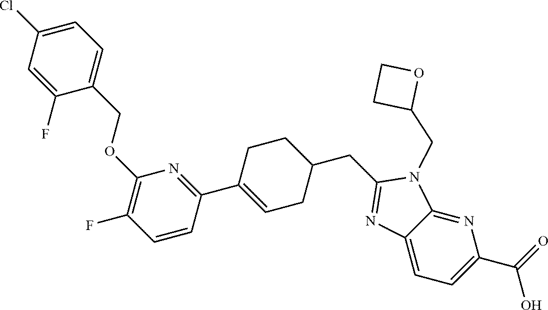 [SMILES: O=C(O)c1ccc2nc(CC3CC=C(c4ccc(F)c(OCc5ccc(Cl)cc5F)n4)CC3)n(CC3CCO3)c2n1] |
| 114 | 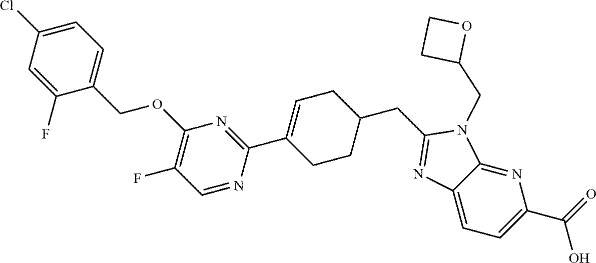 [SMILES: O=C(O)c1ccc2nc(CC3CC=C(c4ncc(F)c(OCc5ccc(Cl)cc5F)n4)CC3)n(CC3CCO3)c2n1] |
| 115 | 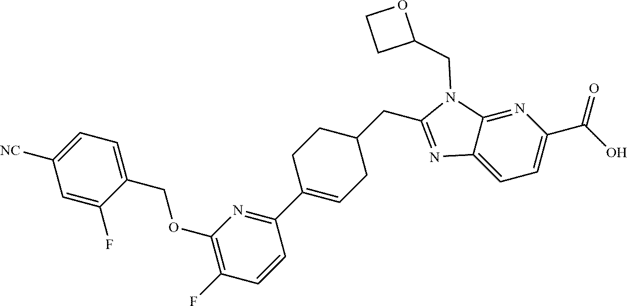 [SMILES: N#Cc1ccc(COc2nc(C3=CCC(Cc4nc5ccc(C(=O)O)nc5n4CC4CCO4)CC3)ccc2F)c(F)c1] |
| 116 | 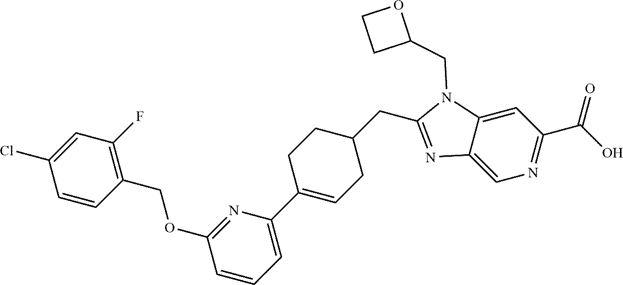 [SMILES: O=C(O)c1cc2c(cn1)nc(CC1CC=C(c3cccc(OCc4ccc(Cl)cc4F)n3)CC1)n2CC1CCO1] |
| 117 | 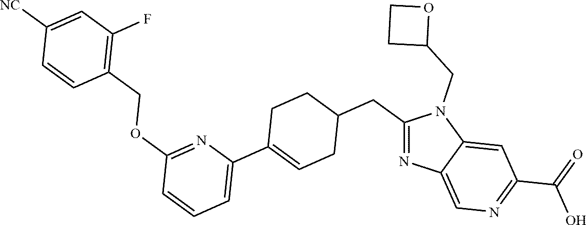 [SMILES: N#Cc1ccc(COc2cccc(C3=CCC(Cc4nc5cnc(C(=O)O)cc5n4CC4CCO4)CC3)n2)c(F)c1] |
| 118 | 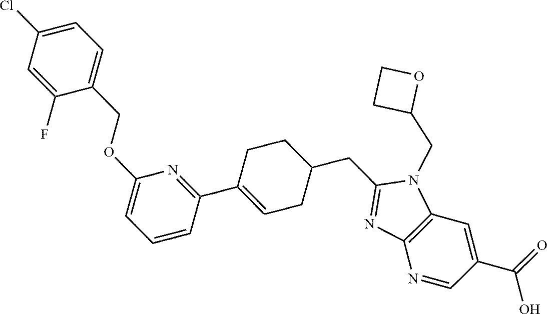 [SMILES: O=C(O)c1cnc2nc(CC3CC=C(c4cccc(OCc5ccc(Cl)cc5F)n4)CC3)n(CC3CCO3)c2c1] |
| 119 | 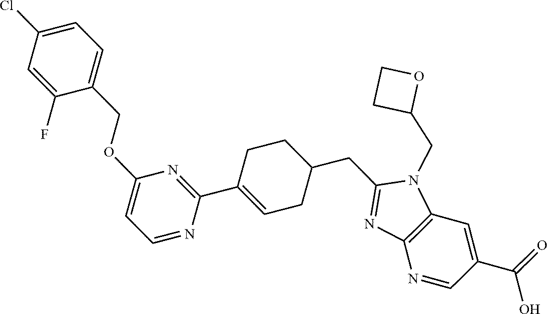 [SMILES: O=C(O)c1cnc2nc(CC3CC=C(c4nccc(OCc5ccc(Cl)cc5F)n4)CC3)n(CC3CCO3)c2c1] |
| 120 | 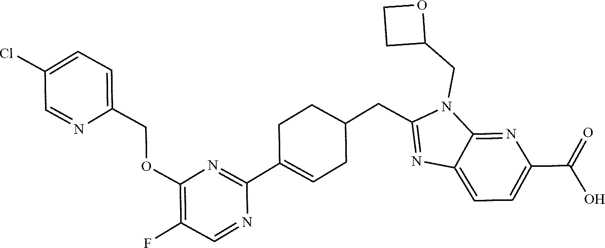 [SMILES: O=C(O)c1ccc2nc(CC3CC=C(c4ncc(F)c(OCc5ccc(Cl)cn5)n4)CC3)n(CC3CCO3)c2n1] |
| 121 | 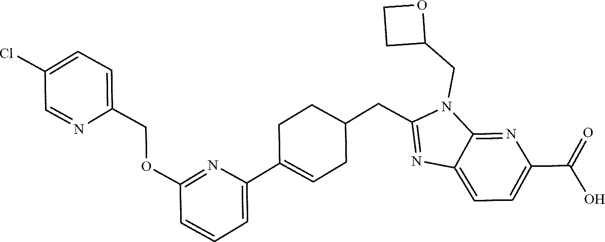 [SMILES: O=C(O)c1ccc2nc(CC3CC=C(c4cccc(OCc5ccc(Cl)cn5)n4)CC3)n(CC3CCO3)c2n1] |
| 122 | 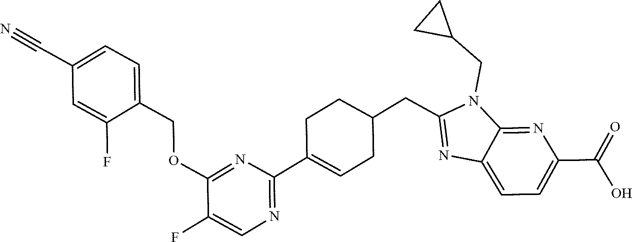 [SMILES: N#Cc1ccc(COc2nc(C3=CCC(Cc4nc5ccc(C(=O)O)nc5n4CC4CC4)CC3)ncc2F)c(F)c1] |
| 123 | 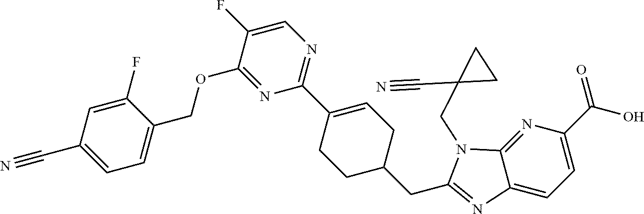 [SMILES: N#Cc1ccc(COc2nc(C3=CCC(Cc4nc5ccc(C(=O)O)nc5n4CC4(C#N)CC4)CC3)ncc2F)c(F)c1] |
| 124 | 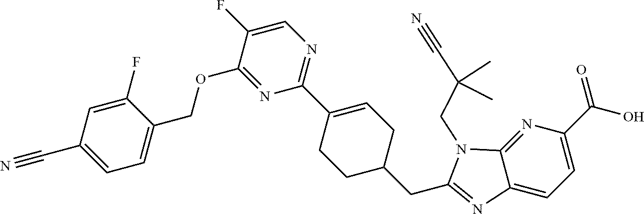 [SMILES: CC(C)(C#N)Cn1c(CC2CC=C(c3ncc(F)c(OCc4ccc(C#N)cc4F)n3)CC2)nc2ccc(C(=O)O)nc21] |
| 125 | 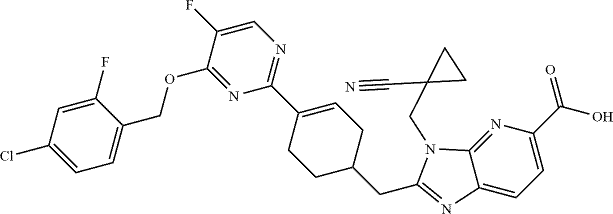 [SMILES: N#CC1(Cn2c(CC3CC=C(c4ncc(F)c(OCc5ccc(Cl)cc5F)n4)CC3)nc3ccc(C(=O)O)nc32)CC1] |
| 126 | 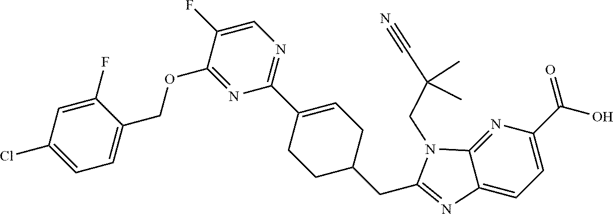 [SMILES: CC(C)(C#N)Cn1c(CC2CC=C(c3ncc(F)c(OCc4ccc(Cl)cc4F)n3)CC2)nc2ccc(C(=O)O)nc21] |
| 127 | 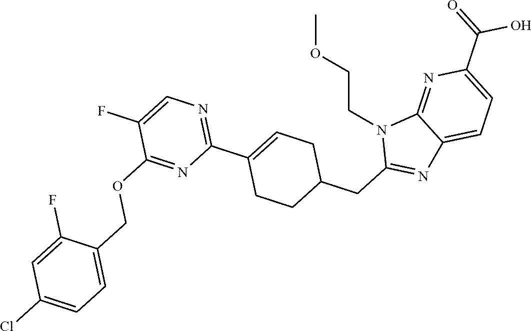 [SMILES: COCCn1c(CC2CC=C(c3ncc(F)c(OCc4ccc(Cl)cc4F)n3)CC2)nc2ccc(C(=O)O)nc21] |
| 128 | 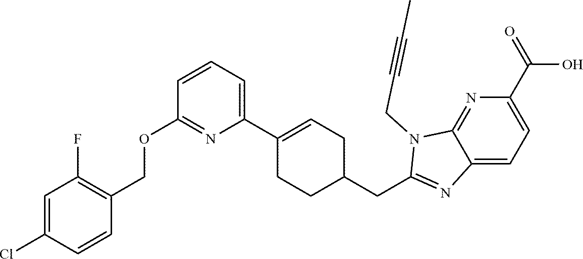 [SMILES: CC#CCn1c(CC2CC=C(c3cccc(OCc4ccc(Cl)cc4F)n3)CC2)nc2ccc(C(=O)O)nc21] |

In some embodiments, the compound is selected from the group consisting of the compounds in Table C2 or a pharmaceutically acceptable salt or solvate thereof.

TABLE C2

| Compound # | Structure |
|---|---|
| 101a | 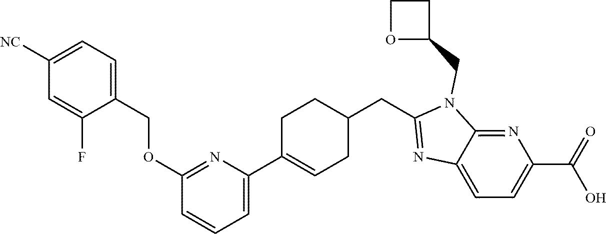 |
| 101b | 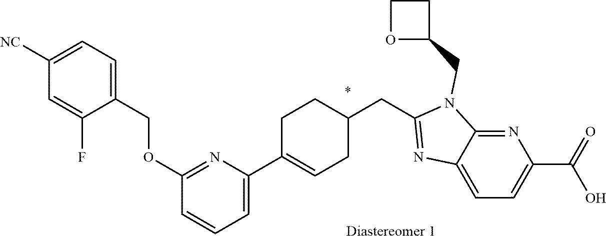 |
| 101c | 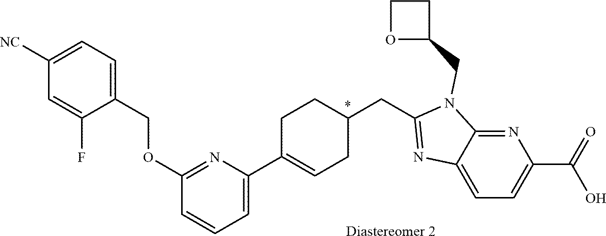 |
| 102a | 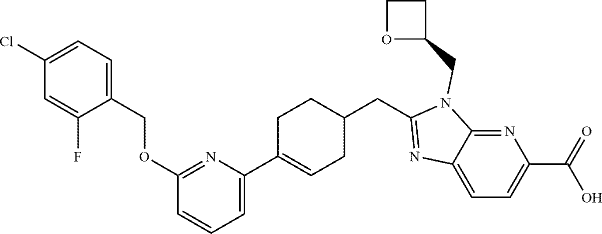 [SMILES: O=C(O)c1ccc2nc(CC3CC=C(c4cccc(OCc5ccc(Cl)cc5F)n4)CC3)n(C[C@@H]3CCO3)c2n1] |
| 102b | 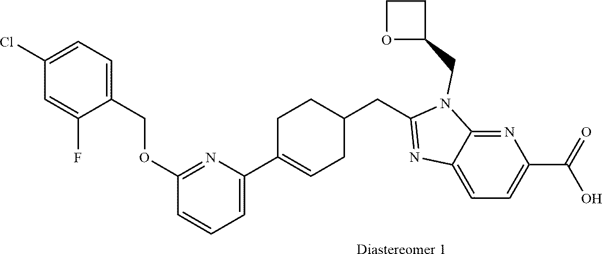 |
| 102c | 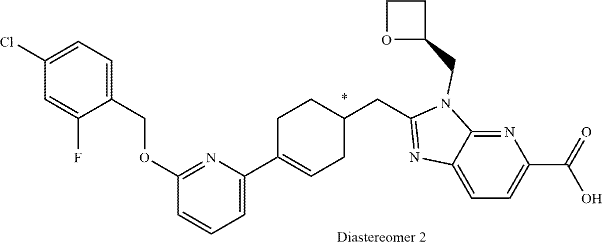 |
| 103a | 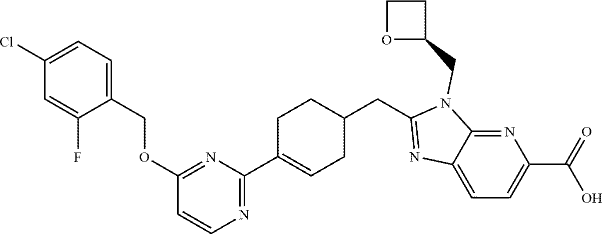 [SMILES: O=C(O)c1ccc2nc(CC3CC=C(c4nccc(OCc5ccc(Cl)cc5F)n4)CC3)n(C[C@@H]3CCO3)c2n1] |
| 104a | 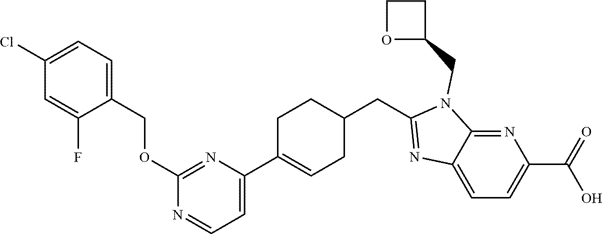 [SMILES: O=C(O)c1ccc2nc(CC3CC=C(c4ccnc(OCc5ccc(Cl)cc5F)n4)CC3)n(C[C@@H]3CCO3)c2n1] |
| 105a | 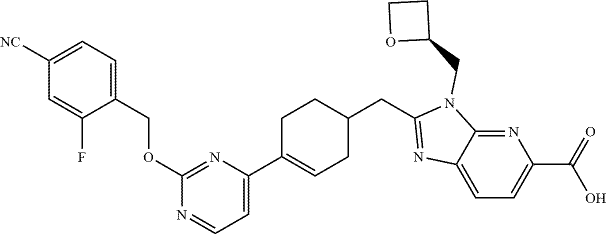 [SMILES: N#Cc1ccc(COc2nccc(C3=CCC(Cc4nc5ccc(C(=O)O)nc5n4C[C@@H]4CCO4)CC3)n2)c(F)c1] |
| 106a | 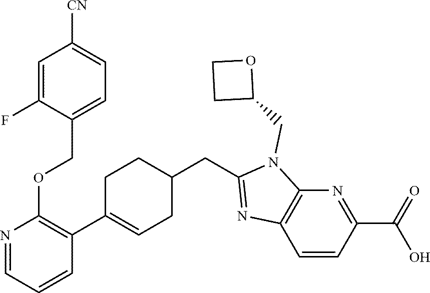 [SMILES: N#Cc1ccc(COc2ncccc2C2=CCC(Cc3nc4ccc(C(=O)O)nc4n3C[C@@H]3CCO3)CC2)c(F)c1] |
| 107a | 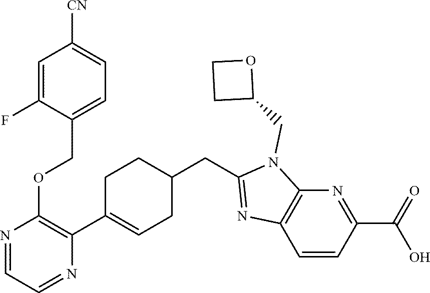 [SMILES: N#Cc1ccc(COc2nccnc2C2=CCC(Cc3nc4ccc(C(=O)O)nc4n3C[C@@H]3CCO3)CC2)c(F)c1] |
| 108a | 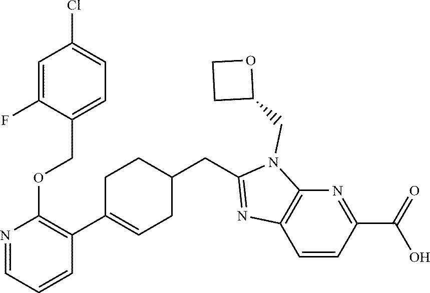 [SMILES: O=C(O)c1ccc2nc(CC3CC=C(c4cccnc4OCc4ccc(Cl)cc4F)CC3)n(C[C@@H]3CCO3)c2n1] |
| 109a | 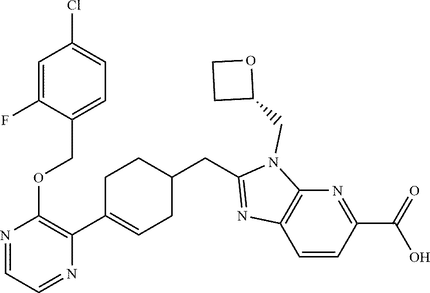 [SMILES: O=C(O)c1ccc2nc(CC3CC=C(c4nccnc4OCc4ccc(Cl)cc4F)CC3)n(C[C@@H]3CCO3)c2n1] |
| 110a | 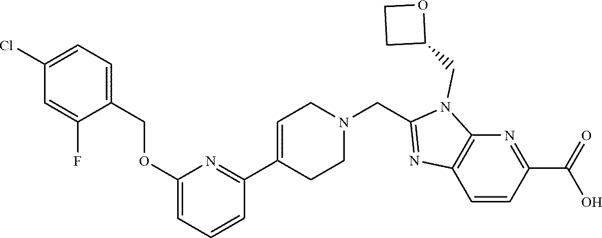 [SMILES: O=C(O)c1ccc2nc(CN3CC=C(c4cccc(OCc5ccc(Cl)cc5F)n4)CC3)n(C[C@@H]3CCO3)c2n1] |
| 111a | 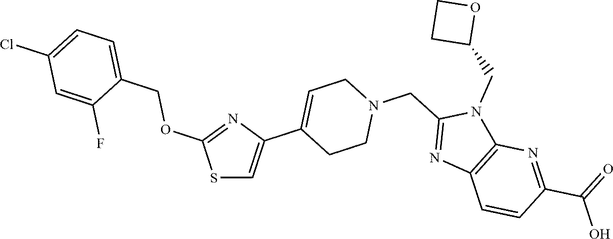 [SMILES: O=C(O)c1ccc2nc(CN3CC=C(c4csc(OCc5ccc(Cl)cc5F)n4)CC3)n(C[C@@H]3CCO3)c2n1] |
| 112a | 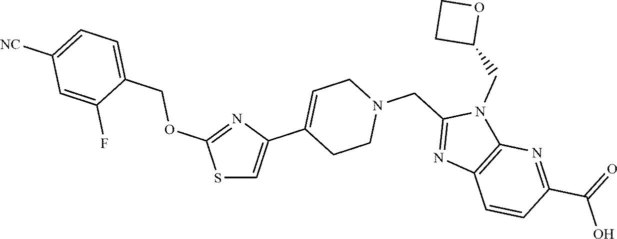 [SMILES: N#Cc1ccc(COc2nc(C3=CCN(Cc4nc5ccc(C(=O)O)nc5n4C[C@@H]4CCO4)CC3)cs2)c(F)c1] |
| 113a | 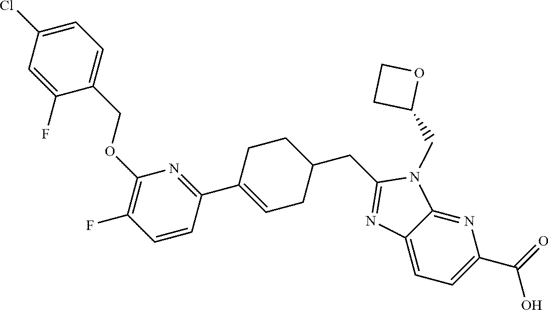 [SMILES: O=C(O)c1ccc2nc(CC3CC=C(c4ccc(F)c(OCc5ccc(Cl)cc5F)n4)CC3)n(C[C@@H]3CCO3)c2n1] |
| 114a | 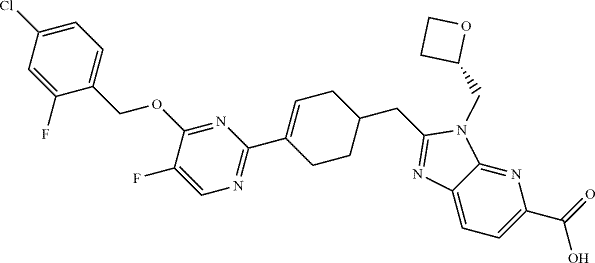 [SMILES: O=C(O)c1ccc2nc(CC3CC=C(c4ncc(F)c(OCc5ccc(Cl)cc5F)n4)CC3)n(C[C@@H]3CCO3)c2n1] |
| 114b | 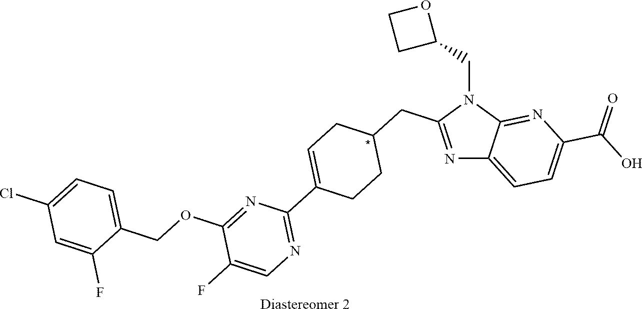 |
| 114c | 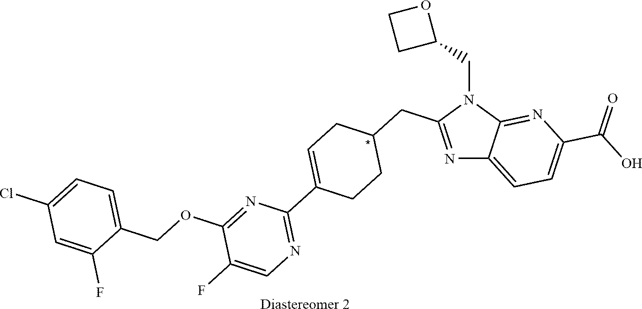 |
| 115a | 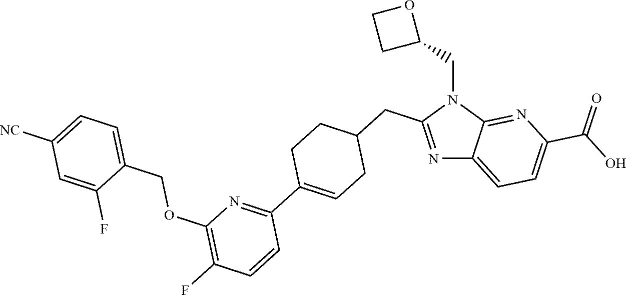 |
| 116a | 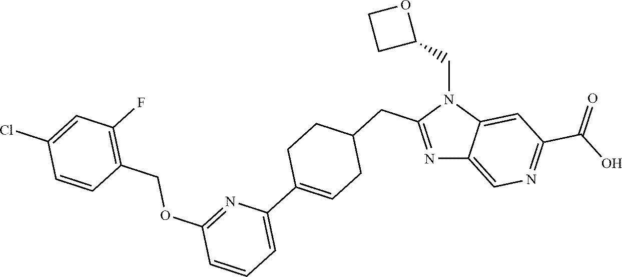 [SMILES: O=C(O)c1cc2c(cn1)nc(CC1CC=C(c3cccc(OCc4ccc(Cl)cc4F)n3)CC1)n2C[C@@H]1CCO1] |
| 117a | 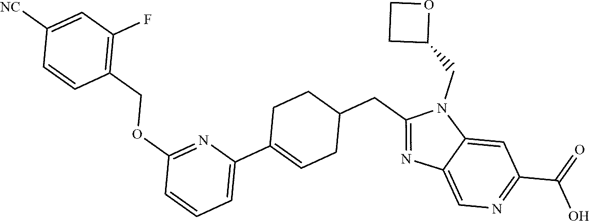 |
| 118a | 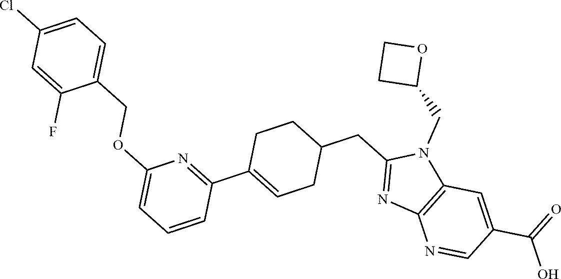 [SMILES: O=C(O)c1cnc2nc(CC3CC=C(c4cccc(OCc5ccc(Cl)cc5F)n4)CC3)n(C[C@@H]3CCO3)c2c1] |
| 119a | 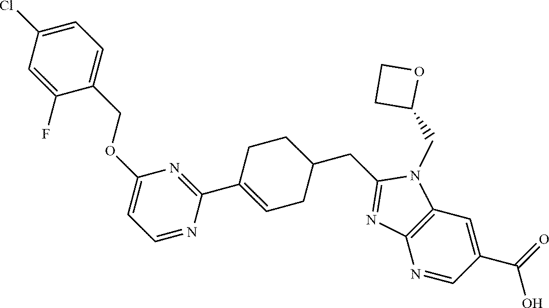 [SMILES: O=C(O)c1cnc2nc(CC3CC=C(c4nccc(OCc5ccc(Cl)cc5F)n4)CC3)n(C[C@@H]3CCO3)c2c1] |
| 120a | 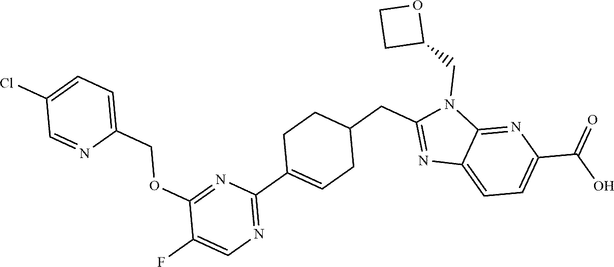 [SMILES: O=C(O)c1ccc2nc(CC3CC=C(c4ncc(F)c(OCc5ccc(Cl)cn5)n4)CC3)n(C[C@@H]3CCO3)c2n1] |
| 121a | 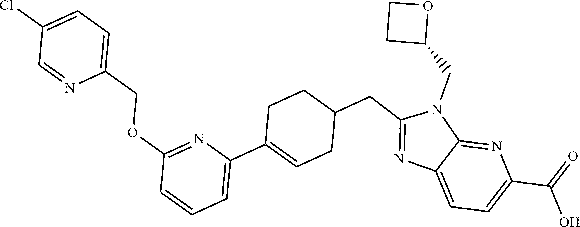 [SMILES: O=C(O)c1ccc2nc(CC3CC=C(c4cccc(OCc5ccc(Cl)cn5)n4)CC3)n(C[C@@H]3CCO3)c2n1] |
| 122 | 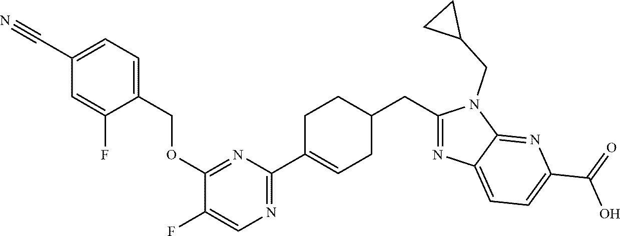 [SMILES: N#Cc1ccc(COc2nc(C3=CCC(Cc4nc5ccc(C(=O)O)nc5n4CC4CC4)CC3)ncc2F)c(F)c1] |
| 123 | 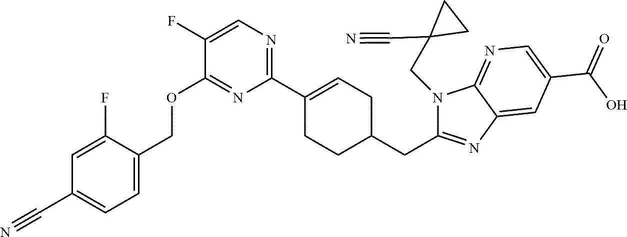 [SMILES: N#Cc1ccc(COc2nc(C3=CCC(Cc4nc5cc(C(=O)O)cnc5n4CC4(C#N)CC4)CC3)ncc2F)c(F)c1] |
| 124 | 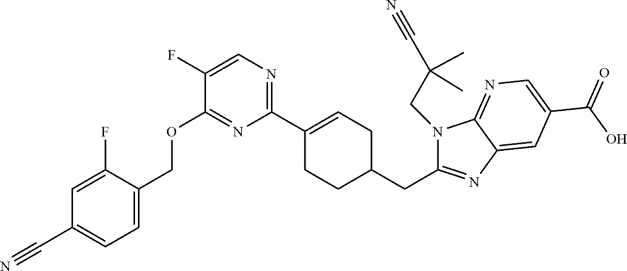 [SMILES: CC(C)(C#N)Cn1c(CC2CC=C(c3ncc(F)c(OCc4ccc(C#N)cc4F)n3)CC2)nc2cc(C(=O)O)cnc21] |
| 125 | 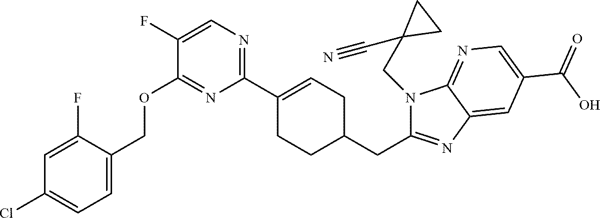 [SMILES: N#CC1(Cn2c(CC3CC=C(c4ncc(F)c(OCc5ccc(Cl)cc5F)n4)CC3)nc3cc(C(=O)O)cnc32)CC1] |
| 126 | 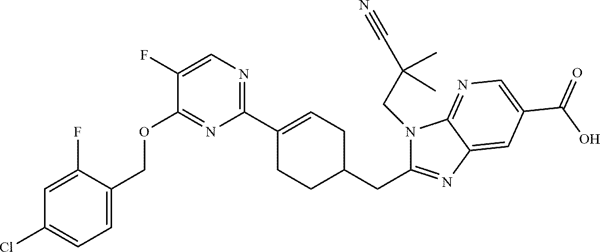 [SMILES: CC(C)(C#N)Cn1c(CC2CC=C(c3ncc(F)c(OCc4ccc(Cl)cc4F)n3)CC2)nc2cc(C(=O)O)cnc21] |
| 127 | 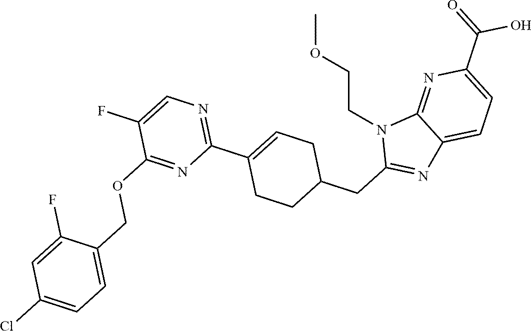 [SMILES: COCCn1c(CC2CC=C(c3ncc(F)c(OCc4ccc(Cl)cc4F)n3)CC2)nc2ccc(C(=O)O)nc21] |
| 128 | 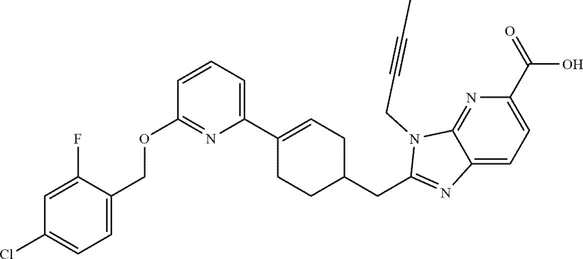 [SMILES: CC#CCn1c(CC2CC=C(c3cccc(OCc4ccc(Cl)cc4F)n3)CC2)nc2ccc(C(=O)O)nc21] |

In another aspect, provided herein are compounds of Formula II:

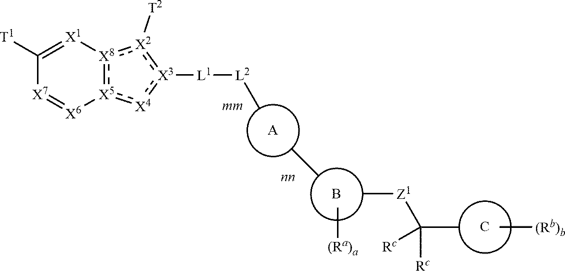

or a pharmaceutically acceptable salt or solvate thereof, wherein:

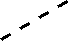

indicates an optional single or double bond, as allowed by valence;
each of X¹, X², X³, X⁴, X⁵, X⁶, X⁷, and X⁸ is independently selected from the group consisting of C, CH, and N, provided that at least two and no more than four of X¹, X², X³, X⁴, X⁵, X⁶, X⁷, and X⁸ are N;
T¹ is C(═O)OH or a carboxylic acid bioisostere;
T² is a (C₁-C₆)alkyl optionally substituted with (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{x};
each R^{x} is independently selected from the group consisting of OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino;
L¹ is (C₁-C₃)alkylene, which is optionally substituted with 1-3 R^{L};
L² is a bond, —O—, -S(O)₀₋₂-, or —NH—;
each R^{L} is independently selected from the group consisting of: halogen, (C₁-C₃)alkyl, and (C₁-C₃)haloalkyl; or
a pair of R^{L} on the same or on adjacent carbon atoms, taken together with the atom(s) to which each is attached, forms a (C₃-C₆)cycloalkyl ring;

Ring A is selected from the group consisting of:
phenylene optionally substituted with 1-4 R^{Y};
5- to 6-membered heteroarylene optionally substituted with 1-3 R^{Y};
wherein mm represents point of attachment to L², and nn represents point of attachment to Ring B; and
each R^{Y} is independently selected from the group consisting of halogen, cyano, —O, oxo, (C₁-C₃)alkyl, (C₁-C₃)haloalkyl, (C₁-C₃)alkoxy, and (C₁-C₃)haloalkoxy;

Ring B is selected from the group consisting of:

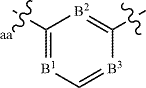

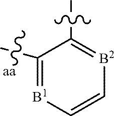

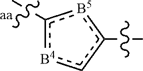

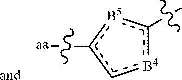

wherein aa represents the point of attachment to Ring A;
each of B¹, B², and B³ is independently selected from the group consisting of CR¹ and N;
each of B⁴ and B⁵ is independently selected from the group consisting of N, NR¹, C, CR¹, O, and S, provided that the ring containing B⁴ and B⁵ is heteroaryl;
R¹ is selected from the group consisting of H, halogen, and (C₁-C₆)alkyl;
each R^{a} is independently selected from the group consisting of (C₁-C₆)alkyl, (C₁-C₃)alkyl(C₃-C₆)cycloalkyl, (C₁-C₃)alkyl(3- to 5-membered heterocycloalkyl), —C(O)NR²R³, and (C₁-C₆)fluoroalkyl;
each R² and R³ is independently selected from the group consisting of H and (C₁-C₆)alkyl;
a is an integer selected from 0-3;
Z¹ is —O— or —NH—;
each R^{c} is independently selected from the group consisting of H, (C₁-C₆)alkyl, and (C₁-C₃)haloalkyl;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)alkoxy, halogen, (C₃-C₆)cycloalkyl, and CN; and
b is an integer selected from 0-3.

Embodiments of Formula II can include any one or more of the features delineated below and/or in the claims.

In some embodiments, X⁸ is C; and X⁵ is C.

In some embodiments, X³ is C.

In some embodiments, X² is N.

In some embodiments, X⁴ is N.

In some embodiments, X³ is C; X² is N; and X⁴ is N.

In some embodiments, X⁷ is CH.

In some embodiments, X⁸ is C; X⁵ is C; and X⁷ is CH.

In some embodiments, X⁸, X⁵, and X³ are C; X² and X⁴ are N; X⁷ is CH; and X¹ and X⁶ are independently CH or N. For example, X¹ and X⁶ are CH. As another non-limiting example, X¹ is N; and X⁶ is CH. As yet another non-limiting example, X¹ is CH; and X⁶ is N.

In some embodiments, X⁸, X⁵, and X³ are C; X⁷ and X⁶ are CH; X¹ is N; and X² and X⁴ is N.

In some embodiments, X⁸, X⁵, and X³ are C; X⁷, X⁶, and X¹ are CH; and X² and X⁴ is N.

In some embodiments, the

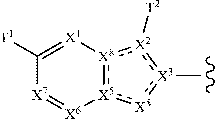

moiety has the formula:

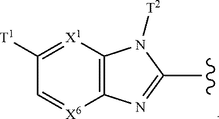

In some embodiments, the

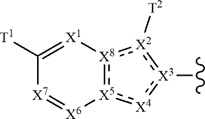

moiety has the formula:

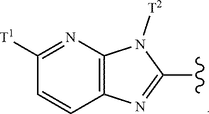

In some embodiments, the

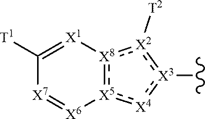

moiety has the formula:

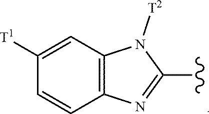

In some embodiments, T¹ is C(═O)OH.

In some embodiments, T¹ is a carboxylic acid bioisostere.

In some embodiments (when T¹ is a carboxylic acid bioisostere) , T¹ is a 5-membered heteroaryl including from 2-4 heteroatoms each independently selected from the group consisting of N, O, and S, wherein the heteroaryl is optionally substituted with from 1-4 substituents each independently selected from the group consisting of hydroxy, (C₁-C₆)alkyl, (C₁-C₆)haloalkyl, and halogen.

In some embodiments, T¹ is tetrazolyl, which is optionally substituted with from 1-2 substituents each independently selected from the group consisting of hydroxy, (C₁-C₆)alkyl, (C₁-C₆)haloalkyl, and halogen. For example, T¹ is selected from the group consisting of:

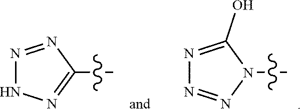

In some embodiments, T¹ is triazolyl or oxadiazolyl, which is optionally substituted with from 1-2 substituents each independently selected from (C₁-C₆)alkyl and hydroxy. For example, T¹ is

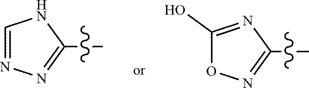

In some embodiments, T¹ is a ring (e.g., a 4-6 membered ring, e.g., a 5-membered ring) including from 0-3 heteroatoms each independently selected from the group consisting of N, O, and S, wherein the ring is substituted with from 1-2 oxo and further optionally substituted from 1-2 substituent each independently selected from the group consisting of hydroxy, (C₁-C₆)alkyl, (C₁-C₆)haloalkyl, and halogen. For example, T¹ is

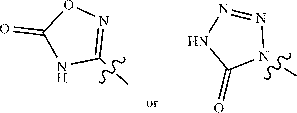

In some embodiments, T¹ is (C₁-C₆)alkyl which is substituted with from 1-3 hydroxy and further optionally substituted with from 1-10 fluoro. In certain of these embodiments, T¹ is (C₁-C₆)alkyl which is substituted with from 1-3 hydroxy and further substituted with from 1-10 fluoro. For example, T¹ is

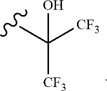

In some embodiments, T¹ is C(=O)NHS(O)₂(C₁-C₄)alkyl. For example, T¹ is C(=O)NHS(O)₂Me.

In some embodiments, T¹ is selected from the group consisting of the following:

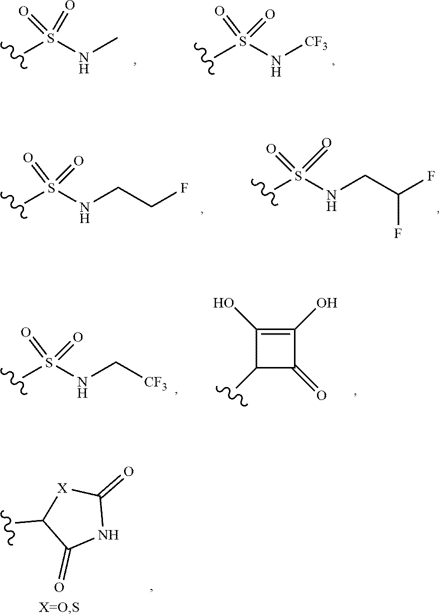

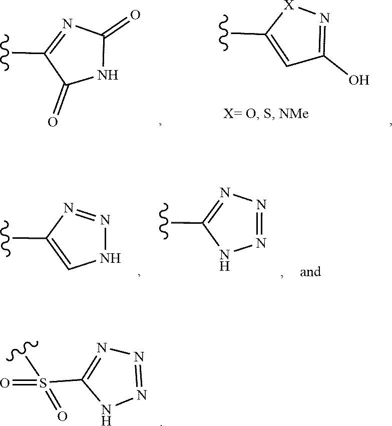

In some embodiments, T² is (C₁-C₃)alkyl which is substituted with (C₃-C₆)cycloalkyl, 3- to 6-membered heterocycloalkyl, phenyl, or 5- to 6-membered heteroaryl.

In some embodiments, T² is (C₁-C₃)alkyl which is substituted with (C₃-C₆)cycloalkyl or 3- to 6-membered heterocycloalkyl.

In some embodiments, T² is (C₁-C₃)alkyl which is substituted with 3- to 6-membered heterocycloalkyl.

In some embodiments, T² is (C₁-C₃)alkyl which is substituted with 4- to 6-membered heterocycloalkyl.

In some embodiments, T² is (C₁-C₃)alkyl which is substituted with oxetanyl.

In some embodiments, T² is

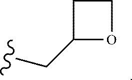

In some embodiments, T² is

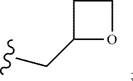

and the stereogenic center of T² has (S)-configuration. As a non-limiting example, the

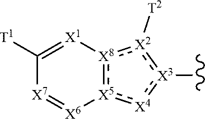

moiety can be:

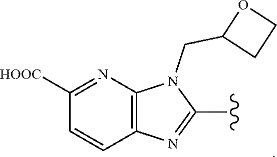

As another non-limiting example, the

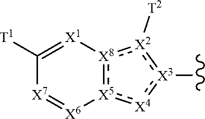

moiety can be:

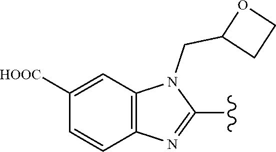

In some embodiments, L² is a bond. In some embodiments, L² is —O—.

In some embodiments, L¹ is (C₁-C₂)alkylene, which is optionally substituted with 1-3 R^{L}. In some embodiments, L¹ is CH₂. In some embodiments, L¹ is CH₂CH₂. In some embodiments, L¹ is CH₂CH₂, which is substituted with 1-3 R^{L}. In some embodiments, L¹ is CH₂CH₂, which is substituted with two R^{L}, wherein the pair of R^{L} on adjacent carbon atoms, taken together with the atoms to which each is attached, forms a C₃-C₅ cycloalkyl ring.

In some embodiments, L² is a bond; and L¹ is (C₁--C₃) (e.g., C₁, C₂, or C₃) alkylene, which is optionally substituted with 1-3 R^{L}. In some embodiments, L² is a bond; and L¹ is CH₂. In some embodiments, L² is a bond; and L¹ is CH₂CH₂. In some embodiments, L² is a bond; and L¹ is

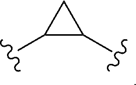

In some embodiments, L² is -0-; and L¹ is (C₁-C₂) alkylene, which is optionally substituted with 1-3 R^{L}. As a non-limiting example, L² is —O—; and L¹ is CH₂.

In some embodiments, mm is para to nn. In some embodiments, mm is meta to nn.

In some embodiments, L² is a bond; L¹ is CH₂; and mm is para to nn.

In some embodiments, L² is a bond; L¹ is CH₂CH₂ or

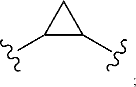

and mm is meta to nn.

In some embodiments, L² is —O—; L¹ is CH₂; and mm is meta to nn.

In some embodiments, Ring A is phenylene optionally substituted with 1-4 R^{Y}.

In some embodiments, Ring A is 1,4-phenylene or 1,3-phenylene optionally substituted with 1-2 R^{Y}.

In some embodiments, Ring A is 1,4-phenylene optionally substituted with 1-2 R^{Y}. As a non-limiting example, Ring A can be

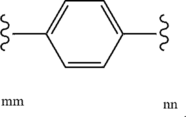

In some embodiments, Ring A is 5- to 6-membered heteroarylene optionally substituted with 1-3 R^{Y}. In some embodiments, Ring A is 6-membered heteroarylene optionally substituted with 1-3 R^{Y}.

In some embodiments, Ring A is 2,4-pyridinylene or 3,5-pyridinylene optionally substituted with 1-2 R^{Y}. In some embodiments, Ring A is 2,4-pyridinylene optionally substituted with 1-2 R^{Y}. As a non-limiting example, Ring A can be selected from the group consisting of:

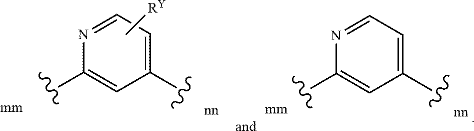

In some embodiments, Ring A is 6-membered heteroarylene substituted with 1-3 R^{Y}, provided that at least one R^{Y} is oxo. In some embodiments, Ring A is pyridonylene which is further optionally substituted with 1-2 R^{Y}. In some embodiments, Ring A is 1,4-pyridonylene which is further optionally substituted with 1-2 R^{Y}. As a non-limiting example, Ring A can be selected from the group consisting of:

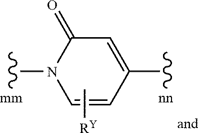

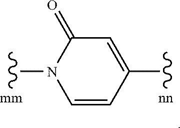

In some embodiments, Ring A is 5-membered heteroarylene optionally substituted with 1-2 R^{Y}. In some embodiments, Ring A is pyrazolylene optionally substituted with 1-2 R^{Y}. In some embodiments, Ring A is selected from the group consisting of:

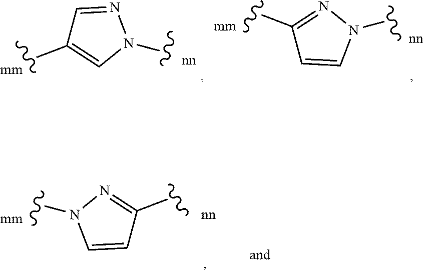

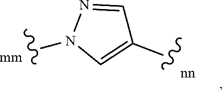

each of which is optionally substituted with one R^{Y}.

In some embodiments, each R^{Y} is independently selected from the group consisting of: halogen and (C₁-C₃)alkyl.

In some embodiments, Ring B is

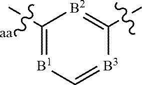

In some embodiments of (B—I), B² is N.

In some embodiments of (B—I), B¹ and B³ are independently CR¹. For example, B² can be N; and B¹ and B³ can be independently selected CR¹.

In some embodiments of (B—I), one of B¹ and B³ is N; and the other one of B¹ and B³ is CR¹. In some embodiments of (B—I), B¹ is N; and B³ is CR¹. In some embodiments of (B—I), B¹ is CR¹; and B³ is N.

In some embodiments of (B—I), B² is N; B¹ is N; and B³ is CR¹. In some embodiments of (B—I), B² is N; B¹ is CR¹; and B³ is N.

In some embodiments of (B—I), B² is CR¹.

In some embodiments of (B—I), B¹ and B³ are independently CR¹.

In some embodiments of (B—I), B² is CR¹; and B¹ and B³ are independently selected CR¹.

In some embodiments, Ring B is

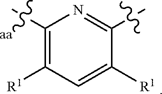

In some embodiments, Ring B is

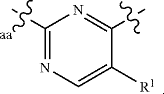

In some embodiments, Ring B is

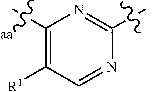

In some embodiments, Ring B is

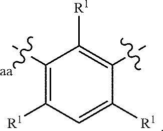

In some embodiments, Ring B is

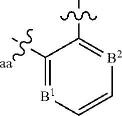

In some embodiments of (B—II), B² is CR¹.

In some embodiments of (B—II), B² is N.

In some embodiments of (B—II), B¹ is N.

In some embodiments of (B—II), B¹ is CR¹.

In some embodiments of (B—II), B¹ and B² are N.

In some embodiments, Ring B is

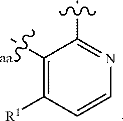

In some embodiments, Ring B is

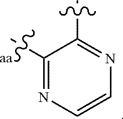

In some embodiments, each R¹ is independently H or halogen.

In some embodiments, each R¹ is H.

In some embodiments, a is 0.

In some embodiments, Z¹ is —O—.

In some embodiments, Z¹ is —NH—.

In some embodiments, each R^{c} is H.

In some embodiments, each R^{c} is an independently selected (C₁-C₆)alkyl or (C₁-C₃)haloalkyl.

In some embodiments, Z¹ is O; and each R^{c} is H.

In some embodiments, Ring C is selected from the group consisting of: phenyl, 5- to 6-membered heteroaryl, and 5- to 10-membered bicycloheteroaryl.

In some embodiments, Ring C is phenyl.

In some embodiments, b is 1-3. For example, b can be 2.

In some embodiments, b is 0.

In some embodiments, Ring C is phenyl; and b is 2.

In some embodiments, the

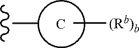

moiety is

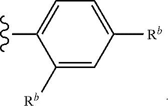

In some embodiments, Ring C is phenyl; and b is 0.

In some embodiments, each occurrence of R^{b} is independently selected from the group consisting of: (C₁-C₆)alkyl, (C₁-C₆)alkoxy, halogen, and CN.

In some embodiments, each occurrence of R^{b} is independently selected from the group consisting of —F, —Cl, and CN. As non-limiting examples, the

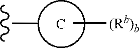

moiety can be

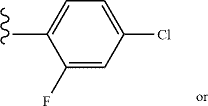

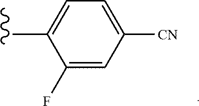

In some embodiments, the compound of Formula II is a compound of Formula IIA:

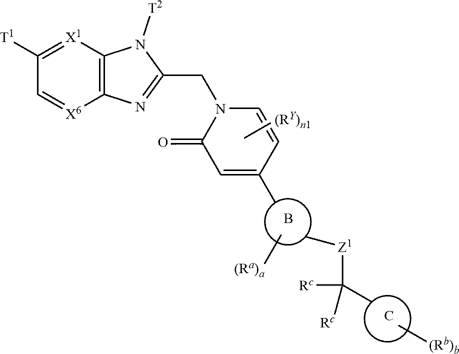

or a pharmaceutically acceptable salt or solvate thereof, wherein n1 is 0 or 1.

In some embodiments of Formula IIA, n1 is 0.

In some embodiments of Formula IIA, n1 is 1. For example, the

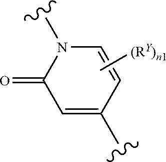

moiety can be:

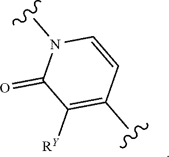

As another non-limiting example, the

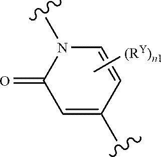

moiety can be:

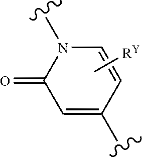

In some embodiments, the compound of Formula II is a compound of Formula IIB:

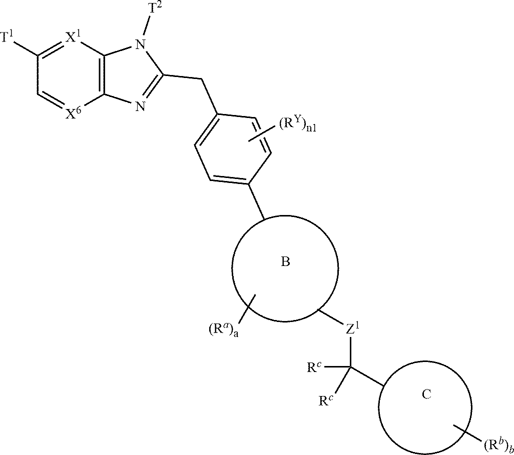

or a pharmaceutically acceptable salt or solvate thereof, wherein n1 is 0 or 1.

In some embodiments of Formula IIB, n1 is 0.

In some embodiments of Formula IIB, n1 is 1.

In some embodiments, the compound of Formula II is a compound of Formula IIC:

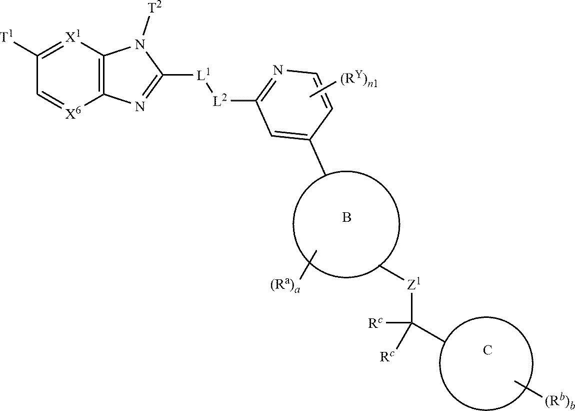

or a pharmaceutically acceptable salt or solvate thereof, wherein n1 is 0 or 1.

In some embodiments of Formula IIC, L¹ is CH₂; and L² is —O—.

In some embodiments of Formula IIC, L¹ is CH₂CH₂; and L² is a bond.

In some embodiments of Formula IIC, L¹ is

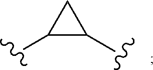

and L² is a bond.

In some embodiments of Formula IIC, n1 is 0.

In some embodiments of Formula IIC, n1 is 1. For example, the

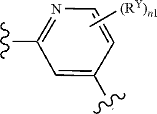

moiety can be:

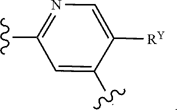

In some embodiments of Formulae IIA, IIB, or IIC, X¹ is N.

In some embodiments of Formulae IIA, IIB, or IIC, X¹ is CH.

In some embodiments of Formulae IIA, IIB, or IIC, X⁶ is CH.

In some embodiments of Formulae IIA, IIB, or IIC, X¹ is N; and X⁶ is CH.

In some embodiments of Formulae IIA, IIB, or IIC, X¹ and X⁶ are independently selected CH.

In some embodiments of Formulae IIA, IIB, or IIC, T¹ is C(═O)OH.

In some embodiments of Formulae IIA, IIB, or IIC, T² is (C₁-C₃)alkyl which is substituted with 3- to 6-membered heterocycloalkyl. In some embodiments of Formulae IIA, IIB, or IIC, T² is (C₁-C₃)alkyl which is substituted with oxetanyl. In some embodiments of Formulae IIA, IIB, or IIC, T² is is

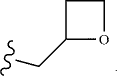

In some embodiments of Formulae IIA, IIB, or IIC, R^{Y} when present is independently selected from the group consisting of: halogen and (C₁-C₃)alkyl.

In some embodiments of Formulae IIA, IIB, or IIC, R^{Y} when present is selected from the group consisting of: —F and methyl.

In some embodiments of Formulae IIA, IIB, or IIC, Ring B is

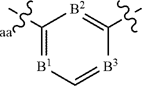

In some embodiments of Formulae IIA, IIB, or IIC, Ring B is

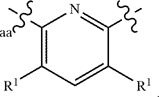

In some embodiments of Formulae IIA, IIB, or IIC, Ring B is selected from the group consisting of:

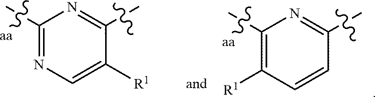

In some embodiments of Formulae IIA, IIB, or IIC, Ring B is

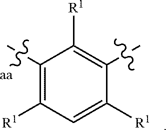

In some embodiments of Formulae IIA, IIB, or IIC, Ring B is

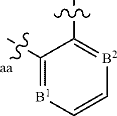

In some embodiments of Formulae IIA, IIB, or IIC, Ring B is

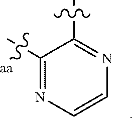

In some embodiments of Formulae IIA, IIB, or IIC, each R¹ is independently H or halogen. In some embodiments of Formulae IIA, IIB, or IIC, each R¹ is H.

In some embodiments of Formulae IIA, IIB, or IIC, a is 0.

In some embodiments of Formulae IIA, IIB, or IIC, Z¹ is —O—.

In some embodiments of Formulae IIA, IIB, or IIC, each R^{c} is H.

In some embodiments of Formulae IIA, IIB, or IIC, Ring C is phenyl.

In some embodiments of Formulae IIA, IIB, or IIC, b is 1-3.

In some embodiments of Formulae IIA, IIB, or IIC, b is 2.

In some embodiments of Formulae IIA, IIB, or IIC, b is 0.

In some embodiments of Formulae IIA, IIB, or IIC, Ring C is phenyl; and b is 2.

In some embodiments of Formulae IIA, IIB, or IIC,

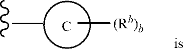

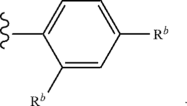

In some embodiments of Formulae IIA, IIB, or IIC, Ring C is phenyl and b is 0.

In some embodiments of Formulae IIA, IIB, or IIC, each occurrence of R^{b} is independently selected from the group consisting of: (C₁-C₆)alkyl, (C₁-C₆)alkoxy, halogen, and CN. In some embodiments of Formulae IIA, IIB, or IIC, each occurrence of R^{b} is independently selected from the group consisting of —F, —Cl, and CN.

In some embodiments, a compound of Formula II is selected from the group consisting of the compounds in Table C1—W or a pharmaceutically acceptable salt or solvate thereof.

TABLE C1-W

| | |
|---|---|
| Compound # | Structure |
| 101w | 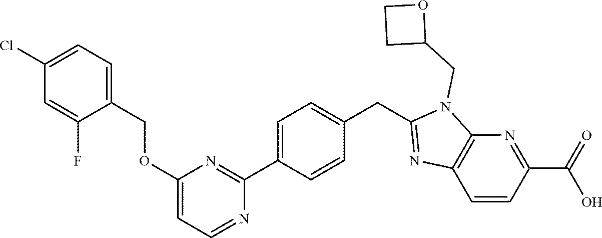 [SMILES: O=C(O)c1ccc2nc(Cc3ccc(-c4nccc(OCc5ccc(Cl)cc5F)n4)cc3)n(CC3CCO3)c2n1] |
| 102w | 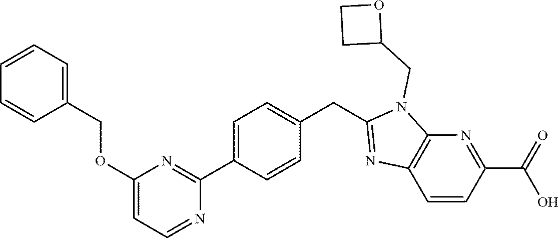 [SMILES: O=C(O)c1ccc2nc(Cc3ccc(-c4nccc(OCc5ccccc5)n4)cc3)n(CC3CCO3)c2n1] |
| 103w | 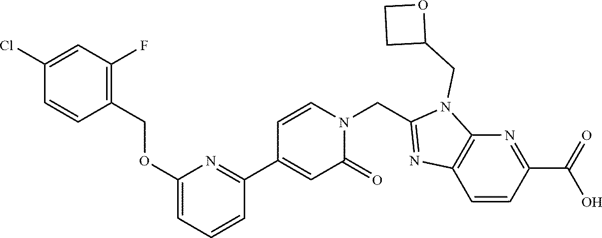 [SMILES: O=C(O)c1ccc2nc(Cn3ccc(-c4cccc(OCc5ccc(Cl)cc5F)n4)cc3=O)n(CC3CCO3)c2n1] |
| 104w | 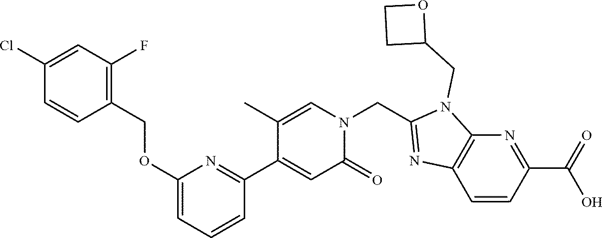 [SMILES: Cc1cn(Cc2nc3ccc(C(=O)O)nc3n2CC2CCO2)c(=O)cc1-c1cccc(OCc2ccc(Cl)cc2F)n1] |
| 105w | 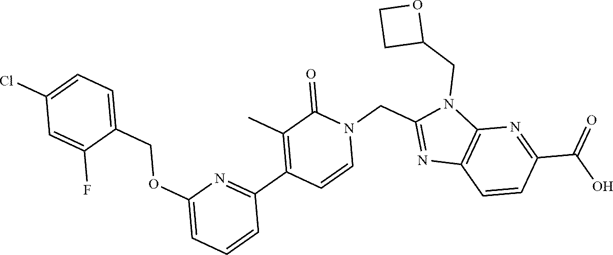 [SMILES: Cc1c(-c2cccc(OCc3ccc(Cl)cc3F)n2)ccn(Cc2nc3ccc(C(=O)O)nc3n2CC2CCO2)c1=O] |
| 106w | 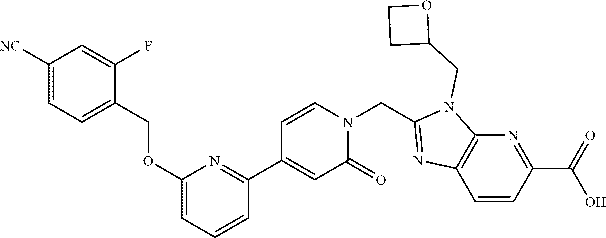 [SMILES: N#Cc1ccc(COc2cccc(-c3ccn(Cc4nc5ccc(C(=O)O)nc5n4CC4CCO4)c(=O)c3)n2)c(F)c1] |
| 107w | 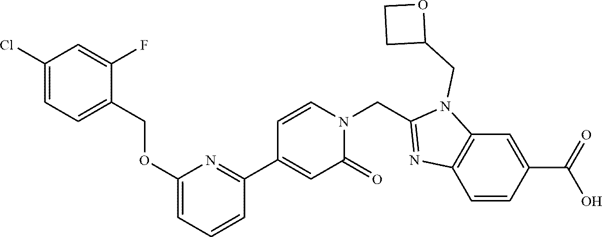 [SMILES: O=C(O)c1ccc2nc(Cn3ccc(-c4cccc(OCc5ccc(Cl)cc5F)n4)cc3=O)n(CC3CCO3)c2c1] |
| 108w | 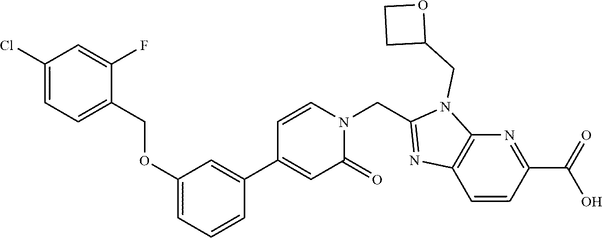 [SMILES: O=C(O)c1ccc2nc(Cn3ccc(-c4cccc(OCc5ccc(Cl)cc5F)c4)cc3=O)n(CC3CCO3)c2n1] |
| 109w | 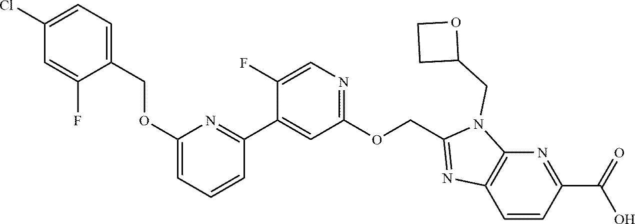 [SMILES: O=C(O)c1ccc2nc(COc3cc(-c4cccc(OCc5ccc(Cl)cc5F)n4)c(F)cn3)n(CC3CCO3)c2n1] |
| 110w | 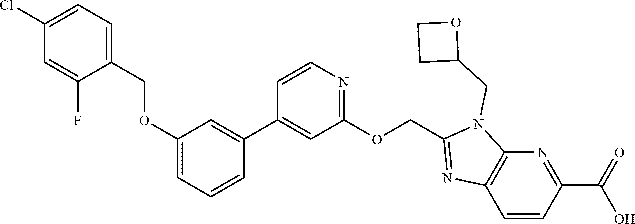 [SMILES: O=C(O)c1ccc2nc(COc3cc(-c4cccc(OCc5ccc(Cl)cc5F)c4)ccn3)n(CC3CCO3)c2n1] |
| 111w | 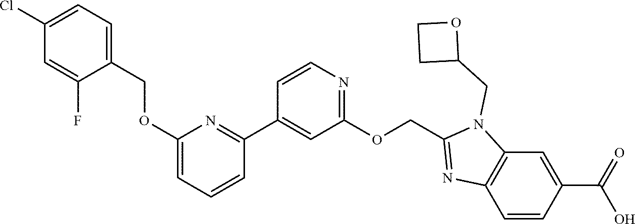 [SMILES: O=C(O)c1ccc2nc(COc3cc(-c4cccc(OCc5ccc(Cl)cc5F)n4)ccn3)n(CC3CCO3)c2c1] |
| 112w | 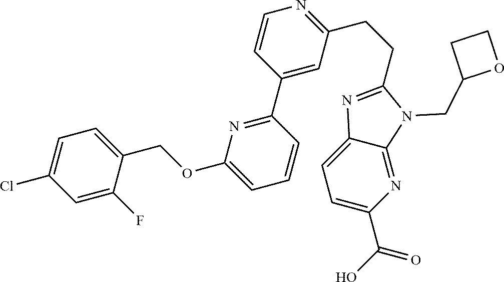 [SMILES: O=C(O)c1ccc2nc(CCc3cc(-c4cccc(OCc5ccc(Cl)cc5F)n4)ccn3)n(CC3CCO3)c2n1] |
| 113w | 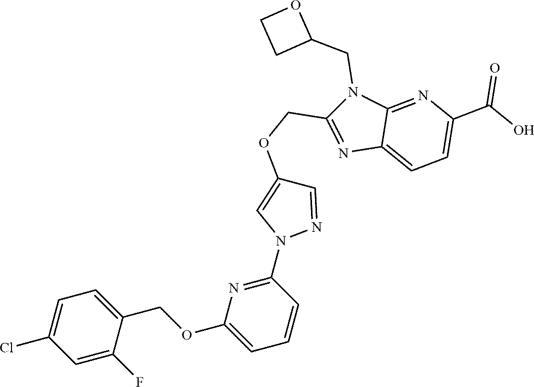 [SMILES: O=C(O)c1ccc2nc(COc3cnn(-c4cccc(OCc5ccc(Cl)cc5F)n4)c3)n(CC3CCO3)c2n1] |
| 114w | 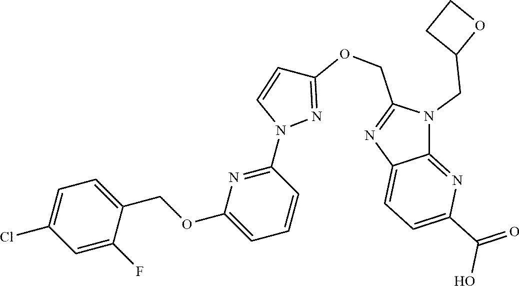 [SMILES: O=C(O)c1ccc2nc(COc3ccn(-c4cccc(OCc5ccc(Cl)cc5F)n4)n3)n(CC3CCO3)c2n1] |
| 115w | 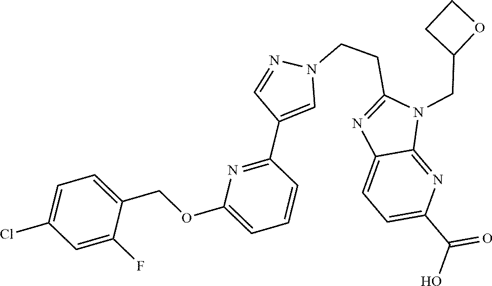 [SMILES: O=C(O)c1ccc2nc(CCn3cc(-c4cccc(OCc5ccc(Cl)cc5F)n4)cn3)n(CC3CCO3)c2n1] |
| 116w | 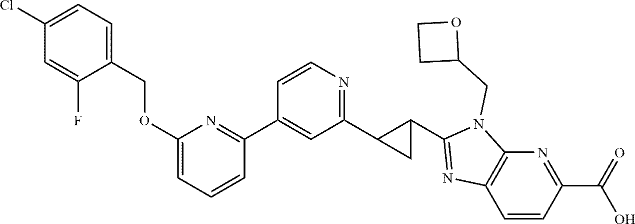 [SMILES: O=C(O)c1ccc2nc(C3CC3c3cc(-c4cccc(OCc5ccc(Cl)cc5F)n4)ccn3)n(CC3CCO3)c2n1] |
| 117w | 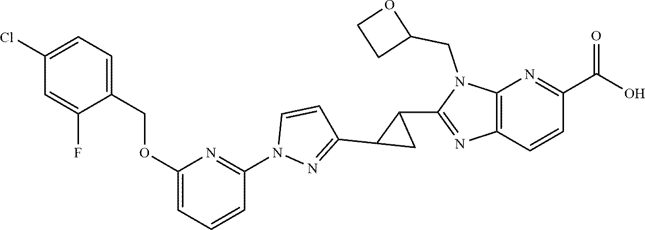 [SMILES: O=C(O)c1ccc2nc(C3CC3c3ccn(-c4cccc(OCc5ccc(Cl)cc5F)n4)n3)n(CC3CCO3)c2n1] |
| 118w | 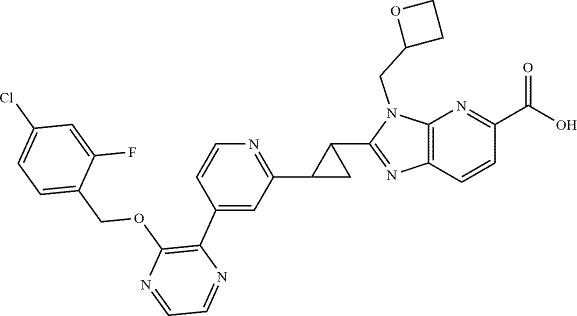 [SMILES: O=C(O)c1ccc2nc(C3CC3c3cc(-c4nccnc4OCc4ccc(Cl)cc4F)ccn3)n(CC3CCO3)c2n1] |
| 119w | 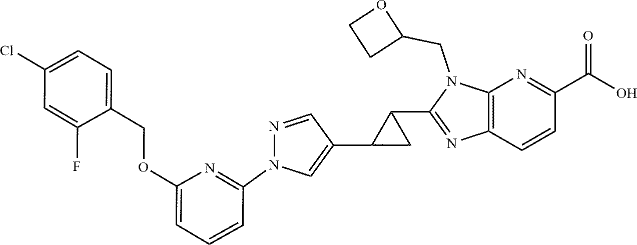 [SMILES: O=C(O)c1ccc2nc(C3CC3c3cnn(-c4cccc(OCc5ccc(Cl)cc5F)n4)c3)n(CC3CCO3)c2n1] |
| 120w | 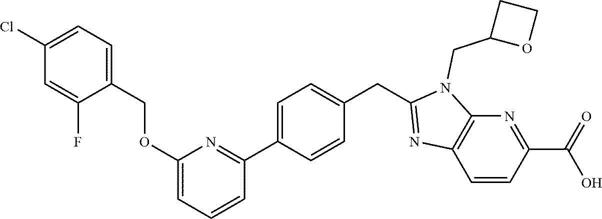 [SMILES: O=C(O)c1ccc2nc(Cc3ccc(-c4cccc(OCc5ccc(Cl)cc5F)n4)cc3)n(CC3CCO3)c2n1] |

In some embodiments, the compound is selected from the group consisting of the compounds in Table C2—W or a pharmaceutically acceptable salt or solvate thereof.

TABLE C2-W

| | |
|---|---|
| Compound # | Structure |
| 101aw | 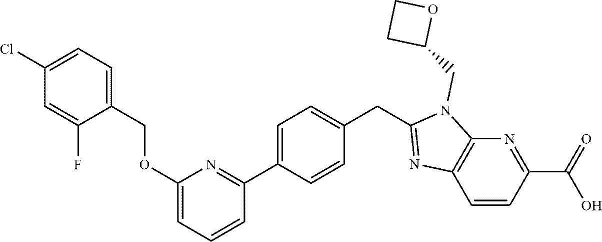 [SMILES: O=C(O)c1ccc2nc(Cc3ccc(-c4cccc(OCc5ccc(Cl)cc5F)n4)cc3)n(C[C@@H]3CCO3)c2n1] |
| 102aw | 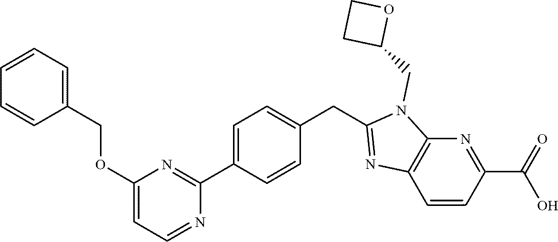 [SMILES: O=C(O)c1ccc2nc(Cc3ccc(-c4nccc(OCc5ccccc5)n4)cc3)n(C[C@@H]3CCO3)c2n1] |
| 103aw | 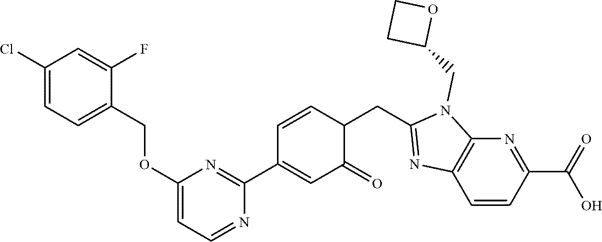 [SMILES: O=C(O)c1ccc2nc(CC3C=CC(c4nccc(OCc5ccc(Cl)cc5F)n4)=CC3=O)n(C[C@@H]3CCO3)c2n1] |
| 104aw | 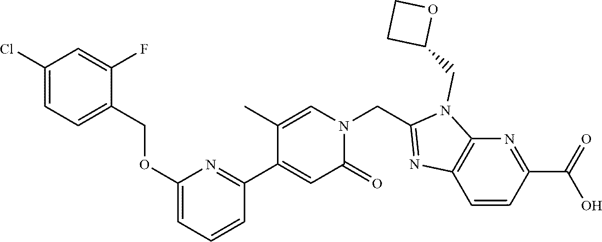 [SMILES: Cc1cn(Cc2nc3ccc(C(=O)O)nc3n2C[C@@H]2CCO2)c(=O)cc1-c1cccc(OCc2ccc(Cl)cc2F)n1] |
| 105aw | 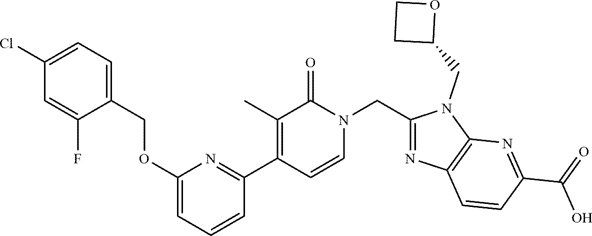 [SMILES: Cc1c(-c2cccc(OCc3ccc(Cl)cc3F)n2)ccn(Cc2nc3ccc(C(=O)O)nc3n2C[C@@H]2CCO2)c1=O] |
| 106aw | 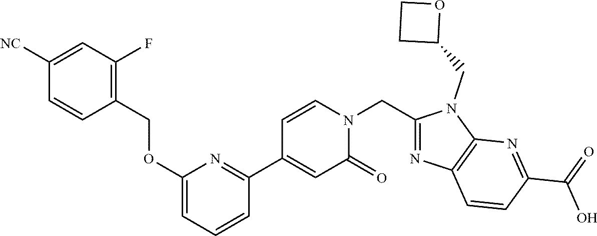 [SMILES: N#Cc1ccc(COc2cccc(-c3ccn(Cc4nc5ccc(C(=O)O)nc5n4C[C@@H]4CCO4)c(=O)c3)n2)c(F)c1] |
| 107aw | 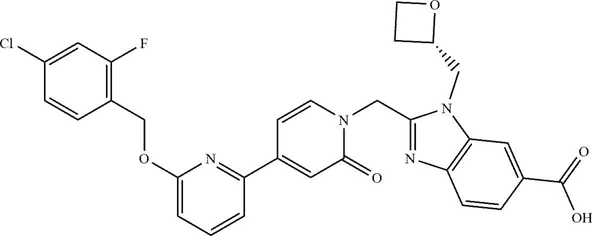 [SMILES: O=C(O)c1ccc2nc(Cn3ccc(-c4cccc(OCc5ccc(Cl)cc5F)n4)cc3=O)n(C[C@@H]3CCO3)c2c1] |
| 108aw | 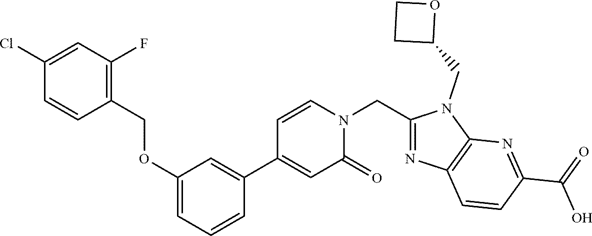 [SMILES: O=C(O)c1ccc2nc(Cn3ccc(-c4cccc(OCc5ccc(Cl)cc5F)c4)cc3=O)n(C[C@@H]3CCO3)c2n1] |
| 109aw | 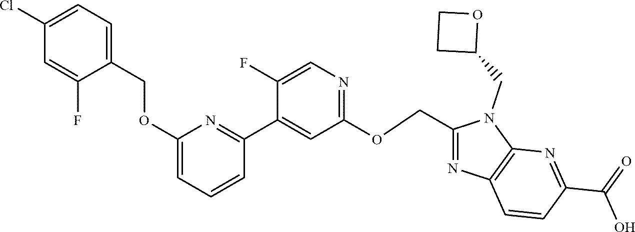 [SMILES: O=C(O)c1ccc2nc(COc3cc(-c4cccc(OCc5ccc(Cl)cc5F)n4)c(F)cn3)n(C[C@@H]3CCO3)c2n1] |
| 110aw | 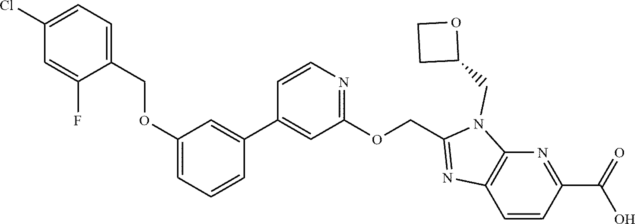 [SMILES: O=C(O)c1ccc2nc(COc3cc(-c4cccc(OCc5ccc(Cl)cc5F)c4)ccn3)n(C[C@@H]3CCO3)c2n1] |
| 111aw | 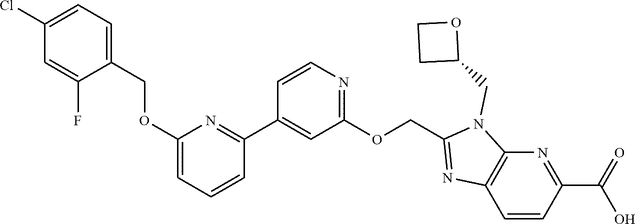 [SMILES: O=C(O)c1ccc2nc(COc3cc(-c4cccc(OCc5ccc(Cl)cc5F)n4)ccn3)n(C[C@@H]3CCO3)c2n1] |
| 112aw | 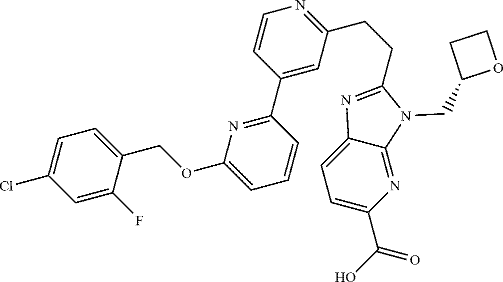 [SMILES: O=C(O)c1ccc2nc(CCc3cc(-c4cccc(OCc5ccc(Cl)cc5F)n4)ccn3)n(C[C@@H]3CCO3)c2n1] |
| 113aw | 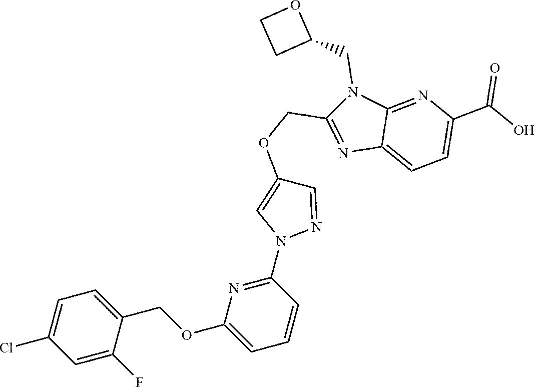 [SMILES: O=C(O)c1ccc2nc(COc3cnn(-c4cccc(OCc5ccc(Cl)cc5F)n4)c3)n(C[C@@H]3CCO3)c2n1] |
| 114aw | 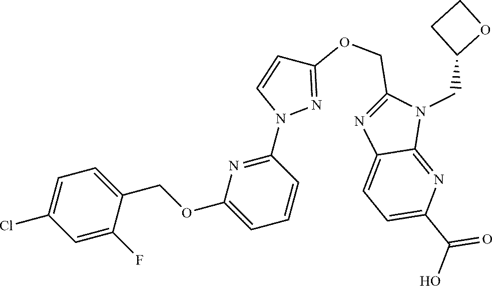 [SMILES: O=C(O)c1ccc2nc(COc3ccn(-c4cccc(OCc5ccc(Cl)cc5F)n4)n3)n(C[C@@H]3CCO3)c2n1] |
| 115aw | 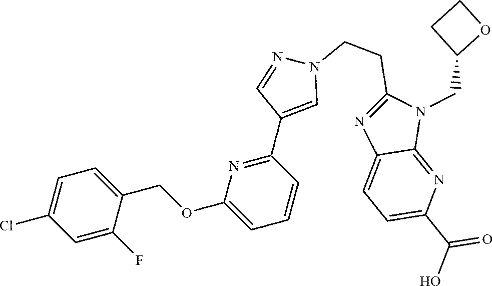 [SMILES: O=C(O)c1ccc2nc(CCn3cc(-c4cccc(OCc5ccc(Cl)cc5F)n4)cn3)n(C[C@@H]3CCO3)c2n1] |
| 116aw | 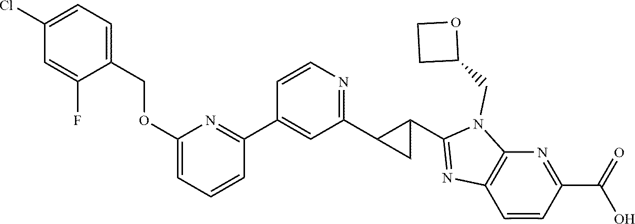 [SMILES: O=C(O)c1ccc2nc(C3CC3c3cc(-c4cccc(OCc5ccc(Cl)cc5F)n4)ccn3)n(C[C@@H]3CCO3)c2n1] |
| 117aw | 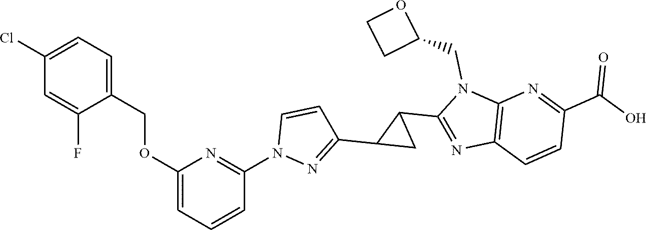 [SMILES: O=C(O)c1ccc2nc(C3CC3c3ccn(-c4cccc(OCc5ccc(Cl)cc5F)n4)n3)n(C[C@@H]3CCO3)c2n1] |
| 118aw | 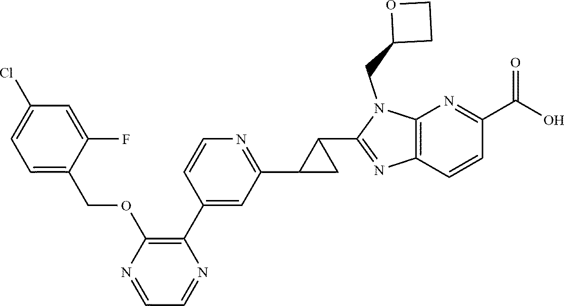 [SMILES: O=C(O)c1ccc2nc(C3CC3c3cc(-c4nccnc4OCc4ccc(Cl)cc4F)ccn3)n(C[C@@H]3CCO3)c2n1] |
| 119aw | 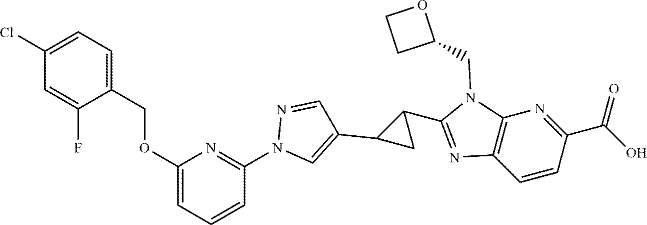 [SMILES: O=C(O)c1ccc2nc(C3CC3c3cnn(-c4cccc(OCc5ccc(Cl)cc5F)n4)c3)n(C[C@@H]3CCO3)c2n1] |
| 120aw | 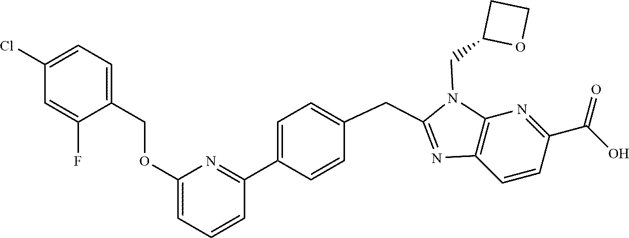 [SMILES: O=C(O)c1ccc2nc(Cc3ccc(-c4cccc(OCc5ccc(Cl)cc5F)n4)cc3)n(C[C@@H]3CCO3)c2n1] |

The compounds of Formula I or II include pharmaceutically acceptable salts thereof. In addition, the compounds of Formula I or II also include other salts of such compounds which are not necessarily pharmaceutically acceptable salts, and which may be useful as intermediates for preparing and/or purifying compounds of Formula I or II and/or for separating enantiomers of compounds of Formula I or II. Non-limiting examples of pharmaceutically acceptable salts of compounds of Formula I include trifluoroacetic acid salts.

It will further be appreciated that the compounds of Formula I or II or their salts may be isolated in the form of solvates, and accordingly that any such solvate is included within the scope of the present invention. For example, compounds of Formula I or II and salts thereof can exist in unsolvated as well as solvated forms with pharmaceutically acceptable solvents such as water, ethanol, and the like.

### Pharmaceutical Compositions and Administration

When employed as pharmaceuticals, the compounds of Formula I or II, including pharmaceutically acceptable salts or solvates thereof can be administered in the form of a pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided herein are pharmaceutical compositions which contain, as the active ingredient, a compound of Formula I or II, or a pharmaceutically acceptable salt or solvate thereof, in combination with one or more pharmaceutically acceptable excipients (carriers). For example, a pharmaceutical composition prepared using a compound of Formula I or II, or a pharmaceutically acceptable salt or solvate thereof. In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing a compound of Formula I or II, or a pharmaceutically acceptable salt or solvate thereof with a pharmaceutically acceptable excipient. Pharmaceutical compositions containing a compound of Formula I or II, or a pharmaceutically acceptable salt or solvate thereof as the active ingredient can be prepared by intimately mixing the compound of Formula I or II, or a pharmaceutically acceptable salt or solvate thereof with a pharmaceutical carrier according to conventional pharmaceutical compounding techniques. The carrier can take a wide variety of forms depending upon the desired route of administration (e.g., oral, parenteral). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In some embodiments, the compound or pharmaceutical composition can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

In some embodiments, the compounds and pharmaceutical compositions described herein or a pharmaceutical composition thereof can be administered to patient in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal (e.g., intranasal), nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In some embodiments, a preferred route of administration is parenteral (e.g., intratumoral).

In some embodiments, a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof) as described herein or pharmaceutical compositions thereof can be formulated for parenteral administration, e.g., formulated for injection via the intraarterial, intrasternal, intracranial, intravenous, intramuscular, sub-cutaneous, or intraperitoneal routes. For example, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure. In some embodiments, devices are used for parenteral administration. For example, such devices may include needle injectors, microneedle injectors, needle-free injectors, and infusion techniques.

In some embodiments, the pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In some embodiments, the form must be sterile and must be fluid to the extent that it may be easily injected. In some embodiments, the form should be stable under the conditions of manufacture and storage and must be preserved against the contaminating action of microorganisms, such as bacteria and fungi.

In some embodiments, the carrier also can be a solvent or dispersion medium containing, for example, water, ethanol, polyol (for example, glycerol, propylene glycol, and liquid polyethylene glycol, and the like), suitable mixtures thereof, and vegetable oils. In some embodiments, the proper fluidity can be maintained, for example, by the use of a coating, such as lecithin, by the maintenance of the required particle size in the case of dispersion, and by the use of surfactants. In some embodiments, the prevention of the action of microorganisms can be brought about by various antibacterial and antifungal agents, for example, parabens, chlorobutanol, phenol, sorbic acid, thimerosal, and the like. In some embodiments, isotonic agents, for example, sugars or sodium chloride are included. In some embodiments, prolonged absorption of the injectable compositions can be brought about by the use in the compositions of agents delaying absorption, for example, aluminum monostearate and gelatin.

In some embodiments, sterile injectable solutions are prepared by incorporating a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof) in the required amount in the appropriate solvent with various of the other ingredients enumerated above, as required, followed by filtered sterilization. In some embodiments, dispersions are prepared by incorporating the various sterilized active ingredients into a sterile vehicle which contains the basic dispersion medium and the required other ingredients from those enumerated above. In some embodiments, sterile powders are used for the preparation of sterile injectable solutions. In some embodiments, the methods of preparation are vacuum-drying and freeze-drying techniques, which yield a powder of the active ingredient, plus any additional desired ingredient from a previously sterile-filtered solution thereof.

In some embodiments, pharmacologically acceptable excipients usable in a rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol, Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM) , lactic acid, glycine, vitamins, such as vitamin A and E and potassium acetate.

In some embodiments, suppositories can be prepared by mixing a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof) or pharmaceutical compositions as described herein with suitable non-irritating excipients or carriers such as cocoa butter, polyethylene glycol or a suppository wax which are solid at ambient temperature but liquid at body temperature and therefore melt in the rectum and release the active compound. In some embodiments, compositions for rectal administration are in the form of an enema.

In some embodiments, a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof) as described herein or a pharmaceutical composition thereof is formulated for local delivery to the digestive or GI tract by way of oral administration (e.g., solid or liquid dosage forms.).

In some embodiments, solid dosage forms for oral administration include capsules, tablets, pills, powders, and granules. In some embodiments, a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof)is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For example, in the case of capsules, tablets and pills, the dosage form may also comprise buffering agents. In some embodiments, solid compositions of a similar type may also be employed as fillers in soft and hard-filled gelatin capsules using such excipients as lactose or milk sugar as well as high molecular weight polyethylene glycols and the like.

In some embodiments, the pharmaceutical compositions will take the form of a unit dosage form such as a pill or tablet and thus the composition may contain, along with a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof) as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In some embodiments, another solid dosage form, a powder, marume, solution or suspension (e.g., in propylene carbonate, vegetable oils, PEG’s, poloxamer 124 or triglycerides) is encapsulated in a capsule (gelatin or cellulose base capsule). In some embodiments, unit dosage forms in which one or more compounds and pharmaceutical compositions as provided herein or additional active agents are physically separated are also contemplated; e.g., capsules with granules (or tablets in a capsule) of each drug; two-layer tablets; two-compartment gel caps, etc. In some embodiments, enteric coated or delayed release oral dosage forms are also contemplated.

In some embodiments, other physiologically acceptable compounds may include wetting agents, emulsifying agents, dispersing agents or preservatives that are particularly useful for preventing the growth or action of microorganisms. For example, various preservatives are well known and include, for example, phenol and ascorbic acid.

In some embodiments, the excipients are sterile and generally free of undesirable matter. For example, these compositions can be sterilized by conventional, well-known sterilization techniques. In some embodiments, for various oral dosage form excipients such as tablets and capsules, sterility is not required. For example, the United States Pharmacopeia/National Formulary (USP/NF) standard can be sufficient.

In some embodiments, a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof) as described herein or a pharmaceutical composition thereof is formulated for ocular administration. In some embodiments, ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

In some embodiments, a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof) as described herein or a pharmaceutical composition thereof is formulated for topical administration to the skin or mucosa (e.g., dermally or transdermally). In some embodiments, topical compositions can include ointments and creams. In some embodiments, ointments are semisolid preparations that are typically based on petrolatum or other petroleum derivatives. In some embodiments, creams containing the selected active agent are typically viscous liquid or semisolid emulsions, often either oil-in-water or water-in-oil. For example, cream bases are typically water-washable, and contain an oil phase, an emulsifier and an aqueous phase. For example, the oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. In some embodiments, the emulsifier in a cream formulation is generally a nonionic, anionic, cationic or amphoteric surfactant. In some embodiments, as with other carriers or vehicles, an ointment base should be inert, stable, nonirritating and nonsensitizing.

In any of the foregoing embodiments, pharmaceutical compositions as described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

In some embodiments, the dosage for a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof), is determined based on a multiple factors including, but not limited to, type, age, weight, sex, medical condition of the patient, severity of the medical condition of the patient, route of administration, and activity of the compound or pharmaceutically acceptable salt or solvate thereof. In some embodiments, proper dosage for a particular situation can be determined by one skilled in the medical arts. In some embodiments, the total daily dosage may be divided and administered in portions throughout the day or by means providing continuous delivery.

In some embodiments, a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof), is administered at a dose from about 0.01 to about 1000 mg. For example, from about 0.1 to about 30 mg, about 10 to about 80 mg, about 0.5 to about 15 mg, about 50 mg to about 200 mg, about 100 mg to about 300 mg, about 200 to about 400 mg, about 300 mg to about 500 mg, about 400 mg to about 600 mg, about 500 mg to about 800 mg, about 600 mg to about 900 mg, or about 700 mg to about 1000 mg. In some embodiments, the dose is a therapeutically effective amount.

In some embodiments, a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof) as described herein is administered at a dosage of from about 0.0002 mg/Kg to about 100 mg/Kg (e.g., from about 0.0002 mg/Kg to about 50 mg/Kg; from about 0.0002 mg/Kg to about 25 mg/Kg; from about 0.0002 mg/Kg to about 10 mg/Kg; from about 0.0002 mg/Kg to about 5 mg/Kg; from about 0.0002 mg/Kg to about 1 mg/Kg; from about 0.0002 mg/Kg to about 0.5 mg/Kg; from about 0.0002 mg/Kg to about 0.1 mg/Kg; from about 0.001 mg/Kg to about 50 mg/Kg; from about 0.001 mg/Kg to about 25 mg/Kg; from about 0.001 mg/Kg to about 10 mg/Kg; from about 0.001 mg/Kg to about 5 mg/Kg; from about 0.001 mg/Kg to about 1 mg/Kg; from about 0.001 mg/Kg to about 0.5 mg/Kg; from about 0.001 mg/Kg to about 0.1 mg/Kg; from about 0.01 mg/Kg to about 50 mg/Kg; from about 0.01 mg/Kg to about 25 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 50 mg/Kg; from about 0.1 mg/Kg to about 25 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg). In some embodiments, a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof) as described herein is administered as a dosage of about 100 mg/Kg.

In some embodiments, the foregoing dosages of a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof), can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof) as described herein is for 1 day, 2 days, 3 days, 4 days, 5 days, 6 days, 7 days, 8 days, 9 days, 10 days, 11 days, 12 days,13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof) is administered to a patient for a period of time followed by a separate period of time where administration of the compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof)is stopped. In some embodiments, a compound of Formula I—II, or a pharmaceutically acceptable salt or solvate thereof (e.g., a compound of any one of Formulas IA and IB, or any one of Formulas IIA, IIB, and IIC, or a pharmaceutically acceptable salt or solvate thereof)is administered for a first period and a second period following the first period, with administration stopped during the second period, followed by a third period where administration of the compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof) is started and then a fourth period following the third period where administration is stopped. For example, the period of administration of a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof) followed by a period where administration is stopped is repeated for a determined or undetermined period of time. In some embodiments, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks,10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In some embodiments, a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof), is orally administered to the patient one or more times per day (e.g., one time per day, two times per day, three times per day, four times per day per day or a single daily dose).

In some embodiments, a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof), is administered by parenteral administration to the patient one or more times per day (e.g., 1 to 4 timesone time per day, two times per day, three times per day, four times per day or a single daily dose).

In some embodiments, a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof), is administered by parenteral administration to the patient weekly.

### Methods of Treatment

In some embodiments, this disclosure features methods for treating a patient (e.g., a human) having a disease, disorder, or condition in which modulation of GLP-1R (e.g., repressed or impaired and/or elevated or unwanted GLP-1R) is beneficial for the treatment of the underlying pathology and/or symptoms and/or progression of the disease, disorder, or condition. In some embodiments, the methods described herein can include or further include treating one or more conditions associated, co-morbid or sequela with any one or more of the conditions described herein.

Provided herein is a method for treating a GLP-1 associated disease, disorder, or condition, the method comprising administering to a patient in need thereof an effective amount of a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof), or a pharmaceutical composition as disclosed herein.

In some embodiments, the disease, disorder, or condition includes, but is not limited to type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity (including hypothalamic obesity and monogenic obesity), weight gain from use of other agents, idiopathic intracranial hypertension, Wolfram syndrome, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), nonalcoholic fatty liver disease (NAFLD), ulcerative colitis, inflammatory bowel disease, colitis, irritable bowel syndrome, Crohn’s disease, short bowel syndrome, Parkinson’s, Alzheimer’s disease, impaired cognition, schizophrenia, and Polycystic Ovary Syndrome (PCOS).

In some embodiments, the disease, disorder, or condition includes, but is not limited to type 2 diabetes mellitus, early onset type 2 diabetes mellitus, obesity, idiopathic intracranial hypertension, Wolfram syndrome, weight gain from use of other agents, gout, excessive sugar craving, hypertriglyceridemia, dyslipidemia, gestational diabetes, kidney disease (e.g., acute kidney disorder, tubular dysfunction, proinflammatory changes to the proximal tubules), adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson’s disease, Polycystic Ovary Syndrome (PCOS), or any combination thereof.

In some embodiments, the disease, disorder, or condition includes, but is not limited to, type 2 diabetes mellitus, early onset type 2 diabetes mellitus, obesity, idiopathic intracranial hypertension, Wolfram syndrome, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.

In some embodiments, the compounds and pharmaceutical compositions and methods for treating a patient described herein induce one or more of a reduction of blood glucose levels (e.g., reduce blood glucose levels), a reduction of blood hemoglobin Alc (HbAlc) levels, a promotion of insulin synthesis, a stimulation of insulin secretion, an increase in the mass of β-cells, a modulation of gastric acid secretion, a modulation of gastric emptying, a decrease in the body mass index (BMI), and/or a decrease in glucagon production (e.g., level). In some embodiments, the compounds and pharmaceutical compositions and methods for treating a patient described herein can reduce blood glucose levels, reduce blood hemoglobin Alc (HbAlc) levels, promote insulin synthesis, stimulate insulin secretion, increase the mass of β-cells, modulate gastric acid secretion, modulate gastric emptying, decrease the body mass index (BMI), decrease glucagon production (e.g., level), or any combination thereof. In certain embodiments, the compounds and pharmaceutical compositions and methods for treating a patient described herein stabilize serum glucose and serum insulin levels (e.g., serum glucose and serum insulin concentrations).Also provided herein are methods for modulating glucose or insulin levels in a patient in need of such modulating, the method comprising administering to the patient an effective amount of a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof), or a pharmaceutical composition as disclosed herein.

In some embodiments, provided herein is a method for reducing the risk (e.g., by about at least 20%, at least 30%, at least 40%, at least 50%, at least 60%, at least 70%, or at least 80%) of major adverse cardiovascular events (MACE) in a patient in need thereof, the method comprising administering to the patient an effective amount of a compound of Formula I or II, or a pharmaceutically acceptable salt or solvate thereof (e.g., a compound of any one of Formulas IA and IB, or any one of Formulas IIA, IIB, and IIC, or a pharmaceutically acceptable salt or solvate thereof), or a pharmaceutical composition as disclosed herein. In certain of these embodiments, the patient is an adult that has been diagnosed with type 2 diabetes (T2D). In certain embodiments, the patient is an adult that has been diagnosed with a heart disease. In certain embodiments, the patient is an adult that has been diagnosed with type 2 diabetes (T2D) and a heart disease. In certain embodiments, the patient is an adult that has type 2 diabetes (T2D). In certain embodiments, the patient is an adult that has a heart disease. In certain embodiments, the patient has type 2 diabetes (T2D) and a heart disease.

### Indications

### Obesity

In some embodiments, the condition, disease or disorder is obesity and conditions, diseases or disorders that are associated with or related to obesity. Non-limiting examples of obesity and obesity related conditions include symptomatic obesity, simple obesity, childhood obesity, morbid obesity, and abdominal obesity (central obesity characterized by abdominal adiposity). Non-limiting examples of symptomatic obesity include endocrine obesity (e.g., Cushing syndrome, hypothyroidism, insulinoma, obese type II diabetes, pseudohypoparathyroidism, hypogonadism), hypothalamic obesity, hereditary obesity (e.g., Prader-Willi syndrome, Laurence-Moon-Biedl syndrome), and drug-induced obesity (e.g., steroid, phenothiazine, insulin, sulfonylurea agent, or β-blocker-induced obesity).

In some embodiments, the condition, disease or disorder is associated with obesity. Examples of such conditions, diseases or disorders include, without limitation, glucose tolerance disorders, diabetes (e.g., type 2 diabetes, obese diabetes), lipid metabolism abnormality, hyperlipidemia, hypertension, cardiac failure, hyperuricemia, gout, fatty liver (including non-alcoholic steatohepatitis (NASH)), coronary heart disease (e.g., myocardial infarction, angina pectoris), cerebral infarction (e.g., brain thrombosis, transient cerebral ischemic attack), bone or articular disease (e.g., knee osteoarthritis, hip osteoarthritis, spondylitis deformans, lumbago), sleep apnea syndrome, obesity hypoventilation syndrome (Pickwickian syndrome), menstrual disorder (e.g., abnormal menstrual cycle, abnormality of menstrual flow and cycle, amenorrhea, abnormal catamenial symptom), visceral obesity syndrome, urine incontinence, and metabolic syndrome. In some embodiments, the chemical compound and pharmaceutical compositions described herein can be used to treat patients exhibiting symptoms of both obesity and insulin deficiency.

### Diabetes

In some embodiments, the condition, disease or disorder is diabetes. Non-limiting examples of diabetes include type 1 diabetes mellitus, type 2 diabetes mellitus (e.g., diet-treated type 2-diabetes, sulfonylurea-treated type 2-diabetes, a far-advanced stage type 2-diabetes, long-term insulin-treated type 2-diabetes), diabetes mellitus (e.g., non-insulin-dependent diabetes mellitus, insulin-dependent diabetes mellitus), gestational diabetes, obese diabetes, autoimmune diabetes, and borderline type diabetes. In some embodiments, the condition, disease or disorder is type 2 diabetes mellitus (e.g., diet-treated type 2-diabetes, sulfonylurea-treated type 2-diabetes, a far-advanced stage type 2-diabetes, long-term insulin-treated type 2-diabetes).

Provided herein is a method of treating a diabetes mellitus in a patient, the method comprising (a) determining that the patient has type 2 diabetes mellitus, and (b) administering to the patient a therapeutically effective amount of a compound of Formula I or II, or a pharmaceutically acceptable salt or solvate thereof (e.g., a compound of any one of a compound of any one of Formulas IA and IB, or any one of Formulas IIA, IIB, and IIC, or a pharmaceutically acceptable salt or solvate thereof, or a pharmaceutically acceptable salt or solvate thereof) or a pharmaceutical composition as disclosed herein.

Provided herein is a method for treating type 2 diabetes mellitus in a patient, the method comprising administering to a patient identified or diagnosed as having type 2 diabetes mellitus a therapeutically effective amount of a compound of Formula I or II, or a pharmaceutically acceptable salt or solvate thereof (e.g., a compound of any one of a compound of any one of Formulas IA and IB, or any one of Formulas IIA, IIB, and IIC, or a pharmaceutically acceptable salt or solvate thereof, or a pharmaceutically acceptable salt or solvate thereof), or a pharmaceutical composition as disclosed herein.

Also provided herein is a method of treating type 2 diabetes mellitus in a patient in need thereof, the method comprising administering to the patient a therapeutically effective amount of a compound of Formula I or II, or a pharmaceutically acceptable salt or solvate thereof (e.g., a compound of any one of a compound of any one of Formulas IA and IB, or any one of Formulas IIA, IIB, and IIC, or a pharmaceutically acceptable salt or solvate thereof, or a pharmaceutically acceptable salt or solvate thereof), or a pharmaceutical composition as disclosed herein.

In some embodiments, the compounds and pharmaceutical compositions and methods for treating a patient with a condition, disease, or disorder (e.g., type 2 diabetes mellitus) described herein reduce fasting plasma glucose levels. In some embodiments, the compounds and pharmaceutical compositions and methods for treating a patient with a condition, disease, or disorder (e.g., type 2 diabetes mellitus) described herein reduce non-fasting plasma glucose levels. In some embodiments, the compounds and pharmaceutical compositions and methods for treating a patient with a condition, disease, or disorder (e.g., type 2 diabetes mellitus) described herein reduce HbAlc levels. In some embodiments, the compounds and pharmaceutical compositions and methods for treating a patient with a condition, disease, or disorder (e.g., type 2 diabetes mellitus) described herein reduce glucagon levels. In some embodiments, the compounds and pharmaceutical compositions and methods for treating a patient with a condition, disease, or disorder (e.g., type 2 diabetes mellitus) described herein increase insulin levels. In some embodiments, the compounds and pharmaceutical compositions and methods for treating a patient with a condition, disease, or disorder (e.g., type 2 diabetes mellitus) described herein reduce BMI.

In some embodiments, a reduction in fasting plasma glucose levels of about 5% to about 95% indicates treatment of type 2 diabetes mellitus. In some embodiments, a reduction in fasting plasma glucose levels of about 15% to about 80% indicates treatment of type 2 diabetes mellitus. In some embodiments, a reduction in fasting plasma glucose levels of about 25% to about 60% indicates treatment of type 2 diabetes mellitus. In some embodiments, a reduction in fasting plasma glucose levels to about or below 126 mg/dL, about or below 110 mg/dL, or about or below 90 mg/dL indicates treatment of the type 2 diabetes mellitus.

In some embodiments, a reduction in non-fasting plasma glucose levels of about 5% to about 95% indicates treatment of type 2 diabetes mellitus. In some embodiments, a reduction in non-fasting plasma glucose levels of about 15% to about 80% indicates treatment of type 2 diabetes mellitus. In some embodiments, a reduction in non-fasting plasma glucose levels of about 25% to about 60% indicates treatment of type 2 diabetes mellitus. In some embodiments, a reduction in non-fasting plasma glucose levels to about or below 200 mg/dL, about or below 150 mg/dL, or about or below 130 mg/dL indicates treatment of type 2 diabetes mellitus.

In some embodiments, a reduction in HbAlc levels of about 5% to about 95% indicates treatment of type 2 diabetes mellitus. In some embodiments, a reduction in HbAlc levels of about 15% to about 80% indicates treatment of type 2 diabetes mellitus. In some embodiments, a reduction in HbAlc levels of about 25% to about 60% indicates treatment of type 2 diabetes mellitus. In some embodiments, reduction in HbAlc levels to about or below 6.5%, about or below 6.0%, or about or below 5.0% indicates treatment of type 2 diabetes mellitus.

In some embodiments, a reduction in glucagon levels of about 5% to about 95% indicates treatment of type 2 diabetes mellitus. In some embodiments, a reduction in glucagon levels of about 15% to about 80% indicates treatment of type 2 diabetes mellitus. In some embodiments, a reduction in glucagon levels of about 25% to about 60% indicates treatment of type 2 diabetes mellitus. In some embodiments, an increase in insulin levels of about 5% to about 95% indicates treatment of type 2 diabetes mellitus. In some embodiments, an increase in insulin levels of about 15% to about 80% indicates treatment of type 2 diabetes mellitus. In some embodiments, an increase in insulin levels of about 25% to about 60% indicates treatment of type 2 diabetes mellitus.

In some embodiments, a reduction in BMI of about 5% to about 95% indicates treatment of type 2 diabetes mellitus. In some embodiments, a reduction in BMI of about 15% to about 80% indicates treatment of the type 2 diabetesmellitus. In some embodiments, a reduction in BMI of about 25% to about 60% indicates treatment of type 2 diabetes mellitus. In some embodiments, a reduction in BMI of about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of type 2 diabetes mellitus. In some embodiments, a reduction in BMI to about or below 40, about or below 30, or about or below 20 indicates treatment of type 2 diabetes mellitus.

In some embodiments, the condition, disease or disorder is associated with diabetes (e.g., a complication of diabetes). Non-limiting examples of disorders associated with diabetes include obesity, obesity-related disorders, metabolic syndrome, neuropathy, nephropathy (e.g., diabetic nephropathy), retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, diabetic cachexia, delayed wound healing, diabetic dyslipidemia peripheral blood circulation disorder, cardiovascular risk factors. (e.g., coronary artery disease, peripheral artery disease, cerebrovascular disease, hypertension, and risk factors related to unmanaged cholesterol and/or lipid levels, and/or inflammation), NASH, bone fracture, and cognitive dysfunction

Other non-limiting examples of disorders related to diabetes include pre-diabetes, hyperlipidemia (e.g., hypertriglyceridemia, hypercholesterolemia, high LDL-cholesterolemia, low HDL-cholesterolemia, postprandial hyperlipemia), metabolic syndrome (e.g., metabolic disorder where activation of GLP-1R is beneficial, metabolic syndrome X), hypertension, impaired glucose tolerance (IGT), insulin resistance, and sarcopenia.

In some embodiments, the condition, disease or disorder is diabetes and obesity (diabesity). In some embodiments, the compounds described herein are useful in improving the therapeutic effectiveness of metformin.

### Disorders of Metabolically Important Tissues

In some embodiments, the condition, disease or disorder is a disorder of a metabolically important tissue. Non-limiting examples of metabolically important tissues include liver, fat, pancreas, kidney, and gut.

In some embodiments, the condition, disease or disorder is a fatty liver disease. Fatty liver diseases include, but are not limited to, non-alcoholic fatty acid liver disease (NAFLD), steatohepatitis, non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, hyperlipoproteinemia, glycogen storage diseases, Weber-Christian disease, Wolman disease, acute fatty liver of pregnancy, and lipodystrophy.

Non-alcoholic fatty liver disease (NAFLD) represents a spectrum of disease occurring in the absence of alcohol abuse and is typically characterized by the presence of steatosis (fat in the liver). NAFLD is believed to be linked to a variety of conditions, e.g., metabolic syndrome (including obesity, diabetes and hypertriglyceridemia) and insulin resistance. It can cause liver disease in adults and children and can ultimately lead to cirrhosis (Skelly et al., J Hepatol 2001; 35: 195-9; Chitturi et al., Hepatology 2002; 35(2):373-9). The severity of NAFLD ranges from the relatively benign isolated predominantly macrovesicular steatosis (i.e., nonalcoholic fatty liver or NAFL) to nonalcoholic steatohepatitis (NASH) (Angulo et al., J Gastroenterol Hepatol 2002; 17 Suppl: S186-90).

Other non-limiting examples of disorders in metabolically important tissues include joint disorders (e.g., osteoarthritis, secondary osteoarthritis), steatosis (e.g., in the liver); fibrosis (e.g., in the liver); cirrhosis (e.g., in the liver); gall stones; gallbladder disorders; gastroesophageal reflux; sleep apnea; hepatitis; fatty liver; bone disorder characterized by altered bone metabolism, such as osteoporosis, including postmenopausal osteoporosis, poor bone strength, osteopenia, Paget’s disease, osteolytic metastasis in cancer patients, osteodistrophy in liver disease and the altered bone metabolism caused by renal failure or haemodialysis, bone fracture, bone surgery, aging, pregnancy, protection against bone fractures, and malnutritionpolycystic ovary syndrome; renal disease (e.g., chronic renal failure, glomerulonephritis, glomerulosclerosis, nephrotic syndrome, hypertensive nephrosclerosis, end-stage renal disease); muscular dystrophy, angina pectoris, acute or chronic diarrhea, testicular dysfunction, respiratory dysfunction, frailty, sexual dysfunction (e.g., erectile dysfunction), and geriatric syndrome. In some embodiments, the compounds and pharmaceutical compositions described herein can be used for treating surgical trauma by improving recovery after surgery and/or by preventing the catabolic reaction caused by surgical trauma.

### Cardiovascular and Vascular Diseases

In some embodiments, the condition, disease or disorder is a cardiovascular disease. Non-limiting examples of cardiovascular disease include congestive heart failure, atherosclerosis, arteriosclerosis, coronary heart disease, coronary artery disease, congestive heart failure, coronary heart disease, hypertension, cardiac failure, cerebrovascular disorder (e.g., cerebral infarction), vascular dysfunction, myocardial infarction, elevated blood pressure (e.g., 130/85 mm Hg or higher), and prothrombotic state (exemplified by high fibrinogen or plasminogen activator inhibitor in the blood).

In some embodiments, the condition, disease or disorder is related to a vascular disease. Non-limiting examples of vascular diseases include peripheral vascular disease, macrovascular complications (e.g., stroke), vascular dysfunction, peripheral artery disease, abdominal aortic aneurysm, carotid artery disease, cerebrovascular disorder (e.g., cerebral infarction), pulmonary embolism, chronic venous insufficiency, critical limb ischemia, retinopathy, nephropathy, and neuropathy.

### Neurological Diseases

In some embodiments, the condition, disease or disorder is a neurological disorder (e.g., neurodegenerative disorder) or a psychiatric disorder. Non-limiting examples of neurological disorders include idiopathic intracranial hypertension (IIH), brain insulin resistance, mild cognitive impairment (MCI), Alzheimer’s disease (AD), Parkinson’s disease (PD), anxiety, dementia (e.g., senile dementia), traumatic brain injury, Huntington’s chores, tardive dyskinesia, hyperkinesia, mania, Morbus Parkinson, steel-Richard syndrome, Down’s syndrome, myasthenia gravis, nerve trauma, brain trauma, vascular amyloidosis, cerebral hemorrhage I with amyloidosis, brain inflammation, Friedrich’s ataxia, acute confusion disorder, amyotrophic lateral sclerosis (ALS), glaucoma, and apoptosis-mediated degenerative diseases of the central nervous system (e.g., Creutzfeld-Jakob Disease, bovine spongiform encephalopathy (mad cow disease), and chronic wasting syndrome). See, e.g., U.S Publication No. 20060275288A1.

In some embodiments, the condition, disease or disorder is idiopathic intracranial hypertension. Idiopathic intracranial hypertension is characterized by increased intracranial pressure and papilloedema. See, e.g., Virdee et al. Ophthalmol Ther. 2020; 9(4):767-781. In some embodiments, the compounds and pharmaceutical compositions and methods described herein reduce cerebrospinal fluid secretion in a patient with idiopathic intracranial hypertension. In some embodiments, the compounds and pharmaceutical compositions and methods described herein reduce intracranial pressure in a patient with idiopathic intracranial hypertension. In some embodiments, the compounds and pharmaceutical compositions and methods described herein reduce one or more symptoms in a patient with idiopathic intracranial hypertension. Symptoms of idiopathic intracranial hypertension can include severe headaches and visual impairment. In some embodiments, the patient with idiopathic intracranial hypertension is female. In some embodiments, the patient with idiopathic intracranial hypertension is about 20 to about 30 years old. In some embodiments, the patient with idiopathic intracranial hypertension is obese.

In some embodiments, the condition, disease or disorder is Wolfram syndrome. Wolfram syndrome is caused by biallelic mutations of the Wolframin ER transmembrane glycoprotein (Wfs1) gene. See, e.g., Seppa et al. Sci Rep 9, 15742 (2019). Wolfram syndrome can first appear as diabetes mellitus, followed by optic nerve atrophy, deafness, and symptoms of neurodegeneration. Patients with Wolfram syndrome can have symptoms of ataxia, sleep apnea, dysphagia, hearing loss, and loss of taste due to brainstem atrophy. In some embodiments, the compounds and pharmaceutical compositions and methods described herein reduce neuroinflammation in a patient with Wolfram syndrome. In some embodiments, the neuroinflammation is reduced in the inferior olive in the patient. In some embodiments, the compounds and pharmaceutical compositions and methods described herein reduce retinal ganglion cell death in a patient with Wolfram syndrome. In some embodiments, the compounds and pharmaceutical compositions and methods described herein reduce axonal degeneration in a patient with Wolfram syndrome. In some embodiments, the compounds and pharmaceutical compositions and methods described herein reduce one or more symptoms (e.g., any of the symptoms described herein) in a patient with Wolfram syndrome.

Non-limiting examples of psychiatric disorders include drug dependence/addiction (narcotics and amphetamines and attention deficit/hyperactivity disorder (ADHD). The compounds and pharmaceutical compositions described herein can be useful in improving behavioral response to addictive drugs, decreasing drug dependence, prevention drug abuse relapse, and relieving anxiety caused by the absence of a given addictive substance. See, e.g., U.S. Publication No. 20120021979A1.

In some embodiments, the compounds and pharmaceutical compositions described herein are useful in improving learning and memory by enhancing neuronal plasticity and facilitation of cellular differentiation, and also in preserving dopamine neurons and motor function in Morbus Parkinson.

### Insulin-Related

In some embodiments, the condition, disease or disorder is impaired fasting glucose (IFG), impaired fasting glycemia (IFG), hyperglycemia, insulin resistance (impaired glucose homeostasis), hyperinsulinemia, elevated blood levels of fatty acids or glycerol, a hypoglycemic condition, insulin resistant syndrome, paresthesia caused by hyperinsulinemia, hyperlipidaemia, hypercholesteremia, impaired wound healing, leptin resistance, glucose intolerance, increased fasting glucose, dyslipidemia (e.g., hyperlipidemia, atherogenic dyslipidemia characterized by high triglycerides and low HDL cholesterol), glucagonoma, hyperuricacidemia, hypoglycemia (e.g., nighttime hypoglycemia), and concomitant comatose endpoint associated with insulin.

In some embodiments, the compounds and pharmaceutical compositions described herein can reduce or slow down the progression of borderline type, impaired fasting glucose or impaired fasting glycemia into diabetes.

### Autoimmune Disorders

In some embodiments, the condition, disease or disorder is an autoimmune disorder. Non-limiting examples of autoimmune disorders include multiple sclerosis, experimental autoimmune encephalomyelitis, autoimmune disorder is associated with immune rejection, graft versus host disease, uveitis, optic neuropathies, optic neuritis, transverse myelitis, inflammatory bowel disease, rheumatoid arthritis, ankylosing spondylitis, systemic lupus erythematosus, myasthenia gravis, and Graves disease. See, e.g., U.S. Publication No. 20120148586A1.

### Stomach and Intestine-Related Disorders

In some embodiments, the condition, disease or disorder is a stomach or intestine related disorder. Non-limiting examples of these disorders include ulcers of any etiology (e.g. peptic ulcers, Zollinger-Ellison syndrome, drug-induced ulcers, ulcers related to infections or other pathogens), digestion disorders, malabsorption, short bowel syndrome, cul-de-sac syndrome, inflammatory bowel diseases (Crohn’s disease and ulcerative colitis), celiac sprue, hypogammaglobulinemic sprue, chemotherapy and/or radiation therapy-induced mucositis and diarrhea, gastrointestinal inflammation, short bowel syndrome, colitis ulcerosa, gastric mucosal injury (e.g., gastric mucosal injury caused by aspirin), small intestinal mucosal injury, and cachexia (e.g., cancerous cachexia, tuberculous cachexia, cachexia associated with blood disease, cachexia associated with endocrine disease, cachexia associated with infectious disease, and cachexia caused by acquired immunodeficiency syndrome).

### Body Weight

In some embodiments, the compounds and pharmaceutical compositions described herein can be used to reduce body weight (e.g., excess body weight), prevent body weight gain, induce weight loss, decrease body fat, or reduce food intake in a patient (e.g., a patient in need thereof). In some embodiments, the weight increase in a patient may be attributed to excessive ingestion of food or unbalanced diets, or may be weight increase derived from a concomitant drug (e.g., insulin sensitizers having a PPARγ agonist-like action, such as troglitazone, rosiglitazone, englitazone, ciglitazone, pioglitazone and the like). In some embodiments, the weight increase may be weight increase before reaching obesity, or may be weight increase in an obese patient. In some embodiments, the weight increase may also be medication-induced weight gain or weight gain subsequent to cessation of smoking. In some embodiments, the weight gain is induced by the use of steroids or antipsychotics.

In some embodiments, the condition, disease or disorder is an eating disorder, such as hyperphagia, binge eating, bulimia, compulsive eating, or syndromic obesity such as Prader-Willi and Bardet-Biedl syndromes.

### Inflammatory Diseases

In some embodiments, the condition, disease or disorder is an inflammatory disorder. Non-limiting examples of inflammatory disorders include chronic rheumatoid arthritis, spondylitis deformans, arthritis deformans, lumbago, gout, post-operational or post-traumatic inflammation, bloating, neuralgia, laryngopharyngitis, cystitis, pneumonia, pancreatitis, enteritis, inflammatory bowel disease (including inflammatory large bowel disease), inflammation in metabolically important tissues including liver, fat, pancreas, kidney and gut, and a proinflammatory state (e.g., elevated levels of proinflammatory cytokines or markers of inflammation-like C-reactive protein in the blood).

### Cancer

In some embodiments, the condition, disease or disorder is cancer. Suitable examples of cancer include breast cancer (e.g., invasive ductal breast cancer, noninvasive ductal breast cancer, inflammatory breast cancer), prostate cancer (e.g., hormone-dependent prostate cancer, hormone-independent prostate cancer), pancreatic cancer (e.g., ductal pancreatic cancer), gastric cancer (e.g., papillary adenocarcinoma, mucous adenocarcinoma, adenosquamous carcinoma), lung cancer (e.g., non-small cell lung cancer, small-cell lung cancer, malignant mesothelioma), colon cancer (e.g., gastrointestinal stromal tumor), rectal cancer (e.g., gastrointestinal stromal tumor), colorectal cancer (e.g., familial colorectal cancer, hereditary non-polyposis colorectal cancer, gastrointestinal stromal tumor), small intestinal cancer (e.g., non-Hodgkin’s lymphoma, gastrointestinal stromal tumor), esophageal cancer, duodenal cancer, tongue cancer, pharyngeal cancer (e.g., nasopharyngeal cancer, oropharynx cancer, hypopharyngeal cancer), salivary gland cancer, brain tumor (e.g., pineal astrocytoma, pilocytic astrocytoma, diffuse astrocytoma, anaplastic astrocytoma), neurilemmoma, liver cancer (e.g., primary liver cancer, extrahepatic bile duct cancer), renal cancer (e.g., renal cell cancer, transitional cell cancer of the renal pelvis and ureter), bile duct cancer, endometrial cancer, uterine cervical cancer, ovarian cancer (e.g., epithelial ovarian cancer, extragonadal germ cell tumor, ovarian germ cell tumor, ovarian tumor of low malignant potential), bladder cancer, urethral cancer, skin cancer (e.g., intraocular (ocular) melanoma, Merkel cell carcinoma), hemangioma, malignant lymphoma, malignant melanoma, thyroid cancer (e.g., medullary thyroid cancer), parathyroid cancer, nasal cavity cancer, sinus cancer, bone tumor (e.g., osteosarcoma, Ewing tumor, uterine sarcoma, soft tissue sarcoma), angiofibroma, sarcoma of the retina, penis cancer, testicular tumor, pediatric solid tumor (e.g., Wilms’ tumor, childhood kidney tumor), Kaposi’s sarcoma, Kaposi’s sarcoma caused by AIDS, tumor of maxillary sinus, fibrous histiocytoma, leiomyosarcoma, rhabdomyosarcoma, and leukemia (e.g., acute myeloid leukemia, acute lymphoblastic leukemia).

### Hypothalamic-Pituitary Disorders

In some embodiments, the condition, disease or disorder is related to the hypothalamic-pituitary-gonadal axis. For example, the condition, disease or disorder is related to the hypothalamus-pituitary-ovary axis. In another example, the condition, disease or disorder is related to the hypothalamus-pituitary-testis axis. Hypothalamic-pituitary-gonadal axis diseases include, but are not limited to, hypogonadism, polycystic ovary syndrome, hypothyroidism, hypopituitarism, sexual dysfunction, and Cushing’s disease.

In some embodiments, the condition, disease or disorder associated with diabetes is related to the hypothalamic-pituitary-gonadal axis.

### Pulmonary Disease

In some embodiments, the condition, disease or disorder is related to a pulmonary disease. Pulmonary diseases include, but are not limited to, asthma, idiopathic pulmonary fibrosis, pulmonary hypertension, obstructive sleep apnoea-hypopnoea syndrome, and chronic obstructive pulmonary disease (COPD) (e.g., emphysema, chronic bronchitis, and refractory (non-reversible) asthma).

In some embodiments, the condition, disease or disorder associated with diabetes is a pulmonary disease.

### Combination Therapy

In some embodiments, this disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In some embodiments, the methods described herein include administering a compound described herein in combination with one or more of a diet therapy (e.g., dietary monitoring, diet therapy for diabetes), an exercise therapy (e.g., physical activity), blood sugar monitoring, gastric electrical stimulation (e.g., TANTALUS®), and diet modifications.

In some embodiments, the compounds of Formula I or II, or a pharmaceutically acceptable salt or solvate thereof as described herein can be administered in combination with one or more additional therapeutic agents.

Representative additional therapeutic agents include, but are not limited to, anti-obesity agents, therapeutic agents for diabetes, therapeutic agents for diabetic complications, therapeutic agents for hyperlipidemia, antihypertensive agents, diuretics, chemotherapeutics, immunotherapeutics, anti-inflammatory drugs, antithrombotic agents, anti-oxidants, therapeutic agents for osteoporosis, vitamins, antidementia drugs, erectile dysfunction drugs, therapeutic drugs for urinary frequency or urinary incontinence, therapeutic agents for NAFLD, therapeutic agents for NASH, and therapeutic agents for dysuria.

In some embodiments, the one or more additional therapeutic agents include those useful, for example, as anti-obesity agents. Non-limiting examples include monoamine uptake inhibitors (e.g., tramadol, phentermine, sibutramine, mazindol, fluoxetine, tesofensine), serotonin 2C receptor agonists (e.g., lorcaserin), serotonin 6 receptor antagonists, histamine H3 receptor modulator, GABA modulator (e.g., topiramate), including GABA receptor agonists (e.g., gabapentin, pregabalin), neuropeptide Y antagonists (e.g., velneperit), peptide YY or an analogue thereof, cannabinoid receptor antagonists (e.g., rimonabant, taranabant), ghrelin antagonists, ghrelin receptor antagonists, ghrelin acylation enzyme inhibitors, opioid receptor antagonists (e.g., GSK-1521498, naltrexone), orexin receptor antagonists, melanocortin 4 receptor agonists, 11β-hydroxysteroid dehydrogenase inhibitors (e.g., AZD-4017, BVT-3498, INCB-13739), pancreatic lipase inhibitors (e.g., orlistat, cetilistat), β3 agonists (e.g., N-5984), diacylglycerol acyltransferase 1 (DGAT1) inhibitors, acetylCoA carboxylase (ACC) inhibitors (e.g., compounds described in WO2020/234726, WO2020/044266, and U.S. Pat. No. 8,859,577), stearoyl-CoA desaturated enzyme inhibitors, microsomal triglyceride transfer protein inhibitors (e.g., R-256918), sodium-glucose cotransporter 2 (SGLT-2) inhibitors (e.g., JNJ-28431754, dapagliflozin, AVE2268, TS-033, YM543, TA-7284, ASP1941, remogliflozin) , empagliflozin, canagliflozin, ipragliflozin, tofogliflozin, sergliflozin etabonate, remogliflozin etabonate, or ertugliflozin), SGLT-1 inhibitors, MCR-4 agonists, monoamine reuptake inhibitors, melanocytestimulating hormone analogs, 5HT2c agonists, galanin antagonists, anorectic agents (such as a bombesin agonist), thyromimetic agents, dehydroepiandrosterone or analogs thereof, human agouti-related protein (AGRP) inhibitors, neuromedin U agonists, NFK inhibitors (e.g., HE-3286), PPAR agonists (e.g., GFT-505, DRF-11605, gemfibrozil, fenofibrate, balaglitazone, ciglitazone, darglitazone, englitazone, isaglitazone, pioglitazone, rosiglitazone, CLX-0940, GW-1536, GW-1 929, GW-2433, KRP-297, L-796449, LR-90, MK-0767, and SB-21 9994), phosphotyrosine phosphatase inhibitors (e.g., sodium vanadate, trodusquemin), GPR119 agonists (e.g., PSN-821, MBX-2982, APD597, compounds described in WO2010/140092, WO2010/128425, WO2010/128414, WO2010/106457), glucokinase activators (e.g., piragliatin, AZD-1656, AZD6370, TTP-355, TTP-399, TTP547, ARRY403, MK-0599, TAK-329, AZD5658 or GKM-001 compounds described in WO2010/10343, WO2010/103438, WO2010/013161, WO2007/122482, WO006/112549, WO007/028135, WO008/047821, WO008/050821, WO008/136428 and WO008/156757), leptin, leptin derivatives (e.g., metreleptin), leptin resistance improving drugs, CNTF (ciliary neurotrophic factor), BDNF (brain-derived neurotrophic factor), cholecystokinin agonists, amylin preparations (e.g., pramlintide, AC-2307), neuropeptide Y agonists (e.g., PYY3-36, derivatives of PYY3-36, obineptide, TM-30339, TM-30335), oxyntomodulin (OXM) preparations, appetite suppressants (e.g. ephedrine), FGF21 preparations (e.g., animal FGF21 preparations extracted from the pancreas of bovine or swine; human FGF21 preparations genetically synthesized using Escherichia coli or yeast; fragments or derivatives of FGF21), anorexigenic agents (e.g., P-57), human proislet peptide (HIP), melanocortin receptor 4 agonist (e.g., setmelanotide), melanin concentrating hormone receptor 1 antagonist, serotonergic agents (e.g. sibutramine, lorcaserin), farnesoid X receptor (FXR) agonist(e.g., obeticholic acid, tropifexor, cilofexor, LY2562175, Met409, TERN-101, EDP305, compounds described in WO2020/234726 and WO2020/044266), phentermine, zonisamide, norepinephrine/dopamine reuptake inhibitor, GDF-15 analog, methionine aminopeptidase 2 (MetAP2) inhibitor, diethylpropion, phendimetrazine, benzphetamine, fibroblast growth factor receptor (FGFR) modulator, biotin, a MAS receptor modulator, glucagon receptor agonist, CCKa agonists (e.g., compounds described in WO2005/116034 and U.S. Publication No. 2005/0287100), and AMP-activated protein kinase (AMPK) activator.

In some embodiments, the one or more additional therapeutic agents include those useful, for example, as anti-diabetic agents. Non-limiting examples include insulin and insulin preparations (e.g., animal insulin preparations extracted from the pancreas of bovine or swine; human insulin preparations genetically synthesized using Escherichia coli or yeast; zinc insulin; protamine zinc insulin; fragment or derivative of insulin (e.g., INS- 1), oral insulin preparation, synthetic human insulin), insulin sensitizers (e.g., pioglitazone or a salt thereof), biguanides (e.g., metformin, buformin or a salt thereof (e.g., hydrochloride, fumarate, succinate)), glucagon analogs (e.g., any of glucagon analogs described, e.g., in WO 2010/011439), agents which antagonize the actions of or reduce secretion of glucagon, sulfonylurea agents (e.g., chlorpropamide, tolazamide, glimepiride, tolbutamide, glibenclamide, gliclazide, acetohexamide, glyclopyramide, glybuzole, glyburide, glipizide), thiazolidinedione agents (e.g. rosiglitazone, lobeglitazone, troglitazone, balaglitazone, rivoglitazone, lobeglitazone or pioglitazone), glitazars (e.g., aleglitazar, chiglitazar, saroglitazar, muraglitazar, tesaglitazar), SGLT2 inhibitors (e.g., JNJ-28431754, dapagliflozin, AVE2268, TS-033, YM543, TA-7284, ASP1941, THR1474, TS-071, ISIS388626, LX4211, remogliflozin, empagliflozin, canagliflozin, ipragliflozin, tofogliflozin, sergliflozin etabonate, remogliflozin etabonate, ertugliflozin, compounds described in WO2010/023594), GPR40 agonists (e.g., a FFAR1/FFA1 agonist, e.g. fasiglifam), α-glucosidase inhibitors (e.g., adiposin, camiglibose, pradimicin-Q, salbostatin, voglibose, acarbose, miglitol, emiglitate), insulin secretagogues, such as prandial glucose regulators (sometimes called “short-acting secretagogues”), e.g., meglitinides (e.g. repaglinide and nateglinide), cholinesterase inhibitors (e.g., donepezil, galantamine, rivastigmine, tacrine), NMDA receptor antagonists, dual GLP-⅟GIP receptor agonists (e.g., LBT-2000, ZPD1-70), GLP-1R agonists (e.g., exenatide, liraglutide, albiglutide, dulaglutide, abiglutide, taspoglutide, lixisenatide, semaglutide, AVE-0010, S4P and Boc5), and dipeptidyl peptidase IV (DPP-4) inhibitors (e.g., vildagliptin, dutogliptin, gemigliptin, alogliptin, saxagliptin, sitagliptin, linagliptin, berberine, adogliptin, anagliptin (SK-0403), teneligliptin, omarigliptin, BI1356, GRC8200, MP-513, PF-00734200, PHX1149, SK-0403, ALS2-0426, TA-6666, TS-021, KRP-104, trelagliptin).

In some embodiments, the one or more additional therapeutic agents include those useful, for example, for treating NAFL and NASH. Non-limiting examples include FXR agonists (e.g., obeticholic acid), PF-05221304, PPAR α/δ agonists (e.g., elafibranor), a synthetic fatty acid-bile conjugate (e.g., aramchol), an anti-lysyl oxidase homologue 2 (LOXL2) monoclonal antibody (e.g., simtuzumab), a caspase inhibitor (e.g., emricasan), a MAPK5 inhibitor (e.g., GS-4997), a galectin 3 inhibitor (e.g., GR-MD-02), a fibroblast growth factor 21 (FGF21) (e.g., BMS-986036), a niacin analogue (e.g., ARJ 3037MO), a leukotriene D4 (LTD4) receptor antagonist (e.g., tipelukast), an acetyl-CoA carboxylase (ACC) inhibitor (e.g., NDI 010976 amd compounds described in WO2009/144554, WO2003/072197, WO2009/144555, and WO2008/065508), a ketohexokinase (KHK) inhibitor, an apoptosis signal-regulating kinase 1 (ASK1) inhibitor, an ileal bile acid transporter (IBAT) inhibitor, a dual antagonist of chemokine receptor 2 (CCR2) and CCR5 (e.g., cenicriviroc), diacylglyceryl acyltransferase 2 (DGAT2) inhibitor (e.g., compounds described in WO2020/234726 and U.S. Publication No. 20180051012), a CB1 receptor antagonist, an anti-CB1R antibody, glycyrrhizin, schisandra extract, ascorbic acid, glutathione, silymarin, lipoic acid, and d-alpha-tocopherol, ascorbic acid, glutathione, vitamin B-complex, glitazones/thiazolidinediones (e.g., troglitazone, rosiglitazone, pioglitazone, balaglitazone, rivoglitazone, lobeglitazone), metformin, cysteamine, sulfonylureas, alpha-glucosidase inhibitors, meglitinides, vitamin E, tetrahydrolipstatin, milk thistle protein, anti-virals, and anti-oxidants.

In some embodiments, the one or more additional therapeutic agents include those useful, for example, for treating diabetic complications. Non-limiting examples include aldose reductase inhibitors (e.g., tolrestat, epalrestat, zopolrestat, fidarestat, CT-112, ranirestat, lidorestat), neurotrophic factor and increasing agents thereof (e.g., NGF, NT-3, BDNF, neurotrophic production/secretion promoting agents described in WO01/14372 (e.g., 4-(4-chlorophenyl)-2-(2-methyl-1-imidazolyl)-5-[3-(2-methylphenoxyl)propyl]oxazole), compounds described in WO2004/039365), PKC inhibitors (e.g., ruboxistaurin mesylate), AGE inhibitors (e.g., ALT946, N-phenacylthiazolium bromide (ALT766), EXO-226, pyridorin, pyridoxamine), serotonin and noradrenalin reuptake inhibitors (e.g., duloxetine), sodium channel inhibitors (e.g., lacosamide), active oxygen scavengers (e.g., thioctic acid), cerebral vasodilators (e.g., tiapuride, mexiletine), somatostatin receptor agonists (e.g., BIM23190), and_apoptosis signal regulating kinase-1 (ASK-1) inhibitors.

In some embodiments, the one or more additional therapeutic agents include those useful, for example, for treating hyperlipidemia. Non-limiting examples include_HMG-COA reductase inhibitors (e.g., pravastatin, simvastatin, lovastatin, atorvastatin, fluvastatin, rosuvastatin, pitavastatin or a salt thereof (e.g., sodium salt, calcium salt)), squalene synthase inhibitors (e.g., compounds described in WO97/10224, e.g., N-[[(3R,5S)- 1-(3-acetoxy-2,2-dimethylpropyl)-7-chloro-5-(2,3-dimethoxyphenyl)-2-oxo-1,2,3,5-tetrahydro-4, 1-benzoxazepin-3-yl]acetyl]piperidin-4-acetic acid), fibrate compounds (e.g., bezafibrate, clofibrate, simfibrate, clinofibrate), anion exchange resin (e.g., colestyramine), nicotinic acid drugs (e.g., nicomol, niceritrol, niaspan), phytosterols (e.g., soysterol, gamma oryzanol (y-oryzanol)), cholesterol absorption inhibitors (e.g., zechia), CETP inhibitors (e.g., dalcetrapib, anacetrapib) and ω-3 fatty acid preparations (e.g., ω-3-fatty acid ethyl esters 90).

In some embodiments, the one or more additional therapeutic agents include those useful, for example, as anti-hypertensive agents. Non-limiting examples include angiotensin converting enzyme inhibitors (e.g., captopril, zofenopril, fbsinopril, enalapril, ceranopril, cilazopril, delapril, pentopril, quinapril, ramipril, lisinopril), angiotensin II antagonists (e.g., candesartan cilexetil, candesartan, losartan, losartan potassium, eprosartan, valsartan, telmisartan, irbesartan, tasosartan, olmesartan, olmesartan medoxomil, azilsartan, azilsartan medoxomil), calcium antagonists (e.g., manidipine, nifedipine, amlodipine, efonidipine, nicardipine, cilnidipine) and_β-blockers (e.g., metoprolol, atenolol, propranolol, carvedilol, pindolol). Further non-limiting examples of antihypertensive agents include: diruetics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, torsemide, furosemide, musolimine, bumetanide, triamtrenene, amiloride, spironolactone), alpha adrenergic blockers, beta adrenergic blockers, calcium channel blockers (e.g., diltiazem, verapamil, nifedipine and amlodipine), vasodilators (e.g., hydralazine), renin inhibitors, AT-1 receptor antagonists (e.g., losartan, irbesartan, valsartan), ET receptor antagonists (e.g., sitaxsentan, atrsentan, compounds disclosed in U.S. Pat. Nos. 5,612,359 and 6,043,265), dual ET/AII antagonist (e.g., compounds disclosed in WO2000/01389), neutral endopeptidase (NEP) inhibitors, If channel blocker ivabradinand, vasopepsidase inhibitors (dual NEP-ACE inhibitors) (e.g., gemopatrilat and nitrates).

In some embodiments, the one or more additional therapeutic agents include those useful, for example, as diuretics. Non-limiting examples include_xanthine derivatives (e.g., theobromine sodium salicylate, theobromine calcium salicylate), thiazide preparations (e.g., ethiazide, cyclopenthiazide, trichloromethiazide, hydrochlorothiazide, hydroflumethiazide, benzylhydrochlorothiazide, penfluthiazide, polythiazide, methyclothiazide), antialdosterone preparations (e.g., spironolactone, triamterene), carbonic anhydrase inhibitors (e.g., acetazolamide) and chlorobenzenesulfonamide agents (e.g., chlortalidone, mefruside, indapamide).

In some embodiments, the one or more additional therapeutic agents include those useful, for example, as immunotherapeutic agents. Non-limiting examples include microbial or bacterial compounds (e.g., muramyl dipeptide derivative, picibanil), polysaccharides having immunoenhancing activity (e.g., lentinan, sizofiran, krestin), cytokines obtained by genetic engineering approaches (e.g., interferon, interleukin (IL) such as IL-1, IL-2, IL-12), and colony-stimulating factors (e.g., granulocyte colony-stimulating factor, erythropoietin).

In some embodiments, the one or more additional therapeutic agents include those useful, for example, as anti-thrombotic agents. Non-limiting examples include heparins (e.g., heparin sodium, heparin calcium, enoxaparin sodium, dalteparin sodium) warfarin (e.g., warfarin potassium); anti-thrombin drugs (e.g., aragatroban, dabigatran, boroarginine derivatives, boropeptides, heparins, hirudin, and melagatran) FXa inhibitors (e.g., rivaroxaban, apixaban, edoxaban, YM150, compounds described in WO02/06234, WO2004/048363, WO2005/030740, WO2005/058823, and WO2005/113504) thrombolytic agents (e.g., anistreplase, streptokinase, tenecteplase (TNK), lanoteplase (nPA), urokinase, tisokinase, alteplase, nateplase, monteplase, pamiteplase, factor VIla inhibitors, PAI-1 inhibitors, alpha2-antiplasmin inhibitors, and anisoylated plasminogen streptokinase activator complex), and platelet aggregation inhibitors (e.g., ticlopidine hydrochloride, clopidogrel, prasugrel, E5555, SHC530348, cilostazol, ethyl icosapentate, beraprost sodium, and sarpogrelate hydrochloride).

In some embodiments, the one or more additional therapeutic agents include those useful, for example, for treating osteoporosis. Non-limiting examples include alfacalcidol, calcitriol, elcatonin, calcitonin salmon, estriol, ipriflavone, pamidronate disodium, alendronate sodium hydrate, incadronate disodium, and risedronate disodium. Suitable examples of vitamins include vitamin B1 and vitamin B12. Suitable examples of erectile dysfunction drugs include apomorphine and sildenafil citrate. Suitable examples of therapeutic agents for urinary frequency or urinary incontinence include flavorxate hydrochloride, oxybutynin hydrochloride and propiverine hydrochloride. Suitable examples of therapeutic agents for dysuria include acetylcholine esterase inhibitors (e.g., distigmine). Suitable examples of anti-inflammatory agents include nonsteroidal anti-inflammatory drugs such as aspirin, acetaminophen, indomethacin.

Other exemplary additional therapeutic agents include agents that modulate hepatic glucose balance (e.g., fructose 1,6-bisphosphatase inhibitors, glycogen phosphorylase inhibitors, glycogen synthase kinase inhibitors, glucokinase activators), agents designed to treat the complications of prolonged hyperglycemia, such as aldose reductase inhibitors (e.g. epalrestat and ranirestat), agents used to treat complications related to micro-angiopathies, anti-dyslipidemia agents, such as HMG-CoA reductase inhibitors (statins, e.g. rosuvastatin pravastatin, pitavastatin, lovastatin, atorvastatin, simvastatin, fluvastatin, itavastatin, ZD-4522), HMG-CoA synthase inhibitors, cholesterol-lowering agents, bile acid sequestrants (e.g., cholestyramine, questran, colestipol, and colesevelam), cholesterol absorption inhibitors (e.g. plant sterols such as phytosterols), cholesteryl ester transfer protein (CETP) inhibitors, inhibitors of the ileal bile acid transport system (IBAT inhibitors), diacylglyceryl acyltransferase 1 (DGAT1) inhibitors (e.g., AZD7687, LCQ908, compounds described in WO2009/016462, WO2010/086820), monoacylglycerol O-acyltransferase inhibitors, α-amylase inhibitors (e.g., tendamistat, trestatin, AL-3688), α-glucoside hydrolase inhibitors, SIRT-1 activators, c-Jun N-terminal kinase (JNK) inhibitors, a VPAC2 receptor agonist, TGR5 receptor modulators (e.g., compounds described in ), GPBAR1 receptor modulators, GPR120 modulators, high affinity nicotinic acid receptor (HM74A) activators, carnitine palmitoyl transferase enzyme inhibitors, mineralocorticoid receptor inhibitors, inhibitors of TORC2, fatty acid synthetase inhibitors, serine palmitoyl transferase inhibitors, GPR81 modulators, GPR39 modulators, GPR43 modulators, GPR41 modulators, GPR105 modulators, Kv1.3 modulators, retinol binding protein 4 modulators, somatostain receptor modulators, PDHK2 modulators, PDHK4 modulators, MAP4K4 inhibitors, IL1 family modulators (e.g., ILI beta modulators), ACAT inhibitors, MTP inhibitors (e.g., diriotapide, mitratapide, and implitapide), lipooxygenase inhibitors, PCSK9 modulators (e.g., alirocumab and evolocumab), RXRalpha modulators, cysteamine, cystamine, an RNA antisense construct to inhibit protein tyrosine phosphatase PTPRU, vitamin B complex, pentraxin proteins, a protein tyrosine phosphatase-1 B (PTP-1 B) inhibitor (e.g., trodusquemine, hyrtiosal extract, and compounds described by Zhang et al. Drug Discovery Today. 2007, 12(9-10): 373-381), ezitimbe, betaine, pentoxifylline, alpha delta-9 desaturase, BCKDK inhibitors, branched-chain alpha keto acid dehydrogenase kinase (BCBK) inhibitors, PNPLA3 inhibitors, FGF1 9 analogs, SCD1 inhibitors, bile acid binding resins, nicotinic acid (niacin) and analogues thereof, anti-oxidants (e.g., probucol), omega-3 fatty acids, antihypertensive agents, including adrenergic receptor antagonists, such as beta blockers (e.g. atenolol), alpha blockers (e.g. doxazosin), and mixed alpha/beta blockers (e.g. labetalol), adrenergic receptor agonists, including alpha-2 agonists (e.g. clonidine), angiotensin converting enzyme (ACE) inhibitors (e.g. lisinopril), calcium channel blockers, such as dihydropridines (e.g. nifedipine), phenylalkylamines (e.g. verapamil), and benzothiazepines (e.g. diltiazem), angiotensin II receptor antagonists (e.g. candesartan), aldosterone receptor antagonists (e.g. eplerenone, spironolactone), centrally acting adrenergic drugs, such as central alpha agonists (e.g. clonidine), diuretic agents (e.g. furosemide, torsemide, bemetanide, ethacrynic acid, thiazide-type diuretics (e.g., chlorothiazide, hydrochlorothiazide, benzthiazide, hydroflumethiazide, bendroflumethiazide, methychlorthiazide, polythiazide, trichlormethiazide, indapamide), phthalimidine-type diuretics (e.g., chlorthalidone, metolazone), quinazoline-type diuretics (e.g., quinethazone), potassium-sparing diuretics (e.g., triamterene and amiloride), thyroid receptor agonists (e.g., compounds described in WO2020/117987), haemostasis modulators, including antithrombotics (e.g., activators of fibrinolysis), thrombin antagonists, factor VIIa inhibitors, anticoagulants (e.g., vitamin K antagonists such as warfarin), heparin and low molecular weight analogues thereof, factor Xa inhibitors, and direct thrombin inhibitors (e.g. argatroban), antiplatelet agents (e.g., cyclooxygenase inhibitors (e.g. aspirin), non-steroidal anti-inflammatory drugs (NSAIDS), thromboxane-A2-receptor antagonists (e.g., ifetroban), thromboxane-A2-synthetase inhibitors, PDE inhibitors (e.g., Pletal, dipyridamole)), antagonists of purinergic receptors (e.g., P2Y1 and P2Y12), adenosine diphosphate (ADP) receptor inhibitors (e.g. clopidogrel), phosphodiesterase inhibitors (e.g. cilostazol), glycoprotein IIB/IIA inhibitors (e.g. tirofiban, eptifibatide, and abcixima), adenosine reuptake inhibitors (e.g. dipyridamole), noradrenergic agents (e.g. phentermine), serotonergic agents (e.g. sibutramine, lorcaserin), diacyl glycerolacyltransferase (DGAT) inhibitors, feeding behavior modifying agents, pyruvate dehydrogenase kinase (PDK) modulators, serotonin receptor modulators, monoamine transmission-modulating agents, such as selective serotonin reuptake inhibitors (SSRI) (e.g. fluoxetine), noradrenaline reuptake inhibitors (NARI), noradrenaline-serotonin reuptake inhibitors (SNRI), and monoamine oxidase inhibitors (MAOI) (e.g. toloxatone and amiflamine), compounds described in WO007/013694, WO2007/018314, WO2008/093639 and WO2008/099794, GPR40 agonists (e.g., fasiglifam or a hydrate thereof, compounds described in WO2004/041266, WO2004/106276, WO2005/063729,WO2005/063725, WO2005/087710, WO2005/095338, WO2007/013689 and WO2008/001931), SGLT1 inhibitors, adiponectin or agonist thereof, IKK inhibitors (e.g., AS-2868), somatostatin receptor agonists, ACC2 inhibitors, cachexia-ameliorating agents, such as a cyclooxygenase inhibitors (e.g., indomethacin), progesterone derivatives (e.g., megestrol acetate), glucocorticoids (e.g., dexamethasone), metoclopramide agents, tetrahydrocannabinol agents, agents for improving fat metabolism (e.g., eicosapentaenoic acid), growth hormones, IGF-1, antibodies against a cachexia-inducing factor TNF-α, LIF, IL-6, and oncostatin M, metabolism-modifying proteins or peptides such as glucokinase (GK), glucokinase regulatory protein (GKRP), uncoupling proteins 2 and 3 (UCP2 and UCP3), peroxisome proliferator-activated receptor α (PPARα), MC4r agonists, insulin receptor agonist, PDE 5 inhibitors, glycation inhibitors (e.g., ALT-711), nerve regeneration-promoting drugs (e.g., Y-128, VX853, prosaptide), antidepressants (e.g., desipramine, amitriptyline, imipramine), antiepileptic drugs (e.g., lamotrigine, trileptal, keppra, zonegran, pregabalin, harkoseride, carbamazepine), antiarrhythmic drugs (e.g., K⁺ channel openers, mexiletine, propafenone, metoprolol, atenolol, carvadiol, propranolol, sotalol, dofetilide, amiodarone, azimilide, ibutilide, ditiazem, and verapamil), acetylcholine receptor ligands (e.g., ABT-594), endothelin receptor antagonists (e.g., ABT-627), narcotic analgesics (e.g., morphine), α2 receptor agonists (e.g., clonidine), local analgesics (e.g., capsaicin), antianxiety drugs (e.g., benzothiazepine), phosphodiesterase inhibitors (e.g., sildenafil), dopamine receptor agonists (e.g., apomorphine), cytotoxic antibodies (e.g., T-cell receptor and IL-2 receptor-specific antibodies), B cell depleting therapies (e.g., anti-CD20 antibody (e.g., rituxan), i-BLyS antibody), drugs affecting T cell migration (e.g., anti-integrin alpha 4/beta 1 antibody (e.g., tysabri), drugs that act on immunophilins (e.g., cyclosporine, tacrolimus, sirolimus, rapamicin), interferons (e.g., IFN-β), immunomodulators (e.g., glatiramer), TNF-binding proteins (e.g., circulating receptors), immunosupressants (e.g., mycophenolate), metaglidasen, AMG-131, balaglitazone, MBX-2044, rivoglitazone, aleglitazar, chiglitazar, saroglitazar, muraglitazar, tesaglitazar, lobeglitazone, PLX-204, PN-2034, GFT-505, THR-0921, exenatide, exendin-4, memantine, midazolam, ketoconazole, ethyl icosapentate, clonidine, azosemide, isosorbide, ethacrynic acid, piretanide, bumetanide, etoposide, piroxicam, NO donating agents (e.g., organonitrates), NO promoting agents (e.g., phosphodiesterase inhibitors).

In some embodiments, the additional therapeutic agent or regimen is administered to the patient prior to contacting with or administering the compounds and pharmaceutical compositions (e.g., about one hour prior, or about 6 hours prior, or about 12 hours prior, or about 24 hours prior, or about 48 hours prior, or about 1 week prior, or about 1 month prior).

In some embodiments, the additional therapeutic agent or regimen is administered to the patient at about the same time as contacting with or administering the compounds and pharmaceutical compositions. By way of example, the additional therapeutic agent or regimen and the compounds and pharmaceutical compositions are provided to the patient simultaneously in the same dosage form. As another example, the additional therapeutic agent or regimen and the compounds and pharmaceutical compositions are provided to the patient concurrently in separate dosage forms.

In some embodiments, the methods described herein further include the step of identifying a patient (e.g., a subject) in need of such treatment (e.g., by way of blood assay, body mass index, or other conventional method known in the art).

In some embodiments, the methods described herein further include the step of identifying a patient (e.g., patient) that has a disease, disorder, or condition as provided here (e.g., a GLP-1 associated disease, disorder, or condition).

In some embodiments, the methods described herein further include the step of identifying a patient (e.g., patient) that has type 2 diabetes mellitus. In some embodiments, determining if the patient has type 2 diabetes mellitus includes performing an assay to determine the level of hemoglobin Alc (HbAlc), fasting plasma glucose, non-fasting plasma glucose, or any combination thereof. In some embodiments, the level of HbAlc is about 6.5% to about 24.0%. In some embodiments, the level of HbAlc is greater than or about 6.5%. In some embodiments, the level of HbAlc is greater than or about 8.0%. In some embodiments, the level of HbAlc is greater than or about 10.0%. In some embodiments, the level of HbAlc is greater than or about 12.0%. In some embodiments, the level of HbAlc is greater than or about 14.0%. In some embodiments, the level of HbAlc is greater than or about 16.0%. In some embodiments, the level of HbAlc is greater than or about 18.0%. In some embodiments, the level of HbAlc is greater than or about 20.0%. In some embodiments, the level of HbAlc is greater than or about 22.0%. In some embodiments, the level of HbAlc is greater than or about 24.0%.

In some embodiments, the level of fasting plasma glucose is greater than or about 120 mg/dL to greater than or about 750 mg/dL. In some embodiments, the level of fasting plasma glucose is greater than or about 200 mg/dL to greater than or about 500 mg/dL. In some embodiments, the level of fasting plasma glucose is greater than or about 300 mg/dL to greater than or about 700 mg/dL.

In some embodiments, the level of non-fasting plasma glucose is greater than or about 190 mg/dL to greater than or about 750 mg/dL. In some embodiments, the level of non-fasting plasma glucose is greater than or about 250 mg/dL to greater than or about 450 mg/dL. In some embodiments, the level of non-fasting plasma glucose is greater than or about 400 mg/dL to greater than or about 700 mg/dL.

In some embodiments, determining if the patient has type 2 diabetes mellitus further includes determining the patient’s BMI. In some embodiments, the BMI of the patient is greater than or about 22 kg/m² to greater than or about 100 kg/m². In some embodiments, the BMI of the patient is greater than or about 30 kg/m² to greater than or about 90 kg/m². In some embodiments, the BMI of the patient is greater than or about 40 kg/m² to greater than or about 80 kg/m². In some embodiments, the BMI of the patient is greater than or about 50 kg/m² to greater than or about 70 kg/m².

In some embodiments, additional factors (e.g. risk factors) used for determining if the patient has type 2 diabetes mellitus further includes age and ethnicity of the patient. In some embodiments, the patient’s age is greater than or about 10 years. In some embodiments, the patient’s age is greater than or about 15 years. In some embodiments, the patient’s age is greater than or about 20 years. In some embodiments, the patient’s age is greater than or about 25 years. In some embodiments, the patient’s age is greater than or about 30 years. In some embodiments, the patient’s age is greater than or about 35 years. In some embodiments, the patient’s age is greater than or about 40 years. In some embodiments, the patient’s age is greater than or about 42 years. In some embodiments, the patient’s age is greater than or about 44 years. In some embodiments, the patient’s age is greater than or about 46 years. In some embodiments, the patient’s age is greater than or about 48 years. In some embodiments, the patient’s age is greater than or about 50 years. In some embodiments, the patient’s age is greater than or about 52 years. In some embodiments, the patient’s age is greater than or about 54 years. In some embodiments, the patient’s age is greater than or about 56 years. In some embodiments, the patient’s age is greater than or about 58 years. In some embodiments, the patient’s age is greater than or about 60 years. In some embodiments, the patient’s age is greater than or about 62 years. In some embodiments, the patient’s age is greater than or about 64 years. In some embodiments, the patient’s age is greater than or about 66 years. In some embodiments, the patient’s age is greater than or about 68 years. In some embodiments, the patient’s age is greater than or about 70 years. In some embodiments, the patient’s age is greater than or about 72 years. In some embodiments, the patient’s age is greater than or about 74 years. In some embodiments, the patient’s age is greater than or about 76 years. In some embodiments, the patient’s age is greater than or about 78 years. In some embodiments, the patient’s age is greater than or about 80 years. In some embodiments, the patient’s age is greater than or about 85 years. In some embodiments, the patient’s age is greater than or about 90 years. In some embodiments, the patient’s age is greater than or about 95 years. In some embodiments, the ethnicity of the patient may be African American, American Indian or Alaska Native, Asian American, Hispanics or Latinos, or Native Hawaiian or Pacific Islander.

In some embodiments, the patient is a pediatric patient. The term “pediatric patient” as used herein refers to a patient under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman RE, Kliegman R, Arvin AM, Nelson WE. Nelson Textbook of Pediatrics, 15th Ed. Philadelphia: W.B. Saunders Company, 1996; Rudolph AM, et al. Rudolph’s Pediatrics, 21st Ed. New York: McGraw-Hill, 2002; and Avery MD, First LR. Pediatric Medicine, 2nd Ed. Baltimore: Williams & Wilkins; 1994. In some embodiments, a pediatric patient is from birth through the first 28 days of life, from 29 days of age to less than two years of age, from two years of age to less than 12 years of age, or 12 years of age through 21 years of age (up to, but not including, the twenty-second birthday). In some embodiments, a pediatric patient is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age. In some embodiments, the patient is an adult patient.

### EXAMPLES

The invention is further described in the following examples, which do not limit the scope of the invention described in the claims.

General information: All evaporations were carried out in vacuo with a rotary evaporator. Analytical samples were dried in vacuo (1-5 mmHg) at rt. Thin layer chromatography (TLC) was performed on silica gel plates, spots were visualized by UV light (214 and 254 nm). Purification by column and flash chromatography was carried out using silica gel (200-400 mesh). Solvent systems were reported as mixtures by volume. All NMR spectra were recorded on a Bruker 400 or VARIAN (400 MHz) spectrometer. 1 H chemical shifts were reported in δ values in ppm with the deuterated solvent as the internal standard. Data were reported as follows: chemical shift, multiplicity (s = singlet, d = doublet, t = triplet, q = quartet, br = broad, m = multiplet), coupling constant (Hz), integration. LCMS spectra were obtained on an Agilent 1200 series 6110 or 6120 mass spectrometer with electrospray ionization and excepted as otherwise indicated, the general LCMS condition was as follows: Waters X Bridge C18 column (50 mm*4.6 mm*3.5 um), Flow Rate: 2.0 mL/min, the column temperature: 40° C.

Example 1: 2-((4-(6-((4-chloro-2-fluorobenzyl)oxy)pyridin-2-yl)cyclohex-3-en-1-yl)methyl)-3-(((S)-oxetan-2-yl)methyl)-3H-imidazo[4,5-b]pyridine-5-carboxylic acid (Compound 102a)

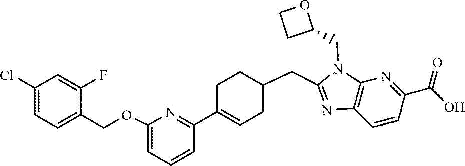 [SMILES: O=C(O)c1ccc2nc(CC3CC=C(c4cccc(OCc5ccc(Cl)cc5F)n4)CC3)n(C[C@@H]3CCO3)c2n1]

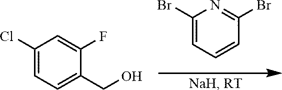

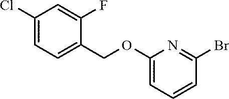 [SMILES: Fc1cc(Cl)ccc1COc1cccc(Br)n1]

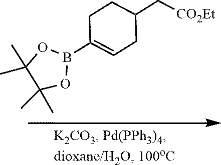

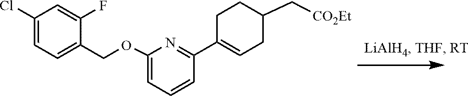

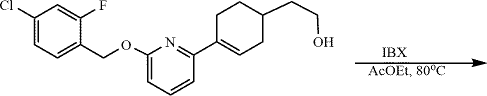

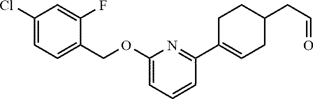 [SMILES: O=CCC1CC=C(c2cccc(OCc3ccc(Cl)cc3F)n2)CC1]

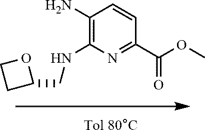

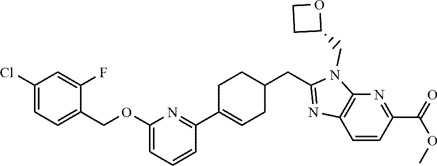 [SMILES: COC(=O)c1ccc2nc(CC3CC=C(c4cccc(OCc5ccc(Cl)cc5F)n4)CC3)n(C[C@@H]3CCO3)c2n1]

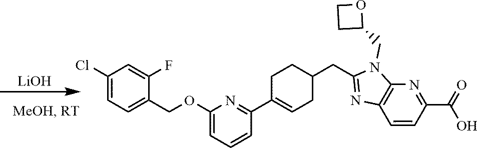

### Step A: The Synthesis of 2-Bromo-6-((4-Chloro-2-Fluorobenzyl)Oxy)Pyridine

To a suspension of NaH (4.2 g, 108 mmol) in dried THF (200 mL) was added (4-chloro-2-fluorophenyl)methanol (17.2 g, 108 mmol) at 0° C. under N₂. The mixture was stirred at room temperature for 30 min. Then 2,6-dibromopyridine (21.2 g, 90 mmol) was added at 0° C. The mixture was stirred at room temperature for 16 h. After the reaction was completed, the mixture was quenched with water and extracted with ethyl acetate (50 ml x 3), washed with brine (50 ml x 3), dried over sodium sulfate, filtered, and concentrated in vacuum, the residue was purified by column chromatography to give 2-bromo-6-((4-chloro-2-fluorobenzyl)oxy)pyridine (27 g, yield: 94%) as white solid. MS Calcd.: 314.9; MS Found: 316.0 [M+H]⁺.

### Step B: The Synthesis of Ethyl 2-(4-(6-((4-Chloro-2-Fluorobenzyl)Oxy)Pyridin-2-yl)Cyclohex-3-en-1-yl)Acetate

A mixture of 2-bromo-6-((4-chloro-2-fluorobenzyl)oxy)pyridine (400 mg, 1.26 mmol), Pd(PPh₃)₄ (15 mg, 0.12 mmol), ethyl 2-(4-(4,4,5,5-tetramethyl-1,3,2-dioxaborolan-2-yl)cyclohex-3-en-1-yl) acetate (483 mg, 1.64 mmol) and potassium carbonate (349 mg, 2.53 mmol) in dioxane (5 ml) and water (1 ml) was stirred 100° C. for 12 hours under nitrogen atmosphere. The mixture was poured into cold water and extracted with EtOAc (3 x 15 ml). The combined organic layer was washed with water (30 ml), dried over sodium sulfate, filtered, and concentrated under reduced pressure; the residue was purified by silica gel column chromatography to afford ethyl 2-(4-(6-((4-chloro-2-fluorobenzyl)oxy)pyridin-2-yl)cyclohex-3-en-1-yl)acetate (490 mg, yield: 82%) as yellow oil. MS Calcd.: 403.1; MS Found: 404.2 [M+H]⁺.

### Step C: The Synthesis of 2-(4-(6-((4-Chloro-2-Fluorobenzyl)Oxy)Pyridin-2-yl)Cyclohex-3-en-1-yl) Ethan-1-ol

To a two-neck RBF was added LiAlH₄ (188 mg, 4.95 mmol) under N₂. Then a solution of ethyl 2-(4-(6-((4-chloro-2-fluorobenzyl)oxy)pyridin-2-yl)cyclohex-3-en-1-yl)acetate (500 mg, 1.24 mmol) in THF (5 ml) was added at 0° C. The resulting mixture was stirred at room temperature for 2 hours. The reaction was quenched with H₂O— NaOH (aq., 15%): H₂O=1:3:1. The resulting mixture was filtered, and the filtrate was extracted with EtOAc (15 ml x 3). The combined EtOAc layers were washed with brine (10 ml x 3), dried over sodium sulfate, filtered, and concentrated in vacuo. The residue was purified by silica gel to give 2-(4-(6-((4-chloro-2-fluorobenzyl)oxy)pyridin-2-yl)cyclohex-3- en-1-yl)ethan-1-ol (170 mg, yield: 40%) as yellow oil. MS Calcd.: 361.1; MS Found: 362.0 [M+H]⁺.

### Step D: The Synthesis of 2-(4-(6-((4-Chloro-2-Fluorobenzyl)Oxy)Pyridin-2-yl)Cyclohex-3-en-1-yl) Acetaldehyde

### Step E: The Synthesis of Methyl 2-((4-(6-((4-Chloro-2-Fluorobenzyl)Oxy)Pyridin-2-yl)Cyclohex- 3-en-1-yl)Methyl)-3-(((S)-Oxetan-2-yl)Methyl)-3H-Imidazo[4,5-b]Pyridine-5-carboxylate

A mixture of 2-(4-(6-((4-chloro-2-fluorobenzyl)oxy)pyridin-2-yl)cyclohex-3-en-1-yl)acetaldehyde (200 mg, 0.58 mmol) and methyl (S)-5-amino-6-((oxetan-2-ylmethyl)amino)picolinate (138 mg, 0.58 mmol) were mixed in toluene (5 ml). The mixture was stirred at 80° C. for 16 hours and then concentrated in vacuo. The resulting residue was purified by silica gel chromatography to give methyl 2-((4-(6-((4-chloro-2-fluorobenzyl)oxy)pyridin-2-yl)cyclohex-3-en-1-yl)methyl)-3-(((S)-oxetan-2-yl)methyl)-3H-imidazo[4,5-b]pyridine-5-carboxylate (150 mg, yield: 45%) as yellow oil. MS Calcd.: 576.2; MS Found: 577.2 [M+H]⁺.

### Step F: The Synthesis of 2-((4-(6-((4-Chloro-2-Fluorobenzyl)Oxy)Pyridin-2-yl)Cyclohex-3-en-1-yl) Methyl)-3-(((S)-Oxetan-2-yl)Methyl)-3H-Imidazo[4,5-b]Pyridine-5-Carboxylic Acid

A mixture of methyl 2-((4-(6-((4-chloro-2-fluorobenzyl)oxy)pyridin-2-yl)cyclohex-3-en-1-yl) methyl)-3-(((S)-oxetan-2-yl)methyl)-3H-imidazo[4,5-b]pyridine-5-carboxylate (150 mg, 0.26 mmol) and lithium hydroxide (12 mg, 0.52 mmol) in methanol (3 ml) and water (0.5 ml) was stirred at room temperature for 3 hours. The reaction mixture was purified by prep-HPLC directly to give 2-((4-(6-((4-chloro-2-fluorobenzyl)oxy)pyridin-2-yl)cyclohex-3-en-1-yl)methyl)-3-(((S)-oxetan-2-yl)methyl)-3H-imidazo[4,5-b]pyridine-5-carboxylic acid (49.2 mg, yield: 34%) as white solid. MS Calcd.: 562.2; MS Found: 563.1 [M+H]⁺.

¹H NMR (400 MHz, CD₃OD) δ 8.13 (d, J= 8.2 Hz, 1 H), 8.06 (d, J= 8.2 Hz, 1 H), 7.60 (t, J= 7.8 Hz, 1 H), 7.50 (t, J= 8.0 Hz, 1 H), 7.22 (dd, J= 14.2, 4.8 Hz, 2 H), 7.05 (d, J= 7.4 Hz, 1 H), 6.77 (s, 1 H), 6.66 (d, J= 8.1 Hz, 1 H), 5.44 (s, 2 H), 5.27 (d, J= 7.0 Hz, 1 H), 4.71 (ddd, J= 47.7, 25.0, 7.8 Hz, 3 H), 4.47 - 4.38 (m, 1 H), 3.18 (dd, J= 15.6, 8.6 Hz, 2 H), 2.83 - 2.66 (m, 2 H), 2.57 - 2.41 (m, 4 H), 2.11 (m, 2 H), 1.61 (d, J= 6.7 Hz, 1 H).

Example 2: 2-((4-(6-((4-chloro-2-fluorobenzyl)oxy)pyridin-2-yl)cyclohex-3-en-1-yl)methyl)-3-(((S)-oxetan-2-yl)methyl)-3H-imidazo[4,5-b]pyridine-5-carboxylic acid diastereomer-1 (Compound 102b)

Example 3: 2-((4-(6-((4-chloro-2-fluorobenzyl)oxy)pyridin-2-yl)cyclohex-3-en-1-yl)methyl)-3-(((S)-oxetan-2-yl)methyl)-3H-imidazo[4,5-b]pyridine-5-carboxylic acid diastereomer-2 (Compound 102c)

Separation of Compound 102a (400 mg) into the component diastereomers as the cyclohexene was carried out via supercritical fluid chromatography [Instrument: (Gilson-281, Column: IG 20*250, 10 um, Mobile Phase: n-Hexane (0.1%FA): EtOH (0.1%FA)= 7:3]. The first eluting diastereomer was assigned as Compound 102b. It was further purified using reversed-phase HPLC [column: SunFire C18, 10um,Mobile phase A: 0.05% ammonium bicarbonate, Mobile phase B: Acetonitrile, 20-70% B in 8 min, stop at 16 min]. Yield: 80 mg, 20%. LCMS: m/z 563.1 [M+H]⁺.

¹H NMR (400 MHz, CD₃OD) δ 8.08 (d, J= 8.2 Hz, 1H), 8.01 (d, J= 8.2 Hz, 1 H), 7.67 -7.57 (m, 1 H), 7.51 (t, J= 8.0 Hz, 1 H), 7.29 - 7.15 (m, 2 H), 7.05 (d, J= 7.4 Hz, 1 H), 6.77 (s, 1 H), 6.66 (d, J= 8.1 Hz, 1 H), 5.44 (s, 2 H), 5.35 - 5.23 (m, 1 H), 4.81 - 4.67 (m, 2 H), 4.64 - 4.55 (m, 1 H), 4.49 - 4.36 (m, 1 H), 3.17 (d, J= 6.9 Hz, 2 H), 2.86 - 2.74 (m, 1 H), 2.73 - 2.62 (m, 1 H), 2.60 - 2.38 (m, 4 H), 2.23 - 2.00 (m, 2 H), 1.68 - 1.52 (m, 1 H).

The second eluting diastereomer was designated as Compound 102c. It was further purified using reversed-phase HPLC [column: SunFire C18, 10um, Mobile phase A: 0.05% ammonium bicarbonate, Mobile phase B: Acetonitrile, 20-70% B in 8 min, stop at 16 min]. Yield: 53 mg, 13%. LCMS: m/z 563.1 [M+H]⁺.

¹H NMR (400 MHz, CD₃OD) δ 8.16 (d, J= 8.3 Hz, 1 H), 8.09 (d, J= 8.3 Hz, 1 H), 7.67 -7.55 (m, 1 H), 7.51 (t, J= 7.9 Hz, 1 H),7.29 - 7.15 (m, 2 H), 7.05 (d, J= 7.4 Hz, 1 H), 6.78 (s, 1 H), 6.66 (d, J= 8.1 Hz, 1 H), 5.44 (s, 2 H), 5.31 - 5.19 (m, 1 H), 4.85 - 4.68 (m, 2 H), 4.65 - 4.53 (m, 1 H), 4.41 (dt, J= 9.1, 6.0 Hz, 1 H), 3.28 - 3.10 (m, 2 H), 2.85 - 2.75 (m, 1 H), 2.74 - 2.62 (m, 1 H), 2.60 - 2.43 (m, 4 H), 2.23 - 1.99 (m, 2 H), 1.67 - 1.55 (m, 1 H).

Example 4: 2-((4-(6-((4-cyano-2-fluorobenzyl)oxy)pyridin-2-yl)cyclohex-3-en-1-yl)methyl)-3-(((S)-oxetan-2-yl)methyl)-3H-imidazo[4,5-b]pyridine-5-carboxylic acid (Compound 101a)

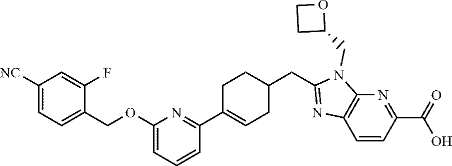

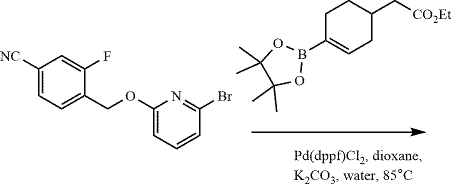

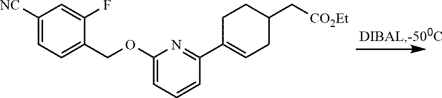

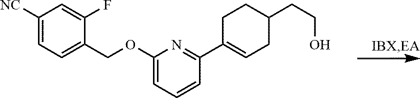

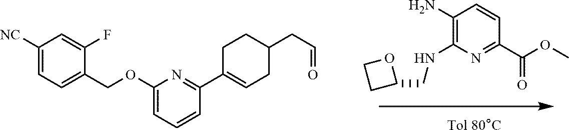

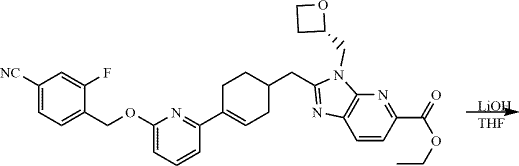

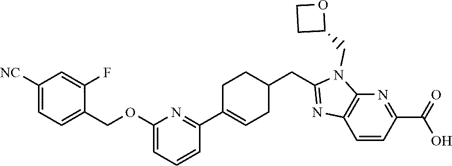

### Step A: The Synthesis of Ethyl 2-(4-(6-((4-Cyano-2-Fluorobenzyl)Oxy)Pyridin-2-yl)Cyclohex-3- en-1-vl)Acetate

A mixture of 4-(((6-bromopyridin-2-yl)oxy)methyl)-3-fluorobenzonitrile (1.2 g, 4 mmol), ethyl 2-(4-(4,4,5,5-tetramethyl-1,3,2-dioxaborolan-2-yl)cyclohex-3-en-1-yl)acetate (1.2 g, 4 mmol), K₂CO₃ (1.1 g, 8 mmol) in dioxane (5 mL) and water (1 mL) was degassed with N₂ for 10 min. Pd(dppf)Cl₂ (330 mg, 0.4 mmol) was then added. The mixture was stirred at 85⁰C for 15 h, and the resulting mixture was filtered. The filtrate was concentrated in vacuo. The residue was purified by column chromatography to give ethyl 2-(4-(6-((4-cyano-2-fluorobenzyl)oxy)pyridin-2-yl) cyclohex-3-en-1-yl)acetate (1.4 g, yield: 86.5%) as colorless oil. MS Calcd.: 394.2; MS Found: 395.1 [M+H]⁺.

### Step B: The Synthesis of 3-Fluoro-4-(((6-(4-(2-Hydroxyethyl)Cyclohex-1-en-1-yl)Pyridin-2-yl)Oxy) Methyl)Benzonitrile

To a mixture of ethyl 2-(4-(6-((4-cyano-2-fluorobenzyl)oxy)pyridin-2-yl)cyclohex-3-en-1-yl)acetate (1.1 g, 2.79 m mol) in THF (15 mL) was added DIBAL-H (9.78 mmol) at -78° C. After stirred for 4 h at -78° C., the reaction was quenched by addition of saturated ammonium chloride aqueous solution (10 mL). The mixture was extracted with EtOAc (3 x 15 mL). The combined organic layer was washed with water (30 mL), dried over sodium sulfate, filtered, and concentrated under reduced pressure. The residue was purified by column chromatography to give desired product 3-fluoro-4-(((6-(4-(2-hydroxyethyl)cyclohex-1-en-1-yl)pyridin-2-yl)oxy)methyl)benzonitrile (0.5 g, yield: 50%) as white solid. MS Calcd.: 352.2; MS Found: 353.0 [M+H]⁺.

### Step C: The Synthesis of 3-Fluoro-4-(((6-(4-(2-Oxoethyl)Cyclohex-1-en-1-yl)Pyridin-2-yl)Oxy)Methyl) Benzonitrile

To a solution of 3-fluoro-4-(((6-(4-(2-hydroxyethyl)cyclohex-1-en-1-yl)pyridin-2-yl)oxy) methyl)benzonitrile (500 mg, 1.42 mmol) in EtOAc (20 mL) were added IBX (1100 mg, 3.46 mmol) slowly. The mixture was stirred at 80° C. for 15 hours. The mixture was filtered, and the filtrate was concentrated to give crude product 3-fluoro-4-(((6-(4-(2-oxoethyl)cyclohex-1-en-1-yl)pyridin-2-yl) oxy)methyl)benzonitrile (400 mg, yield: 80%) as white solid. MS Calcd.: 350.1; MS Found: 351.0 [M+H]⁺

### Step D: The Synthesis of Ethyl 2-((4-(6-((4-Cyano-2-fluoRobenzyl)Oxy)Pyridin-2-yl) Cyclohex-3-en-1-yl)Methyl)-3-(((S)-Oxetan-2-yl)Methyl)-3H-Imidazo[4,5-b]Pyridine-5-Carboxylate

A mixture of 3-fluoro-4-(((6-(4-(2-oxoethyl)cyclohex-1-en-1-yl)pyridin-2-yl)oxy) methyl) benzonitrile (0.6 g, 1.71 mmol) and methyl (S)-5-amino-6-((oxetan-2-ylmethyl)amino)picolinate (0.4 g, 1.71 mmol) in toluene (20 mL) was stirred at 80° C. for 48 hours. The mixture was concentrated in vacuo, the residue was purified by column chromatography (silica, DCM/MeOH=20/1) to give desired product ethyl 2-((4-(6-((4-cyano-2-fluorobenzyl)oxy)pyridin-2-yl)cyclohex-3-en-1-yl) methyl)-3-(((S)-oxetan-2-yl)methyl)-3H-imidazo[4,5-b]pyridine-5-carboxylate (460 mg, yield: 46%) as brown solid. MS Calcd.: 581.2; MS Found: 582.2 [M+H]⁺.

### Step E: The Synthesis of 2-((4-(6-((4-Cyano-2-Fluorobenzyl)Oxy)Pyridin-2-yl)Cyclohex-3-en-1-yl) Methyl)-3-(((S)-Oxetan-2-yl)Methyl)-3H-Imidazo[4,5-b]Pyridine-5-Carboxylic Acid

A solution of ethyl 2-((4-(6-((4-cyano-2-fluorobenzyl)oxy)pyridin-2-yl)cyclohex-3-en-1-yl)methyl)- 3-(((S)-oxetan-2-yl)methyl)-3H-imidazo[4,5-b]pyridine-5-carboxylate (270 mg, 0.47 m mol) and lithium hydroxide (78 mg, 1.86 m mol) in THF (2 mL) and water (1 mL) was stirred at room temperature for 10 hours. The reaction mixture was purified by prep-HPLC directly to give desired product 2-((4-(6-((4-cyano-2-fluorobenzyl)oxy)pyridin-2-yl)cyclohex-3-en-1-yl)methyl) -3-(((S)oxetan-2-yl)methyl)-3H-imidazo[4,5-b]pyridine-5-carboxylic acid (120 mg, yield: 47%) as white solid. MS Calcd.: 553.2; MS Found: 554.0 [M+H]⁺.

¹H NMR (400 MHz, CD₃OD) δ 8.13 (d, J = 8.4 Hz, 1 H), 8.07 (d, J = 8.0 Hz, 1 H), 7.71-7.55 (m, 4 H), 7.07 (d, J = 8.4 Hz, 1 H), 6.74-6.70 (m, 2 H), 5.55 (s, 2 H), 5.27-5.25 (m, 1 H), 4.83-4.77 (m, 1 H), 4.72-4.68 (m, 1 H), 4.62-4.60 (m, 1 H), 4.46-4.39 (m, 1 H), 3.25-3.12 (m, 2 H), 2.83-2.78 (m, 1 H), 2.68-2.63 (m, 1 H), 2.54-2.47 (m, 4 H), 2.15- 2.05 (m, 2 H), 1.63-1.56 (m, 1 H).

Example 5: 2-((4-(6-((4-cyano-2-fluorobenzyl)oxy)pyridin-2-yl)cyclohex-3-en-1-yl)methyl)-3-(((S)-oxetan-2-yl)methyl)-3H-imidazo[4,5-b]pyridine-5-carboxylic acid diastereomer-1

Example 6: 2-((4-(6-((4-cyano-2-fluorobenzyl)oxy)pyridin-2-yl)cyclohex-3-en-1-yl)methyl)-3-(((S)-oxetan-2-yl)methyl)-3H-imidazo[4,5-b]pyridine-5-carboxylic acid diastereomer-2

Separation of Compound 101a (300 mg) into the component diastereomoers as the cyclohexene was carried out via supercritical fluid chromatography [Instrument: (Gilson-281, Column: IG 20*250, 10 um, Mobile Phase: n-Hexane (0.1%FA):EtOH (0.1%FA)= 7:3].

The first eluting diastereomer was assigned as Compound 101b. It was further purified using reversed-phase HPLC [column: SunFire C18, 10 um, Mobile phase A: 0.05% ammonium bicarbonate, Mobile phase B: Acetonitrile, 20-70% B in 8 min, stop at 16 min]. Yield: 50 mg, 17%. LCMS: m/z 554.0 [M+H]⁺.

1 H NMR (400 MHz, CD₃OD) δ 8.14 (d, J = 8.3 Hz, 1 H), 8.07 (d, J = 8.2 Hz, 1 H), 7.72 -7.50 (m, 4 H), 7.05 (d, J = 7.5 Hz, 1 H), 6.70 (d, J = 8.2 Hz, 2 H), 5.54 (s, 2 H), 5.34 - 5.20 (m, 1 H), 4.83 - 4.74 (m, 1 H), 4.74 - 4.65 (m, 1 H), 4.65 - 4.55 (m, 1 H), 4.48 - 4.36 (m, 1 H), 3.17 (d, J = 6.9 Hz, 2 H), 2.86 - 2.74 (m, 1 H), 2.69 - 2.60 (m, 1 H), 2.57 - 2.36 (m, 4 H), 2.18 - 1.99 (m, 2 H), 1.68 - 1.50 (m, 1 H).

The second eluting diastereomer was designated as Compound 101c. It was further purified using reversed-phase HPLC [column: SunFire C18, 10um, Mobile phase A: 0.05% ammonium bicarbonate, Mobile phase B: Acetonitrile, 20-70% B in 8 min, stop at 16 min]. Yield: 50 mg, 17%. LCMS: m/z 554.0 [M+H]⁺.

1H NMR (400 MHz, CD₃OD) δ 8.15 (d, J = 8.3 Hz, 1 H), 8.08 (d, J = 8.2 Hz, 1 H), 7.75 -7.50 (m, 4 H), 7.07 (d, J = 7.5 Hz, 1 H), 6.80 - 6.62 (m, 2 H), 5.55 (s, 2 H), 5.27 (d, J = 4.5 Hz, 1 H), 4.79 (dd, J = 15.0, 6.8 Hz, 1 H), 4.70 (dd, J = 14.9, 3.0 Hz, 1 H), 4.60 (dd, J = 13.8, 7.8 Hz, 1 H), 4.41 (dt, J = 9.0, 5.9 Hz, 1 H),3.18 (qd, J = 15.5, 7.0 Hz, 2 H), 2.90 -2.74 (m, 1 H), 2.65 (d, J = 16.9 Hz, 1 H), 2.48 (d, J = 13.2 Hz, 4 H), 2.24 - 1.97 (m, 2 H), 1.59 (d, J = 7.0 Hz, 1H).

Example 7: 2-((4-(4-((4-chloro-2-fluorobenzyl)oxy)pyrimidin-2-yl)cyclohex-3-en-1-yl)methyl)-3-(((S)-oxetan-2-yl)methyl)-3H-imidazo[4,5-b]pyridine-5-carboxylic acid (Compound 103a)

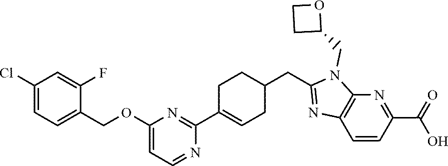 [SMILES: O=C(O)c1ccc2nc(CC3CC=C(c4nccc(OCc5ccc(Cl)cc5F)n4)CC3)n(C[C@@H]3CCO3)c2n1]

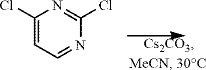

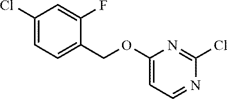 [SMILES: Fc1cc(Cl)ccc1COc1ccnc(Cl)n1]

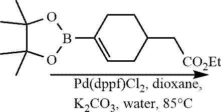

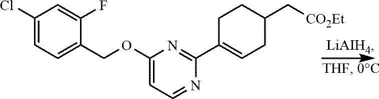

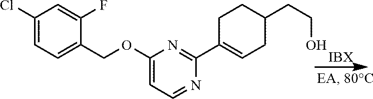

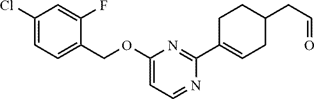 [SMILES: O=CCC1CC=C(c2nccc(OCc3ccc(Cl)cc3F)n2)CC1]

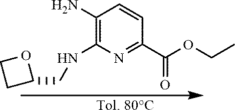

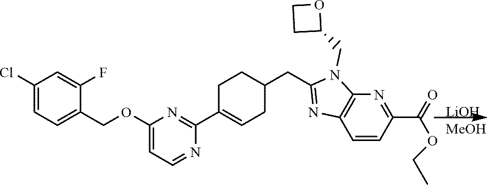

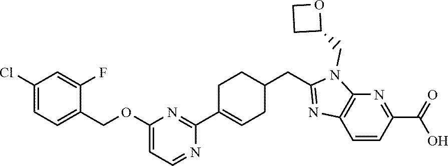 [SMILES: O=C(O)c1ccc2nc(CC3CC=C(c4nccc(OCc5ccc(Cl)cc5F)n4)CC3)n(C[C@@H]3CCO3)c2n1]

### Step A: The Synthesis of 2-Chloro-4-((4-Chloro-2-Fluorobenzyl)Oxy)Pyrimidine

To a solution of compound 2,4-dichloropyrimidine (5.0 g, 33.5 mmol) and 4-chloro-2-fluorobenzyl alcohol (5.1 g, 31.8 mmol) in CH₃CN (50 mL) was added Cs₂CO₃ (16.3 g, 50.3 mmol) in portions over 10 minutes with the cooling of ice-water. The mixture was stirred at 30° C. for 16 h. The mixture was diluted with EtOAc (50 mL) and stirred for 15 min. The mixture was filtered, and the filtrate was concentrated to dryness. The residue was diluted with a mixture of PE/EtOAc (12 mL/1 mL) and stirred at RT for 1 h. The mixture was filtered, and the filter cake was washed with PE (8 mL). The solid was then diluted with PE (8 mL) and stirred at RT for 1 h. The precipitate was collected by filter and dried to give desired product 2-chloro-4-((4-chloro-2-fluorobenzyl)oxy)pyrimidine (4.6 g, yield: 54%) as gray solid. MS Calcd.: 272.0; MS Found: 273.0 [M+H]⁺.

### Step B: The Synthesis of Ethyl 2-(4-(4-((4-Chloro-2-Fluorobenzyl)Oxy)Pyrimidin-2-yl)Cyclohex-3-en-1-yl)Acetate

A mixture of 2-chloro-4-((4-chloro-2-fluorobenzyl)oxy)pyrimidine (400 mg, 1.46 mmol), Pd(dppf)Cl₂ (15 mg, 0.12 mmol), ethyl 2-(4-(4,4,5,5-tetramethyl-1,3,2-dioxaborolan-2-yl) cyclohex-3-en-1-yl)acetate (560 mg, 1.9 mmol) and potassium carbonate (404 mg, 2.93 mmol) in dioxane (3 mL) and water (1 mL) was stirred at 85° C. for 12 hour under nitrogen atmosphere. The mixture was poured into cold water and extracted with EtOAc(3 x 15 mL). The combined organic layer was washed with water (30 mL), dried over sodium sulfate, filtered, and concentrated under reduced pressure. The resulting residue was purified by silica gel column chromatography to furnish ethyl 2-(4-(4-((4-chloro-2-fluorobenzyl)oxy)pyrimidin-2-yl)cyclohex-3-en-1-yl)acetate (400 mg, yield: 67%) as yellow oil. MS Calcd.: 404.1; MS Found: 405.1 [M+H]⁺.

### Step C: The Synthesis of 2-(4-(4-((4-Chloro-2-Fluorobenzyl)Oxy)Pyrimidin-2-yl)Cyclohex-3-en-1-yl) Ethan-1-ol

LiAlH₄ (187 mg, 4.94 mmol) was placed in a two-neck bottom under N₂. Then ethyl 2-(4-(4-((4-chloro-2-fluorobenzyl)oxy)pyrimidin-2-yl)cyclohex-3-en-1-yl)acetate (400 mg, 0.98 mmol) in THF (5 mL) was added at 0° C. The mixture was stirred at 0° C. for 2 hours. The reaction was quenched with H₂O:NaOH(aq., 15%):H₂O=1:3:1. The resulting mixture was extracted with EtOAc (15 mL x 3), washed with brine (10 mL x 3), dried over sodium sulfate, filtered, and concentrated in vacuo. The residue was purified by silica gel to give desired product 2-(4-(4-((4-chloro-2-fluorobenzyl)oxy)pyrimidin-2-yl)cyclohex-3-en-1-yl)ethan-1-ol (240 mg, yield: 67%) as yellow oil. MS Calcd.: 362.1; MS Found: 363.1 [M+H]⁺.

### Step D: The Synthesis of 2-(4-(4-((4-Chloro-2-Fluorobenzyl)Oxy)Pyrimidin-2-yl)Cyclohex-3-en- 1-yl)Acetaldehyde

A mixture of 2-(4-(4-((4-chloro-2-fluorobenzyl)oxy)pyrimidin-2-yl)cyclohex-3-en-1-yl)ethan-1-ol (240 mg, 0.6 mmol) and IBX (370 mg, 1.3 mmol) were mixed in EtOAc (5 mL). The mixture was stirred at 80° C. for 30 hours. Residual IBX was removed by filtration. The filtrate was concentrated to give crude 2-(4-(4-((4-chloro-2-fluorobenzyl)oxy)pyrimidin-2-yl)cyclohex-3-en-1-yl) acetaldehyde (330 mg, yield: 95%) as yellow oil. MS Calcd.: 360.1; MS Found: 361.1 [M+H]⁺.

### Step E: The Synthesis of Ethyl 2-((4-(4-((4-Chloro-2-Fluorobenzyl)Oxy)Pyrimidin-2-yl) Cyclohex-3-en-1-yl)Methyl)-3-(((S)-Oxetan-2-yl)Methyl)-3H-Imidazo[4,5-b]Pyridine-5-Carboxylate

A mixture of 2-(4-(4-((4-chloro-2-fluorobenzyl)oxy)pyrimidin-2-yl)cyclohex-3-en-1-yl)acetaldehyde (330 mg, 0.9 mmol) and ethyl (S)-5-amino-6-((oxetan-2-ylmethyl)amino)picolinate (230 mg, 0.9 mmol) were mixed in toluene (6 mL). The mixture was stirred at 80° C. for 16 hours and concentrated in vacuo. The resulting residue was purified by silica gel to give ethyl 2-((4-(4-((4-chloro-2-fluorobenzyl)oxy)pyrimidin-2-yl)cyclohex-3-en-1-yl)methyl)-3-(((S)-oxetan-2-yl)methyl)-3H-imidazo[4,5-b]pyridine-5-carboxylate (80 mg, yield: 15%) as yellow oil. MS Calcd.: 591.2; MS Found: 591.9 [M+H]⁺.

### Step F: The Synthesis of 2-((4-(4-((4-Chloro-2-Fluorobenzyl)Oxy)Pyrimidin-2-yl)Cyclohex-3-en-1-yl) Methyl)-3-(((S)-Oxetan-2-yl)Methyl)-3H-Imidazo[4,5-b]Pyridine-5-Carboxylic Acid

A mixture of ethyl 2-((4-(4-((4-chloro-2-fluorobenzyl)oxy)pyrimidin-2-yl)cyclohex-3-en-1- yl)methyl)-3-(((S)-oxetan-2-yl)methyl)-3H-imidazo[4,5-b]pyridine-5-carboxylate (80 mg, 0.13 mmol) and lithium hydroxide (10 mg, 0.30 mmol) in methanol (3 mL) and water (0.5 mL) was stirred at room temperature for 3 hours. The reaction mixture was purified by prep-HPLC directly to give desired product 2-((4-(4-((4-chloro-2-fluorobenzyl)oxy)pyrimidin-2-yl)cyclohex-3-en-1-yl) methyl)-3-(((S)-oxetan-2-yl)methyl)-3H-imidazo[4,5-b]pyridine-5-carboxylic acid (22.5 mg, yield: 30%) as white solid. MS Calcd.: 563.1; MS Found: 564.1 [M+H]⁺.

¹H NMR (400 MHz, CD₃OD) δ 8.40 (d, J = 5.8 Hz, 1 H), 8.13 (d, J = 8.2 Hz, 1 H), 8.07 (d, J = 8.3 Hz, 1 H), 7.53 (t, J = 8.0 Hz, 1 H), 7.25 (t, J = 9.7 Hz, 3 H), 6.72 (d, J = 5.8 Hz, 1 H), 5.53 (s, 2 H), 5.28 (d, J = 4.0 Hz, 1 H), 4.77 (dt, J = 29.5, 9.7 Hz, 2 H), 4.60 (dd, J = 14.5, 7.3 Hz, 1 H), 4.47 - 4.37 (m, 1 H), 3.26 - 3.12 (m, 2 H), 2.81 (d, J = 11.4 Hz, 2 H), 2.52 (d, J= 7.7 Hz, 4 H), 2.18 (s, 1 H), 2.07 (s, 1 H), 1.61 (s, 1 H).

Example 8: 2-((4-(2-((4-chloro-2-fluorobenzyl)oxy)pyrimidin-4-yl)cyclohex-3-en-1-yl)methyl)-3-(((S)-oxetan-2-yl)methyl)-3H-imidazo[4,5-b]pyridine-5-carboxylic acid (Compound 104a)

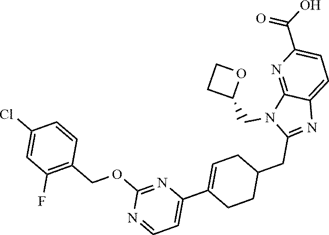 [SMILES: O=C(O)c1ccc2nc(CC3CC=C(c4ccnc(OCc5ccc(Cl)cc5F)n4)CC3)n(C[C@@H]3CCO3)c2n1]

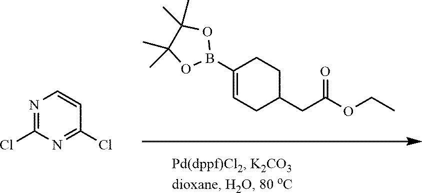

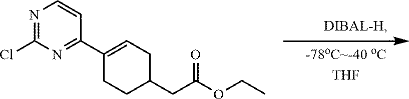

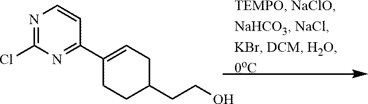

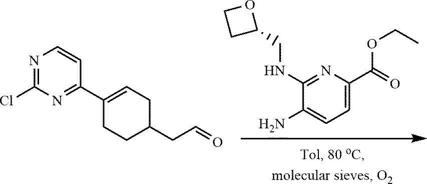

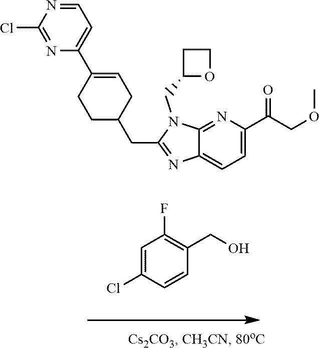

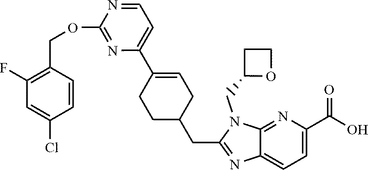 [SMILES: O=C(O)c1ccc2nc(CC3CC=C(c4ccnc(OCc5ccc(Cl)cc5F)n4)CC3)n(C[C@@H]3CCO3)c2n1]

### Step A: The Synthesis of Ethyl 2-(4-(2-Chloropyrimidin-4-yl)Cyclohex-3-en-1-yl)Acetate

To a mixture of 2,4-dichloropyrimidine (5 g, 33.5 mL), ethyl 2-(4-(4,4,5,5-tetramethyl-1,3,2- dioxaborolan-2-yl)cyclohex-3-enyl)acetate (9.87 g, 33.5 mmol) and K₂CO₃ (9.26 g, 67.1 mmol) in 1,4-dioxane (70 mL) and H₂O (15 mL) was added Pd(dppf)Cl₂ (1.23 g, 1.67 mmol). The mixture was stirred at 80° C. for 16 h under N₂. The mixture was cooled to RT and diluted with brine (40 mL). It was extracted with EtOAc (35 mL*3). The combined organic layers were dried over Na₂SO₄, filtered and concentrated. The residue was purified by flash column chromatography eluting with PE/EtOAc (12/1∼8/1) to give ethyl 2-(4-(2-chloropyrimidin-4-yl)cyclohex-3-en-1-yl)acetate (6.3 g, yield: 67.1%) as pale-yellow oil. MS Calcd.: 280.1; MS Found: 281.1 [M+H]⁺.

### Step B: The Synthesis of 2-(4-(2-Chloropyrimidin-4-yl)Cyclohex-3-en-1-yl)Ethan-1-ol

To a solution of ethyl 2-(4-(2-chloropyrimidin-4-yl)cyclohex-3-en-1-yl)acetate (4.0 g, 14.3 mmol) in THF (100 mL) was added DIBAl—H (42.9 mL, 42.9 mmol) dropwise at -78° C. The mixture was then warmed to -40° C. and stirred for 1 h. It was quenched with sat. NH₄Cl (20 mL) and stirred at 0° C. for 15 min. It was filtered and the filtrate was diluted with EtOAc (100 mL). The organic layer was separated, dried over Na₂SO₄, filtered and concentrated to give 2-(4-(2-chloropyrimidin-4-yl)cyclohex-3-en-1-yl)ethan-1-ol (3.2 g, yield: 94.1%) as colorless oil. MS Calcd.: 238.1; MS Found: 239.1 [M+H]⁺.

### Step C: The Synthesis of 2-(4-(2-Chloropyrimidin-4-yl)Cyclohex-3-en-1-yl)Acetaldehyde

To a mixture of 2-(4-(2-chloropyrimidin-4-yl)cyclohex-3-en-1-yl)ethan-1-ol (3.2 g, 13.4 mmol), TEMPO (21 mg, 0.13 mmo), NaHCO₃ (1.13 g, 13.4 mmol), NaCl (780 mg, 13.4 mmol) and KBr (160 mg, 1.34 mmol) in DCM (15 mL) and H₂O (15 mL) was added NaClO (13.9 mL, 14.1 mmol) dropwise over 30 min at 0° C. The resulting mixture was stirred at 0° C. for 30 min. The aqueous layer was separated and extracted with DCM (15 mL*3). The combined organic layers were washed with sat. Na₂S₂O₃ (25 mL), sat. NaHCO₃ (25 mL) and brine (25 mL), dried over Na₂SO₄, filtered and concentrated. The residue was purified by flash column chromatography eluting with PE/EtOAc (10/1∼6/1) to give 2-(4-(2-chloropyrimidin-4-yl)cyclohex-3-en-1-yl)acetaldehyde (2 g, yield: 63.0%) as pale-yellow oil. MS Calcd.: 236.1; MS Found: 236.9 [M+H]⁺.

### Step D: The Synthesis of Ethyl 2-((4-(2-Chloropyrimidin-4-yl)Cyclohex-3-en-1-yl)Methyl) -3-(((S)-Oxetan-2-yl)Methyl)-3H-Imidazo[4,5-b]Pyridine-5-Carboxylate

To a solution of 2-(4-(2-chloropyrimidin-4-yl)cyclohex-3-en-1-yl)acetaldehyde (2 g, 8.47 mmol) in toluene (20 mL) were added (S)-ethyl 5-amino-6-(oxetan-2-ylmethylamino)picolinate (2.13 g, 8.47 mmol) and molecular sieves (2.13 g). The mixture was stirred at 80° C. for 40 h under O₂ atmosphere. It was cooled to RT and filtered. The filtrate was concentrated, and the residue was purified by flash column chromatography eluting with DCM/MeOH (80/1~60/1) to give ethyl 2-((4-(2-chloropyrimidin-4-yl)cyclohex-3-en-1-yl)methyl)-3—(((S)—oxetan-2-yl)methyl)-3H-imidazo[4,5-b]pyridine-5-carboxylate (3.2 g, yield: 80.8 %) as yellow solid. MS Calcd.: 467.2; MS Found: 468.0 [M+H]⁺.

### Step E: The Synthesis of 2-((4-(2-((4-Chloro-2-Fluorobenzyl)Oxy)Pyrimidin-4-yl)Cyclohex-3-en-1-yl) Methyl)-3-(((S)-Oxetan-2-yl)Methyl)-3H-Imidazo[4,5-b]Pyridine-5-Carboxylic Acid

To a solution of ethyl 2-((4-(2-chloropyrimidin-4-yl)cyclohex-3-en-1-yl)methyl)-3— (((S)—oxetan-2-yl)methyl)-3H-imidazo[4,5-b]pyridine-5-carboxylate (500 mg, 1.07 mmol) in CH₃CN (15 mL) were added (4-chloro-2-fluorophenyl)methanol (214 mg, 1.34 mmol) and Cs₂CO₃ (696 mg, 2.14 mmol). The mixture was stirred at 80° C. for 16 h. It was then cooled to RT and filtered. The filtrate was concentrated. It was diluted with H₂O (5 mL) and acidified to pH=5 with AcOH solution (10%). It was then extracted with DCM (50 mL*3). The combined organic layers were dried over Na₂SO₄, filtered and concentrated. The residue was purified by prep-HPLC (high pH) to give 2-((4-(2-((4-chloro-2-fluorobenzyl)oxy)pyrimidin-4-yl)cyclohex-3-en-1-yl)methyl)-3-(((S)-oxetan-2-yl)methyl)-3H-imidazo[4,5-b]pyridine-5-carboxylic acid (13.5 mg, yield: 2.2%) as pale yellow solid. MS Calcd.: 563.2; MS Found: 564.0 [M+H]⁺.

1 H NMR (400 MHz, CD₃OD) δ 8.45 (d, J = 5.2 Hz, 1 H), 8.14 (d, J = 8.0 Hz, 1 H), 8.08 (d, J = 8.0 Hz, 1 H), 7.56 (t, J = 8.0 Hz, 1 H), 7.27∼7.21 (m, 3 H), 7.10 (s, 1 H),5.50 (s, 2 H), 5.27∼5.25 (m, 1 H), 4.82~4.77 (m, 1 H), 4.72~4.68 (m, 1 H), 4.63~4.58 (m, 1 H), 4.44∼4.40 (m, 1 H), 3.22∼3.17(m, 2 H), 2.82∼2.77 (m, 1 H), 2.72∼2.67 (m, 1 H), 2.57∼2.46 (m, 4 H), 2.21∼2.06 (m, 2 H, 1.63∼1.59 (m, 1 H).

Example 9: (S)-2-((6-((4-chloro-2-fluorobenzyl)oxy)-3′,6′-dihydro-[2,4′-bipyridin]-1′(2′H)-yl)methyl)-3-(oxetan-2-ylmethyl)-3H-imidazo[4,5-b]pyridine-5-carboxylic acid (Compound 110a)

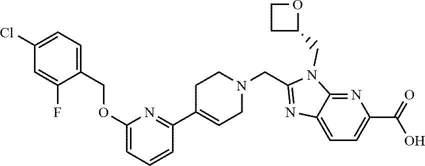 [SMILES: O=C(O)c1ccc2nc(CN3CC=C(c4cccc(OCc5ccc(Cl)cc5F)n4)CC3)n(C[C@@H]3CCO3)c2n1]

### Step A: The Synthesis of Tert-Butyl 6-((4-Chloro-2-Fluorobenzyl)Oxy)-3′,6′-Dihydro- [2,4-Bipyridine]-1 ′(2′H)-Carboxylate

A mixture of 2-bromo-6-((4-chloro-2-fluorobenzyl)oxy)pyridine (500 mg, 1.58 mmol), Pd(dppf)Cl₂ (15 mg, 0.12 mmol), tert-butyl 4-(4,4,5,5-tetramethyl-1,3,2-dioxaborolan-2-yl)-3,6- dihydropyridine-1(2H)-carboxylate (732 mg, 2.37 mmol) and potassium carbonate (654 mg, 4.74 mmol) in dioxane (5 mL) and water (1 mL) was stirred at 85° C. for 12 hour under nitrogen atmosphere. The mixture was poured into cold water and extracted with EtOAc(3 x 15 mL). The combined organic layer was washed with water (30 mL), dried over sodium sulfate, filtered and concentrated under reduced pressure, the residue was purified by silica gel column chromatography to furnish tert-butyl 6-((4-chloro-2-fluorobenzyl)oxy)-3′,6′-dihydro-[2,4′- bipyridine]-1′(2′H)-carboxylate (800 mg, yield: 96%) as yellow oil. MS Calcd.: 418.1; MS Found: 419.1 [M+H]⁺.

### Step B: The Synthesis of 6-((4-Chloro-2-Fluorobenzyl)oxy)-1′,2′,3′,6′-Tetrahydro-2,4′-Bipyridine

A solution of tert-butyl 6-((4-chloro-2-fluorobenzyl)oxy)-3′,6′-dihydro-[2,4′-bipyridine]-1′(2′H)- carboxylate (800 mg, 1.96 mmol) and TFA (1.5 mL) in DCM (3 mL) was stirred at room temperature for 2 hours. The reaction mixture was concentrated in vacuo to give crude product 6-((4-chloro-2-fluorobenzyl)oxy)-1′,2′,3′,6′-tetrahydro-2,4′-bipyridine (800 mg, yield: 99%) as nut-brown oil, which was used directly in the next step. MS Calcd.: 318.1; MS Found: 319.0 [M+H]⁺.

### Step C: The Synthesis of Ethyl (S)-2-((6-((4-Chloro-2-Fluorobenzyl)oxy)-3′,6′-Dihydro-[2,4′- Bipyridin]-1′(2′H)-yl)Methyl)-3-(Oxetan-2-ylMethyl)-3H-Imidazo[4,5-b-]Pyridine-5-Carboxylate

A mixture of 6-((4-chloro-2-fluorobenzyl)oxy)-1′,2′,3′,6′-tetrahydro-2,4′-bipyridine (500 mg, 1.5 mmol), ethyl (S)-2-(chloromethyl)-3-(oxetan-2-ylmethyl)-3H-imidazo[4,5-b]pyridine-5-carboxylate (630 mg, 2.0 mmol) and cesium carbonate (1.5 g, 4.7 mmol) in DMF (6 mL) was stirred at room temperature for 5 hours. The reaction mixture was extracted with EtOAc (15 mL x 3), washed with brine (10 mL x 3), dried over sodium sulfate, filtered and concentrated in vacuo. The residue was purified by silica gel to give ethyl (S)-2-((6-((4-chloro-2-fluorobenzyl)oxy)-3′,6′-dihydro- [2,4′-bipyridin]-1′(2′H)-yl)methyl)-3-(oxetan-2-ylmethyl)-3H-imidazo[4,5-b]pyridine-5-carboxylate (260 mg, yield: 32%) as yellow oil. MS Calcd.: 591.2; MS Found: 591.9 [M+H]⁺.

### Step D: The Synthesis of (S)-2-((6-((4-Chloro-2-Fluorobenzyl)oxy)-3′,6′-Dihydro- [2,4′-Bipyridin]-1′(2′H)-yl)Methyl)-3-(Oxetan-2-ylMethyl)-3H-Imidazo[4,5-b]Pyridine-5-Carboxylic Acid

A mixture of ethyl (S)-2-((6-((4-chloro-2-fluorobenzyl)oxy)-3′,6′-dihydro-[2,4′-bipyridin]-1′(2′H)- yl)methyl)-3-(oxetan-2-ylmethyl)-3H-imidazo[4,5-b]pyridine-5-carboxylate (260 mg, 0.5 mmol) and lithium hydroxide (25 mg, 1.0 mmol) in methanol (3 mL) and water (0.5 mL) was stirred at room temperature for 4 hours. The reaction mixture was purified by prep-HPLC directly to give (S)-2-((6-((4-chloro-2-fluorobenzyl)oxy)-3′,6′-dihydro-[2,4′-bipyridin]-1′(2′H)-yl)methyl)-3-(oxetan-2-ylmethyl)-3H-imidazo[4,5-b]pyridine-5-carboxylic acid (39.9 mg, yield: 14%) as white solid. MS Calcd.: 563.1; MS Found: 564.1 [M+H]⁺.

¹H NMR (400 MHz, CD₃OD) δ 8.21 - 8.06 (m, 2 H), 7.64 (t, J = 7.8 Hz, 1 H), 7.51 (t, J= 8.0 Hz, 1 H), 7.22 (t, J = 9.4 Hz, 2 H), 7.08 (d, J = 7.5 Hz, 1 H), 6.79 - 6.63 (m, 2 H), 5.45 (s, 2 H), 5.35 - 5.26 (m, 1 H), 5.04 (dd, J = 14.9, 6.8 Hz, 1H), 4.88 (dd, J = 14.9, 3.0 Hz, 1 H), 4.67 - 4.58 (m, 1 H), 4.49 - 4.40 (m, 1 H), 4.32 (d, J = 14.1 Hz, 1 H), 4.21 (d, J = 14.0 Hz, 1 H), 3.42 (s, 2 H), 2.96 (t, J = 5.5 Hz, 2 H), 2.85 - 2.75 (m, 1 H), 2.70 (s, 2 H), 2.59 - 2.49 (m, 1 H).

Example 10: (S)-2-((4-(2-((4-cyano-2-fluorobenzyl)oxy)thiazol-4-yl)-3,6-dihydropyridin-1 (2H)-yl)methyl)-3-(oxetan-2-ylmethyl)-3H-imidazo[4,5-b]pyridine-5-carboxylic acid (Compound 112a)

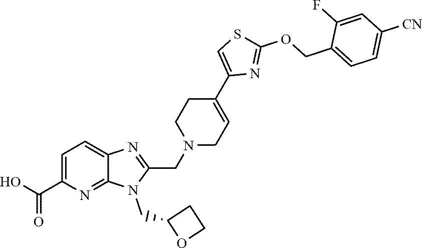 [SMILES: N#Cc1ccc(COc2nc(C3=CCN(Cc4nc5ccc(C(=O)O)nc5n4C[C@@H]4CCO4)CC3)cs2)c(F)c1]

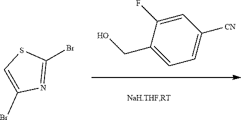

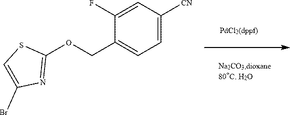

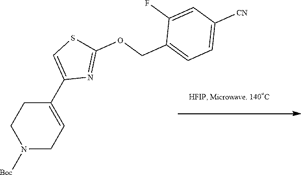

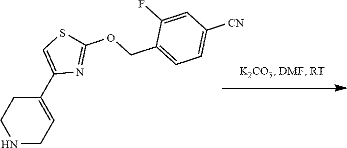

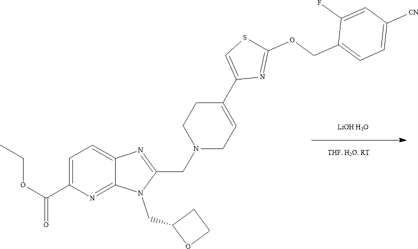

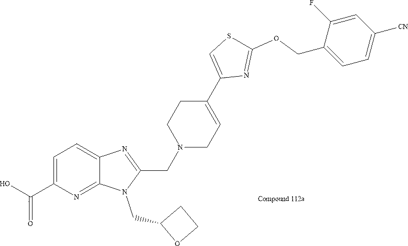

### Step A: The Synthesis of 4-((4-Bromothiazol-2-ylOxy)Methyl)-3-Fluorobenzonitrile

To a solution of 3-fluoro-4-(hydroxymethyl)benzonitrile (1.51 g, 10 mmol) in dry THF (20 mL) were added to a solution of NaH (600 mg, 15 mmol) in dry THF (15 mL) at 0° C. The mixture was stirred at 0° C. for 0.5h. 2, 4-dibromothiazole (2.42 g, 10 mmol) was added and the mixture was stirred at 25° C. for 6 h. The reaction was diluted with water (20 mL) and extracted with ethyl acetate (45 mL x 3). The combined organic layers were dried over sodium sulfate, filtered, and concentrated in vacuo. The residue was purified by column chromatography to give 4-((4-bromothiazol-2-yloxy)methyl)-3-fluorobenzonitrile (1.9 g, yield: 61%) as white solid. MS Calcd.: 311.9; MS Found: 312.8 [M+H]⁺.

### Step B: The Synthesis of Tert-Butyl 4-(2-(4-Cyano-2-Fluorobenzyloxy)Thiazol-4-yl)-5,6-Dihydropyridine- 1 (2H)-Carboxylate

To a solution of 4-((4-bromothiazol-2-yloxy)methyl)-3-fluorobenzonitrile (1.8 g, 5.77 mmol ), tert-butyl 4-(4,4,5,5-tetramethyl-1,3,2-dioxaborolan-2-yl)-3,6-dihydropyridine-1(2H)-carboxylate (2.67 g, 8.66 mmol) and Na₂CO₃ (1.22 g, 11.54 mmol) in Dox/H₂O (20 mL/4 mL) was added Pd(dppf)Cl₂ (230 mg, 0.31 mmol) at RT. The mixture was stirred at 85° C. overnight under N₂ atmosphere. The mixture was filtered, and the filtrate was concentrated under reduced pressure. The resulting residue was purified by silica gel column chromatography to afford tert-butyl 4-(2-(4-cyano-2-fluorobenzyloxy)thiazol-4-yl)-5,6-dihydropyridine-1(2H)-carboxylate (1.1 g, yield: 50%) as white solid. MS Calcd.: 415.1; MS Found: 438.0 [M+Na]⁺.

### Step C: 3-Fluoro-4-((4-(1,2,3,6-Tetrahydropyridin-4-yl)Thiazol-2-yloxy)Methyl)Benzonitrile

To a solution of tert-butyl 4-(2-(4-cyano-2-fluorobenzyloxy)thiazol-4-yl)-5,6-dihydropyridine-1(2 H)-carboxylate (300 mg, 0.72 mmol) in HFIP (2 mL). The mixture was stirred 140° C. under microwave for 4 hours. The reaction mixture was concentrated to give the crude product 3-fluoro-4-((4-(1,2,3,6-tetrahydropyridin-4-yl)thiazol-2-yloxy)methyl)benzonitrile (210 mg crude) as white solid, which was used for next step directly. MS Calcd.: 315.1; MS Found: 316.0 [M+H]⁺.

### Step D: The Synthesis of (S)-Ethyl 2-((4-(2-(4-Cyano-2-Fluorobenzyloxy)Thiazol-4-yl)-5,6-Dihydropyridin-1(2H)-yl)Methyl)-3-(Oxetan-2-ylMethyl)-3H-Imidazo[4,5-b]Pyridine-5-Carboxylate

To a solution of (S)-ethyl 2-(chloromethyl)-3-(oxetan-2-ylmethyl)-3H-imidazo[4,5-b]pyridine-5- carboxylate (185 mg, 0.6 mmol) in dry DMF (5 mL) were added 3-fluoro-4-((4-(1,2,3,6- tetrahydropyridin-4-yl)thiazol-2-yloxy)methyl)benzonitrile (210 crude) and K₂CO₃ (696 mg, 2.14 mmol). The mixture was stirred at 25° C. for 2 h. The mixture was then filtered. The filtrate was concentrated, and the resulting residue was purified by prep-HPLC (high pH) to give (S)-ethyl 2-((4-(2-(4-cyano-2-fluorobenzyloxy)thiazol-4-yl)-5,6-dihydropyridin-1(2 H)-yl)methyl)-3-(oxetan-2-ylmethyl)-3H-imidazo[4,5-b]pyridine-5-carboxylate (80 mg) as white solid. MS Calcd.: 588.2; MS Found: 589.1 [M+H]⁺.

### Step E: The Synthesis of (S)-2-((4-(2-((4-Cyano-2-Fluorobenzyl)Oxy)Thiazol-4-yl)-3,6-Dihydropyridin-1 (2H)-yl)Methyl)-3-(Oxetan-2-ylMethyl)-3H-Imidazo[4,5-b]Pyridine-5-Carboxylic Acid

A mixture of (S)-ethyl 2-((4-(2-(4-cyano-2-fluorobenzyloxy)thiazol-4-yl)-5,6-dihydropyridin- 1(2H)-yl)methyl)-3-(oxetan-2-ylmethyl)-3H-imidazo[4,5-b]pyridine-5-carboxylate (80 mg, 0.14 mmol) and lithium hydroxide (29.4 mg, 0.7 mmol) in THF (3 mL) and water (1 mL) was stirred at room temperature for 6 hours. The reaction mixture was purified by prep-HPLC directly to give (S)-2-((4-(2-((4-cyano-2-fluorobenzyl)oxy)thiazol-4-yl)-3,6-dihydropyridin-1(2H)-yl)methyl)-3-(oxetan-2-ylmethyl)-3H-imidazo[4,5-b]pyridine-5-carboxylic acid (25 mg, yield: 32%) as white solid. MS Calcd.: 560.2; MS Found: 561.1 [M+H]⁺.

¹H NMR (400 MHz, CD₃OD) δ 8.11-8.05 (m, 2 H), 7.76 (t, J = 6.0 Hz, 1 H), 7.62 (t, J = 6.0 Hz, 2 H), 6.69 (s, 1 H),6.52(s, 1 H), 5.61 (s, 2 H), 5.32-5.30 (m,1 H), 5.06-5.01 (m, 1 H), 4.64-4.60 (m, 2 H), 4.46-4.42 (m, 1 H),4.23 (d, J = 11.2 Hz, 1 H), 4.11 (d, J = 10.8 Hz, 1 H), 3.33 - 3.29 (m, 2 H), 2.85-2.76 (m, 3 H), 2.56-2.51 (m, 3 H).

Example 11: 2-((4-(2-((4-cyano-2-fluorobenzyl)oxy)pyrimidin-4-yl)cyclohex-3-en-1-yl)methyl)-3-(((S)-oxetan-2-yl)methyl)-3H-imidazo[4,5-b]pyridine-5-carboxylic acid (Compound 105a)

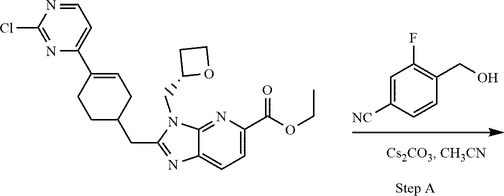

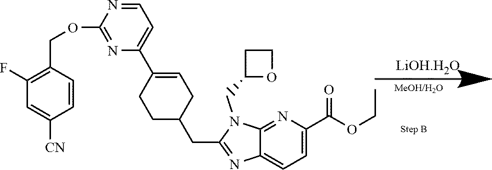

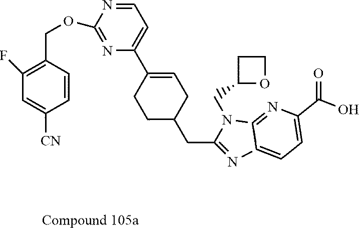

### Step A_{:} The Synthesis of Ethyl 2-((4-(2-((4-Cyano-2-Fluorobenzyl)Oxy)Pyrimidin-4-yl)Cyclohex-3-en-1-yl)Methyl)-3-(((S)-Oxetan-2-yl)Methyl)-3H-Imidazo[4,5-b]Pyridine-5-Carboxylate

To a solution of ethyl 2-((4-(2-chloropyrimidin-4-yl)cyclohex-3-en-1-yl)methyl)-3-(((S)-oxetan-2-yl)methyl)-3H-imidazo[4,5-b]pyridine-5-carboxylate (700 mg, 1.50 mmol) in CH₃CN (20 mL) were added 3-fluoro-4-(hydroxymethyl)benzonitrile (283 mg, 1.87 mmol) and Cs₂CO₃ (974 mg, 3.0 mmol). The mixture was stirred at 80° C. for 3 h. It was then cooled to RT and filtered. The filtrate was concentrated, and the residue was purified by prep-HPLC (high pH) to give ethyl 2-((4-(2-((4-cyano-2-fluorobenzyl)oxy)pyrimidin-4-yl)cyclohex-3-en-1-yl)methyl)-3-(((S)-oxetan-2-yl)methyl)-3H-imidazo[4,5-b]pyridine-5-carboxylate (320 mg, yield: 36.7%) as yellow solid. MS Calcd.: 582.2; MS Found: 582.9 [M+H]⁺.

### Step B: The Synthesis of 2-((4-(2-((4-Cyano-2-Fluorobenzyl)Oxy)Pyrimidin-4-yl)Cyclohex-3-en-1-yl)Methyl)-3-(((S)-Oxetan-2-yl)Methyl)-3H-Imidazo[4,5-b]Pyridine-5-Carboxylic Acid

Xo a solution of ethyl 2-((4-(2-((4-cyano-2-fluorobenzyl)oxy)pyrimidin-4-yl)cyclohex-3-en-1-yl)methyl)-3-(((S)-oxetan-2-yl)methyl)-3H-imidazo[4,5-b]pyridine-5-carboxylate (320 mg, 0.55 mmol) in CH₃OH (9 mL) and H2O (3 mL) was added LiOH.H₂O (69 mg, 1.65 mmol). The mixture was stirred at RT for 2 h. It was acidified to pH=5 with AcOH (10 %) and then extracted with DCM (15 mL*3). The combined organic layers were dried over Na₂SO₄, filtered, and concentrated. The residue was purified by prep-HPLC to give 2-((4-(2-((4-cyano-2-fluorobenzyl)oxy)pyrimidin-4-yl)cyclohex-3-en-1-yl)methyl)-3-(((S)-oxetan-2-yl)methyl)-3H-imidazo[4,5-b]pyridine-5-carboxylic acid (182.5 mg, yield: 59.9%) as white solid. MS Calcd.: 554.2; MS Found: 555.3 [M+H]⁺.

1 H NMR (400 MHz, CD₃OD) δ 8.46 (d, J = 4.4 Hz, 1 H), 8.13 (d, J = 6.8 Hz, 1 H), 8.05 (d, J = 6.8 Hz, 1 H), 7.75 (t, J = 6.0 Hz, 1 H), 7.63~7.58 (m, 2 H), 7.22 (d, J = 4.4 Hz, 1 H), 7.09 (s, 1 H), 5.61 (s, 2 H), 5.29∼5.25 (m, 1 H), 4.81~4.76 (m, 1 H), 4.72~4.68 (m, 1 H), 463~4.58 (m, 1 H), 4.44~4.38 (m, 1 H), 3.24~3.15 (m, 2 H), 2.81∼2.78 (m, 1 H), 2.69∼2.62 (m, 1 H), 2.57∼2.48 (m, 4 H), 2.23∼2.07 (m, 2 H), 1.65∼1.58 (m, 1 H).

Example 12: Synthetic Scheme of 2-((4-(6-((4-chloro-2-fluorobenzyl)oxy)-5-fluoropyridin-2-yl)cyclohex-3-en-1-yl)methyl)-3-(((S)-oxetan-2-yl)methyl)-3H-imidazo[4,5-b]pyridine-5-carboxylic acid (Compound 113a)

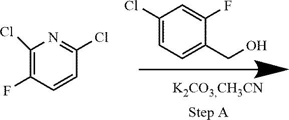

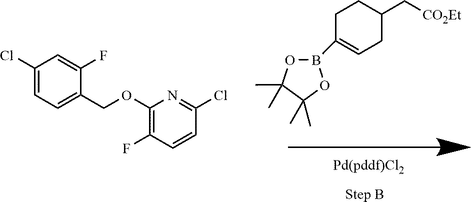

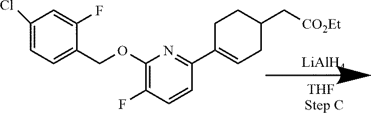

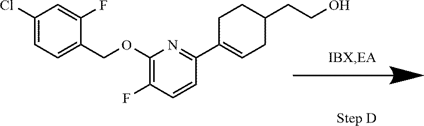

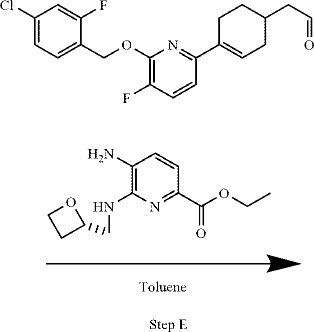

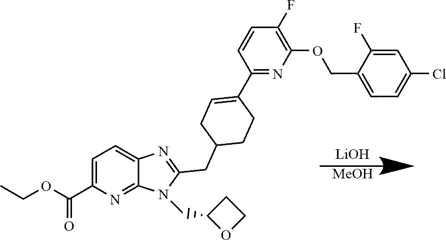 [SMILES: "MeOH" ; CCOC(=O)c1ccc2nc(CC3CC=C(c4ccc(F)c(OCc5ccc(Cl)cc5F)n4)CC3)n(C[C@@H]3CCO3)c2n1 ; "LiOH"]

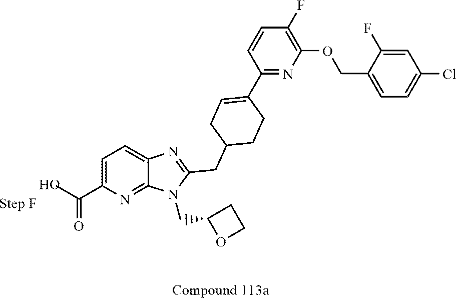

### Step A: The Synthesis of 6-Chloro-2-((4-Chloro-2-Fluorobenzyl)Oxy)-3-Fluoropyridine

To a solution of 2,6-dichloro-3-fluoropyridine (3.2 g, 20 mmol) and (4-chloro-2-fluorophenyl)methanol (3.3 g, 20 mmol) in CH₃CN (60 mL) was added K₂CO₃ (5.5 g, 40 mmol) at 80° C. stirred for 13 hr. The mixture was cooled to RT. The mixture was diluted with H₂O (100 mL) and extracted with EtOAc (100 mL) twice. The organic layer was washed with brine (50 mL), dried over anhydrous Na₂SO₄, and filtered. The filtrate was concentrated to give crude product. The crude was purified by Combi flash (silica gel, eluted Ethyl / Petroleum from 0% to 25%) to give 6-chloro-2-((4-chloro-2-fluorobenzyl)oxy)-3-fluoropyridine (0.9 g, 5 mmol, 25% yield) as a white solid. LCMS: m/z 290.0 [M+H]⁺.

### Step B: The Synthesis of Ethyl 2-(4-(6-((4-Chloro-2-Fluorobenzyl)Oxy)-5-Fluoropyridin-2-yl)Cyclohex-3 -en-1 -yl)Acetate

To a suspension of 6-chloro-2-((4-chloro-2-fluorobenzyl)oxy)-3-fluoropyridine (0.9 g, 3.1 mmol) and ethyl 2-(4-(4,4,5,5-tetramethyl-1,3,2-dioxaborolan-2-yl)cyclohex-3-en-1-yl)acetate (1 g, 3.4 mmol) in dioxane (20 mL) and H2O (7 mL) was added K₂CO₃ (0.9 g, 6 mmol) and Pd(dppf)C12 (245 mg, 0.3 mmol) at 25° C. The mixture was stirred at 80° C. for 16 hours under N₂ atmosphere. The mixture was diluted H₂O (50 mL) and extracted with EtOAc (80 mL) twice. The organic layer was washed brine (100 mL), dried over anhydrous Na₂SO₄ and filtered. The filtrate was concentrated to give a crude product as brown oil. The crude was purified by Combi flash (silica gel, eluted Ethyl / Petroleum from 5 to 25%) to afford ethyl 2-(4-(6-((4-chloro-2-fluorobenzyl)oxy)-5-fluoropyridin-2-yl)cyclohex-3-en-1-yl)acetate (0.6 g, 1.5 mmol, 50% yield). LCMS: m/z 422.1 [M+H]⁺

### Step C: The Synthesis of 2-(4-(6-((4-Chloro-2-Fluorobenzyl)Oxy)-5-Fluoropyridin-2-yl)Cyclohex-3 -en-1 -yl)Ethan-1 -ol

To a mixture of LiAlH₄ (182 mg, 4.8 mmol) in dry THF (10 mL) was added a solution of ethyl 2-(4-(6-((4-chloro-2-fluorobenzyl)oxy)-5-fluoropyridin-2-yl)cyclohex-3-en-1-yl)acetate (340 mg, 0.81 mmol) in dry THF (2 mL) dropwise at -20° C. The mixture was stirred at -20° C. for 1 h and then cooled to -78° C. H₂O (0.206 mL) was added dropwise to quench the reaction. NaOH aqueous solution (15%, 0.206 mL) was then added followed by addition of H₂O (0.618 mL). The mixture was warmed to 0° C. and stirred for 30 min. The resulting white suspension was filtered, and the filtrate was dried over Na₂SO₄. The mixture was filtered, and the filtrate was concentrated to give 2-(4-(6-((4-chloro-2-fluorobenzyl)oxy)-5-fluoropyridin-2-yl)cyclohex-3-en-1-yl)ethan-1-ol (0.4 g, 70%) as orange oil. LCMS: m/z 380.1 [M+H]⁺

### Step D: The Synthesis of 2-(4-(6-((4-Chloro-2-Fluorobenzyl)Oxy)-5-Fluoropyridin-2-yl)Cyclohex-3-en-1-yl)Acetaldehyde

2-(6-(chloro-2-fluorobenzyl)oxy)-5-fluoropyridin-2-yl)cyclohex-3-en-1-yl)ethan-1-ol (300 mg, 0.79 mmol) and IBX (665 mg, 2.37 mmol) were dissolved in EtOAc (20 ml). The mixture was stirred at 80° C. for 15 hours. The mixture was filtered, the filtrate was concentrated to give 2-(4-(6-((4-chloro-2-fluorobenzyl)oxy)-5-fluoropyridin-2-yl)cyclohex-3-en-1-yl)acetaldehyde (300 mg) as a white solid (crude product). MS Found: 378.1 [M+H]⁺.

### Step E: The Synthesis of Ethyl 2-((4-(6-((4-Chloro-2-Fluorobenzyl)Oxy)-5-Fluoropyridin-2-yl)Cyclohex-3-en-1-yl)Methyl)-3-(((S)-Oxetan-2-yl)Methyl)-3H-Imidazo[4,5-b]Pyridine-5-Carboxylate

A mixture of 2-(4-(6-((4-chloro-2-fluorobenzyl)oxy)-5-fluoropyridin-2-yl)cyclohex-3-en-1-yl)acetaldehyde (130 mg) and ethyl (S)-5-amino-6-((oxetan-2-ylmethyl)amino)picolinate (86 mg, 0.34 mmol) were in toluene (20 ml). The mixture was stirred at 80° C. for 48 hours. Concentrated in vacuum, the residue was purified by column chromatography to give ethyl 2-((4-(6-((4-chloro-2-fluorobenzyl)oxy)-5-fluoropyridin-2-yl)cyclohex-3-en-1-yl)methyl)-3-(((S)-oxetan-2-yl)methyl)-3H-imidazo[4,5-b]pyridine-5-carboxylate (140 mg, yield: 60%) as brown oil. LCMS: m/z609.2 [M+H]

### Step F: The Synthesis of 2-((4-(6-((4-Chloro-2-Fluorobenzyl)Oxy)-5-Fluoropyridin-2-yl)Cyclohex-3-en-1-yl)Methyl)-3-(((S)-Oxetan-2-yl)Methyl)-3H-Imidazo[4,5-b]Pyridine-5-Carboxylic Acid

A solution of ethyl 2-((4-(6-((4-chloro-2-fluorobenzyl)oxy)-5-fluoropyridin-2-yl)cyclohex-3-en-1-yl)methyl)-3-(((S)-oxetan-2-yl)methyl)-3H-imidazo[4,5-b]pyridine-5-carboxylate (140 mg, 0.25 m mol) and lithium hydroxide (42 mg, 1 m mol) in THF (2 ml) and water (1 ml) was stirred at room temperature for 10 hours. The reaction mixture was purified by prep-HPLC directly to give 2-((4-(6-((4-chloro-2-fluorobenzyl)oxy)-5-fluoropyridin-2-yl)cyclohex-3-en-1-yl)methyl)-3-(((S)-oxetan-2-yl)methyl)-3H-imidazo[4,5-b]pyridine-5-carboxylic acid (66 mg, yield: 46%) as white solid. LCMS: m/z 581.2 [M+H]⁺

¹H NMR (400 MHz, MeOD) δ 8.13 (d, J = 8.0 Hz, 1 H), 8.07 (d, J = 8.0 Hz, 1 H),7.51 (t, J = 8.0 Hz, 1 H), 7.44-7.40 (m, 1 H), 7.26-7.22 (m, 2 H), 7.06-7.03 (m, 1 H), 6.75-6.68 (m, 1 H), 5.52 (s, 2 H), 5.30-5.25(m, 1 H), 4.83-4.77 (m, 1 H), 4.72-4.68 (m, 1 H), 4.62-4.58 (m, 1 H),4.46-4.39 (m, 1 H) 3.19-3.12 (m, 2 H), 2.83-2.78 (m, 1 H), 2.68-2.63 (m, 1 H), 2.54-2.47 (m, 4 H), 2.13- 2.05 (m, 2 H), 1.62-1.55 (m, 1 H).

Example 13: 2-((4-(4-((4-chloro-2-fluorobenzyl)oxy)-5-fluoropyrimidin-2-yl)cyclohex-3-en-1-yl)methyl)-3-(((S)-oxetan-2-yl)methyl)-3H-imidazo[4,5-b]pyridine-5-carboxylic acid (Compound 114a)

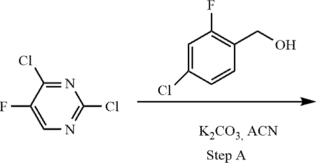

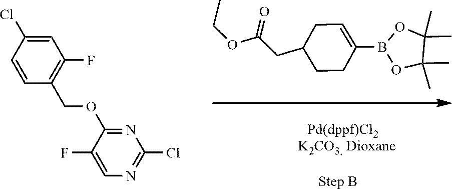

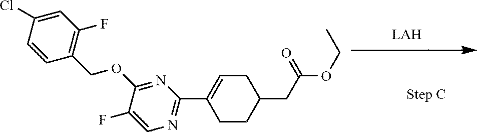

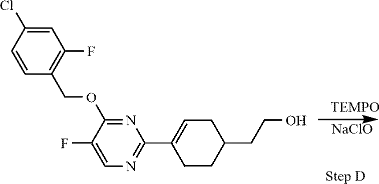

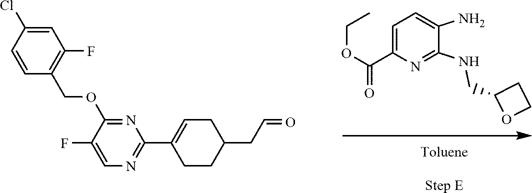

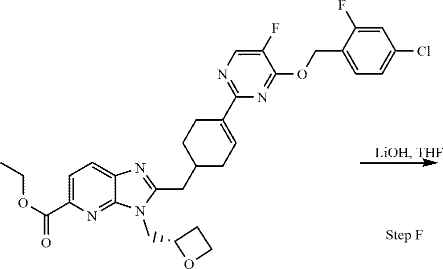

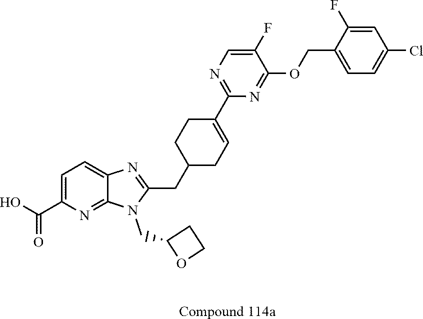

### Step A: The Synthesis of 2-Chloro-4-((4-Chloro-2-Fluorobenzyl)Oxy)-5-Fluoropyrimidine

To a solution of 2,4-dichloro-5-fluoropyrimidine (6.0 g, 35.9 mmol) and (4-chloro-2-fluorophenyl) methanol (5.65 g, 35.21 mmol) in CH₃CN (50 mL) were added to K₂CO₃ (6.4 g, 46.67 mmol). The mixture was stirred at 80° C. for 16 h. LCMS showed 2,4-dichloro-5-fluoropyrimidine was consumed completely and desire product formed. The mixture was filtered, and the filtrate was concentrated under reduced pressure to afford the title product 2-chloro-4-((4-chloro-2-fluorobenzyl)oxy)-5-fluoropyrimidine (9 g, crude) as a white solid, which was used for next step directly. MS Calcd.: 290.0; MS Found: 291.0 [M+H]⁺.

### Step B: The Synthesis of Ethyl-2-(4-(4-((4-Chloro-2-Fluorobenzyl)Oxy)-5-Fluoropyrimidin-2-yl)Cyclohex-3-en -1-yl) Acetate

To a solution of 2-chloro-4-((4-chloro-2-fluorobenzyl)oxy)-5-fluoropyrimidine (5.5 g), tert-butyl 4-(4,4,5,5-tetramethyl-1,3,2-dioxaborolan-2-yl)-3,6-dihydropyridine-1 (2H)-carboxylate (5.55 g, 18.89 mmol) and K₂CO₃ (5.2 g, 37.78 mmol) in dioxane/H₂O (85 mL/ 17 mL) was added Pd(dppf)Cl₂ (691 mg, 0.94 mmol) at RT. The mixture was stirred at 85° C. overnight under N₂ atmosphere. LCMS showed 2-chloro-4-((4-chloro-2-fluorobenzyl)oxy)-5-fluoropyrimidine was consumed completely and desire product formed. The mixture was filtered, and the filtrate was concentrated under reduced pressure. The residue was purified by silica gel column chromatography to afford ethyl-2-(4-(4-((4-chloro-2-fluorobenzyl)oxy) -5-fluoropyrimidin-2-yl)cyclohex-3-en-1-yl)acetate (5 g, yield: 63%) as white solid. MS Calcd.: 422.1; MS Found: 422.9 [M+1]⁺.

### Step C: The Synthesis of 2-(4-(4-((4-Chloro-2-Fluorobenzyl)Oxy)-5-Fluoropyrimidin-2-yl)Cyclohex-3-en-1-yl) Ethan-1-ol

To a solution of ethyl-2-(4-(4-((4-chloro-2-fluorobenzyl)oxy)-5-fluoropyrimidin-2-yl)cyclohex-3-en-1-yl)acetate (2.5 g, 5.9 mmol) in Dry THF (50 mL) was added LAH (448.7 mg, 11.8 mmol) at 0° C. The mixture was stirred at 0° C. for 2 h. LCMS showed 2-chloro-4-((4-chloro-2-fluorobenzyl)oxy)-5-fluoro pyrimidine was consumed completely and desire product formed. The reaction was quenched by addition of 448 mg of H₂O and 448 mg of NaOH (15% aqueous solution). Then the resulting suspension was filtered, washed with EA (10 mL) and the filtrate was extracted with EA (20 mL*3). Combined EA layers were concentrated under reduced pressure. The residue was purified by silica gel column chromatography to afford 2-(4-(4-((4-chloro-2-fluorobenzyl)oxy)-5-fluoropyrimidin-2-yl)cyclohex-3-en-1-yl)ethan-1-ol (1.6 g, crude) as colorless oil. MS Calcd.: 380.1; MS Found: 380.9 [M+H]⁺.

### Step D: The Synthesis of 2-(4-(4-((4-Chloro-2-Fluorobenzyl)oxy)-5-Fluoropyrimidin-2-yl)Cyclohex-3-en-1-yl) Acetaldehyde

To the solution of 2-(4-(4-((4-chloro-2-fluorobenzyl)oxy)-5-fluoropyrimidin-2-yl)cyclohex-3-en-1-yl) ethan-1-ol (1 g, 2.62 mmol), TEMPO (4 mg, 0.026 mmol), NaCl (153.1 mg, 2.62 mmol), NaHCO₃ (220.6 mg, 2.62 mmol), and KBr (31 mg, 0.262 mmol) in dichloromethane/water mixture (20 mL/20 mL) was added NaClO aqueous solution (7.5%, 2.6 mL) dropwise over 20 min at 0° C. LCMS showed 2-(4-(4-((4-chloro-2-fluorobenzyl)oxy)-5-fluoropyrimidin-2-yl)cyclohex-3-en-1-yl)ethan-1-ol was consumed completely and desire product formed. The reaction mixture was extracted with dichloromethane (30 mL*2), washed with saturated Na₂S₂O₃ aqueous solution (25 mL), saturated NaHCO₃ aqueous solution (30 mL) and brine (30 mL). The resulting DCM solution was concentrated and purified on silica gel (0-3% methanol in dichloromethane) to give the title product 2-(4-(4-((4-chloro-2-fluorobenzyl)oxy)-5-fluoropyrimidin-2-yl)cyclohex-3-en-1-yl)acetaldehyde (750 mg, 1.98 mmol). MS Calcd.:378.1; MS Found: 378.9 [M+H]⁺.

### Step E: The Synthesis of Ethyl 2-((4-(4-((4-Chloro-2-Fluorobenzyl)Oxy)-5-Fluoropyrimidin-2-yl)Cyclohex-3-en-^{______}1-yl)Methyl)-3-(((S)-Oxetan-2-yl)Methyl)-3H-Imidazo[4,5-b-]Pyridine-5-Carboxylate

To a mixture of 2-(4-(4-((4-chloro-2-fluorobenzyl)oxy)-5-fluoropyrimidin-2-yl)cyclohex-3-en-1-yl)acetaldehyde (210 mg, 0.57 mmol) and (S)-ethyl 5-amino-6-(oxetan-2-ylmethylamino)picolinate (171.4 mg, 0.68 mmol) in dry toluene (5 mL) was added 4A molecular sieves (130 mg). The mixture was stirred at 100° C. for 40 h under O₂ atmosphere. LCMS showed starting material 2-(4-(4-((4-chloro-2-fluorobenzyl)oxy)-5-fluoropyrimidin-2-yl)cyclohex-3-en-1-yl)acetaldehyde was consumed completely and desire product formed. The reaction mixture was concentrated and purified on silica gel (DCM\MeOH=20\1, UV254 nm) to give title product (210 mg, 0.34 mmol) as brown solid. MS Calcd.: 609.2; MS Found: 609.9 [M+H]⁺.

### Step F: The Synthesis of 2-((4-(4-((4-Chloro-2-Fluorobenzvl)Oxy)-5-Fluoropvrimidin-2-yl)Cyclohex-3-en-1-yl) Methyl)-3-(((S)-Oxetan-2-yl)Methyl)-3H-Imidazo[4,5-b]Pyridine-5-Carboxylic Acid

To a solution of ethyl 2-((4-(4-((4-chloro-2-fluorobenzyl)oxy)-5-fluoropyrimidin-2-yl)cyclohex-3-en-1-yl)methyl)-3-(((S)-oxetan-2-yl)methyl)-3H-imidazo[4,5-b]pyridine-5-carboxylate (150 mg, 0.24 mmol) in THF (4 mL) and H₂O (2 mL) was added LiOH.H₂O (125.88 mg, 0.5 M). The mixture was stirred at 20° C. for 4 h. LCMS showed starting material was consumed completely and desire product formed. The reaction mixture was purified directly by Prep-HPLC to give 2-((4-(4-((4-chloro-2-fluorobenzyl)oxy)-5-fluoropyrimidin-2-yl)cyclohex-3-en-1-yl) methyl)-3-(((S)-oxetan-2-yl)methyl)-3H-imidazo[4,5-b]pyridine-5-carboxylic acid (69 mg, 0.011 mmol). MS Calcd.: 581.2; MS Found: 582.1 [M+H]⁺. ¹H NMR (400 MHz, DMSO-d₆) δ 8.58 (d, J = 2.8 Hz, 1 H), 7.99 (d, J = 8.2 Hz, 1 H), 7.92 (d, J = 8.2 Hz, 1 H), 7.62 (t, J = 8.2 Hz, 1 H), 7.53 (dd, J = 9.9, 1.8 Hz, 1 H), 7.36 (d, J = 1.7 Hz, 1 H),7.16 (s, 1 H), 5.58 (s, 2 H), 5.17 -5.04 (m, 1 H), 4.70 - 4.58 (m, 1 H), 4.56 - 4.40 (m, 2 H), 4.35 - 4.24 (m, 1 H), 3.23 - 2.95 (m, 2 H), 2.77 - 2.61 (m, 2 H), 2.45 - 2.29 (m, 4 H), 2.19 - 2.04 (m, 1 H), 2.04 - 1.93 (m, 1 H), 1.55 - 1.41 (m, 1 H).

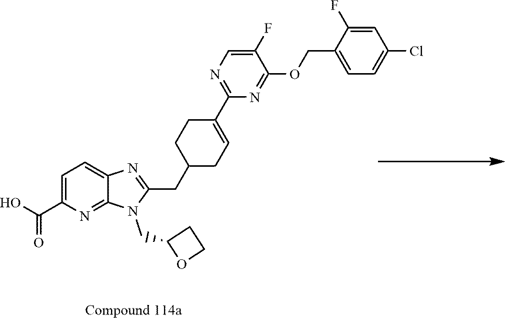

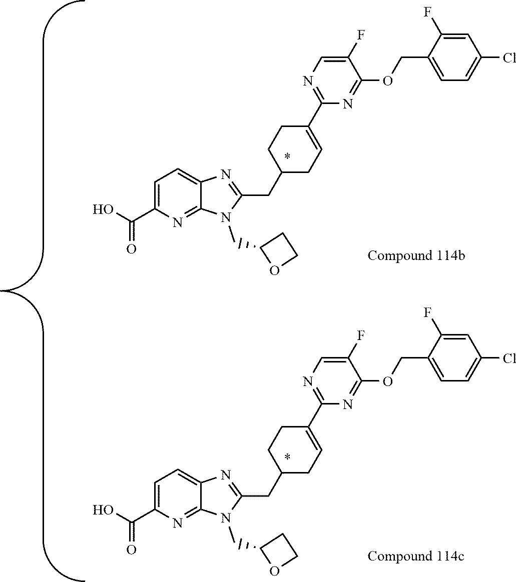

The compound 2-((4-(4-((4-chloro-2-fluorobenzyl)oxy)-5-fluoropyrimidin-2-yl)cyclohex-3-en-1-yl)methyl)-3-(((S)-oxetan-2-yl)methyl)-3H-imidazo[4,5-b]pyridine-5-carboxylic acid (Compound 114a) was purified by SFC (Column: IH, elution: 30% MeOH [0.2%Methanol Ammonia]; Flow: 4ml/min; Temperature: 40° C.; PB: 120 bar) to give the product diastereomer 1 (2.38 g, 4.01 mmol) and diastereomer 2 (2.68 g, 4.6 mmol). diastereomer -1 (Compound 114b) MS Calcd.: 581.2; MS Found: 582.2 [M+H]⁺. ¹H NMR (400 MHz, DMSO-d₆) δ 8.58 (d, J = 2.8 Hz, 1 H), 8.04 (d, J = 8.2 Hz, 1 H), 7.95 (d, J = 8.2 Hz, 1 H), 7.62 (t, J = 8.2 Hz, 1 H), 7.53 (dd, J = 10.0, 1.9 Hz, 1 H), 7.35 (d, J = 8.2, 1.8 Hz, 1 H), 7.16 (s, 1 H),5.58 (s, 2 H), 5.10-5.04 (m, 1 H), 4.70 - 4.58 (m, 1 H), 4.56 - 4.40 (m, 2 H), 4.35 - 4.24 (m, 1 H), 3.23 - 2.95 (m, 2 H), 2.77 - 2.61 (m, 2 H), 2.45 -2.29 (m, 4 H), 2.19 - 2.04 (m, 1 H), 2.04 - 1.93 (m, 1 H), 1.55 - 1.41 (m, 1 H). diastereomer -2 (Compound 114c) MS Calcd.: 581.2; MS Found: 582.2 [M+H]⁺. ¹H NMR (400 MHz, DMSO-d₆) δ 8.58 (d, J = 2.8 Hz, 1 H), 8.04 (d, J = 8.2 Hz, 1 H), 7.96 (d, J = 8.2 Hz, 1 H), 7.62 (t, J = 8.2 Hz, 1 H), 7.52 (dd, J = 9.9, 1.8 Hz, 1 H), 7.35 (d, J = 8.2, 1.6 Hz, 1 H), 7.16 (s, 1 H), 5.57 (s, 2 H), 5.20 - 4.95 (m, 1 H), 4.74 - 4.49 (m, 2 H), 4.42 - 4.32 (m, 1 H), 4.29-4.17 (m, 1 H), 3.20-2.92 (m, 2 H), 2.82-2.69 (m, 1 H), 2.62 -2.53 (m, 2 H),2.45 - 2.29 (m, 3 H), 2.19 - 2.04 (m, 1 H), 2.04 - 1.93 (m, 1 H),1.55 - 1.41 (m, 1 H).

Example 14: 2-((4-(6-((4-cyano-2-fluorobenzyl)oxy)-5-fluoropyridin-2-yl)cyclohex-3-en-1-yl)methyl)-3-(((S)-oxetan-2-yl)methyl)-3H-imidazo[4,5-b]pyridine-5-carboxylic acid (Compound 115a)

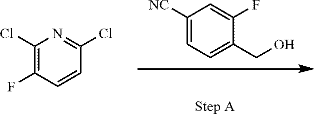

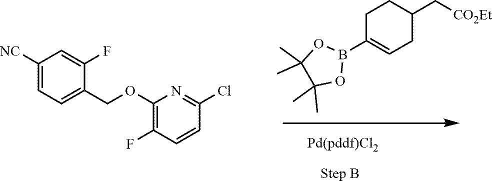

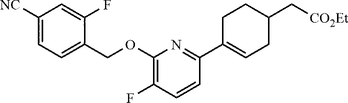

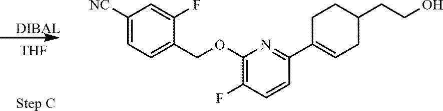

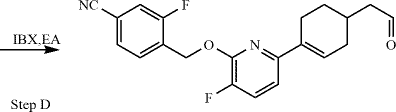

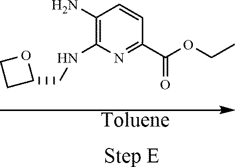

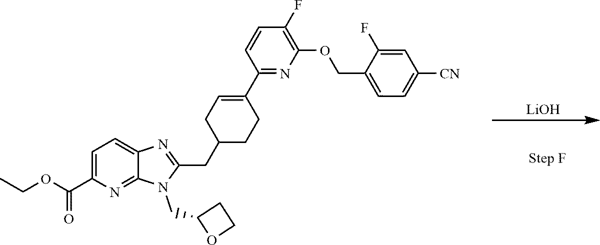

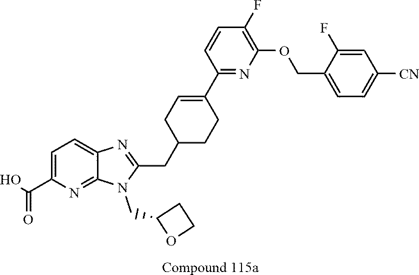

### Step A: The Synthesis of 4-(((6-Chloro-3-Fluoropyridin-2-yl)Oxy)Methyl)-3-Fluorobenzonitrile

To a solution of 2,6-dichloro-3-fluoropyridine (1.1 g, 6.7 mmol) and 3-fluoro-4-(hydroxymethyl)benzonitrile (1.0 g, 6.71 mmol) in CH₃CN (60 mL) was added Cs₂CO₃ (4.4 g, 14 mmol) in one portion. The mixture was stirred at 80° C. for 13 hrs. The mixture was cooled to RT. The mixture was diluted with H₂O (100 mL) and extracted with EtOAc (100 mL) for twice. The organic layers were combined, washed with brine (50 mL), and dried over anhydrous Na₂SO₄. After filter, the filtrate was concentrated and purified by flash (silica gel, eluted with Ethyl acetate / Petroleum ether =⅕, UV254 nm) to give 4-(((6-chloro-3-fluoropyridin-2-yl)oxy)methyl)-3-fluorobenzonitrile (0.7 g, 2.5 mmol, 38% yield) as a white solid. LCMS: m/z 280.9 [M+H]+.

### Step B: The Synthesis of Ethyl 2-(4-(6-((4-Cyano-2-Fluorobenzvl)oxy)-5-Fluoropyridin-2-yl)Cvclohex-3 -en-1 -yl)Acetate

To a suspension of 4-(((6-chloro-3-fluoropyridin-2-yl)oxy)methyl)-3-fluorobenzonitrile (0.6 g, 2 mmol) and ethyl 2-(4-(4,4,5,5-tetramethyl-1,3,2-dioxaborolan-2-yl)cyclohex-3-en-1-yl)acetate (0.6 g, 2 mmol) in dioxane (20 mL) and H₂O (7 mL) was added K₂CO₃ (0.9 g, 6 mmol) and Pd(dppf)Cl₂ (165 mg, 0.2 mmol) at 25° C. The mixture was stirred at 80° C. for 16 hours under nitrogen atmosphere. TLC (Petroleum ether: Ethyl acetate =5:1 UV, 254 nm) showed that the starting material was consumed. The mixture was diluted with H₂O (50 mL), extracted with EtOAc (80 mL) for twice. The organic layers were combined, washed with brine (100 mL), and dried over anhydrous Na₂SO₄. After filtration, the filtrate was concentrated to give crude product as brown oil. This crude was purified by Combi-flash (silica gel, eluted with Ethyl acetate/ Petroleum ether =⅕) to give ethyl 2-(4-(6-((4-cyano-2-fluorobenzyl)oxy)-5-fluoropyridin-2-yl)cyclohex-3-en-1-yl)acetate (0.5 g, 1.2 mmol, 60% yield). LCMS: m/z 413.1 [M+H]⁺

### Step C: The Synthesis of 3-Fluoro-4-(((3-Fluoro-6-(4-(2-Hydroxyethyl)Cyclohex-1-en-1-yl)Pyridin-2-yl)oxy)Methyl)Benzonitrile

To a solution of DIBAL-H (4 mmol, 1 M solution in toluene, 4 mL) was added a solution of ethyl 2-(4-(6-((4-cyano-2-fluorobenzyl)oxy)-5-fluoropyridin-2-yl)cyclohex-3-en-1-yl)acetate (320 mg, 0.78 mmol) in anhydrous THF (2 mL) dropwise at -50° C. The mixture was stirred at -50° C. for 1 h and then cooled to -78° C. Water (0.206 mL) was added dropwise to quench the reaction at -78° C. The resulting white suspension was filtered, and the filtrate was dried over Na₂SO₄. The mixture was filtered, and the filtrate was concentrated to give 3-fluoro-4-(((3-fluoro-6-(4-(2-hydroxyethyl)cyclohex-1-en-1-yl)pyridin-2-yl)oxy)methyl)benzonitrile (0.2 g, 70%) as orange oil. LCMS: m/z 371.0 [M+H]⁺

### Step D: The Synthesis of 3-Fluoro-4-(((3-Fluoro-6-(4-(2-Oxoethyl)Cyclohex-1-en-1-yl)Pyridin-2-yl)Oxy)Methyl)Benzonitrile

To a mixture of 3-fluoro-4-(((3-fluoro-6-(4-(2-hydroxyethyl)cyclohex-1-en-1-yl)pyridin-2-yl)oxy)methyl)benzonitrile (300 mg) in EtOAc (20 mL) were added IBX (665 mg, 2.37 mmol) in portions. The resulting mixture was stirred at 80° C. for 15 hours. The mixture was cooled to room temperature and filtered. The resulting filtrated was concentrated to afford crude 3-fluoro-4-(((3-fluoro-6-(4-(2-oxoethyl)cyclohex-1-en-1-yl)pyridin-2-yl)oxy)methyl)benzonitrile (300 mg, yield: 80%) as white solid. LCMS: m/z 369.0 [M+H]⁺.

### Step E: The Synthesis of Ethyl 2-((4-(6-((4-Cyano-2-Fluorobenzyl)Oxy)-5-Fluoropyridin-2-yl)Cyclohex-3-en-1-yl)Methyl)-3-(((S)-Oxetan-2-yl)Methyl)-3H-Imidazo[4,5-b]Pyridine-5-Carboxylate

A mixture of 3-fluoro-4-(((3-fluoro-6-(4-(2-oxoethyl)cyclohex-1-en-1-yl)pyridin-2-yl)oxy)methyl)benzonitrile (130 mg) and ethyl (S)-5-amino-6-((oxetan-2-ylmethyl)amino)picolinate (86 mg, 0.34 mmol) were in anhydrous toluene (10 mL) was stirred at 80° C. for 48 hours. After concentration in vacuum, the crude product was purified by column chromatography to give ethyl 2-((4-(6-((4-cyano-2-fluorobenzyl)oxy)-5-fluoropyridin-2-yl)cyclohex-3-en-1-yl)methyl)-3-(((S)-oxetan-2-yl)methyl)-3H-imidazo[4,5-b]pyridine-5-carboxylate (130 mg, yield: 60%) as brown oil. LCMS: m/z 600.2 [M+H]⁺.

### Step F: The Synthesis of 2-((4-(6-((4-Cyano-2-Fluorobenzyl)Oxy)-5-Fluoropyridin-2-yl)Cyclohex-3-en-1-yl)Methyl)-3-(((S)-Oxetan-2-yl)Methyl)-3H-Imidazo[4,5-b]Pyridine-5-Carboxylic Acid

To a solution of ethyl 2-((4-(6-((4-cyano-2-fluorobenzyl)oxy)-5-fluoropyridin-2-yl)cyclohex-3-en-1-yl)methyl)-3-(((S)-oxetan-2-yl)methyl)-3H-imidazo[4,5-b]pyridine-5-carboxylate (100 mg, 0.25 m mol) in THF (2 mL) were added lithium hydroxide (42 mg, 1 m mol) and water (1 mL). The mixture was stirred at room temperature for 10 hours. The reaction mixture was purified by prep-HPLC directly to give 2-((4-(6-((4-cyano-2-fluorobenzyl)oxy)-5-fluoropyridin-2-yl)cyclohex-3-en-1-yl)methyl)-3-(((S)- oxetan-2-yl)methyl)-3H-imidazo[4,5-b]pyridine-5-carboxylic acid (30 mg, yield: 46%) as white solid. LCMS: m/z 571.9 [M+H]⁺

¹H NMR (400 MHz, MeOD) δ 8.13 (d, J = 8.0 Hz, 1 H), 8.07 (d, J = 8.0 Hz, 1 H), 7.73 (t, J = 8.0 Hz, 1 H), 7.62-7.57 (m, 2 H), 7.46-7.41 (m, 1 H), 7.07-7.04 (m, 1 H), 6.67-6.66 (m, 1 H), 5.61 (s, 2 H), 5.28-5.27(m, 1 H), 4.77-4.60 (m, 2 H), 4.61-4.59 (m, 1 H), 4.43-4.39 (m, 1 H), 3.19-3.14 (m, 2 H), 2.83-2.78 (m, 1 H), 2.68-2.58 (m, 1 H), 2.54-2.37 (m, 4 H), 2.13- 2.05 (m, 2 H), 1.62-1.60 (m, 1 H),

Example 15: 2-((4-(6-((4-chloro-2-fluorobenzyl)oxy)pyridin-2-yl)cyclohex-3-en-1-yl)methyl)-1-(((S)-oxetan-2-yl)methyl)-1H-imidazo[4,5-c]pyridine-6-carboxylic acid (Compound 116a)

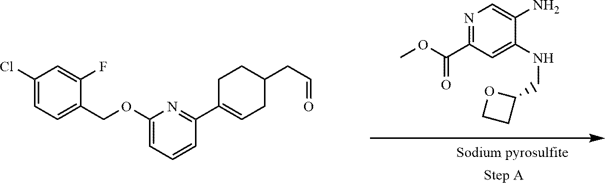

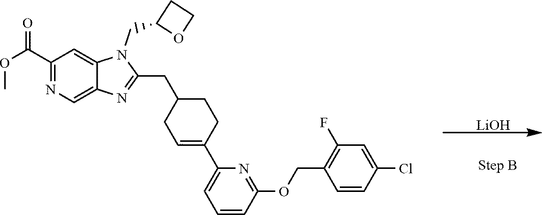

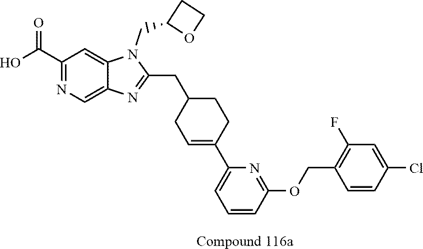

### Step A: The Synthesis of Methyl 2-((4-(6-((4-Chloro-2-Fluorobenzyl)Oxy)Pyridin-2-yl)Cyclohex-3-en-1-yl)Methyl)-1-(((S)-Oxetan-2-yl)Methyl)-1H-Imidazo[4,5-c]Pyridine-6-Carboxylate

To a solution of 2-(4-(6-((4-chloro-2-fluorobenzyl)oxy)pyridin-2-yl)cyclohex-3-en-1-yl)acetaldehyde (180 mg, 0.5 mmol) in EtOH (1.5 mL) was added a solution of sodium metabisulfite (48 mg, 0.25 mmol) in H₂O (1.5 mL). The mixture was stirred at RT for 3 h and then diluted with EtOH (2 mL). The resulting suspension was kept in a refrigerator for 12 h. The suspension was filtered. The precipitate was collected and dried. This resulting precipitate was added to a solution of methyl (S)-5-amino-4-((oxetan-2-ylmethyl)amino)picolinate (120 mg, 0.5 mmol) in DMF (5 mL). The mixture was stirred at 110° C. for 3 h. The reaction mixture was then cooled and poured into water (20 mL). The resulting suspension was extracted with EtOAc (100 mL) for twice. The organic layers were combined, washed with brine (10 mL), and dried over anhydrous Na₂SO₄. After filter, the filtrate was concentrated to give crude product. The crude was purified by Combi-flash (silica gel, eluted with Ethyl acetate/ Petroleum ether from 0% to 50%) to give methyl 2-((4-(6-((4-chloro-2-fluorobenzyl)oxy)pyridin-2-yl)cyclohex-3-en-1-yl)methyl)-1-(((S)-oxetan-2-yl)methyl)-1H-imidazo[4,5-c]pyridine-6-carboxylate (0.2 g, 56% yield) as brown oil. LCMS: m/z 577.1 [M+H]⁺.

### Step B: The Synthesis of 2-((4-(6-((4-Chloro-2-Fluorobenzyl)Oxy)Pyridin-2-yl)Cyclohex-3-en-1-yl)Methyl)-1-(((S)-Oxetan-2-yl)Methyl)-1H-Imidazo[4,5-c]Pyridine-6-Carboxylic Acid

A mixture of methyl 2-((4-(6-((4-chloro-2-fluorobenzyl)oxy)pyridin-2-yl)cyclohex-3-en-1-yl)methyl)-1-(((S)-oxetan-2-yl)methyl)-1H-imidazo[4,5-c]pyridine-6-carboxylate (90 mg, 0.25 mmol) and lithium hydroxide (42 mg, 1 m mol) in THF (2 mL) and water (1 mL) was stirred at room temperature for 10 hours. The reaction mixture was purified by prep-HPLC directly to give 2-((4-(6-((4-chloro-2-fluorobenzyl)oxy)pyridin-2-yl)cyclohex-3-en-1-yl)methyl)-1-(((S)-oxetan-2-yl)methyl)-1H-imidazo[4,5-c]pyridine-6-carboxylic acid (40 mg, yield: 46%) as white solid. LCMS: m/z 563.1 [M+H]⁺¹H NMR (400 MHz, DMSO-d₆) δ 8.97(s, 1 H),8.42 (s, 1 H), 7.68 (t, J= 8.0 Hz, 1 H), 7.56 (t, J = 8.0 Hz, 1 H), 7.48 (dd, J₁ = 2 Hz, J₂ = 10 Hz ,1 H), 7.30(dd, J₁ = 2 Hz, J₂ = 8 Hz ,1 H), 7.08 (d, J = 8 Hz, 1 H),6.82-6.76 (m, 1 H), 6.72-6.70 (m, 1 H), 5.40 (s, 2 H), 5.04-5.02(m, 1 H), 4.77-4.72 (m, 1 H), 4.61-4.59 (m, 1 H), 4.43-4.39 (m, 1 H), 4.35-4.29 (m, 1 H), 3.14-3.02(m, 2 H), 2.72-2.58 (m, 2 H), 2.42-2.33 (m, 4 H), 2.13- 2.05 (m, 2 H), 1.60-1.42 (m, 1 H),

Example 16: 2-((4-(6-((4-cyano-2-fluorobenzyl)oxy)pyridin-2-yl)cyclohex-3-en-1-yl)methyl)-1-(((S)-oxetan-2-yl)methyl)-1H-imidazo[4,5-c]pyridine-6-carboxylic acid (Compound 117a)

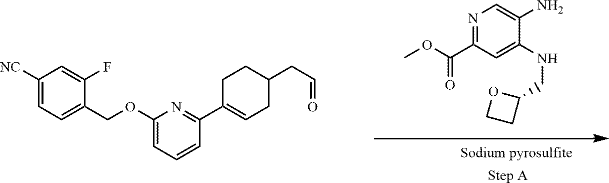

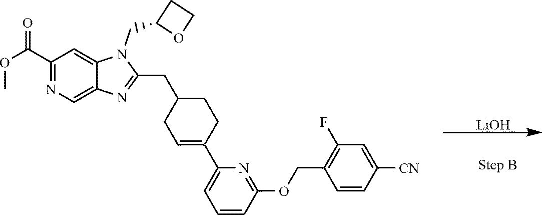

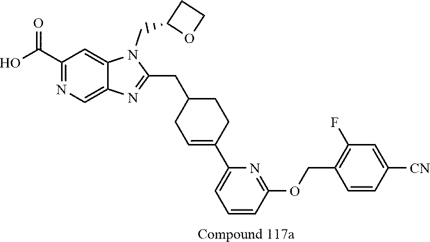

### Step A: The Synthesis of Methyl 2-((4-(6-((4-Cyano-2-Fluorobenzyl)Oxy)Pyridin-2-yl)Cyclohex-3-en-1-yl)Methyl)-1-(((S)-Oxetan-2-yl)Methyl)-1H-Imidazo[4,5-c]Pyridine-6-Carboxylate

To a solution of 3-fluoro-4-(((6-(4-(2-oxoethyl)cyclohex-1-en-1-yl)pyridin-2-yl)oxy)methyl)benzonitrile (330 mg, 0.94 mmol) in EtOH (1.5 mL) were added a solution of sodium metabisulfite (48 mg, 0.25 mmol) in H₂O (1.5 mL). The mixture was stirred at RT for 3 h and then diluted with EtOH (2 mL). The resulting suspension was kept in a refrigerator for 12 h. The suspension was filtered. The precipitate was collected and dried. This resulting precipitate was added to a solution of methyl (S)-5-amino-4-((oxetan-2-ylmethyl)amino)picolinate (120 mg, 0.5 mmol) in DMF (5 mL). The mixture was stirred at 110° C. for 3 h. The reaction mixture was then cooled and poured into water (20 mL). The resulting suspension was extracted with EtOAc (100 mL) for twice. The organic layers were combined, washed with brine (10 mL), and dried over anhydrous Na₂SO₄. After filter, the filtrate was concentrated to give crude product. The crude was purified by Combi-flash (silica gel, eluted with Ethyl acetate/ Petroleum ether from 0% to 50%) to give methyl 2-((4-(6-((4-cyano-2-fluorobenzyl)oxy)pyridin-2-yl)cyclohex-3-en-1-yl)methyl)-1-(((S)-oxetan-2-yl)methyl)-1H-imidazo[4,5-c]pyridine-6-carboxylate (0.2 g, 35% yield) as brown oil. LCMS: m/z 568.0 [M+H]⁺.

### Step B: The Synthesis of 2-((4-(6-((4-Cyano-2-Fluorobenzyl)Oxy)Pyridin-2-yl)Cyclohex-3-en-1-yl)Methyl)-1-(((S)-Oxetan-2-yl)Methyl)-1H-Imidazo[4,5-c]Pyridine-6-Carboxylic Acid

A mixture of methyl 2-((4-(6-((4-cyano-2-fluorobenzyl)oxy)pyridin-2-yl)cyclohex-3-en-1-yl)methyl)-1-(((S)-oxetan-2-yl)methyl)-1H-imidazo[4,5-c]pyridine-6-carboxylate (100 mg, 0.25 mmol) and lithium hydroxide (42 mg, 1 m mol) in THF (2 mL) and water (1 mL) was stirred at room temperature for 10 hours. The reaction mixture was purified by prep-HPLC directly to give 2-((4-(6-((4-cyano-2-fluorobenzyl)oxy)pyridin-2-yl)cyclohex-3-en-1-yl)methyl)-1-(((S)-oxetan-2-yl)methyl)-1H-imidazo[4,5-c]pyridine-6-carboxylic acid (60 mg, yield: 60%) as a white solid. LCMS: m/z 554.1 [M+H]+ ¹H NMR (400 MHz, DMSO-d₆) δ 8.97(s, 1 H),8.44 (s, 1 H), 7.92 (d, J = 10 Hz, 1 H),7.73-7.67 (m, 3 H), 7.10(d, J = 8.0 Hz, 1 H), 6.75 (m ,2 H), 5.50 (s, 2 H), 5.04-5.02(m, 1 H), 4.77-4.72 (m, 1 H), 4.63-4.57 (m, 1 H), 4.43-4.39 (m, 1 H), 4.30-4.25 (m, 1 H), 3.20-3.00(m, 2 H), 2.75-2.50 (m, 2 H), 2.42-2.33 (m, 4 H), 2.13- 1.90 (m, 2 H), 1.62-1.40 (m, 1 H),

Example 17: 2-((4-(6-((4-chloro-2-fluorobenzyl)oxy)pyridin-2-yl)cyclohex-3-en-1-yl)methyl)-1-(((S)-oxetan-2-yl)methyl)-1H-imidazo[4,5-b]pyridine-6-carboxylic acid (Compound 118a)

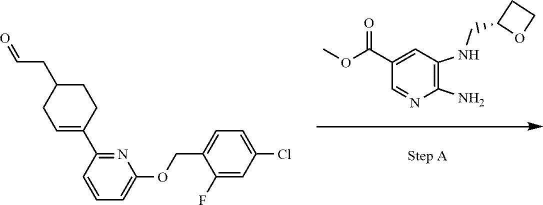

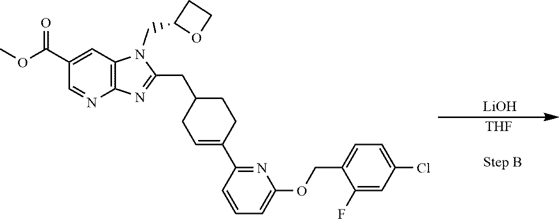

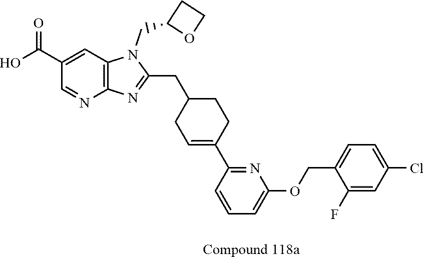

### Step A: The Synthesis of Methyl 2-((4-(6-((4-Chloro-2-Fluorobenzyl)Oxy)Pyridin-2-yl)Cyclohex-3-en-1-yl)Methyl)-1-(((S)-Oxetan-2-yl)Methyl)-1H-Imidazo[4,5-b]Pyridine-6-Carboxylate

To a solution of compound 2-(4-(6-((4-chloro-2-fluorobenzyl)oxy)pyridin-2-yl)cyclohex-3-en-1-yl)acetaldehyde (90 mg, 0.25 mmol) in EtOH (1.5 mL) were added a solution of sodium metabisulfite (25 mg, 013 mmol) in H₂O (1.5 mL). The mixture was stirred at RT for 3 h and then diluted with EtOH (2 mL). The resulting suspension was kept in a refrigerator for 12 h. The suspension was filtered. The precipitate was collected and dried. This resulting precipitate was added to a solution of methyl (S)-6-amino-5-((oxetan-2-ylmethyl)amino)nicotinate (60 mg, 0.25 mmol) in DMF (5 mL). The mixture was stirred at 110° C. for 3 hrs. The reaction mixture was then cooled and poured into water (20 mL). The resulting suspension was extracted with EtOAc (100 mL) for twice. The organic layers were combined, washed with brine (10 mL), and dried over anhydrous Na₂SO₄. After filter, the filtrate was concentrated to give crude product. The crude was purified by Combi-flash (silica gel, eluted with Ethyl acetate/ Petroleum ether from 0% to 50%) to give compound methyl 2-((4-(6-((4-chloro-2-fluorobenzyl)oxy)pyridin-2-yl)cyclohex-3-en-1-yl)methyl)-1-(((S)-oxetan-2-yl)methyl)-1H-imidazo[4,5-b]pyridine-6-carboxylate (0.06 g, 42% yield) as brown oil. LCMS: m/z 577.2 [M+H]⁺.

### Step B: The Synthesis of 2-((4-(6-((4-Chloro-2-Fluorobenzyl)Oxy)Pyridin-2-yl)Cyclohex-3-en-1-yl)Methyl)-1-(((S)-Oxetan-2-yl)Methyl)-1H-Imidazo[4,5-b]Pyridine-6-Carboxylic Acid

A mixture of methyl 2-((4-(6-((4-chloro-2-fluorobenzyl)oxy)pyridin-2-yl)cyclohex-3-en-1-yl)methyl)-1-(((S)-oxetan-2-yl)methyl)-1H-imidazo[4,5-b]pyridine-6-carboxylate (60 mg, 0.10 mmol) and lithium hydroxide (21 mg, 0.5 m mol) in THF (2 mL) and water (1 mL) was stirred at room temperature for 10 hours. The reaction mixture was purified by prep-HPLC directly to give 2-((4-(6-((4-chloro-2-fluorobenzyl)oxy)pyridin-2-yl)cyclohex-3-en-1-yl)methyl)-1-(((S)-oxetan-2-yl)methyl)-1H-imidazo[4,5-b]pyridine-6-carboxylic acid (30 mg, yield: 53%) as white solid. LCMS: m/z 563.2 [M+H]⁺. 1 H NMR (400 MHz, DMSO-d₆) δ 8.97 (s, 1 H),8.50 (s, 1 H), 7.48 (t, J = 8.0 Hz, 1 H), 7.39 (t, J = 8.0 Hz, 1 H), 7.20-7.08 (m, 2 H), 6.92 (d, J = 8 Hz, 1 H), 6.70 - 6.60 (m, 1 H), 6.54 (d, = 8 Hz, 1 H), 5.32 (s, 2 H), 5.14-5.02 (m, 1 H), 4.70-4.58 (m, 1 H), 4.58-4.40 (m, 2 H), 4.38-4.20 (m, 1 H), 3.10-2.90 (m, 2 H), 2.75-2.65 (m, 1 H), 2.60 - 2.50 (m, 1 H), 2.45-2.29 (m, 4 H), 2.10- 1.93 (m, 2 H), 1.54-1.47 (m, 1 H).

Example 18: 2-((4-(4-((4-chloro-2-fluorobenzyl)oxy)pyrimidin-2-yl)cyclohex-3-en-1-yl)methyl)-1-(((S)-oxetan-2-yl)methyl)-1H-imidazo[4,5-b]pyridine-6-carboxylic acid (Compound 119a)

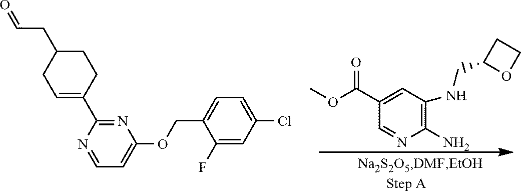

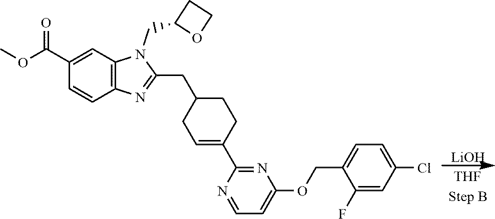

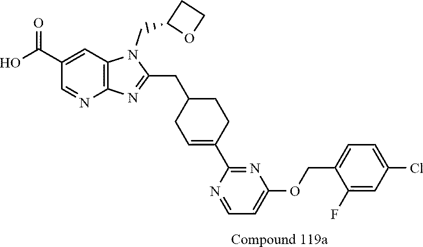

### Step A: The Synthesis of Methyl 2-((4-(4-((4-Chloro-2-Fluorobenzyl)Oxy)Pyrimidin-2-yl)Cyclohex-3-en- 1-yl)Methyl)-1-(((S)-Oxetan-2-yl)-Methyl)-1H-Imidazo[4,5-Blpyridine-6-Carboxylate

To a mixture of 2-(4-(4-((4-chloro-2-fluorobenzyl)oxy)pyrimidin-2-yl)cyclohex-3-en- 1-yl)acetaldehyde (120 mg, 0.3 mmol) in EtOH (3 mL) were added Na₂S₂O₅ (31 mg, 0.1 mmol) in one portion. The mixture was stirred at room temperature for 6 hours. The resulting suspension was filtered. The precipitate was collected, dried, and added to a DMF (3 mL) solution of methyl (S)-6-amino-5-((oxetan-2-ylmethyl)amino)nicotinate (63 mg, 0.2 mmol). The mixture was stirred at 110° C. for 6 hours. The mixture was cooled, diluted with water (10 mL), and extracted with EtOAc (15 mL x 3). Combined organic layers were washed with brine (20 mL), dried over sodium sulfate, filtered, and concentrated in vacuo. The residue was purified by silica gel to give methyl 2-((4-(4-((4-chloro-2-fluorobenzyl)oxy)pyrimidin-2-yl)cyclohex-3-en-1-yl)methyl)-1-(((S)-oxetan-2-yl)methyl)-1H-imidazo[4,5-b]pyridine-6-carboxylate (41 mg, yield: 21%) as yellow oil. MS Calcd.: 577.2; MS Found: 578.2 [M+H]⁺.

### Step B: The Synthesis of 2-((4-(4-((4-Chloro-2-Fluorobenzyl)Oxy)Pyrimidin-2-yl)Cyclohex-3-en-1-yl)Methyl)-1-(((S)-Oxetan-2-yl)Methyl)-1H-Imidazo[4,5-b]Pyridine-6-Carboxylic Acid

A mixture of methyl 2-((4-(4-((4-chloro-2-fluorobenzyl)oxy)pyrimidin-2-yl)cyclohex-3-en-1- yl)methyl)-1-(((S)-oxetan-2-yl)methyl)-1H-imidazo[4,5-b]pyridine-6-carboxylate (41 mg, 0.07 mmol) and lithium hydroxide (10 mg, 0.30 mmol) in THF (3 ml) and water (0.5 ml) was stirred at room temperature for 3 hours. The reaction mixture was purified by prep-HPLC directly to give 2-((4-(4-((4-chloro-2-fluorobenzyl)oxy)pyrimidin-2-yl)cyclohex-3-en-1-yl)methyl)-1-(((S)-oxetan-2-yl)methyl)-1H-imidazo[4,5-b]pyridine-6-carboxylic acid (10 mg, yield: 25%) as white solid. MS Calcd.: 563.1; MS Found: 564.2 [M+H]⁺.

¹H NMR (500 MHz, MeOD) δ 9.06 (d, J= 1.4 Hz, 1 H), 8.59 (d, J= 1.6 Hz, 1 H), 8.40 (d, J= 5.8 Hz, 1 H), 7.52 (t, J= 8.0 Hz, 1 H), 7.30 - 7.20 (m, 3 H), 6.71 (d, J= 5.8 Hz, 1 H), 5.52 (s, 2 H), 5.25 - 5.18 (t, 1 H), 4.78 - 4.70 (m, 1 H), 4.65 - 4.58 (m, 2 H), 4.42 - 4.39 (m, 1 H), 3.20 - 3.15 (m, 2 H), 2.84 - 2.72 (m, 2 H),2.60 - 2.42 (m, 4 H), 2.21 - 2.10 (m, 1 H), 2.10 - 2.02 (m, 1 H), 1.65 - 1.55 (m, 1 H).

Example 19: 2-((4-(4-((5-chloropyridin-2-yl) methoxy)-5-fluoropyrimidin-2-yl)cyclohex-3-en -1-yl)methyl)-3-(((S)-oxetan-2-yl)methyl)-3H-imidazo[4,5-b]pyridine-5-carboxylic acid (Compound 120a)

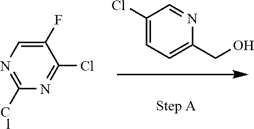

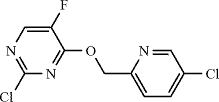 [SMILES: Fc1cnc(Cl)nc1OCc1ccc(Cl)cn1]

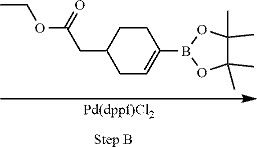

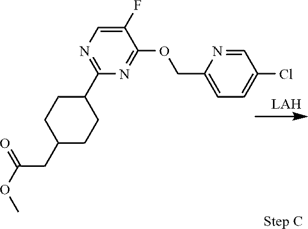

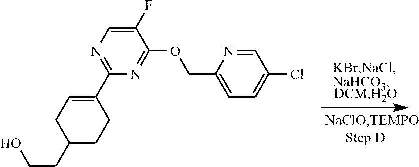

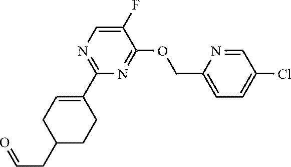 [SMILES: O=CCC1CC=C(c2ncc(F)c(OCc3ccc(Cl)cn3)n2)CC1]

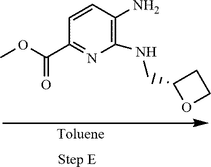

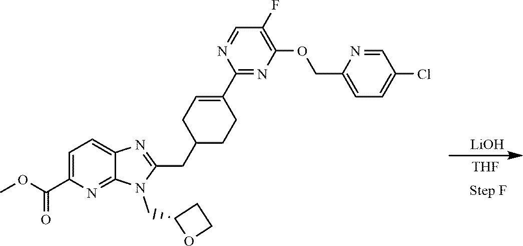

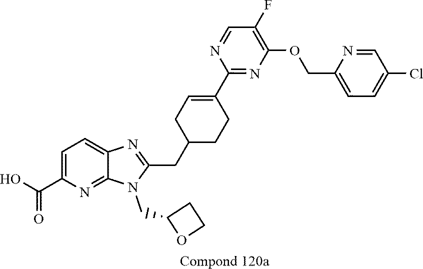

### Step A: The Synthesis of 2-Chloro-4-((5-Chloropyridin-2-yl)Methoxy)-5-Fluoropyrimidine

To a solution of 2, 4-dichloro-5-fluoropyrimidine (1.8 g, 10.78 mmol) and (5-chloropyridin-2-yl) methanol (1.5 g, 10.56 mmol) in CH₃CN (50 mL) were added C_{S2}CO₃ (7.02 g, 21.56 mmol). The mixture was stirred at RT for 16 hours. The mixture was filtered, and the filtrate was concentrated under reduced pressure to afford the title product 2-chloro-4-((5-chloropyridin-2-yl)methoxy)-5-fluoropyrimidine (2.5 g, crude, 85% yield) as a white solid, which was used for next step directly. MS Calcd.: 273.0; MS Found: 274.0 [M+H]⁺.

### Step B: The Synthesis of Ethyl 2-(4-(4-((5-Chloropyridin-2-yl)Methoxy)-5-Fluoropyrimidin-2-yl)Cyclohex-3-en-1-yl)Acetate

To a solution of 2-chloro-4-((5-chloropyridin-2-yl)methoxy)-5-fluoropyrimidine (1 g, 3.65 mmol), tert-butyl-4-(4,4,5,5-tetramethyl-1,3,2-dioxaborolan-2-yl)-3,6-dihydropyridine-1(2H)-carboxylate(1.07 g, 3.65 mmol) and K₂CO₃ (1 g, 7.3 mmol) in Dioxane (32 mL) and H₂O (6 mL) was added Pd(dppf)Cl₂ (133 mg, 0.18 mmol) at RT. The mixture was stirred at 85° C. overnight under N₂ atmosphere. After filter, the filtrate was concentrated to give crude product, which was purified by silica gel column chromatography to afford 2-(4-(4-((5-chloropyridin-2-yl)methoxy)-5-fluoropyrimidin-2-yl) cyclohex-3-en-1-yl)acetate (1 g, 2.46 mmol, 67% yield) as white solid. MS Calcd.: 405.1; MS Found: 406.1 [M+1]⁺.

### Step C: The Synthesis of 2-(4-(4-((5-Chloropyridin-2-yl)Methoxy)-5-Fluoropyrimidin-2-yl)Cyclohex-3-en-1-yl)Ethan-1-ol

To a solution of ethyl 2-(4-(4-((5-chloropyridin-2-yl)methoxy)-5-fluoropyrimidin-2-yl)cyclohex-3-en-1-yl)acetate (1 g, 2.46 mmol) in dry THF (30 mL) was added LAH (187 mg, 4.92 mmol) at -20° C. The mixture was stirred at 0° C. for 2 h. The reaction was quenched by addition of water (0.4 mL) and sodium hydroxide aqueous solution (0.6 mL). The resulting suspension was filtered, and the filter cake was washed with Ethyl acetate (30 mL). The filtrate was concentrated to give crude product, which was purified by silica gel column chromatography to afford the title compound (600 mg, 67% yield) as colorless oil. MS Calcd.: 363.1; MS Found: 364.1 [M+H]⁺.

### Step D: The Synthesis of 2-(4-(4-((5-Chloropyridin-2-yl) Methoxy)-5-Fluoropyrimidin-2-yl)Cyclohex-3-en -1-yl)Acetaldehyde

To a solution of 2-(4-(4-((5-chloropyridin-2-yl)methoxy)-5-fluoropyrimidin-2-yl)cyclohex-3-en-1-yl)ethan-1-ol (300 mg, 0.82 mmol), TEMPO (1.2 mg, 0.08 mmol), NaCl (61.3 mg, 0.82 mmol), NaHCO₃ (47.9 mg, 0.82 mmol), and KBr (9.5 mg, 0.08 mmol) in dichloromethane/water mixture (20 mL/20 mL) was added NaClO aqueous solution (0.98 mL, 7%, 0.82 mmol) dropwise over 20 min at 0° C. The mixture was stirred at that temperature for 15 min. The mixture was diluted with saturated Na₂S₂O₃ aqueous solution (10 mL) and saturated NaHCO₃ aqueous solution (30 mL). The resulting mixture was extracted with dichloromethane (60 mL*3). The combined organic layers were washed with brine (50 mL*2) and dried over Na₂SO₄. After filter, the filtrate was concentrated to give crude product, which was purified on silica gel (0-3% methanol in dichloromethane) to give the title product (280 mg, 0.77 mmol, 93% yield). MS Calcd.:361. 0; MS Found: 362.1 [M+H]⁺.

### Step E: The Synthesis of Methyl 2-((4-(4-((5-Chloropyridin-2-yl) Methoxy)-5-Fluoropvrimidin-2-yl)Cvclohex -3-en-1-yl)Methyl)-3-(((S)-Oxetan-2-yl)Methyl)-3H-Imidazo[4,5-b]Pyridine-5-Carboxylate

To a mixture of 2-(4-(4-((5-chloropyridin-2-yl) methoxy)-5-fluoropyrimidin-2-yl) cyclohex-3-en-1-yl) acetaldehyde (280 mg, 0.77 mmol) and methyl (S)-5-amino-6-((oxetan-2-ylmethyl) amino)picolinate (182.8 mg, 0.77 mmol) in dry toluene (5 mL) was added 4A molecular sieves (150 mg). The mixture was stirred at 100° C. for 40 hours under O₂ atmosphere. The reaction mixture was concentrated and purified on silica gel (DCM\MeOH=20\1, UV 254 nm) to give title product (400 mg, 0.69 mmol, 89% yield) as brown solid. MS Calcd.: 578.2; MS Found: 579.0 [M+H]⁺.

### Step F: The Synthesis of 2-((4-(4-((5-Chloropyridin-2-yl) Methoxy)-5-Fluoropyrimidin-2-yl)Cyclohex-3-en -1-yl)Methyl)-3-(((S)-Oxetan-2-yl)Methyl)-3H-Imidazo[4,5-b]Pyridine-5-Carboxylic Acid

To a solution of methyl 2-((4-(4-((5-chloropyridin-2-yl)methoxy)-5-fluoropyrimidin-2-yl)cyclohex -3-en-1-yl)methyl)-3-(((S)-oxetan-2-yl)methyl)-3H-imidazo[4,5-b]pyridine-5-carboxylate (400 mg, 0.69 mmol) in THF (8 mL) and H₂O (4 mL) was added LiOH.H₂O (252 mg, 0.5 M). The mixture was stirred at 20° C. for 4 hours. The reaction mixture was purified directly by Prep-HPLC (High pH method) to give the desired target product (350 mg, 0.62 mmol, 89% yield). MS Calcd.: 564.1; MS Found: 565.3 [M+H]⁺. ¹H NMR (400 MHz, DMSO-d₆) δ 8.62 (d, J= 2.6 Hz, 1 H), 8.59 (d, J= 2.6 Hz, 1 H), 8.01 - 7.90 (m, 3 H), 7.57 (d, J= 8.4 Hz, 1 H), 7.07 (s, 1 H), 5.61 (s, 2 H), 5.25 - 5.15 (m, 1 H), 4.68 - 4.58 (m, 1 H), 4.56 - 4.39 (m, 2 H), 4.38 - 4.20 (m, 1 H), 3.18 - 2.91 (m, 2 H), 2.74 - 2.57 (m, 2 H), 2.47 - 2.22 (m, 4 H), 2.15 - 2.00(m, 1 H), 2.10 - 1.90 (m, 1 H), 1.57 - 1.34 (m, 1 H).

Example 20: 2-((4-(6-((5-chloropyridin-2-yl) methoxy) pyridin-2-yl) cyclohex-3-en-1-yl)methyl)-3- (((S)-oxetan-2-yl)methyl)-3H-imidazo[4,5-b]pyridine-5-carboxylic acid (Compound 121a)

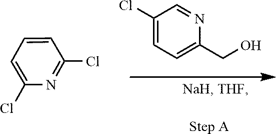

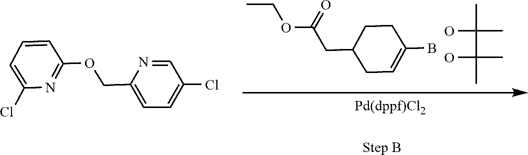

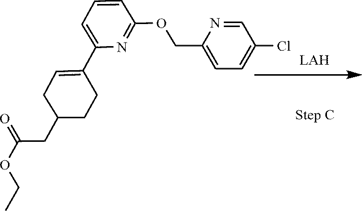

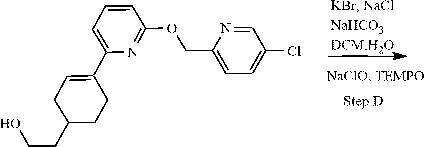

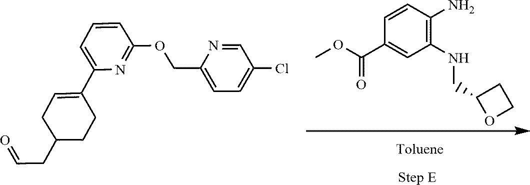

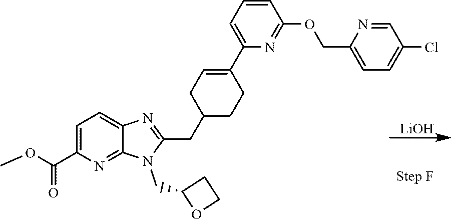

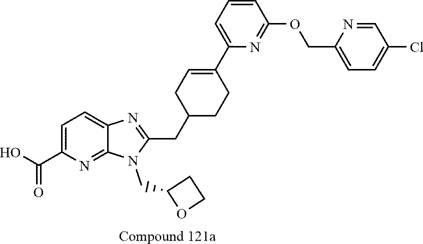

### Step A: The Synthesis of 5-Chloro-2-(((6-Chloropyridin-2-yl)Oxy)Methyl)Pyridine

To a solution of 2, 6-dichloropyridine (1 g, 6.24 mmol) and (5-chloropyridin-2-yl) methanol (865.05 mg, 6.24 mmol) in THF (50 mL) were added to NaH (449 mg). The mixture was stirred at RT for 16 hours. The mixture was diluted with ice water (100 mL). The resulting mixture was extracted with ethyl acetate (80 mL*3). Combined organic layers were washed with brine (80 mL*2) and dried over Na₂SO₄. After filtration, the filtrate was concentrated to give crude product, which was purified by silica gel column chromatography to afford the title product (500 mg, 1.96 mmol, 31% yield) as a white solid. MS Calcd.: 254.0; MS Found: 255.0 [M+H]⁺.

### Step B: The Synthesis of Ethyl 2-(4-(6-((5-Chloropyridin-2-yl)Methoxy)Pyridin-2-yl)Gyclohex-3-en-1-yl) Acetate

To a solution of 5-chloro-2-(((6-chloropyridin-2-yl)oxy)methyl)pyridine (450 mg, 1.76 mmol), tert-butyl-4-(4,4,5,5-tetramethyl-1,3,2-dioxaborolan-2-yl)-3,6-dihydropyridine-1(2H)-carboxylate(519 mg, 1.76 mmol) and K₂CO₃ (486.5 mg, 3.52 mmol) in Dioxane (32 mL) and H₂O (6 mL) was added Pd(dppf)Cl₂ (64.38 mg, 0.09 mmol) at RT. The mixture was stirred at 85° C. overnight under N₂ atmosphere. After filtration, the filtrate was concentrated to give crude product, which was purified by silica gel column chromatography to afford 2-(4-(6-((5-chloropyridin-2-yl)methoxy)pyridine-2-yl)cyclohex -3-en-1-yl)acetate (500 mg, 1.29 mmol, 73% yield) as white solid. MS Calcd.: 386.1; MS Found: 387.0 [M+1]⁺.

### Step C: The Synthesis of 2-(4-(6-((5-Chloropyridin-2-yl)Methoxy)Pyridin-2-yl)Cyclohex-3-en-1-yl)Ethan-1-ol

To a solution of ethyl 2-(4-(6-((5-chloropyridin-2-yl) methoxy) pyridin-2-yl) cyclohex-3-en-1-yl) acetate (500 mg, 1.29 mmol) in anhydrous THF (30 mL) was added LAH (98 mg, 2.58 mmol) at -20° C. The mixture was stirred at 0° C. for 2 hours. The mixture was quenched by addition of water (0.4 mL) and sodium hydroxide aqueous solution (0.6 mL). The resulting suspension was filtered, and the filter cake was washed with ethyl acetate (50 mL). The filtrate was concentrated to give crude product, which was purified by silica gel column chromatography to afford 2-(4-(6-((5-chloropyridin-2-yl)methoxy)pyridin-2-yl)cyclohex-3-en-1-yl) ethan-1-ol (400 mg, 1.1 mmol, 89% yield) as colorless oil. MS Calcd.: 344.1; MS Found: 344.9 [M+H]⁺.

### Step D: The Synthesis of 2-(4-(6-((5-Chloropyridin-2-yl)Methoxy)Pyridin-2-yl)Cyclohex-3-en-1-yl)Acetaldehyde

To the solution of 2-(4-(6-((5-chloropyridin-2-yl)methoxy)pyridin-2-yl)cyclohex-3-en-1-yl)ethan-1-ol (450 mg, 1.31 mmol), TEMPO (1.5 mg, 0.01 mmol), NaCl (76.5 mg, 1.31 mmol), NaHCO₃ (110 mg, 1.31 mmol), and KBr (15.4 mg, 0.13 mmol) in dichloromethane/water mixture (20 mL/20 mL) was added NaClO aqueous solution (1.4 mL, 7%, 1.31 mmol) dropwise over 20 min at 0° C. The mixture was diluted with saturated Na₂S₂O3 aqueous solution (10 mL), saturated NaHCO₃ aqueous solution (30 mL). The resulting mixture was extracted with dichloromethane (60 mL*3). Combined organic layers were washed with brine (50 mL*2) and dried over Na₂SO₄. After filter, the filtrate was concentrated to give crude product, which was purified on silica gel (0-3% methanol in dichloromethane) to give the title product (350 mg, 1.02 mmol, 78% yield). MS Calcd.:342.1; MS Found: 343.1 [M+H]⁺.

### Step E: The Synthesis of Methyl 2-((4-(6-((5-Chloropyridin-2-yl)Methoxy)Pyridin-2-yl)Cyclohex-3-en-1-yl)Methyl)-3-(((S)-Oxetan-2-yl)methyl)-3H-imidazo[4,5-b]pyridine-5-carboxylate

To a mixture of 2-(4-(6-((5-chloropyridin-2-yl) methoxy) pyridin-2-yl) cyclohex-3-en-1-yl) acetaldehyde (350 mg, 1.02 mmol) and methyl (S)-5-amino-6-((oxetan-2-ylmethyl) amino) picolinate (241.2 mg, 1.02 mmol) in dry toluene (10 mL) was added 4A molecular sieves (150 mg). The mixture was stirred at 80° C. for 40 hours under O₂ atmosphere. The reaction mixture was concentrated and purified on silica gel (DCM\MeOH=20\1, UV 254 nm) to give title product (500 mg, 0.89 mmol, 87% yield) as brown solid. MS Calcd.: 559.2; MS Found: 560.2 [M+H]⁺.

### Step F: The Synthesis of 2-((4-(6-((5-chloropyridin-2-yl) Methoxy) Pyridin-2-yl) cyclohex-3-en-1-yl)methyl)-3- (((S)-oxetan-2-yl)methyl)-3H-imidazo[4,5-b]pyridine-5-carboxylic acid

To a solution of methyl 2-((4-(6-((5-chloropyridin-2-yl)methoxy)pyridin-2-yl)cyclohex-3-en-1 -yl)methyl)-3-(((S)-oxetan-2-yl)methyl)-3H-imidazo[4,5-b]pyridine-5-carboxylate(500 mg, 0.89 mmol) in THF (8 mL) and H₂O (4 mL) was added LiOH.H₂O (252 mg, 0.5 M). The mixture was stirred at 20° C. for 4 hours. The reaction mixture was purified directly by Prep-HPLC (High pH method) to give 2-((4-(6-((5-chloropyridin-2-yl) methoxy) pyridin-2-yl) cyclohex-3-en-1-yl)methyl)-3- (((S)-oxetan-2-yl)methyl)-3H-imidazo[4,5-b]pyridine-5-carboxylic acid (210 mg, 0.38 mmol, 43% yield). MS Calcd.: 545.2; MS Found: 546.2 [M+H]⁺. ¹H NMR (400 MHz, DMSO-d₆) δ 8.60 (d, J= 2.3 H_{z}, 1 H), 8.08 (d, J= 8.2 Hz, 1 H), 8.01 - 7.87 (m, 2 H), 7.69 (t, J= 7.8 Hz, 1 H), 7.48 (d, J= 8.4 Hz, 1 H), 7.08 (d, J= 7.4 Hz, 1 H), 6.78 (d, J= 8.1 Hz, 1 H), 6.71 (br.s, 1 H), 5.46 (d, J= 12.5 Hz, 2 H), 5.20 - 5.12 (br.s, 1 H), 4.70 - 4.60 (m, 1 H), 4.60 - 4.40 (m, 2 H), 4.35 - 4.23 (m, 1 H), 3.17 - 2.94 (m, 2 H), 2.76 - 2.60 (m, 1 H), 2.47 - 2.21 (m, 5 H), 2.11 - 1.91 (m, 2H), 1.57 - 1.3 4 (m, 1 H).

Example 21: 2-((4-(4-((4-cyano-2-fluorobenzyl)oxy)-5-fluoropyrimidin-2-yl)cyclohex-3-en-1-yl)methyl)-3-(cyclopropylmethyl)-3H-imidazo[4,5-b]pyridine-5-carboxylic acid (Compound 122)

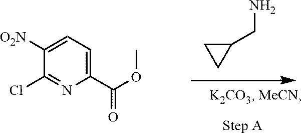

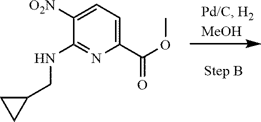

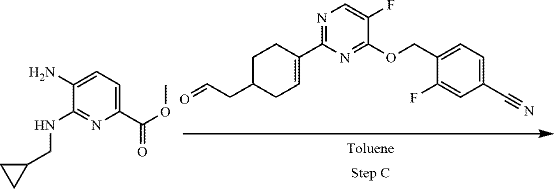

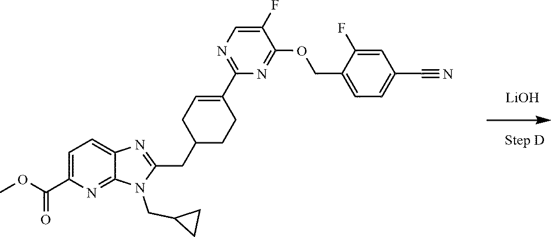

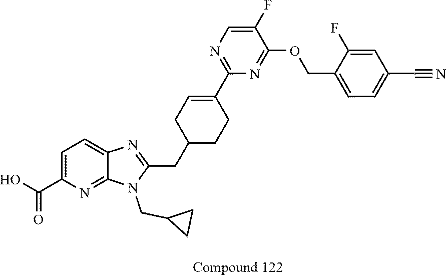

### Step A: The Synthesis of Methyl 6-((cyclopropylmethyl)amino)-5-nitropicolinate

To the solution of methyl 6-chloro-5-nitropicolinate (1.0 g, 4.6 mmol) in acetonitrile (5 mL) was added K₂CO₃ (1.28 g, 9.25 mmol) and cyclopropylmethanamine (0.4 mg, 5.1 mmol) and stirred at 30° C. for 24 h under nitrogen atmosphere. The mixture was diluted with ethyl acetate (20 mL) and washed with water (20 mL), brine (10 mL). The organic layer was concentrated, purified on silica gel (0-35%, ethyl acetate in petroleum ether) to give methyl 6-((cyclopropylmethyl)amino)-5-nitropicolinate (0.8 g, 3.19 mmol). MS Calcd.: 251.1; MS Found: 252.0 [M+H]⁺.

### Step B: The Synthesis of Methyl 5-amino-6-((cyclopropylmethyl)amino)picolinate

To a solution of methyl 6-((cyclopropylmethyl)amino)-5-nitropicolinate (200 mg, 0.797 mmol) in MeOH (10 mL) was added Pd/C (10%, 20 mg) at RT. The mixture was stirred at RT under H₂ atmosphere for 12 hrs. The mixture was filtered, and the filtrate was concentrated under reduced pressure to afford methyl 5-amino-6-((cyclopropylmethyl)amino)picolinate (120 mg, 92% yield) as white solid, which was used in the next step directly. MS Calcd.: 221.0; MS Found 222.1 [M+H]⁺.

### Step C: The Synthesis of Methyl 2-((4-(4-((4-cvano-2-fluorobenzyl)oxy)-5-fluoropyrimidin-2-yl)cvclohex-3-en-l-yl)methyl)-3-(cyclopropylmethyl)-3H-imidazo[4,5-b]pyridine-5-carboxylate

To a suspension of methyl 5-amino-6-((cyclopropylmethyl)amino)picolinate (120 mg) and 3-fluoro-4-(((5-fluoro-2-(4-(2-oxoethyl)cyclohex-1-en-1-yl)pyrimidin-4-yl)oxy)methyl)benzonitrile (182 mg, 0.494 mmol) in dry toluene (5 mL) was added 4A molecular sieves (364 mg). The mixture was stirred at 80° C. for 40 h under O₂ atmosphere. The reaction mixture was concentrated and purified on silica gel (DCM\MeOH=10\1, UV254 nm) to give methyl 2-((4-(4-((4-cyano-2-fluorobenzyl)oxy)-5-fluoropyrimidin-2-yl)cyclohex-3-en-1-yl)methyl)-3-(cyclopropylmethyl)-3H-imidazo[4,5-b]pyridine-5-carboxylate (150 mg, 0.263 mmol) as brown solid. MS Calcd.: 570.0; MS Found: 571.0 [M+H]⁺.

### Step D: The Synthesis of 2-((4-(4-((4-cyano-2-fluorobenzvl)oxy)-5-fluoropyrimidin-2-yl)cyclohex-3-en-l-yl)methyl)-3-(cyclopropylmethyl)-3H-imidazo[4,5-b1pyridine-5-carboxylic acid

To a solution of methyl 2-((4-(4-((4-cyano-2-fluorobenzyl)oxy)-5-fluoropyrimidin-2-yl)cyclohex-3-en-1-yl)methyl)-3-(cyclopropylmethyl)-3H-imidazo[4,5-b]pyridine-5-carboxylate (150 mg, 0.263 mmol) in THF (4 mL) and H₂O (2 mL) was added LiOH.H₂O (22 mg, 0.526 mmol). The mixture was stirred at 20° C. for 4 h. The reaction mixture was purified directly by Prep-HPLC (High pH method) to give 2-((4-(4-((4-cyano-2-fluorobenzyl)oxy)-5-fluoropyrimidin-2-yl)cyclohex-3-en-1-yl)methyl)-3-(cyclopropylmethyl)-3H-imidazo[4,5-b]pyridine-5-carboxylic acid (64 mg, 0.11 mmol). MS Calcd.: 556.2; MS Found 557.3 [M+H]⁺.

¹H NMR (400 MHz, MeOD) δ 8.38 (d, J = 2.9 Hz, 1 H), 8.11 (d, J = 8.3 Hz, 1 H), 8.05 (d, J = 8.3 Hz, 1 H), 7.75 (t, J = 7.4 Hz, 1 H), 7.69 - 7.56 (m, 2 H), 7.17 (br.s, 1 H), 5.69 (s, 2 H), 4.38 (d, J = 7.1 Hz, 2 H), 3.09 (t, J = 11.3 Hz, 2 H), 2.78 (d, J = 17.0 Hz, 1 H), 2.60 -2.40 (m, 3 H), 2.21 - 2.01 (m, 2 H), 1.65 - 1.52 (m, 1 H), 1.44 - 1.24 (m, 1 H), 0.66 - 0.50 (m, 4 H).

Example 22: 2-((4-(4-((4-cyano-2-fluorobenzyl)oxy)-5-fluoropyrimidin-2-yl)cyclohex-3-en-1-yl)methyl)-3-((1-cyanocyclopropyl)methyl)-3H-imidazo[4,5-b]pyridine-5-carboxylic acid (Compound 123)

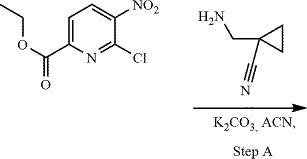

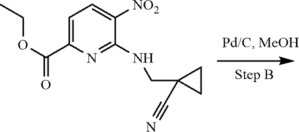

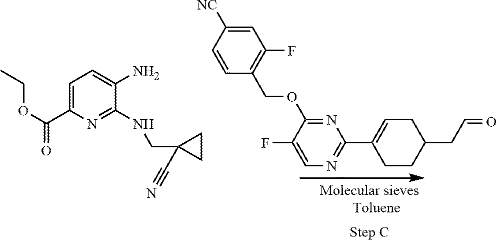

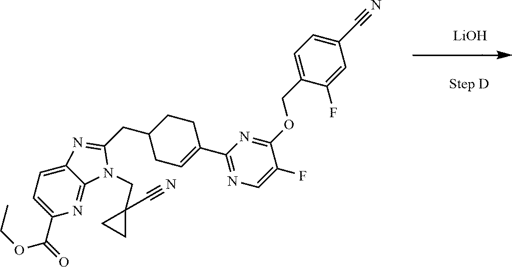

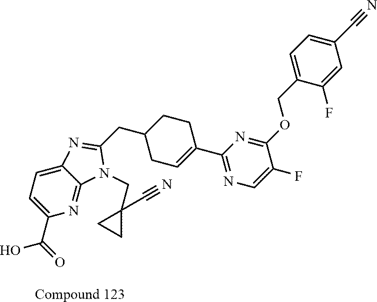

### Step A: The Synthesis of Methyl 6-(((1-cyanocyc1opropyl)methyl)amino)-5-nitropicolinate

To a solution of 1-(aminomethyl) cyclopropane-1-carbonitrile (300 mg, 3.1 mmol), K₂CO₃ (850 mg, 6.2 mmol) in ACN (10 mL) was added ethyl 6-chloro-5-nitropicolinate (700 mg, 3.1 mmol) at RT. The mixture was stirred at 30° C. overnight. The reaction mixture was filtered, and the filtrate was concentrated under reduced pressure. The crude was purified by Prep-TLC (PE/EA=10/1) to give ethyl 6-(((1-cyanocyclopropyl) methyl) amino)-5-nitropicolinate (100 mg, yield: 10%) as yellow oil. MS Calcd.: 290.1; MS Found: 291.0 [M+H] ⁺.

### Step B: The Synthesis of Ethyl 5-amino-6-(((1-cyanocyclopropyl)methyl)amino)picolinate

To a solution give ethyl 6-(((1-cyanocyclopropyl) methyl) amino)-5-nitropicolinate (100 mg, 0.34 mmol) in MeOH (5 mL) was added Pd/C (10%, 60 mg) at RT. The mixture was stirred at room temperature under H₂ for 4 hours. The reaction mixture was filtered, and the filtrate was concentrated under reduced pressure to afford ethyl 5-amino-6-(((1-cyanocyclopropyl) methyl) amino)picolinate (64 mg, yield: 82%) as yellow solid. MS Calcd.: 260.1; MS Found: 261.1 [M+H] ⁺.

### Step C: The Synthesis of Ethyl 2-((4-(4-((4-cyano-2-fluorobenzyl)oxy)-5-fluoropyrimidin-2-yl)cyclohex-3-en-1-yl)methyl)-3-((1-cyanocyclopropyl)methyl)-3H-imidazo[4,5-b]pyridine-5-carboxylate

A mixture of methyl 5-amino-6-(((1-cyanocyclopropyl) methyl) amino) picolinate (64 mg, 0.26 mmol), 3-fluoro-4-(((5-fluoro-2-(4-(2-oxoethyl)cyclohex-1-en-1-yl)pyrimidin-4-yl)oxy)methyl)benzonitrile (121 mg, 0.33 mmol) and Molecular sieves (250 mg) in toluene (5 mL) was stirred at 80° C. under O₂ for 3 days. The reaction mixture was filtered, and the filtrate was concentrated under reduced pressure. The crude was purified by flash silica column chromatography (eluent =5%-80%EA in PE) to give ethyl 2-((4-(4-((4-cyano-2-fluorobenzyl)oxy)-5-fluoropyrimidin-2-yl)cyclohex-3-en-1-yl)methyl)-3-((1-cyanocyclopropyl)methyl)-3H-imidazo[4,5-b]pyridine-5-carboxylate (80 mg, yield: 45%) as yellow solid. MS Calcd.: 609.2; MS Found: 610 [M+H] ⁺.

### Step D: The Synthesis of 2-((4-(4-((4-cyano-2-fluorobenzvl)oxy)-5-fluoropyrimidin-2-yl)cyc1ohex-3-en-1-yl)methyl)-3-((1-cyanocyc1opropyl)methyl)-3H-imidazo[4.5-b]pyridine-5-carboxylic acid

A mixture of ethyl 2-((4-(4-((4-cyano-2-fluorobenzyl)oxy)-5-fluoropyrimidin-2-yl)cyclohex-3-en-1-yl)methyl)-3-((1-cyanocyclopropyl)methyl)-3H-imidazo[4,5-b]pyridine-5-carboxylate (80 mg, 0.13 mmol) and lithium hydroxide (40 mg, 1 mmol) in methanol (3 mL) and water (0.5 mL) was stirred at room temperature for 5 hours. The reaction mixture was purified by prep-HPLC directly to give 2-((4-(4-((4-cyano-2-fluorobenzyl)oxy)-5-fluoropyrimidin-2-yl)cyclohex-3-en-1-yl)methyl)-3-((1-cyanocyclopropyl)methyl)-3H-imidazo[4,5-b]pyridine-5-carboxylic acid (6 mg, yield: 8%) as yellow solid. MS Calcd.: 581.2; MS Found: 582.0[M+H] ⁺. ¹H NMR (400 MHz, CD3OD) δ 8.37 (d, J = 2.8 Hz, 1 H), 8.14 (d, J = 8.4 Hz, 1 H), 8.08 (d, J = 8 Hz, 1 H), 7.75 (t, J = 7.6 Hz,1 H), 7.68 - 7.60 (m, 2 H), 7.18 (br.s, 1 H), 5.73 (s, 2 H), 4.66 (d, J = 2 Hz, 2 H), 3.25 (d, J = 6.8 Hz, 2 H), 2.78 (d, J = 12 Hz, 1 H), 2.60 - 2.40 (m, 3 H), 2.22-2.17 (m, 1 H), 2.17-2.05 (m, 1 H), 1.74-1.68 (m, 2 H), 1.64-1.45 (m, 1 H), 1.39-1.30 (m, 2 H).

Example 23: 2-((4-(4-((4-cyano-2-fluorobenzyl)oxy)-5-fluoropyrimidin-2-yl)cyclohex-3-en-1-yl)methyl)-3-(2-cyano-2-methylpropyl)-3H-imidazo[4,5-b]pyridine-5-carboxylic acid (Compound 124)

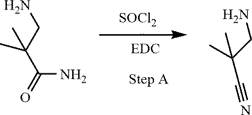

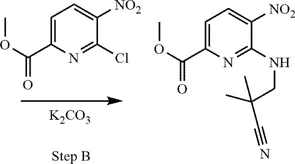

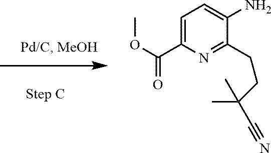

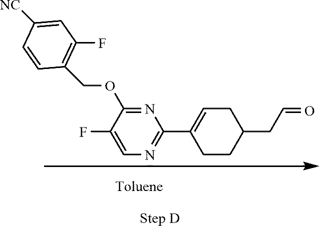

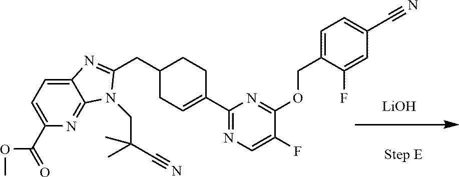

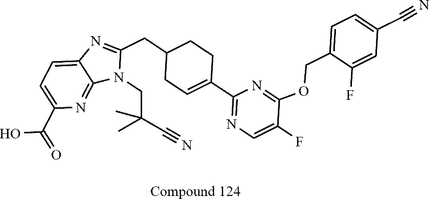

### Step A: The Synthesis of 3-amino-2,2-dimethylpropanenitrile

To the solution of 3-amino-2,2-dimethylpropanamide (1.0 g, 8.62 mmol) in DCE (10 mL) was added SOCl₂ (2.0 g, 17.24 mmol) stirred at 90° C. for 5 h under nitrogen atmosphere. The mixture was diluted with DCM (20 mL) and washed with water (20 mL), brine (10 mL). The organic layer was separated, concentrated, and purified on silica gel (0-35%, ethyl acetate in petroleum ether) to give 3-amino-2,2-dimethylpropanenitrile (0.5 g, 5.10 mmol). MS Calcd.: 98.1; MS Found: 99.1(M+H).

### Step B: The Synthesis of Methyl 6-((2-cvano-2-methylpropyl)amino)-5-nitropicolinate

To a solution of 3-amino-2,2-dimethylpropanenitrile (500 mg, 5.05 mmol) and methyl 6-chloro-5-nitropicolinate (1.09 g, 5.05 mmol) in CH₃CN (30 mL) was added K₂CO₃ (1.394 g, 10.1 mmol) in one portion. The reaction mixture was stirred at 30° C. for 13 hr. The mixture was cooled to room temperature. The mixture was diluted with H₂O (100 mL) and extracted with EtOAc (100 mL) for twice. The organic layers were combined, washed with brine (50 mL), and dried over anhydrous Na₂SO₄. The filtrate was concentrated and purified by Combi flash (silica gel, eluted with Ethyl acetate/ Petroleum ether from 0% to 25%, UV 254 nm) to give methyl 6-((2-cyano-2-methylpropyl)amino)-5-nitropicolinate (300 mg, 1.08 mmol, 21% yield) as a white solid. MS Calcd.: 278.1; MS Found: 279.1 (M+H)

### Step C: The Synthesis of Methyl 5-amino-6-((2-cyano-2-methylpropyl)amino)picolinate

To a solution of methyl 6-((2-cyano-2-methylpropyl)amino)-5-nitropicolinate (300 mg, 1.08 mmol) in methanol (5 mL) was added Pd/C (10%, 100 mg). The mixture was stirred at RT under H₂ atmosphere for 16 h. Then the mixture was filtered, and the filtrate was concentrated under reduced pressure. The residue was purified by prep-TLC (silica, UV254 nm, PE/EA=10/1) to afford methyl 5-amino-6-((2-cyano-2-methylpropyl)amino)picolinate (260 mg, yield: 97%) as yellow oil. MS Calcd.: 248.1; MS Found: 249.1 [M+H]⁺.

### Step D: The Synthesis of Methyl 2-((4-(4-((4-cyano-2-fluorobenzyl)oxy)-5-fluororpyrimidin-2-yl)cyclohex-3-en-1-yl)methyl)-3-(2-cyano-2-methylproryl)-3H-imidazo[4, 5-b]pyridine-5-carboxylate

To a mixture of methyl 5-amino-6-((2-cyano-2-methylpropyl)amino)picolinate (100 mg, 0.40 mmol) and 3-fluoro-4-(((5-fluoro-2-(4-(2-oxoethyl)cyclohex-1-en-1-yl)pyrimidin-4-yl)oxy)methyl)benzonitrile (149 mg, 0.40 mmol) in anhydrous toluene (5 mL) was added 4A molecular sieves (200 mg). The mixture was stirred at 80° C. for 40 hours under O₂ atmosphere. LCMS indicated starting material was consumed completely and desired product was found as major peak. The reaction mixture was concentrated and purified on silica gel (DCM\MeOH=10\1, UV254 nm) to give methyl 2-((4-(4-((4-cyano-2-fluorobenzyl)oxy)-5-fluoropyrimidin-2-yl)cyclohex-3-en-1-yl)methyl)-3-(2-cyano-2-methylpropyl)-3H-imidazo[4,5-b]pyridine-5-carboxylate (120 mg, 0.201 mmol) as brown solid. MS Calcd.: 597.2; MS Found: 597.9 [M+H]⁺.

### Step E: The Synthesis of 2-((4-(4-((4-cyano-2-fluorobenzyl)oxy)-5-fluoropyrimidin-2-yl)cyclohex-3-en-1-yl)methyl)-3-(2-cyano-2-methylpropyl)-3H-imidazo[4,5-b]pyridine-5-carboxylic acid

To a solution of methyl 2-((4-(4-((4-cyano-2-fluorobenzyl)oxy)-5-fluoropyrimidin-2-yl)cyclohex-3-en-l-yl)methyl)-3-(2-cyano-2-methylpropyl)-3H-imidazo[4,5-b]pyridine-5-carboxylate (120 mg, 0.201 mmol) in THF (4 mL) and H₂O (2 mL) was added LiOH.H₂O (34 mg, 0.804 mmol). The mixture was stirred at 20° C. for 4 h. The reaction mixture was purified directly by Prep-HPLC (High pH method) to give 2-((4-(4-((4-cyano-2-fluorobenzyl)oxy)-5-fluoropyrimidin-2-yl)cyclohex-3-en-1-yl)methyl)-3-(2-cyano-2-methylpropyl)-3H-imidazo[4,5-b]pyridine-5-carboxylic acid (10.3 mg, 0.02 mmol). MS Calcd.: 583.2; MS Found 583.9[M+H]⁺.

¹H NMR (400 MHz, MeOD) δ 8.38 (d, J = 2.9 Hz, 1 H), 8.17 (d, J = 8.3 Hz, 1 H), 8.10 (d, J = 8.3 Hz, 1 H), 7.75 (t, J = 7.5 Hz, 1 H), 7.70 - 7.60 (m, 2 H), 7.17 (br.s, 1 H), 5.70 (s, 2 H), 4.73 (s, 2 H), 3.26 (d, J = 6.8 Hz, 2 H), 2.78 (d, J = 15.9 Hz, 1 H), 2.60 - 2.40 (m, 3 H), 2.25 - 2.10 (m, 1 H), 2.10 -2.00 (m, 1 H), 1.65 - 1.50 (m, 1 H),1.52 (s, 6 H).

Example 24: 2-((4-(4-((4-chloro-2-fluorobenzyl)oxy)-5-fluoropyrimidin-2-yl)cyclohex-3-en-1-yl)methyl)-3-((1-cyanocyclopropyl)methyl)-3H-imidazo[4,5-b]pyridine-5-carboxylic acid (Compound 125)

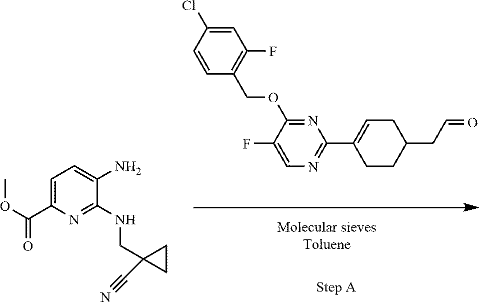

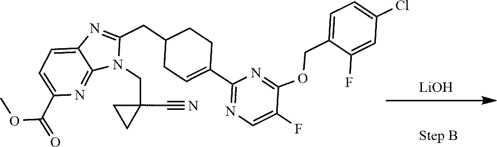

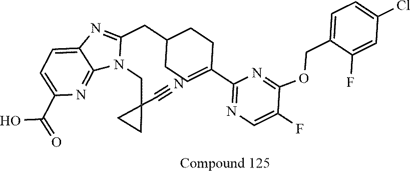

### Step A: The Synthesis of Methyl 2-((4-(4-((4-chloro-2-fluorobenzyl)oxy)-5-fluoropyrimidin-2-yl)cyclohex-3-en-1-yl)methyl)-3-((1-cyanocyclopropyl)methyl)-3H-imidazo[4,5-b]pyridine-5-carboxylate

A mixture of methyl 5-amino-6-(((1-cyanocyclopropyl) methyl) amino) picolinate (100 mg, 0.4 mmol), 2-(4-(4-((4-chloro-2-fluorobenzyl)oxy)-5-fluoropyrimidin-2-yl)cyclohex-3-en-1-yl)acetaldehyde (181 mg, 0.48 mmol) and Molecular sieves (4A, 360 mg) in toluene (10 mL) was stirred at 80° C. under O₂ for 40 hours. The reaction mixture was filtered, and the filtrate was concentrated under reduced pressure. The crude was purified by flash silica column chromatography (eluent =5%-80%EA in PE) to give methyl 2-((4-(4-((4-chloro-2-fluorobenzyl)oxy)-5-fluoropyrimidin-2-yl)cyclohex-3-en-1-yl)methyl)-3-((1-cyanocyclopropyl)methyl)-3H-imidazo[4,5-b]pyridine-5-carboxylate (140 mg, yield: 58%) as yellow solid. MS Calcd.: 604.2; MS Found: 604.9 [M+H] ⁺.

### Step B: The Synthesis of 2-((4-(4-((4-chloro-2-fluorobenzyl)oxy)-5-fluoropyrimidin-2-yl)cyclohex-3-en-1-yl)methyl)-3-((1-cyanocyclopropyl)methyl)-3H-imidazo[4,5-b]pyridine-5-carboxylic acid

A mixture of methyl 2-((4-(4-((4-chloro-2-fluorobenzyl)oxy)-5-fluoropyrimidin-2-yl)cyclohex-3-en-l-yl)methyl)-3-((1-cyanocyclopropyl)methyl)-3H-imidazo[4,5-b]pyridine-5-carboxylate (140 mg, 0.23 mmol) and lithium hydroxide (84 mg, 2.0 mmol) in methanol (4 mL) and water (0.8 mL) was stirred at room temperature for 3 hours. The reaction mixture was purified by prep-HPLC directly to give 2-((4-(4-((4-chloro-2-fluorobenzyl)oxy)-5-fluoropyrimidin-2-yl)cyclohex-3-en-1-yl)methyl)-3-((1-cyanocyclopropyl)methyl)-3H-imidazo[4,5-b]pyridine-5-carboxylic acid (19 mg, yield: 14%) as yellow solid. MS Calcd.: 590.2; MS Found: 591.0 [M+H] ⁺. ¹H NMR (500 MHz, CD₃OD) δ 8.34-8.34 (d, J = 2.5 Hz, 1 H), 8.15 (d, J = 8 Hz, 1 H), 8.10 (d, J = 8 Hz, 1 H), 7.55 (t, J = 8.5 Hz, 1 H), 7.30 - 7.22 (m, 2 H), 7.20 (br.s, 1 H), 5.60 (s, 2 H), 4.66-4.66 (d, J = 3 Hz, 2 H), 3.26 (d, J = 7 Hz, 2 H), 2.82 (d, J = 17.5 Hz, 1 H), 2.60 - 2.45 (m, 3 H), 2.25-2.05 (m, 2 H), 1.70-1.68 (m, 2 H), 1.68 -1.55 (m, 1 H), 1.40-1.32 (m, 2 H).

Example 25: 2-((4-(4-((4-chloro-2-fluorobenzyl)oxy)-5-fluoropyrimidin-2-yl)cyclohex-3-en-1-yl)methyl)-3-(2-cyano-2-methylpropyl)-3H-imidazo[4,5-b]pyridine-5-carboxylic acid (Compound 126)

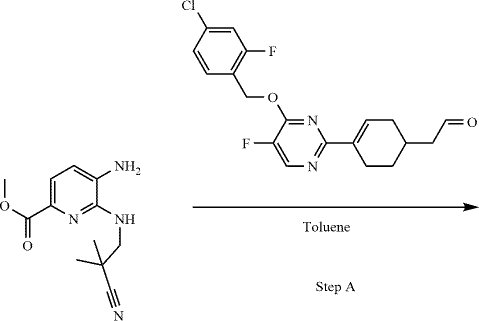

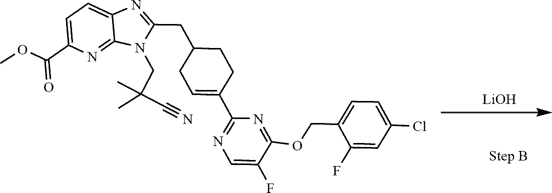

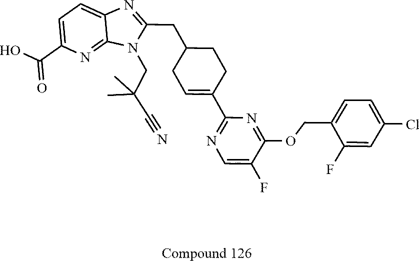

### Step A: The Synthesis of Methyl 2-((4-(4-((4-chloro-2-fluorobenzyl)oxy)-5-fluoropyrimidin-2-yl)cyclohex-3-en-1-yl)methyl)-3-(2-cyano-2-methylpropyl)-3H-imidazo[4, 5-b]pyridine-5-carboxylate

To a mixture of methyl 5-amino-6-((2-cyano-2-methylpropyl)amino)picolinate (100 mg, 0.40 mmol) and 2-(4-(4-((4-chloro-2-fluorobenzyl)oxy)-5-fluoropyrimidin-2-yl)cyclohex-3-en-1-yl)acetaldehyde (152 mg, 0.40 mmol) in anhydrous toluene (5 mL) was added 4A molecular sieves (200 mg). The mixture was stirred at 80° C. for 40 h under O₂ atmosphere. The reaction mixture was concentrated and purified on silica gel (DCM\MeOH=10\1, UV254 nm) to give methyl 2-((4-(4-((4-chloro-2-fluorobenzyl)oxy)-5-fluoropyrimidin-2-yl)cyclohex-3-en-1-yl)methyl)-3-(2-cyano-2-methylpropyl)-3H-imidazo[4,5-b]pyridine-5-carboxylate (74 mg, 0.122 mmol) as brown solid. MS Calcd.: 606.2; MS Found: 607.2 [M+H]⁺.

### Step B: The Synthesis of 2-((4-(4-((4-chloro-2-fluorobenzyl)oxy)-5-fluoropyrimidin-2-yl)cyclohex-3-en-1-yl)methyl)-3-(2-cyano-2-methylpropyl)-3H-imidazo[4,5-b]pyridine-5-carboxylic acid

To a solution of methyl 2-((4-(4-((4-chloro-2-fluorobenzyl)oxy)-5-fluoropyrimidin-2-yl)cyclohex-3-en-l-yl)methyl)-3-(2-cyano-2-methylpropyl)-3H-imidazo[4,5-b]pyridine-5-carboxylate (100 mg, 0.122 mmol) in THF (4 mL) and H₂O (2 mL) was added LiOH.H₂O (21 mg, 0.488 mmol). The mixture was stirred at 20° C. for 4 h. The reaction mixture was purified directly by Prep-HPLC to give the desired target product 2-((4-(4-((4-chloro-2-fluorobenzyl)oxy)-5-fluoropyrimidin-2-yl)cyclohex-3-en-1-yl) methyl)-3-(2-cyano-2-methylpropyl)-3H-imidazo[4,5-b]pyridine-5-carboxylic acid (21.6 mg, 0.04 mmol). MS Calcd.: 592.2; MS Found 592.9[M+H]⁺. ¹H NMR (400 MHz, MeOD) δ 8.35 (d, J = 2.9 Hz, 1 H), 8.17 (dd J = 8.3 Hz, 1 H), 8.11 (dd J = 8.3 Hz, 1 H), 7.56 (t, J = 8.0 Hz, 1 H), 7.35 - 7.10 (m, 3 H), 5.60 (s, 2 H), 4.73 (s, 2 H), 3.27 (d, J = 6.7 Hz, 2 H),2.90 - 2.69 (m, 1 H), 2.60 - 2.40 (m, 3 H), 2.25 - 2.09 (m, 1 H), 2.00 - 2.00 (m, 1 H), 1.70 - 1.58 (m, 1H) 1.52 (s, 6 H).

Example 26: 2-((4-(4-((4-chloro-2-fluorobenzyl)oxy)-5-fluoropyrimidin-2-yl)cyclohex-3-en-1-yl)methyl)-3-(oxetan-2-ylmethyl)-3H-imidazo[4,5-b]pyridine-5-carboxylic acid (Compound 127)

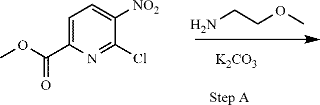

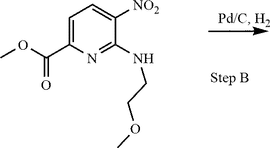

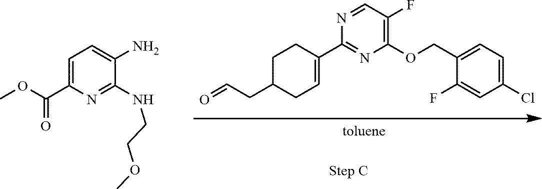

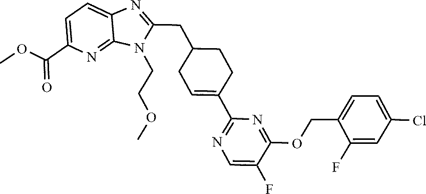 [SMILES: COCCn1c(CC2CC=C(c3ncc(F)c(OCc4ccc(Cl)cc4F)n3)CC2)nc2ccc(C(=O)OC)nc21]

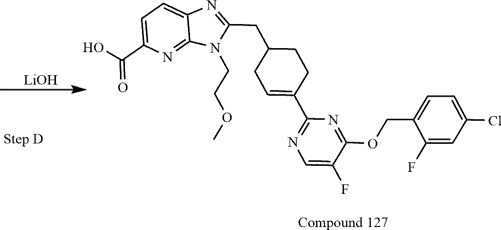

### Step A: The Synthesis of Methyl 6-((2-methoxyethyl)amino)-5-nitropicolinate

To a solution of 2-methoxyethan-1-amine (210 mg, 2.8 mmol) in ACN (10 mL) was added methyl 6-chloro-5-nitropicolinate (600 mg, 2.8 mmol) and K₂CO₃ (1.2 g, 8.4 mmol). The mixture was stirred at RT overnight under N₂ protection. Then the mixture was diluted with water (20 mL) and extracted with EA (30 mL*3). The organic phase was concentrated under vacuum and the residue was purified by flash column (silica, UV254 nm, PE/EA=3/1) to afford methyl 6-((2-methoxyethyl)amino)-5-nitropicolinate (600 mg, yield: 80%) as yellow oil. Calcd.: 255.1; MS Found: 256.1 [M+H]⁺.

### Step B: The Synthesis of Methyl 5-amino-6-((2-methoxyethyl)amino)picolinate

To a solution of methyl 6-((2-methoxyethyl)amino)-5-nitropicolinate (600 mg, 2.35 mmol) in methanol (20 mL) was added Pd/C (10%, 95 mg). The mixture was stirred at RT under H₂ for 4 h. Then the mixture was filtered, and the filtrate was concentrated under vacuum to give desired product methyl 5-amino-6-((2-methoxyethyl)amino)picolinate (500 mg, yield: 92%) as brown solid. MS Calcd.: 225.1; MS Found: 226.1 [M+H]⁺.

### Step C: The Synthesis of Methyl 2-((4-(4-((4-chloro-2-fluorobenzyl)oxy)-5-fluoropyrimidin-2-yl)cyclohex-3-en-1-yl)methyl)-3-(2-methoxyethyl)-3H-imidazo[4,5-blpyridine-5-carboxylate

To a suspension of methyl 5-amino-6-((2-methoxyethyl)amino)picolinate (140 mg) in dry toluene (2 mL) was added 2-(4-(4-((4-chloro-2-fluorobenzyl)fluorobenzyl)oxy)-5-fluoropyrimidin-2-yl)cyclohex-3-en-1-yl)acetaldehyde (208 mg,0.55 mmol) and molecular sieve (416 mg). The mixture was stirred at 80° C. for 48 h under O₂ atmosphere. Then the mixture was filtered through a pad of celite, the solid was washed with ethyl acetate (30 mL) and the filtrate was concentrated under vacuum. The residue was purified by prep-TLC (silica, UV254 nm, DCM/MEOH=30/1) to afford desired product methyl 2-((4-(4-((4-chloro-2-fluorobenzyl)oxy)-5-fluoropyrimidin-2-yl)cyclohex-3-en-1-yl)methyl)-3-(2-methoxyethyl)-3H-imidazo[4,5-b]pyridine-5-carboxylate (160 mg, yield: 50%) as yellow oil. MS Calcd.: 583.2; MS Found: 583.9 [M+H]⁺.

### Step D: The Synthesis of 2-((4-(4-((4-chloro-2-fluorobenzyl)oxy)-5-fluoropyrimidin-2-yl)cyclohex-3-en-1-yl)methyl)-3-(oxetan-2-ylmethyl)-3H-imidazo[4,5-b]pyridine-5-carboxylic acid

To a solution of methyl 2-((4-(4-((4-chloro-2-fluorobenzyl)oxy)-5-fluoropyrimidin-2-yl)cyclohex-3-en-1-yl)methyl)-3-(2-methoxyethyl)-3H-imidazo[4,5-b]pyridine-5-carboxylate (160 mg, 0.27 mmol) in MeOH (3 mL) and water (0.3 mL) was added LiOH (24 mg, 1.0 mmol). The mixture was stirred at RT for 4 h. Then mixture was filtered, and the filtrate was purified by prep-HPLC (high-pH method) to give 2-((4-(4-((4-chloro-2-fluorobenzyl)oxy)-5-fluoropyrimidin-2-yl)cyclohex-3-en-1-yl)methyl)-3-(oxetan-2-ylmethyl)-3H-imidazo[4,5-b]pyridine-5-carboxylic acid (50 mg, yield: 33%) as white solid. MS Calcd.: 569.2; MS Found: 570.0 [M+H]⁺.

¹H NMR (400 MHz, MeOD) δ 8.34 (d, J = 3.2 Hz, 1 H), 8.12 (d, J = 8.4 Hz, 1 H), 8.05 (d, J = 8.4 Hz, 1 H), 7.55 (t, J = 8.0 Hz, 1 H), 7.32-7.20 (m, 2 H), 7.19 (br. s, 1 H), 5.59 (s, 2 H), 4.64 (t, J = 5.0 Hz, 2 H), 3.82 (t, J = 5.0 Hz, 2 H), 3.27 (s, 3 H), 3.11 (d, J = 6.9 Hz, 2 H), 2.85-2.73 (m, 1 H), 2.57 - 2.36 (m, 3 H), 2.20 - 1.95 (m, 2 H), 1.65-1.50 (m, 1 H).

Example 27: 3-(but-2-yn-1-yl)-2-((4-(6-((4-chloro-2-fluorobenzyl)oxy)pyridin-2-yl)cyclohex-3-en-1-yl)methyl)-3H-imidazo[4,5-b]pyridine-5-carboxylic acid (Compound 128)

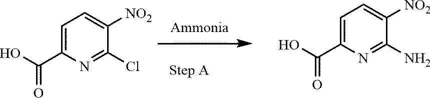

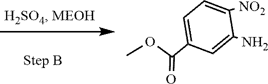

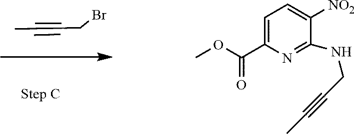

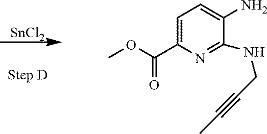

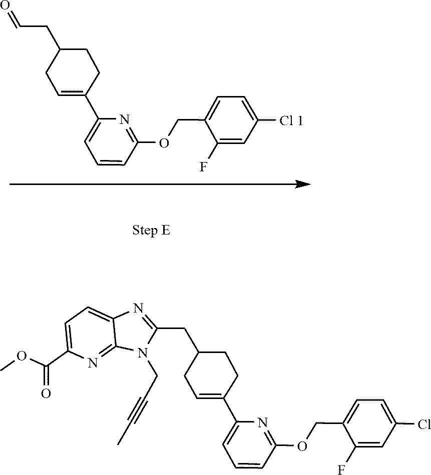

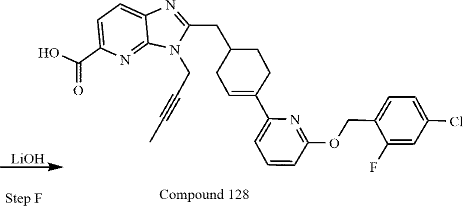

### Step A: The Synthesis of 6-chloro-5-nitropicolinic Acid

To a solution of 6-chloro-5-nitropicolinic acid (2.0 g, 10 mmol) in THF (10 mL) was added NH₃.H2O (10 mL). The mixture was stirred at 50° C. overnight. The mixture was concentrated in vacuum to afford the crude 6-amino-5-nitropicolinic acid (1.9 g) as yellow solid, which was used directly in the next step. MS Calcd.: 183.0; MS Found: 184.0 [M+H]⁺.

### Step B: The Synthesis of Methyl 6-amino-5-nitropicolinate

To a solution of 6-amino-5-nitropicolinic acid (1.85 g, 10 mmol) in anhydrous methanol (20 mL) was added concentrated sulfuric acid (1 mL). The mixture was stirred under reflux for 24 hours. The reaction mixture was cooled to room temperature and diluted with saturated aqueous sodium bicarbonate solution (15 mL). The aqueous layer was extracted with dichloromethane (2 * 100 mL). The combined organic layer was dried over anhydrous sodium sulfate, filtered, and concentrated in vacuo to afford methyl 6-amino-5-nitropicolinate (1.56 g, 78%) as yellow solid. MS Calcd.: 197.0; MS Found197.9 [M+1]⁺.

### Step C: The Synthesis of Methyl 6-(but-2-yn-1-ylamino)-5-nitropicolinate

A mixture of methyl 6-amino-5-nitropicolinate (200 mg, 1 mmol), 1-bromobut-2-yne (132 mg, 1 mmol) and C_{S2}CO₃ (650 mg, 2 mmol) in CH₃CN (10 mL) was stirred at 65° C. under N₂ atmosphere overnight. The mixture was filtered through a pad of silica, eluted with EA (50 mL) and the filtrate was concentrated under reduced pressure. The residue was purified by flash silica column chromatography (eluent=1%-10% MeOH in CH2C12) to afford methyl 6-(but-2-yn-1-ylamino)-5-nitropicolinate (90 mg, 37% yield) as yellow solid. MS Calcd.: 249.1; MS Found: 250.1 [M+H]⁺.

### Step D: The Synthesis of Methyl 5-amino-6-(but-2-yn-l-ylamino)picolinate

A suspension of methyl 6-(but-2-yn-1-ylamino)-5-nitropicolinate (320 mg, 1.28 mmol) and SnCl2 (1.2 g, 6.4 mmol) in MeOH (10 mL) was stirred under reflux overnight. The mixture was cooled and diluted with saturated NaHCO₃ solution (20 mL). The resulting aqueous mixture was extracted with ethyl acetate (30 mL*3). Combined extracts were washed with brine (50 mL), dried and concentrated under reduced pressure. The crude was purified by flash silica column chromatography (eluent= 10% to 30% EA in PE) to afford methyl 5-amino-6-(but-2-yn-1-ylamino)picolinate (230 mg, 82 % yield) as yellow oil. MS Calcd.: 219.1; MS Found: 220 [M+H]⁺.

### Step E: The Synthesis of Methyl 3-(but-2-yn-1-yl)-2-((4-(6-((4-chloro-2-fluorobenzyl)oxy)pyridin-2-yl)cyclohex-3-en-1-yl)methyl)-3H-imidazo[4,5-b1pyridine-5-carboxylate

A suspension of methyl 5-amino-6-(but-2-yn-1-ylamino)picolinate (88 mg, 0.4 mmol) and 2-(4-(6-((4-chloro-2-fluorobenzyl)oxy)pyridin-2-yl)cyclohex-3-en-1-yl)acetaldehyde (72 mg, 0.2 mmol) were in toluene (20 mL) was stirred at 110° C. for 72 hours. The mixture was filtered, and the filter cake was washed with Ethyl acetate (30 mL). The filtrate was concentrated in vacuum, the residue was purified by column chromatography to give product methyl 3-(but-2-yn-1-yl)-2-((4-(6-((4-chloro-2-fluorobenzyl)oxy)pyridin-2-yl)cyclohex-3-en-1-yl)methyl)-3H-imidazo[4,5-b]pyridine-5-carboxylate (200 mg, yield: 66%) as yellow oil. MS Calcd.: 558.2; MS Found: 559.0 [M+H]⁺.

### Step F: The Synthesis of 3-(but-2-yn-1-yl)-2-((4-(6-((4-chloro-2-fluorobenzvl)oxy)pyridin-2-yl)cvclohex-3-en-1-yl)methyl)-3H-imidazo[4,5-b]pyridine-5-carboxylic acid

To a solution of crude methyl 3-(but-2-yn-1-yl)-2-((4-(6-((4-chloro-2-fluorobenzyl)oxy)pyridin-2-yl)cyclohex-3-en-1-yl)methyl)-3H-imidazo[4,5-b]pyridine-5-carboxylate (200 mg, 0.30 mmol) in MeOH (3 mL) and water (0.3 mL) was added LiOH.H₂O (48 mg, 1.2 mmol). The mixture was stirred at RT overnight. The mixture was filtered, and the filtrate was directly purified by prep-HPLC (high-PH) to give 3-(but-2-yn-1-yl)-2-((4-(6-((4-chloro-2-fluorobenzyl)oxy)pyridin-2-yl)cyclohex-3-en-1-yl)methyl)-3H-imidazo[4,5-b]pyridine-5-carboxylic acid (55 mg, yield: 33%) as white solid. MS Calcd.: 544.2; MS Found: 545.1 [M+H]⁺.

¹HNMR (400 MHz, CD₃OD) δ 8.07 (d, J=8.4 Hz, 1 H),7.98 (d, J=8.4 Hz, 1 H), 7.61 (t, J=8.0 Hz, 1 H), 7.50 (t, J=8.0 Hz, 1 H), 7.30-7.16 (m, 2H), 7.05 (d, J=7.6 Hz, 1 H), 6.78 (br.s, 1 H), 6.66 (d, J=8.4 Hz, 1 H),5.45 (s, 2 H), 5.27 (d, J=2.4 Hz, 2 H), 3.18-3.11 (m, 2 H), 2.75-2.65 (m, 1 H), 2.60-2.40 (m, 3 H), 2.25-2.05 (m, 2 H), 1.76 - 1.70 (m, 3 H), 1.65-1.61 (m, 1 H).

Example 1W: (S)-2-((6-((4-chloro-2-fluorobenzyl)oxy)-2′-oxo-[2,4′-bipyridin]-1′(2′H)-yl)methyl)-3-(oxetan-2-ylmethyl)-3H-imidazo[4,5-b]pyridine-5-carboxylic acid (Compound 103aw)

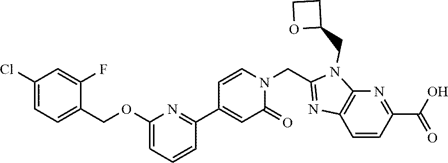 [SMILES: O=C(O)c1ccc2nc(Cn3ccc(-c4cccc(OCc5ccc(Cl)cc5F)n4)cc3=O)n(C[C@@H]3CCO3)c2n1]

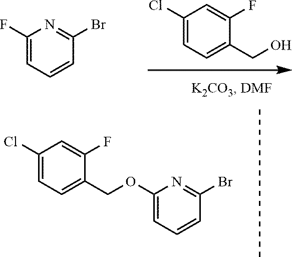

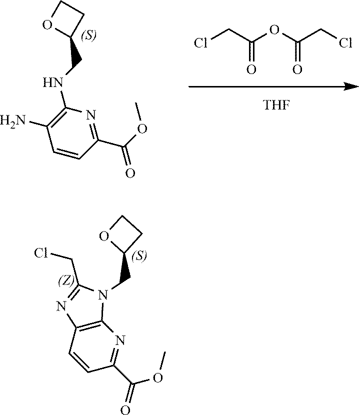

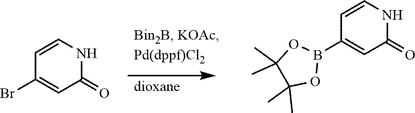

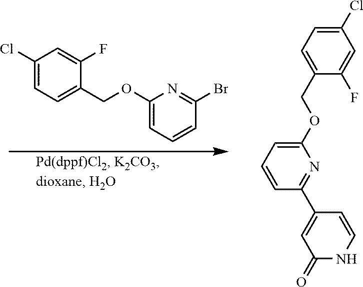

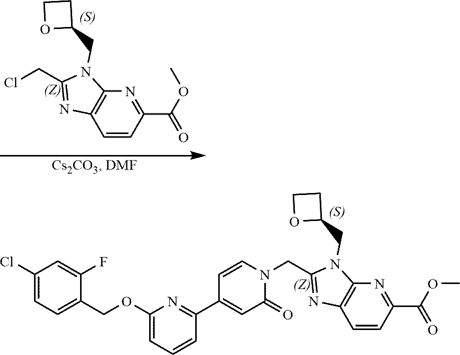

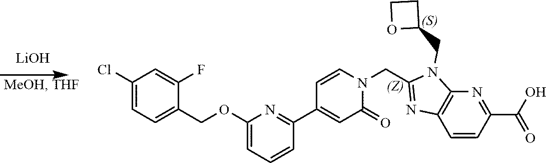

### Step A: Preparation of 2-bromo-6-((4-chloro-2-fluorobenzyl)oxy)pyridine

To a solution of (4-chloro-2-fluorophenyl)methanol (2.00 g, 12.46 mmol) in DMF (25 mL) was added C_{S2}CO₃ (12.17 g, 37.37 mmol) and 2-bromo-6-fluoropyridine (2.19 g, 12.46 mmol). The suspension was stirred at 25° C. for 16 hr. The yellow suspension was diluted with water (50 mL) and extracted with ethyl acetate (35 mL) twice. The organic layer was washed with water (50 mL), brine (50 mL), dried with Na₂SO₄, and filtered. The filtrate was concentrated to give crude product (3.78 g) as a yellow oil. The crude product was purified by Combi-flash (silica gel, ethyl acetate in petrol ether from 0~10%) to give 2-bromo-6-((4-chloro-2-fluorobenzyl)oxy)pyridine (3.18 g, 80.6% yield) as a white solid.

¹H NMR (400 MHz, CDCl₃) δ ppm 7.41 - 7.50 (m, 2 H) 7.08 - 7.18 (m, 3 H) 6.74 (d, J=8.27 Hz, 1 H) 5.39 (s, 2 H)

### Step B: Preparation of 4-(4,4,5,5-tetramethyl-1,3,2-dioxaborolan-2-yl)pyridin-2(1H)-one

To a solution of 4-bromopyridin-2(1H)-one (400.0 mg, 2.30 mmol) in dioxane (5 mL) was added Pin₂B2 (613.0 mg, 2.41 mmol), Pd(dppf)Cl₂ (168.2 mg, 229.87 umol), and KOAc (676.9 mg, 6.90 mmol). The suspension was stirred at 80° C. for 3 hr under N₂. The yellow solution was filtered, and the filtrate was used in next step without further purification.

### Step C: Preparation of 6-((4-chloro-2-fluorobenzvl)oxy)-[2,4′-bipyridin]-2′(1′H)-one

To a solution of 2-bromo-6-((4-chloro-2-fluorobenzyl)oxy)pyridine (350 mg, 1.11 mmol) in dioxane (2 mL) was added 4-(4,4,5,5-tetramethyl-1,3,2-dioxaborolan-2-yl)pyridin-2(1H)-one (508.44 mg, 2.30 mmol), Pd(dppf)Cl₂ (80.90 mg, 110.57 umol), K₂CO₃ (458.43 mg, 3.32 mmol) and H₂O (2 mL). The suspension was stirred at 80° C. for 1.5 hr under N₂. The dark mixture was diluted with water (5 mL) and extracted with ethyl acetate (5 mL) twice. The organic layer was dried with Na₂SO₄ and filtered. The filtrate was concentrated to give crude (863 mg) as a dark gum. The crude was purified by Combi-flash (silica gel, ethyl acetate in petrol ether from 50~100%) to give 6-((4-chloro-2-fluorobenzyl)oxy)-[2,4′-bipyridin]-2′(1′H)-one (316.5 mg, 86.5% yield) as a yellow solid. (The yield was for two steps.)

¹H NMR (400 MHz, DMSO-d₆) δ ppm 11.66 (br s, 1 H) 7.85 (t, J=7.44 Hz, 1 H) 7.64 (d, J=7.15 Hz, 1 H) 7.60 (t, J=7.96 Hz, 1 H) 7.42 - 7.55 (m, 2 H) 7.32 (dd, J=8.19, 1.71 Hz, 1 H) 7.02 (s, 1 H) 6.96 (d, J=8.38 Hz, 1 H) 6.84 (d, J=6.72 Hz, 1 H) 5.49 (s, 2 H); LCMS: m/z 330.9[M+H]⁺.

### Step D: Preparation of (S)-methyl 2-(chloromethyl)-3-(oxetan-2-ylmethyl)-3H-imidazo[4,5-b]pyridine-5-carboxylate

To a solution of methyl (S)-methyl 5-amino-6-((oxetan-2-ylmethyl)amino)picolinate (Intermediate 2, 600.0 mg, 2.53 mmol) in THF (5 mL) was added a solution of 2-chloroacetic anhydride (475.6 mg, 2.78 mmol) in THF (2 mL) dropwise. The solution was stirred at 25° C. for 2 hr under N₂. Then the solution was stirred at 60° C. for 12 hr. The deep yellow solution was diluted with water (6 mL) and extracted with ethyl acetate (5 mL) twice. The organic layer was dried with Na₂SO₄ and filtered. The filtrate was concentrated to give crude product as a yellow gum. The crude product was purified by Combi-flash (silica gel, ethyl acetate in petrol ether from 10~60%) to give (S)-methyl 2-(chloromethyl)-3-(oxetan-2-ylmethyl)-3H-imidazo[4,5-b]pyridine-5-carboxylate (330.5 mg, 44.2% yield) as a white solid.

¹H NMR (400 MHz, CDCl₃) δ ppm 8.11-8.18 (m, 2 H) 5.19 - 5.27 (m, 1 H) 5.09 (q, J=7.13 Hz, 2 H) 4.71 - 4.85 (m, 2 H) 4.57 - 4.64 (m, 1 H) 4.27-4.36 (m, 1 H) 4.02 (s, 3 H 2.73-2.84 (m, 1 H) 2.38-2.50 (m, 1 H)

### Step E: Preparation of (S)-methyl 2-((6-((4-chloro-2-fluorobenzyl)oxy)-2′-oxo-[2,4′-bipyridin]-1′(28.H)-yl)methyl)-3-(oxetan-2-ylmethyl)-3H-imidazo[4,5-b]pyridine-5-carboxylate

To a solution of 6-((4-chloro-2-fluorobenzyl)oxy)-[2,4′-bipyridin]-2′(1′H)-one (150.0 mg, 453.53 umol) and (S)-methyl 2-(chloromethyl)-3-(oxetan-2-ylmethyl)-3H-imidazo[4,5-b]pyridine-5-carboxylate (134.1 mg, 453.47 umol) in DMF (2 mL) was added C_{S2}CO₃ (443.30 mg, 1.36 mmol). The yellow suspension was stirred at 70° C. for 0.5 hr under N₂. The deep yellow suspension was diluted with water (5 mL) and extracted with ethyl acetate (5 mL) twice. The organic layer was washed with water (8 mL), dried with Na₂SO₄, and filtered. The filtrate was concentrated to give crude product as a yellow gum. The crude product was purified by Combi-flash (silica gel, MeOH in DCM from 0~15%) to give (S)-methyl 2-((6-((4-chloro-2-fluorobenzyl)oxy)-2′-oxo-[2,4′-bipyridin]-1′(2′H)-yl)methyl)-3-(oxetan-2-ylmethyl)-3H-imidazo[4,5-b]pyridine-5-carboxylate (156.3 mg, 58.4% yield) as a yellow gum.

¹H NMR (400 MHz, CD₃OD) δ ppm 8.01-8.09 (m, 2 H) 7.96 (s, 1 H) 7.90 (d, J=7.09 Hz, 1 H) 7.76 (t, J=7.18 Hz, 1 H) 7.45 - 7.54 (m, 2 H) 7.14 - 7.24 (m, 3 H) 7.05 (dd, J=7.20, 2.00 Hz, 1 H) 6.88 (d, J=7.80 Hz, 1 H) 5.75 (d, J=16.14 Hz, 1 H) 5.54 (d, J=16.14 Hz, 1 H) 5.47 (s, 2 H) 5.18 - 5.28 (m, 1 H) 4.90 - 5.01 (m, 1 H) 4.78 - 4.85 (m, 1 H) 4.56 - 4.65 (m, 1 H) 4.37-4.46 (m, 1 H) 3.97 (s, 3 H) 2.74 - 2.82 (m, 1 H) 2.40 - 2.50 (m, 1 H)

### Step F: Preparation of (S)-2-((6-((4-chloro-2-fluorobenzyl)oxy)-2′-oxo-[2,4′-bipyridin1-1′(2′H)-yl)methyl)-3-(oxetan-2-ylmethyl)-3H-imidazo[4,5-b]pyridine-5-carboxylic acid

To a suspension of (S)-methyl 2-((6-((4-chloro-2-fluorobenzyl)oxy)-2′-oxo-[2,4′-bipyridin]-1′(2′H)-yl)methyl)-3-(oxetan-2-ylmethyl)-3H-imidazo[4,5-b]pyridine-5-carboxylate (126.0 mg, 213.56 umol) in MeOH (2 mL) and THF (1 mL) was added LiOH (2 M, 427.12 uL). The yellow suspension was stirred at 25° C. for 2 hr. To the yellow solution was added 1N HCl to adjust pH to 8~9. The mixture was concentrated to give crude (209.5 mg) as a yellow gum. The crude was purified by prep-HPLC to give (S)-2-((6-((4-chloro-2-fluorobenzyl)oxy)-2′-oxo-[2,4′-bipyridin]-1′(2′H)-yl)methyl)-3-(oxetan-2-ylmethyl)-3H-imidazo[4,5-b]pyridine-5-carboxylic acid (31.66 mg, 25.7% yield, 100% purity) as a yellow solid. LCMS: m/z 576.2 [M+H]⁺.

¹H NMR (400 MHz, CD₃OD) δ ppm 8.09 (d, J=7.96 Hz, 1 H) 8.03 (d, J=8.45 Hz, 1 H) 7.91 (d, J=7.13 Hz, 1 H) 7.80 (t, J7.81 Hz, 1 H) 7.57 (d, J=7.26 Hz, 1 H) 7.52 (t, J=8.01 Hz, 1 H) 7.16-7.27 (m, 3 H) 7.13 (dd, J=7.20, 2.00 Hz, 2 H) 6.91 (d, J=8.25 Hz, 1 H) 5.77 (d, J=16.13 Hz, 1 H) 5.55 (d, J= 16.13 Hz, 1 H) 5.51 (s, 2 H) 5.22-5.30 (m, 1 H) 4.92 -5.03 (m, 1 H) 4.73 - 4.84 (m, 1 H) 4.59 - 4.65 (m, 1 H) 4.40 - 4.47 (m, 1 H) 2.74 - 2.85 (m, 1 H) 2.43 - 2.53 (m, 1 H).

Example 2W: (S)-2-((6-((4-chloro-2-fluorobenzyl)oxy)-5′-methyl-2′-oxo-[2,4′-bipyridin]-1′(2′H)-yl)methyl)-3-(oxetan-2-ylmethyl)-3H-imidazo[4,5-b]pyridine-5-carboxylic acid (Compound 104aw)

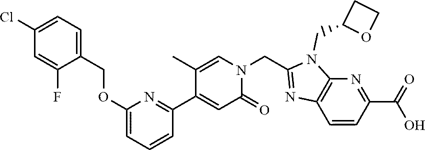 [SMILES: Cc1cn(Cc2nc3ccc(C(=O)O)nc3n2C[C@@H]2CCO2)c(=O)cc1-c1cccc(OCc2ccc(Cl)cc2F)n1]

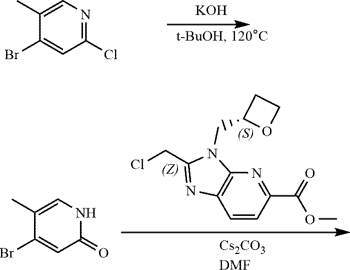

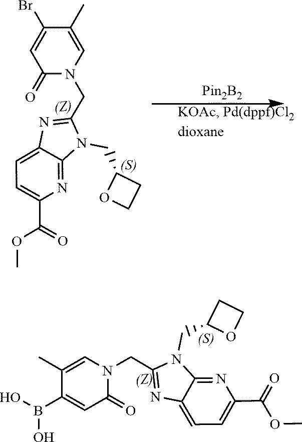

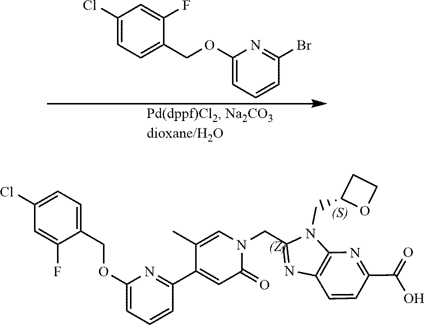

### Step A: Preparation of 4-bromo-5-methylpyridin-2(1H)-one

To a solution of 4-bromo-2-chloro-5-methyl-pyridine (2.0 g, 9.69 mmol) in t-BuOH (25 mL) was added KOH (1.63 g, 29.06 mmol). The mixture was stirred at 110° C. for 12 h. The reaction mixture was diluted with water (50 mL) and extracted with EtOAc (60 mL x 2). The combined organics were collected, dried over anhydrous Na₂SO₄, filtered and concentrated under reduced pressure. The crude product was triturated with a solution of DMF (3 mL) and MeOH (2 mL). The solid was filtered and collected and dried in vacuo. 4-bromo-5-methyl-1H-pyridin-2-one (796 mg, yield: 39.7%) was obtained as a white solid.

LCMS: m/z 187.8 [M+H]⁺Step B: Preparation of (S)-methyl 2-((4-bromo-5-methyl-2-oxopyridin-1(2H)-yl)methyl)-3-(oxetan-2-ylmethyl)-3H-imidazo[4,5-b]pyridine-5-carboxylate

To a solution of 4-bromo-5-methyl-1H-pyridin-2-one (385 mg, 2.05 mmol) in CH₃CN (5 mL) was added methyl 2-(chloromethyl)-3-[[(2S)-oxetan-2-yl]methyl]imidazo[4,5-b]pyridine-5-carboxylate (606 mg, 2.05 mmol) and K₂CO₃ (850 mg, 6.15 mmol). Then the mixture was stirred at 50° C. for 16 h. The reaction mixture was diluted with water (20 mL) and extracted with EtOAc (30 mL x 3). The combined organics were collected, dried over anhydrous Na₂SO₄, filtered, and concentrated under reduced pressure to give a residue. The residue was purified by column chromatography (SiO₂, Ethyl acetate / MeOH = ⅟0 to 15/1). Methyl 2-[(4-bromo-5-methyl-2-oxo-1-pyridyl)methyl]-3-[[(2S)-oxetan-2-yl]methyl]imidazo[4,5-b]pyridine-5-carboxylate (660 mg, yield: 66.7%) was obtained as light yellow solid. LCMS: m/z 446.8 [M+H]⁺.Step C: Preparation of (S)-(1-((5-(methoxycarbonyl)-3-(oxetan-2-ylmethyl)-3H-imidazo[4,5-b]pyridin-2-yl)methyl)-5-methyl-2-oxo-1,2-dihydropyridin-4-yl) boronic acid

To a solution of methyl 2-[(4-bromo-5-methyl-2-oxo-1-pyridyl)methyl]-3-[[(2S)-oxetan-2-yl]methyl]imidazo[4,5-b]pyridine-5-carboxylate (563 mg, 1.26 mmol) in dioxane (5 mL) was added Pin₂B₂ (320 mg, 1.26 mmol), KOAc (371 mg, 3.78 mmol) and Pd(dppf)Cl₂ (92 mg, 125.87 umol). Then the mixture was stirred at 85° C. for 16 h under N₂. The reaction mixture was diluted with water (10 mL) and extracted with EtOAc (15 mL x 3). The combined organics were collected, dried over anhydrous Na₂SO₄, filtered, and concentrated under reduced pressure to give a residue. The residue was purified by column chromatography (SiO₂, Petroleum ether/ Ethyl acetate = 0/1, DCM: MeOH = ⅟0 to 0/1). [1-[[5-methoxycarbonyl-3-[[(2S)-oxetan-2-yl]methyl]imidazo[4,5-b]pyridin-2-yl]methyl]-5-methyl-2-oxo-4-pyridyl]boronic acid (200 mg, yield: 28.5%) was obtained as black solid. LCMS: m/z [M+H]⁺.Step D: Preparation of (S)-2-((6-((4-chloro-2-fluorobenzyl)oxy)-5′-methyl-2′-oxo-[2,4′-bipyridin]-1′(2′H)-yl)methyl)-3-(oxetan-2-ylmethyl)-3H-imidazo[4,5-b]pyridine-5-carboxylic acid

To a solution of [1-[[5-methoxycarbonyl-3-[[(2S)-oxetan-2-yl]methyl]imidazo[4,5-b]pyridin-2-yl]methyl]-5-methyl-2-oxo-4-pyridyl]boronic acid (90 mg, 218.34 umol) in dioxane (2.5 mL) and H₂O (0.5 mL) was added 2-bromo-6-[(4-chloro-2-fluorophenyl)methoxy]pyridine (69.12 mg, 218.34 umol), Na₂CO₃ (69.42 mg, 655.02 umol) and Pd(dppf)Cl₂ (47.93 mg, 65.50 umol). Then the mixture was stirred at 80° C. under N₂ for 16 h. The reaction mixture was filtered and concentrated under reduced pressure to give a residue. The residue was purified by column chromatography (SiO₂, Petroleum ether/Ethyl acetate=0/1 and DCM/MeOH =⅟0 to 0/1). The residue was purified by prep-HPLC (column: Phenomenex Gemini-NX 80 * 30 mm * 3 um; mobile phase: [water (10mM NH₄HCO₃)-ACN]; B%: 10%-80%, 9.5 min). Compound (S)-2-((6-((4-chloro-2-fluorobenzyl)oxy)-5′-methyl-2′-oxo-[2,4′-bipyridin]-1′(2′H)yl)methyl)-3-(oxetan-2-ylmethyl)-3H-imidazo[4,5-b]pyridine-5-carboxylic acid (60.21 mg, 46.48% yield) was obtained as a white solid. LCMS: m/z590.1 [M+H]⁺.

¹H NMR (400 MHz, CD₃OD) δ ppm 8.03 - 8.16 (m, 2 H), 7.82 (t, J=7.78 Hz, 1 H), 7.74 (s, 1 H), 7.50 (t, J=8.28 Hz, 1 H), 7.14 - 7.28 (m, 3 H), 6.92 (d, J=8.28 Hz, 1 H), 6.63 (s, 1 H), 5.77 (d, J=16.06 Hz, 1 H), 5.55 (d, J=16.06 Hz, 1 H), 5.45 (s, 2 H), 5.27 (br s, 1 H), 4.97 - 5.04 (m, 1 H), 4.82 - 4.86 (m, 1 H), 4.58 - 4.68 (m, 1 H), 4.40 - 4.50 (m, 1 H), 2.51 (br d, J=8.28 Hz, 1 H), 2.12 (s, 3 H).

Example 3W: (S)-2-((6-((4-chloro-2-fluorobenzyl)oxy)-3′-methyl-2′-oxo-[2,4′-bipyridin]-1′(2′H)-yl)methyl)-3-(oxetan-2-ylmethyl)-3H-imidazo[4,5-b]pyridine-5-carboxylic acid (Compound 105aw)

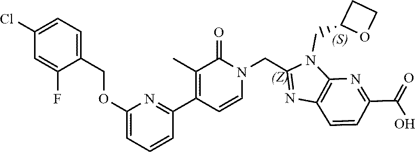

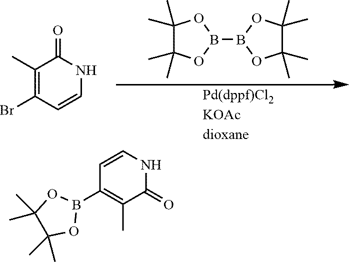

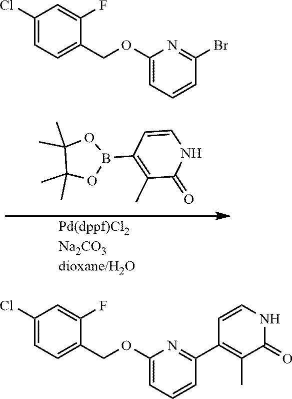

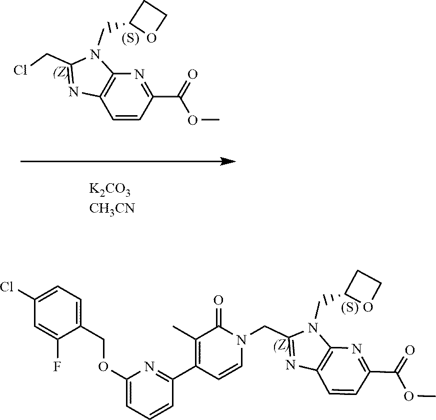

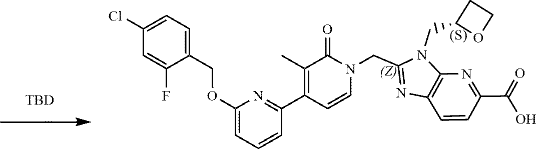

### Step A: Preparation of 3-methyl-4-(4,4,5,5-tetramethyl-1,3,2-dioxaborolan-2-yl)pyridin-2(1H)-one

To a solution of 4-bromo-3-methyl-1H-pyridin-2-one (200 mg, 1.06 mmol) in dioxane (10 mL) was added 4,4,5,5-tetramethyl-2-(4,4,5,5-tetramethyl-1,3,2-dioxaborolan-2-yl)-1,3,2-dioxaborolane (324 mg, 1.28 mmol), Pd(dppf)Cl₂ (78 mg, 106.37 umol) and KOAc (313 mg, 3.19 mmol). The mixture was stirred at 90° C. for 16 hr under N₂. The mixture was filtered, and the filtrate was concentrated under reduced pressure. The residue was used into next step without further purification. Compound 3-methyl-4-(4,4,5,5-tetramethyl-1,3,2-dioxaborolan-2-yl)-1H-pyridin-2-one (662.5 mg, crude) was obtained as a black oil.

### Step B: Preparation of 6-((4-chloro-2-fluorobenzvl)oxy)-3′-methyl-[2,4′-bipyridin]-2′(1′H)-one

To a solution of 3-methyl-4-(4,4,5,5-tetramethyl-1,3,2-dioxaborolan-2-yl)-1H-pyridin-2-one (562.5 mg, 2.39 mmol) in dioxane (6 mL) was added 2-bromo-6-[(4-chloro-2-fluorophenyl) methoxy]pyridine (337 mg, 1.06 mmol), Pd(dppf)Cl₂ (175 mg, 239.27 umol) in H₂O (2 mL) and Na₂CO₃ (761 mg, 7.18 mmol). The mixture was stirred at 90° C. for 16 hr under N₂. The mixture was filtered, and the organic layer was concentrated under reduced pressure. The residue was purification by silica gel chromatography (DCM: MeOH = 1:0 to 10:1). Compound 4-[6-[(4-chloro-2-fluoro-phenyl)methoxy]-2-pyridyl]-3-methyl-1H-pyridin-2-one (280.9 mg, yield: 30.5%) was obtained as a yellow oil. LCMS: m/z345.1 [M+H]⁺.

### Step C: Preparation of (S)-methyl 2-((6-((4-chloro-2-fluorobenzvl)oxy)-3′-methyl-2′-oxo-[2,4′-bipyridinl-1′(2′H)-yl)methyl)-3-(oxetan-2-ylmethyl)-3H-imidazo[4,5-b]pyridine-5-carboxylate

To a solution of 4-[6-[(4-chloro-2-fluoro-phenyl)methoxy]-2-pyridyl]-3-methyl-1H-pyridin-2-one (70 mg, 203.04 umol) in CH₃CN (5 mL) was added K₂CO₃ (84 mg, 609.11 umol) and methyl 2-(chloromethyl)-3-[[(2S)-oxetan-2-yl]methyl]imidazo[4,5-b]pyridine-5-carboxylate (60 mg, 203.04 umol). The mixture was stirred at 50° C. for 16 hr. The mixture reaction was extracted with EA (50mL x 2), H₂O (60 mL x 2), washed with brine (60 mL x 2), dried over anhydrous Na₂SO₄, filtered and concentrated under reduced pressure. The residue was purified by silica gel chromatography (EA: MeOH = 1:0 to 1:1). Compound methyl 2-[[4-[6-[(4-chloro-2-fluoro-phenyl)methoxy]-2-pyridyl]-3-methyl-2-oxo-1-pyridyl]methyl]-3-[[(2S)-oxetan-2-yl]methyl] imidazo[4,5-b]pyridine-5-carboxylate (39 mg, yield: 28.6%) was obtained as yellow oil. LCMS: m/z604.1[M+H]+. ¹H NMR (400 MHz, CDCl₃) δ 8.12 - 8.08 (m, 2 H), 7.67 - 7.64 (m, 2 H), 7.47 - 7.42 (m, 1 H), 7.14 - 7.10 (m, 2 H), 7.06 - 6.98 (m, 1 H), 6.81 - 6.74 (m, 1 H), 6.46 - 6.42 (m, 1 H), 5.72 - 5.67 (m, 1 H), 5.47 - 5.35 (m, 3 H), 5.27 - 5.17 (m, 1 H), 5.14 - 5.06 (m, 1 H), 4.97 -4.92 (m, 1 H), 4.67 - 4.57 (m, 1 H), 4.46 - 4.39 (m, 1 H), 4.01 (s, 3 H), 2.90 - 2.75 (m, 1 H), 2.55 - 2.45 (m, 1 H), 2.16 (s, 3 H).

### Step D: Preparation of (S)-2-((6-((4-chloro-2-fluorobenzyl)oxy)-3′-methyl-2′-oxo-[2,4′-bipyridinl-1′(2′H)-yl)methyl)-3-(oxetan-2-ylmethyl)-3H-imidazo[4,5-b[pyridine-5-carboxylic acid

To a solution of methyl 2-[[4-[6-[(4-chloro-2-fluoro-phenyl)methoxy]-2-pyridyl]-3-methyl-2-oxo-1-pyridyl]methyl]-3-[[(2S)-oxetan-2-yl]methyl]imidazo[4,5-b]pyridine-5-carboxylate (39 mg, 64.57 umol) in CH₃CN (3 mL) was added 3,4,6,7,8,9-hexahydro-2H-pyrimido[1,2-a]pyrimidine (18 mg, 129.13 umol) in H₂O (0.6 mL). The mixture was stirred at 25° C. for 2 hr. The mixture reaction was adjusted to pH=7, extracted with EA (20 mL x 2), washed with H₂O (20 mL x 2), brine (30 mL x 2), dried over anhydrous Na₂SO₄, filtered, and concentrated under reduced pressure. The crude product was purified by reversed-phase HPLC (column: Phenomenex Gemini-NX 80 x 30 mm x 3 um; mobile phase: [water (10 mM NH₄HCO₃) - ACN]; B%: 0%-60%, 9.5 min). (S)-2-((6-((4-chloro-2-fluorobenzyl)oxy)-3′-methyl-2′-oxo-[2,4′-bipyridin]-1′(2′H)-yl)methyl)-3-(oxetan-2-ylmethyl)-3H-imidazo[4,5-b]pyridine-5-carboxylic acid (4.42 mg, yield: 11.4%) was obtained as white solid. LCMS: m/z590.3[M+H]⁺.

¹H NMR (400 MHz, CD₃OD) δ 8.22 - 7.95 (m, 2 H), 7.82 - 7.68 (m, 2 H), 7.52 - 7.44 (m, 1 H), 7.09 - 7.22 (m, 3 H), 6.92 - 6.84 (m, 1 H), 6.55 - 6.45 (m, 1 H), 5.82 - 5.72 (m, 1 H), 5.59 - 5.53 (m, 1 H), 5.43 (s, 2 H), 5.35 - 5.22 (m, 1 H),5.11 - 4.95 (m, 3 H), 4.69 - 4.57 (m, 1 H), 4.49 - 4.40 (m, 1 H), 2.87 - 2.72 (m, 1 H), 2.55 - 2.42 (m, 1 H), 2.07 (s, 3 H).

Example 4W: (S)-2-((6-((4-cyano-2-fluorobenzyl)oxy)-2′-oxo- [2,4′-bipyridin] -1′(2′H)-yl)methyl)-3-(oxetan-2-ylmethyl)-3H-imidazo[4,5-b]pyridine-5-carboxylic acid (Compound 106aw)

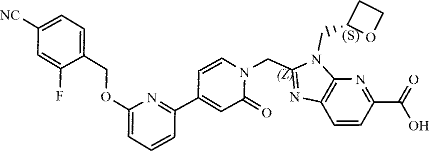

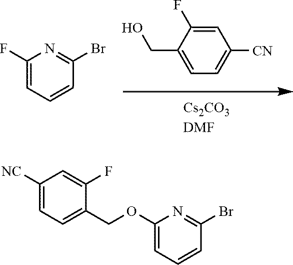

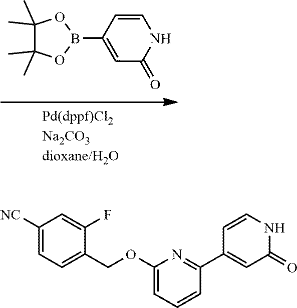

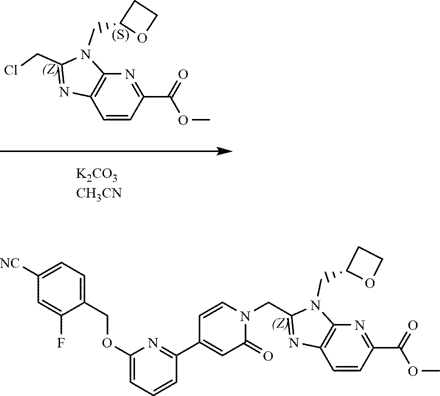

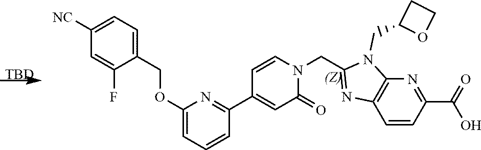

### Step A: Preparation of 4-(((6-bromopyridin-2-yl)oxy)methyl)-3-fluorobenzonitrile

To a solution of 2-bromo-6-fluoro-pyridine (500 mg, 2.84 mmol) in DMF (15 mL) was added 3-fluoro-4-(hydroxymethyl)benzonitrile (472 mg, 3.13 mmol) and C_{S2}CO₃ (1.85 g, 5.68 mmol). The mixture was stirred at 85° C. for 12 h. The reaction mixture was quenched with H₂O (30 mL) and extracted with EtOAc (30 mL x 3). The organic layer was washed with brine (50 mL), dried over Na₂SO₄, filtered and concentrated. The residue was purified by column (SiO₂, Petroleum ether/Ethyl acetate = ⅟0 to 0/1). 4-(((6-bromopyridin-2-yl)oxy)methyl) -3-fluorobenzonitrile (400 mg, yield: 45.8%) was obtained as white solid. LCMS: m/z306.8[M+H]⁺.

### Step B: Preparation of 3-fluoro-4-(((2′-oxo-1′,2′-dihydro-[2,4′-bipyridin]-6-yl)oxy) methyl)benzonitrile

To a solution of 4-[(6-bromo-2-pyridyl)oxymethyl]-3-fluoro-benzonitrile (150 mg, 488.41 umol) in dioxane (1.5 mL) and H₂O (0.5 mL) was added 4-(4,4,5,5-tetramethyl-1,3,2-dioxaborolan-2-yl)-1H-pyridin-2-one (162 mg, 732.62 umol), Na₂CO₃ (155 mg, 1.47 mmol) and Pd(dppf)C1₂ (36 mg, 48.84 umol). The mixture was stirred at 85° C. for 12 h under N₂. The reaction mixture was quenched with H₂O (30 mL) and extracted with EtOAc (30 mL x 3). The organic layer was washed with brine (50 mL), dried over Na₂SO₄, filtered and concentrated. The residue was purified by column (SiO₂, DCM: MeOH = 20:1). 3-fluoro-4-(((2′-oxo-1′,2′-dihydro-[2,4′-bipyridin]-6-yl)oxy)methyl)benzonitrile (130 mg, yield: 82.8%) was obtained as yellow solid. LCMS: m/z321.9[M+H]⁺.

### Step C: Preparation of (S)-methyl 2-((6-((4-cyano-2-fluorobenyl)oxy)-2′-oxo- [2,4′-bipyridin]-1 ′(2′H)-yl)methyl)-3-(oxetan-2-ylmethyl)-3H-imidazo[4,5-b]pyridine-5-carboxylate

To a solution of 3-fluoro-4-[[6-(2-oxo-1H-pyridin-4-yl)-2-pyridyl]oxymethyl]benzonitrile (76 mg, 236.71 umol) in CH₃CN (10 mL) was added methyl2-(chloromethyl)-3-[[(2S)-oxetan-2-yl] methyl]imidazo[4,5-b]pyridine-5-carboxylate (70 mg, 236.71 umol) and K₂CO₃ (98 mg, 710.13 umol). The mixture was stirred at 50° C. for 12 h. The reaction mixture was quenched with H₂O (30 mL) and extracted with EA (30 mL x 3). The organic layer was washed with brine (50 mL), dried over Na₂SO₄, filtered and concentrated. The residue was purified by column (SiO₂, DCM: MeOH = 20:1). (S)-methyl2-((6-((4-cyano-2-fluoroben zyl)oxy)-2′-oxo-[2,4′-bipyridin]-l′(2′H)-yl)methyl)-3-(oxetan-2-ylmethyl)-3H-imidazo[4,5-b]pyridine-5-carboxylate (40 mg, yield: 29.1%) was obtained as yellow solid. LCMS: m/z581.2[M+H]⁺.

¹H NMR (400 MHz, DMSO-d₆) δ 8.19 - 8.08 (m, 1 H), 8.06 - 7.82 (m, 4 H), 7.79 - 7.61 (m, 3 H),7.10 - 6.89 (m, 3 H), 5.69 - 5.42 (m, 4 H), 5.18 - 5.06 (m, 1 H), 4.86 - 4.75 (m, 1 H), 4.72 - 4.61 (m, 1 H), 4.51 - 4.40 (m, 1 H), 4.37 - 4.27 (m, 1 H), 3.87 (s, 3 H), 2.77 - 2.66 (m, 1 H), 2.41 - 2.30 (m, 1 H).

### Step D: Preparation of (S)-2-((6-((4-cyo-2-fluorobenzyl)oxy)-2′-oxo- [2,4′-bipyridin] -1′(2′H)-yl)methyl)-3-(oxetan-2-ylmethyl)-3H-imidazo[4.5-b]pyridine-5-carboxylic acid

To a solution of methyl 2-[[4-[6-[(4-cyano-2-fluoro-phenyl)methoxy]-2-pyridyl]-2-oxo-1-pyridyl]methyl]-3-[[(2S)-oxetan-2-yl]methyl]imidazo[4,5-b]pyridine-5-carboxylate (40 mg, 68.90 umol) in CH₃CN (5 mL) and H₂O (1 mL) was added 3,4,6,7,8,9-hexahydro-2H-pyrimido[1, 2-a]pyrimidine (19 mg, 137.80 umol). The mixture was stirred at 25° C. for 1 h. The mixture was acidified with 1N HCl to pH = 7 and extracted with EtOAc (20 mL x 2). The organic layer was washed with H₂O (50 mL), dried over Na₂SO₄, filtered and concentrated. The residue was purified by prep-HPLC (Neu) (column: Phenomenex Gemini-NX 80 x 30 mm x 3um; mobile phase: [water (10 mM NH₄HCO₃) - ACN]; B%: 10% - 80%, 9.5 min). (S)-2-((6-((4-cyano-2-fluorobenzyl)oxy)-2′-oxo-[2,4′-bipyridin]-1′(2′H)-yl)methyl)-3-(oxetan-2-ylmethyl)-3H-imidazo[4,5-b]pyridine-5-carboxylic acid (11 mg, yield: 28.0%) was obtained as white solid. LCMS: m/z567.1 [M+H]⁺. ¹H NMR (400 MHz, DMSO-d₆) δ 8.05 - 8.03 (m, 1 H), 8.00 - 7.88 (m, 4 H), 7.79 - 7.69 (m, 3 H), 7.12 - 7.08 (m, 1 H), 7.04 - 6.97 (m, 1 H), 6.96 - 6.91 (m, 1 H), 5.68 - 5.60 (m, 3 H), 5.54 - 5.47 (m, 1 H), 5.19 - 5.11 (m, 1 H), 4.88 - 4.80 (m, 1 H),4.75 - 4.68 (m, 1 H), 4.53 - 4.48 (m, 1 H), 4.40 - 4.30 (m, 1 H), 2.78 - 2.69 (m, 1 H), 2.36 - 2.32 (m, 1 H).

Example 5W: Preparation of 2-[[4-[6-[(4-chloro-2-fluorophenyl)methoxy]-2-pyridyl]-2-oxo-1 -pyridyl]methyl]-3-[[(2S)-oxetan-2-yl]methyl]benzimidazole-5-carboxylic acid (Compound 107aw)

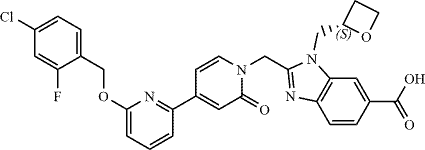

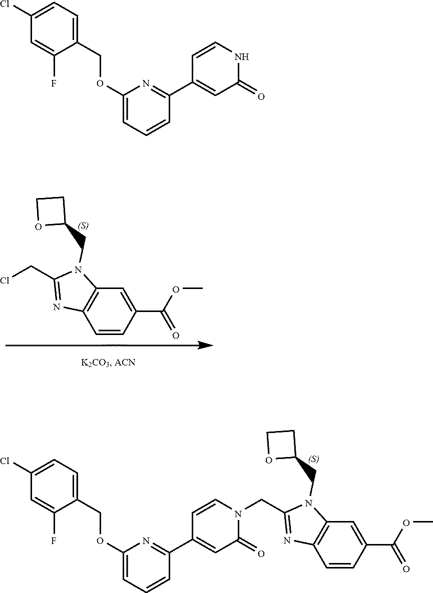

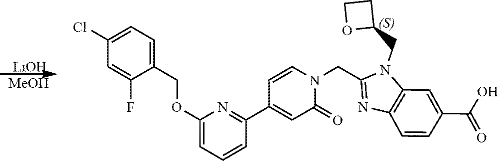

### Step A: Preparation of methyl2-[[4-[6-[(4-chloro-2-fluoro-phenyl)methoxyl-2-pyridyl]-2-oxo-l-pyridyllmethyl]-3-[[(2S)-oxetan-2-yl]methyl]benzimidazole-5-carboxylate

A mixture of 4-[6-[(4-chloro-2-fluoro-phenyl)methoxy]-2-pyridyl]-1H-pyridin-2-one (167 mg, 504.93 umol), methyl 2-(chloromethyl)-3-[[(2S)-oxetan-2-yl]methyl]benzimidazole-5-carboxylate (178.58 mg, 605.91 umol), and K₂CO₃ (348.92 mg, 2.52 mmol) in ACN (2 mL) was degassed and purged with N₂ for 3 times. The mixture was stirred at 20° C. for 16 hr under N₂ atmosphere. The reaction mixture was filtered and concentrated under reduced pressure to give a residue. The residue was purified by column chromatography (SiO₂, Petroleum ether/Ethyl acetate=60% to 75%). Methyl 2-[[4-[6-[(4-chloro-2-fluoro-phenyl)methoxy]-2-pyridyl]-2-oxo-1-pyridyl]methyl]-3-[[(2S)-oxetan-2-yl]methyl]benzimidazole-5-carboxylate (67.3 mg, 22.6% yield) was obtained as a yellow oil.

¹H NMR (400 MHz, CD₃OD) δ ppm 8.31 (s, 1 H) 7.91 - 7.99 (m, 2 H) 7.79 (t, J=7.82 Hz, 1 H) 7.65 (d, J=8.50 Hz, 1 H) 7.49 - 7.60 (m, 2 H) 7.17 - 7.26 (m, 3 H) 7.12 (dd, J=7.13, 1.88 Hz, 1 H) 6.91 (d, J=8.25 Hz, 1 H) 5.68 (d, J=15.88 Hz, 1 H) 5.46 - 5.54 (m, 3 H) 5.15 - 5.26 (m, 1 H) 4.89 - 4.94 (m, 1 H) 4.71 - 4.79 (m, 1 H) 4.58 - 4.65 (m, 1 H) 4.45-4.40 (m, 1 H) 3.92 (s, 3 H) 2.75 - 2.84 (m, 1 H) 2.45 - 2.55 (m, 1 H)

### Step B: Preparation of 2-[[4-[6-[(4-chloro-2-fluorophenyl)methoxy]-2-pyridyl]-2-oxo-1-pyridyl]methyl]3-[[(2S′)-oxetan-2-yl]methyl]benzimidazole-5-carboxylic acid

To a solution of methyl 2-[[4-[6-[(4-chloro-2-fluoro-phenyl)methoxy]-2-pyridyl]-2-oxo-1-pyridyl]-methyl]-3-[[(2S)-oxetan-2-yl]methyl]benzimidazole-5-carboxylate (67.3 mg, 114.26 umol) in MeOH (2 mL) was added LiOH.H₂O (2 M, 114.26 uL). The mixture was stirred at 20° C. for 48 hr. The reaction mixture was filtered and concentrated under reduced pressure to give a residue. The crude product was triturated with MeOH (3 mL) at 20° C. 2-[[4-[6-[(4-chloro-2-fluorophenyl)methoxy]-2-pyridyl]-2-oxo-1-pyridyl]methyl]-3-[[(2S)-oxetan-2-yl]methyl]benzimidazole-5-carboxylic acid (22.3 mg, 32.9% yield) was obtained as a white solid. LCMS: m/z575.1[M+H]⁺.

¹H NMR (400 MHz, DMSO-d₆) δ ppm 8.28 (s, 1 H)7.97 (d, J=7.28 Hz, 1 H) 7.87 (t, J=7.78 Hz, 1 H) 7.80 (dd, J=8.53, 1.25 Hz, 1 H) 7.69 (d, J=7.53 Hz, 1 H) 7.56 - 7.65 (m, 2 H) 7.49 (dd, J=9.91, 1.88 Hz, 1 H) 7.32 (dd, J=8.16, 1.63 Hz, 1 H) 7.11 (d, J=1.51 Hz, 1 H) 7.03 - 6.94 (m, 2 H) 5.37 - 5.70 (m, 4 H) 5.08 (m, 1 H) 4.80 - 4.94 (m, 1 H) 4.67 -4.77 (m, 1 H) 4.43 - 4.54 (m, 1 H) 4.38 - 4.33 (m, 1 H) 2.62 - 2.80 (m, 1 H) 2.49 - 2.49 (m, 1 H) 2.30 - 2.43 (m, 1 H).

Example 6W: 2-[[4-[6-[(4-chloro-2-fluoro-phenyl)methoxy]-2-pyridyl]-2-pyridyl]oxymethyl]-3-[[(2S)-oxetan-2-yl]methyl]benzimidazole-5-carboxylic acid (Compound 111aw)

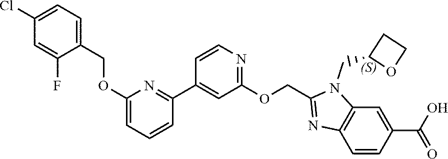

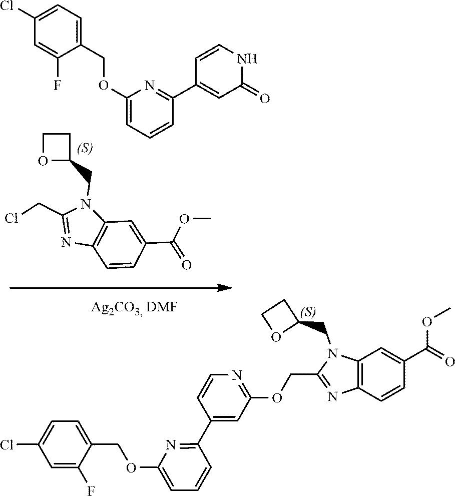

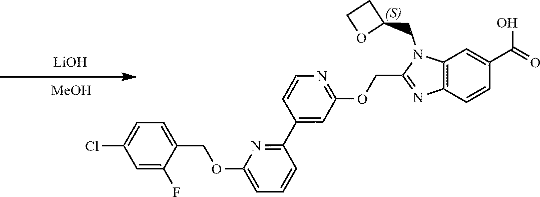

### Step A: Preparation of Methyl 2-[[4-[6-[(4-chloro-2-fluoro-phenyl)methoxy]-2-pyridyl]-2-pyridyl]oxymethyl]-3-[[(2S)-oxetan-2-yl]methyl]benzimidazole-5-carboxylate

A mixture of 4-[6-[(4-chloro-2-fluoro-phenyl)methoxy]-2-pyridyl]-1H-pyridin-2-one (187.6 mg, 567.21 umol), methyl 2-(chloromethyl)-3-[[(2S)-oxetan-2-yl]methyl]benzimidazole-5-carboxylate (183.89 mg, 623.93 umol), Ag₂CO₃ (469.23 mg, 1.70 mmol) was mixed in DMF (2 mL). The mixture was stirred at 30° C. for 48 hr under N₂ atmosphere. The reaction mixture was filtered and concentrated under reduced pressure to give a residue. The residue was purified by column chromatography (SiO₂, Petroleum ether/Ethyl acetate = 40% to 100%). Methyl 2-[[4-[6-[(4-chloro-2-fluorophenyl)-methoxy]-2-pyridyl]-2-pyridyl]oxymethyl]-3-[[(2S)-oxetan-2-yl]methyl]benzimidazole-5-carboxylate (108.9 mg, 32.6% yield) was obtained as a white solid.

### Step B: Preparation of 2-[[4-[6-[(4-chloro-2-fluoro-phenyl)methoxy]-2-pyridyl]-2 -pyridyl]oxymethyl]-3-[[(2S)-oxetan-2-yl]methyl]benzimidazole-5-carboxylic acid

To a solution of methyl 2-[[4-[6-[(4-chloro-2-fluoro-phenyl)methoxy]-2-pyridyl]-2-pyridyl]oxy-methyl]-3-[[(2S)-oxetan-2-yl]methyl]benzimidazole-5-carboxylate (108.9 mg, 184.89 umol) in MeOH (2 mL) was added LiOH.H₂O (2 M, 184.89 uL). The mixture was stirred at 20° C. for 30 hr. The reaction mixture was filtered and concentrated under reduced pressure to give a residue. The residue was purified by prep-HPLC (column: Phenomenex Gemini-NX80*40 mm*3 um; mobile phase: [water (0.05% NH₃H₂O+10 mM NH₄HCO₃)-ACN]; B%:21%-45%, 8 min). 2-[[4-[6-[(4-chloro-2-fluoro-phenyl)methoxy]-2-pyridyl]-2-pyridyl]oxymethyl]-3-[[(2S)-oxetan-2-yl]methyl]benzimidazole-5-carboxylic acid (12.79 mg, 11.9% yield) was obtained as a white solid. LCMS: m/z576.1 [M+H]⁺.

¹H NMR (400 MHz, DMSO-d₆) δ ppm 8.25 - 8.34 (m, 2 H) 7.86 - 7.92 (m, 1 H) 7.84 (dd, J=8.53, 1.25 Hz, 1 H) 7.78 (d, J=7.53 Hz, 1 H) 7.73 (dd, J=5.27, 1.25 Hz, 1 H) 7.57 -7.67 (m, 3 H) 7.49 (dd, J=9.79, 2.01 Hz, 1 H) 7.32 (dd, J=8.28, 1.76 Hz, 1 H) 6.98 (d, J=8.03 Hz, 1 H) 5.64 - 5.82 (m, 2 H) 5.52 (s, 2 H) 5.04 - 5.14 (m, 1 H) 4.74 - 4.85 (m, 1 H) 4.63 - 4.72 (m, 1 H) 4.42 - 4.51 (m, 1 H) 4.33-4.28 (m, 1 H) 2.62 - 2.74 (m, 1 H) 2.31 - 2.42 (m, 1 H);

Example 7W: 2-[[4-[3-[(4-chloro-2-fluoro-phenyl)methoxy]phenyl]-2-pyridyl] oxymethyl]-3-[[(2S)-oxetan-2-yl]methyl]imidazo[4,5-b]pyridine-5-carboxylic acid (Compound 110aw)

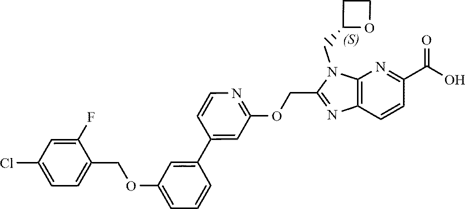

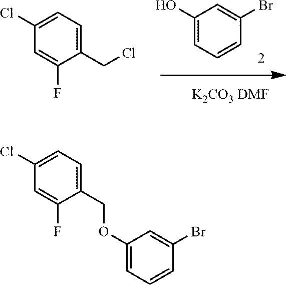

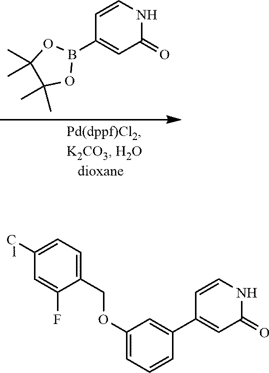

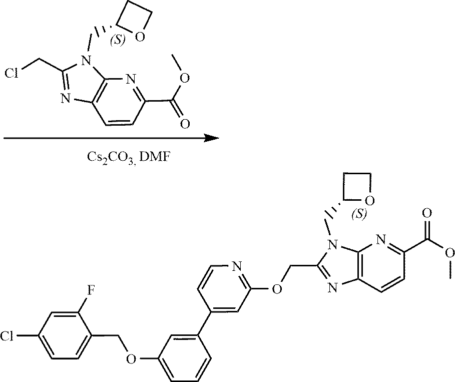

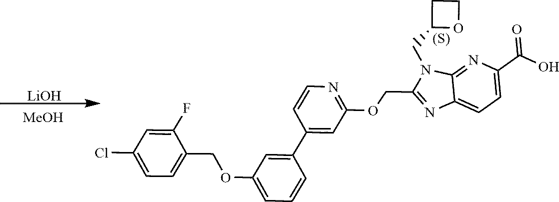

### Step A: Preparation of 1-[(3-bromophenoxy)methyl]-4-chloro-2-fluoro-benzene

A mixture of 4-chloro-1-(chloromethyl)-2-fluoro-benzene (2.00 g, 11.17 mmol), 3-bromophenol (2, 2.13 g, 12.29 mmol), and K₂CO₃ (4.63 g, 33.52 mmol) in DMF (30 mL) was degassed and purged with N₂ for 3 times, and then the mixture was stirred at 20° C. for 16 hr under N₂ atmosphere. The reaction mixture was diluted with H₂O (100 mL) and extracted with EA (30 mL *3). The combined organic layers were washed with aqueous NaCl (20 mL), dried with anhydrous Na₂SO₄, filtered, and concentrated under reduced pressure to give a residue. The residue was purified by column chromatography (SiO2, Petroleum ether/Ethyl acetate=0% to 20%). 1-[(3-bromophenoxy)methyl]-4-chloro- 2-fluoro-benzene (2.43 g, 68.9% yield) was obtained as a white solid.

¹H NMR (400 MHz, CDCl₃) δ ppm 7.44 (t, J=8.00 Hz, 1 H) 7.09 - 7.24 (m, 5 H) 6.87 -6.96 (m, 1 H) 5.07 (s, 2 H)

### Step B: Preparation of 4-[6-[(4-chloro-2-fluoro-phenyl)methoxy]-2-pyridyl]1H-pyridin-2-one

A mixture of 1-[(3-bromophenoxy)methyl]-4-chloro-2-fluoro-benzene (200 mg, 633.78 umol), 4-(4,4,5,5-tetramethyl-1,3,2-dioxaborolan-2-yl)-1H-pyridin-2-one (168.13 mg, 760.54 umol), KOAc (186.60 mg, 1.90 mmol), Pd(dppf)Cl₂ (9.27 mg, 12.68 umol) in H₂O (0.5 mL) and dioxane (2.5 mL) was degassed and purged with N₂ for 3 times, and then the mixture was stirred at 80° C. for 12 hr under N₂ atmosphere. The reaction mixture was filtered and concentrated under reduced pressure to give a residue. The residue was purified by column chromatography (SiO₂, DCM: MeOH = 0% to 10%). 4-[3-[(4-chloro-2-fluoro-phenyl)methoxy]phenyl]-1H-pyridin-2-one (132.5 mg, 62.8% yield) was obtained as a yellow solid. LCMS: m/z330.0[M+H]⁺.

### Step C: Preparation of Methyl 2-[[4-[3-[(4-chloro-2-fluoro-phenyl)methoxy]phenyl]-2-pyridyl]oxymethyl]-3-[[(2S)-oxetan-2-yl]methyl]imidazo[4,5-b]pyridine-5-carboxylate

A mixture of 4-[3-[(4-chloro-2-fluoro-phenyl)methoxy]phenyl]-1H-pyridin-2-one (132.5 mg, 401.82 umol), methyl 2-(chloromethyl)-3-[[(2S)-oxetan-2-yl]methyl]imidazo[4,5-b]pyridine-5-carboxylate (178.24 mg, 602.73 umol) and Cs₂CO₃ (392.76 mg, 1.21 mmol) in DMF (3 mL) was degassed and purged with N₂ for 3 times, and then the mixture was stirred at 25° C. for 16 hr under N₂ atmosphere. The reaction mixture was filtered and concentrated under reduced pressure to give a residue. The residue was purified by column chromatography (SiO₂, Petroleum ether/Ethyl acetate=60% to 100%). Methyl 2-[[4-[3-[(4-chloro-2-fluoro-phenyl) methoxy]phenyl]-2-pyridyl]oxymethyl]-3-[[(2S)-oxetan-2-yl]methyl]imidazo[4,5-b]pyridine-5-carboxylate(7, 72.8 mg, 123.60 umol, 30.8% yield) was obtained as a yellow oil. LCMS: m/z589.1 [M+H]⁺.

### Step D: Preparation of 2-[[4-[3-[(4-chloro-2-fluoro-phenyl)methoxy]phenyl]-2-pyridyl] oxymethyl]-3-[[(2S)-oxetan-2-yl]methyl]imidazo[4,5-b]pyridine-5-carboxylic acid

To a solution of methyl 2-[[4-[3-[(4-chloro-2-fluoro-phenyl)methoxy]phenyl]-2-pyridyl]oxy-methyl]-3-[[(2S)-oxetan-2-yl]methyl]imidazo[4,5-b]pyridine-5-carboxylate (7, 72.80 mg, 123.60 umol) in MeOH (2 mL) was added LiOH.H₂O (2 M, 123.60 uL). The mixture was stirred at 20° C. for 0.5 hr. LCMS showed that the starting material was consumed completely and one main peak with desired mass was detected. The reaction mixture was filtered and concentrated under reduced pressure to give a residue. The residue was purified by prep-HPLC (column: Phenomenex Gemini-NX 80*40 mm*3 um; mobile phase: [water (0.05% NH₃H₂O + 10 Mm NH₄HCO₃)-ACN]; B%: 22%-52%, 8 min). 2-[[4-[3-[(4-chloro-2-fluoro-phenyl)methoxy]phenyl]-2-pyridyl]oxymethyl]-3-[[(2S)-oxetan-2-yl]methyl]imidazo[4,5-b]pyridine-5-carboxylic acid (25.36 mg, 35.3% yield) was obtained as a white solid. LCMS: m/z575.1 [M+H]⁺.

¹H NMR (400 MHz, DMSO-d₆) δ ppm 8.22 - 8.36 (m, 2 H) 7.86 - 7.92 (m, 1 H) 7.83 (dd, J=8.41, 1.38 Hz, 1 H) 7.77 (d, J=7.28 Hz, 1 H) 7.72 (dd, J=5.40, 1.38 Hz, 1 H) 7.57 -7.67 (m, 3 H) 7.49 (dd, J=10.04, 2.01 Hz, 1 H) 7.31 (dd, J=8.03, 2.01 Hz, 1 H) 6.98 (d, J=8.28 Hz, 1 H) 5.66 - 5.80 (m, 2 H) 5.52 (s, 2 H) 5.11-5.06 (m, 1 H) 4.74 - 4.82 (m, 1 H) 4.63 - 4.70 (m, 1 H) 4.43 - 4.50 (m, 1 H) 4.33-4.27 (m, 1 H) 2.62 - 2.73 (m, 1 H) 2.31 -2.44 (m, 1 H);

Example 8W: 2-[[4-[3-[(4-chloro-2-fluoro-phenyl)methoxy]phenyl]-2-oxo-1-pyridyl]methyl]-3-[[(2S)-oxetan-2-yl]methyl]-imidazo[4,5-b]pyridine-5-carboxylic acid (Compound 108aw)

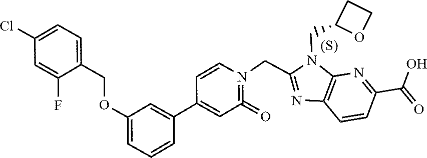

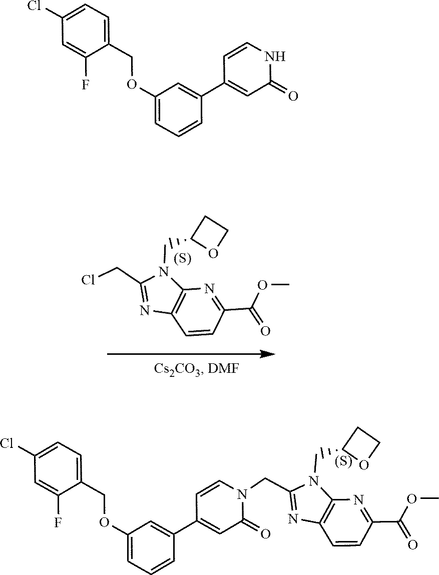

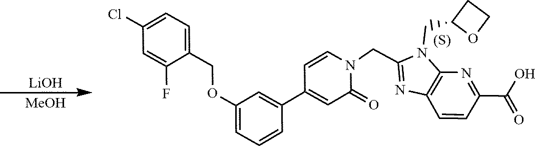

### Step A: Preparation of methyl2-[[4-[3-[(4-chloro-2-fluoro-phenyl)methoxy]phenyl]-2-oxo-1-pyridyl]methyl]-3-[[(2S)-oxetan-2-yl]methyl]imidazo[4,5-b]pyridine-5-carboxylate

A mixture of 4-[3-[(4-chloro-2-fluoro-phenyl)methoxy]phenyl]-1H-pyridin-2-one (213 mg, 645.94 umol), methyl 2-(chloromethyl)-3-[[(2S)-oxetan-2-yl]methyl]imidazo[4,5-b]pyridine-5-carboxylate (229.22 mg, 775.13 umol) and K₂CO₃ (267.83 mg, 1.94 mmol) in ACN (3 mL) was degassed and purged with N₂ for 3 times, and then the mixture was stirred at 25° C. for 16 hr under N₂ atmosphere. The reaction mixture was filtered and concentrated under reduced pressure to give a residue. The residue was purified by column chromatography (SiO₂, Petroleum ether/Ethyl acetate=50% to 100%). Methyl 2-[[4-[3-[(4-chloro-2-fluoro-phenyl)methoxy]phenyl]-2-oxo-1-pyridyl]methyl]-3-[[(2S)-oxetan-2-yl]methyl]imidazo[4,5-b]pyridine-5-carboxylate (129.6 mg, 209.03 umol, 32.4% yield) was obtained as a yellow oil. LCMS: m/z589.0[M+H]⁺.

### Step B: Preparation of 2-[[4-[3-[(4-chloro-2-fluoro-phenyl)methoxy]phenyl]-2-oxo-1-pyridyl]methyl]-3-[[(2S)-oxetan-2-yl]methyl]-imidazo[4,5-b]pyridine-5-carboxylic

To a solution of methyl 2-[[4-[3-[(4-chloro-2-fluoro-phenyl)methoxy]phenyl]-2-oxo-1-pyridyl]-methyl]-3-[[(2S)-oxetan-2-yl]methyl]imidazo[4,5-b]pyridine-5-carboxylate (129.6 mg, 220.03 umol) in MeOH (2 mL) was added LiOH.H₂O (2 M, 220.03 uL). The mixture was stirred at 25° C. for 0.5 hr. The reaction mixture was concentrated under reduced pressure to give a residue. The residue was purified by prep-HPLC (column: Phenomenex Gemini-NX 80*40 mm*3 um; mobile phase: [water (0.05% NH₃H₂O + 10 mM NH₄HCO₃)-ACN]; B%: 22%-44%. 8 min). 2-[[4-[3-[(4-chloro-2-fluorophenyl)methoxy]phenyl]-2-oxo-1-pyridyl]methyl]-3-[[(2S)-oxetan-2-yl]methyl]-imidazo[4,5-b]pyridine-5-carboxylic acid (22.59 mg, 17.5% yield) was obtained as a white solid. LCMS: m/z575.1[M=H]⁺.

¹H NMR (400 MHz, DMSO-d₆) δ ppm 7.87 - 8.01 (m, 3 H) 7.63 (t, J=8.16 Hz, 1 H) 7.52 (dd, J=10.04, 2.01 Hz, 1 H) 7.39 - 7.47 (m, 2 H) 7.37-7.34 (m, 2 H) 7.14-7.12 (m, 1 H) 6.61 - 6.78 (m, 2 H) 5.41 - 5.72 (m, 2 H) 5.24 (s, 2) 5.08 - 5.17 (m, 1 H) 4.78-4.89 (m, 1 H) 4.65 - 4.76 (m, 1 H) 4.44 - 4.56 (m, 1 H) 4.37-4.31 (m, 1 H) 2.62 - 2.77 (m, 1 H) 2.32 - 2.46 (m, 1 H);

Example 9W: (S)-2-(((6-((4-chloro-2-fluorobenzyl)oxy)-5′-fluoro-[2,4′-bipyridin]-2′-yl)oxy)methyl)-3-(oxetan-2-ylmethyl)-3H-imidazo[4,5-b]pyridine-5-carboxylic acid (Compound 109aw)

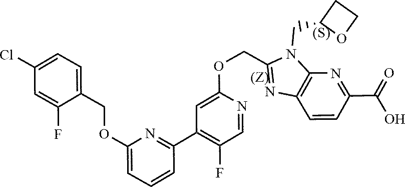

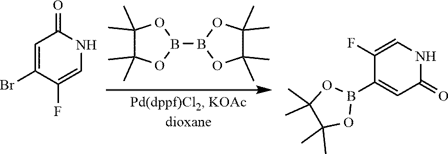

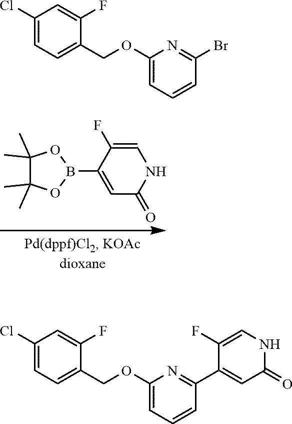

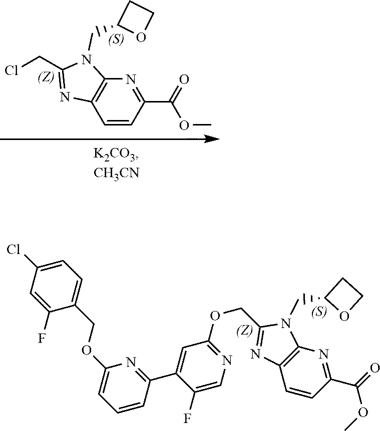

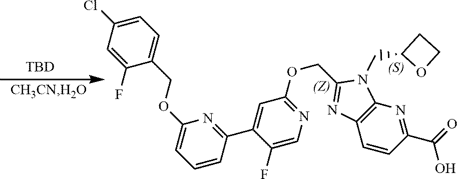

### Step A: Preparation of 5-fluoro-4-(4,4,5,5-tetramethyl-1,3,2-dioxaborolan-2-yl)-1H-pyridin-2-one

To solution of 4-bromo-5-fluoro-1H-pyridin-2-one (200 mg, 1.04 mmol) in 1,4-dioxane (5 mL) were added 4,4,5,5-tetramethyl-2-(4,4,5,5-tetramethyl-1,3,2-dioxaborolan-2-yl) -1,3,2-dioxaborolane (291 mg, 1.15 mmol), Pd(dppf)Cl₂ (76 mg, 104.17 umol), and KOAc (307 mg, 3.13 mmol). The reaction was stirred at 90° C. for 12 h. The reaction mixture was filtered, and the filtrate was concentrated under reduced pressure to give 5-fluoro-4-(4,4,5,5-tetramethyl-1,3,2-dioxaborolan-2-yl)-1H-pyridin-2-one (240 mg, crude) as a brown solid.

### Step B: Preparation of 6-((4-chloro-2-fluorobenzyl)oxy)-5′-fluoro-[2,4′-bipyridin]-2′(1′H)-one

To solution of 2-bromo-6-[(4-chloro-2-fluoro-phenyl)methoxy]pyridine (250 mg, 789.76 umol) in 1,4-dioxane (6 mL) and H₂O (2 mL) were added 5-fluoro-4-(4,4,5,5-tetramethyl-X1.3,2-dioxaborolan-2-yl)-1H-pyridin-2-one (227 mg, 947.71 umol), Pd(dppf)Cl₂ (58 mg, 78.98 umol), and Na₂CO₃ (251 mg, 2.37 mmol). The mixture was de-gassed and then heated to 95° C. for 12 hours under N₂. The reaction mixture was diluted with H₂O (10 mL) and extracted with EA (20 mL x 3). The combined organic layers were washed with brine (20 mL), dried over Na₂SO₄, filtered, and concentrated under reduced pressure to give a residue. The residue was purified by column chromatography (SiO₂, DCM/MeOH = ⅟0 to 10/1) to provide 4-[6-[(4-chloro-2-fluorophenyl)methoxy]-2-pyridyl]-5-fluoro-1H-pyridin-2-one (92 mg, yield: 24.6%) as a brown solid. LCMS: m/z387.0[M+K]⁺.

### Step C: Preparation of (S)-methyl 2-(((6-((4-chloro-2-fluorobenzyl)oxy)-5′-fluoro- [2.4′-bipyridin]-2′-yl)oxy)methyl)-3-(oxetan-2-ylmethyl)-3H-imidazo[4,5-b]pyridine-5-carboxylate

To solution of 4-[6-[(4-chloro-2-fluoro-phenyl)methoxy]-2-pyridyl]-5-fluoro-1H-pyridin-2-one (92 mg, 193.90 umol) in CH₃CN (3 mL) was added methyl 2-(chloromethyl)-3-[[(2S)-oxetan-2-yl]methyl]imidazo[4,5-b]pyridine-5-carboxylate (63 mg, 213.29 umol), KI (3.22 mg, 19.39 umol) and K₂CO₃ (107 mg, 775.61 umol). The reaction was stirred at 80° C. for 12 h. The reaction mixture was diluted with H₂O (20 mL) and extracted with EA (20 mL x 3). The combined organic layers were washed with brine (10 mL x 2), dried over Na₂SO₄, filtered, and concentrated under reduced pressure to give a residue. The residue was purified by column chromatography (SiO₂, DCM/MeOH = ⅟0 to 15/1). Methyl 2-[[4-[6-[(4-chloro-2-fluoro-phenyl)methoxy]-2-pyridyl]-5-fluoro-2-oxo-1-pyridyl]methyl]-3-[[(2S)-oxetan-2-yl]methyl]imidazo[4,5-b]pyridine-5-carboxylate (79 mg, yield: 36.9%) was obtained as yellow oil. LCMS: m/z608.3[M+H]⁺.

### Step D: Preparation of (S)-2-(((6-((4-chloro-2-fluorobenzyl)oxy)-5′-fluoro-[2,4′-bipyridin]-2′-yl)oxy)methyl)-3-(oxetan-2-ylmethyl)-3H-imidazo[4,5-b]pyridine-5-carboxylic acid

To a solution of methyl 2-[[4-[6-[(4-chloro-2-fluoro-phenyl)methoxy]-2-pyridyl] -5-fluoro-2-oxo-1-pyridyl]methyl]-3-[[(2S)-oxetan-2-yl]methyl]imidazo[4,5-b]pyridine-5-carboxylate (79 mg, 71.46 umol) in CH₃CN (2 mL) and H₂O (0.2 mL) was added 3,4,6,7,8,9-hexahydro-2H-pyrimido[1,2-a]pyrimidine (19.90 mg, 142.93 umol). The reaction was stirred at 25° C. for 12 h. The reaction was concentrated to remove CH₃CN, the aqueous layer was acidified to pH 6 with 0.1 N HCl. The mixture was extracted with EA (30 mL x 3). The organic layer was dried over Na₂SO₄, filtered and concentrated under reduced pressure to give a residue. The residue was purified by prep-HPLC (column: Phenomenex Gemini-NX 80 x 30 mm x 3 um; mobile phase: [water (10 mM NH₄HCO₃)-ACN]; B%: 10%-80%, 9.5 min) to provide 2(S)-2-(((6-((4-chloro-2-fluorobenzyl)oxy)-5′-fluoro-[2,4′- bipyridin]-2′-yl)oxy)methyl)-3-(oxetan-2-ylmethyl)-3H-imidazo[4,5-b]pyridine-5-carboxylic acid (7.65 mg, yield: 18.0%) as a white solid. LCMS: m/z594.1 [M+H]⁺.

¹H NMR (CD₃OD, 400 MHz): δ 8.10 - 8.16 (m, 2 H), 8.11 - 8.04 (m, 1 H), 7.79 (t, J = 7.8 Hz,1 H), 7.59 - 7.49 (m, 3 H), 7.23 - 7.17 (m, 2 H), 6.92 (d, J = 8.0 Hz, 1 H), 5.89 - 5.85 (m, 1 H), 5.78 - 5.73 (m, 1 H), 5.49 (s, 2 H), 5.29 - 5.19 (m, 1 H), 4.99 - 4.93 (m, 2 H), 4.63 -4.55 (m, 1 H), 4.41 - 4.34 (m, 1 H), 2.85 - 2.70 (m, 1 H), 2.54 - 2.42 (m, 1 H).

Example 10W: (S)-2-(4-(4-((4-chloro-2-fluorobenzyl)oxy)pyrimidin-2-yl)benzyl)-3-(oxetan-2-ylmethyl)-3H-imidazo[4,5-b]pyridine-5-carboxylic acid (Compound 101aw)

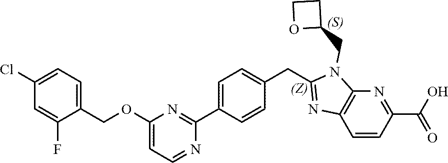

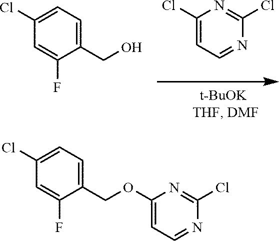

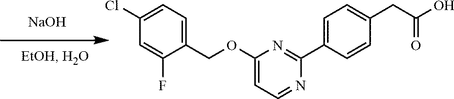

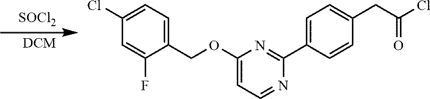

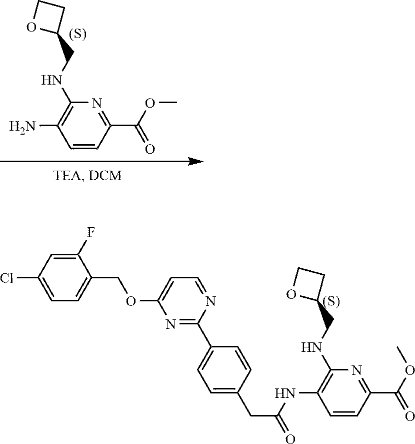

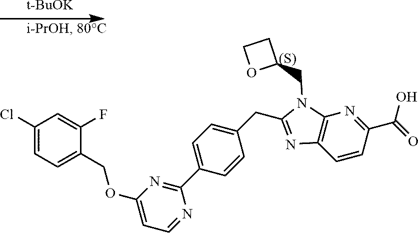

### Step A: Preparation of 2-chloro-4-[(4-chloro-2-fluoro-phenyl)methoxy]pyrimidine

The mixture of (4-chloro-2-fluoro-phenyl) methanol (1.00 g, 6.23 mmol) and t-BuOK (699.08 mg, 6.23 mmol) in THF (3 mL) was heated at 60° C. for 0.5 hr. The mixture was cooled to 0° C. The resulting mixture was slowly added to the mixture of 2,4-dichloropyrimidine (928.13 mg, 6.23 mmol, 724.64 uL) in DMF (5 mL) at -50° C. The mixture was stirred at -50° C. for 1 hr and then warm to 25° C. The reaction mixture was stirred at 25° C. for 16 hr. The mixture was added to 30 mL of cold H₂O dropwise. The mixture was slowly warm to 10° C., and then extracted with Ethyl acetate (15 mL*3). The organic layers were combined, dried over Na₂SO₄ and filtered. The filtrate was concentrated in vacuo to give a crude 2-chloro-4-[(4-chloro-2-fluorophenyl)methoxy]pyrimidine (1.51 g) as a light yellow solid. LCMS: m/z272.9[M+H]⁺.

¹H NMR (400 MHz, CDCl₃) δ ppm 8.33 (d, J=5.75 Hz, 1 H) 7.41 - 7.47 (m, 1 H) 7.13 -7.20 (m, 3 H) 6.71 (d, J=5.63 Hz, 1 H) 5.46 (s, 3 H)

### Step B: Preparation of Methyl 2-(4-(4-((4-chloro-2-fluorobenzyl)oxy)pyrimidin-2-yl) phenyl)acetate

To a solution of 2-chloro-4-[(4-chloro-2-fluoro-phenyl)methoxy]pyrimidine (500 mg, 1.83 mmol) and methyl 2-(4-(4,4,5,5-tetramethyl-1,3,2-dioxaborolan-2-yl)phenyl)acetate (606.69 mg, 2.20 mmol) in dioxane (6 mL) and H₂O (2 mL) was added K₂CO₃ (759.12 mg, 5.49 mmol) and Pd(dppf)Cl₂ (133.97 mg, 183.09 umol). The mixture was stirred at 80° C. under N₂ for 16 hr. The mixture was diluted with water (15 mL) and extracted with EtOAc (10 mL*2). The combined organic layers were dried over anhydrous Na₂SO₄, filtered and concentrated to give a residue (451 mg). The residue was purified by column chromatography (12 g SiO₂, Petroleum ether/Ethyl acetate= 0% to 30%) to give methyl 2-(4-(4-((4-chloro-2-fluorobenzyl)oxy)pyrimidin-2-yl)phenyl)acetate (317 mg, 44.8% yield) as a white solid. LCMS: m/z386.9[M+H]⁺.

¹H NMR (400 MHz, CDCl₃) δ ppm 8.54 (d, J=5.77 Hz, 1 H) 8.40 (d, J=8.28 Hz, 2 H) 7.47 (t, J=8.28 Hz, 1 H) 7.41 (d, J=8.28 Hz, 2 H) 7.16 (d, J=8.28 Hz, 2 H) 6.68 (d, J=5.77 Hz, 1 H) 5.60 (s, 2 H) 3.72 - 3.75 (m, 5 H)

### Step C: Preparation of 2-(4-(4-((4-chloro-2-fluorobenzyl)oxy)pyrimidin-2-yl)phenyl)-acetic acid

To a solution of methyl 2-(4-(4-((4-chloro-2-fluorobenzyl)oxy)pyrimidin-2-yl)phenyl)acetate (5, 317 mg, 819.54 umol) was added NaOH (98.3 mg, 2.46 mmol) in H₂O (1 mL) and EtOH (3 mL) at 25° C. for 1 hr. The mixture was diluted with water (8 mL). To the aqueous layer was added 2N HC1 aq. to adjust to pH = 5. The mixture was diluted with EtOAc (8 mL*2). The combined organic layers were dried over anhydrous Na₂SO₄, filtered and concentrated to give 2-(4-(4-((4-chloro-2-fluorobenzyl)oxy)pyrimidin-2-yl)phenyl)acetic acid (212 mg, crude) as a white solid. It was used for the next step.

¹H NMR (400 MHz, CDCl₃) δ ppm 8.55 (d, J=5.62 Hz, 1 H) 8.39 (d, J=8.31 Hz, 2 H) 7.38 - 7.49 (m, 3 H) 7.16 (d, J=8.31 Hz, 2 H) 6.69 (d, J=5.62 Hz, 1 H) 5.60 (s, 2 H) 3.75 (s, 2 H)

### Step D: Preparation of 2-(4-(4-((4-chloro-2-fluorobenzyl)oxy)pyrimidin-2-yl)phenyl) acetyl chloride

To a solution of 2-(4-(4-((4-chloro-2-fluorobenzyl)oxy)pyrimidin-2-yl)phenyl)acetic acid (212.00 mg, 568.70 umol) in DCM (3 mL) was added SOCl₂ (135.32 mg, 1.14 mmol, 82.51 uL) at 0° C. The mixture was stirred at 25° C. for 1 hr. The reaction solution was concentrated under reduced pressure to give 2-(4-(4-((4-chloro-2-fluorobenzyl)oxy)pyrimidin-2-yl)phenyl)acetyl chloride (231 mg, crude) which was used directly in the next step.

### Step E: Preparation of (S)-methyl 5-(2-(4-(4-((4-chloro-2-fluorobenzyl)oxy)pyrimidin-2-yl)phenyl)acetamido)-6-((oxetan-2-ylmethyl)amino)picolinate

To a solution of 2-(4-(4-((4-chloro-2-fluorobenzyl)oxy)pyrimidin-2-yl)phenyl)acetyl chloride (105 mg, 442.56 umol) in DCM (2 mL) was added Et₃ N (134.35 mg, 1.33 mmol, 184.80 uL) at 0° C. A mixture of (S)-methyl 5-amino-6-((oxetan-2-ylmethyl)amino)picolinate (225.08 mg, 575.33 umol) in DCM (2 mL) was added to the solution and stirred at 25° C. for 16 hr. The mixture was diluted with water (8 mL) and extracted with DCM (10 mL*2). The combined organic layers were dried over anhydrous Na₂SO₄, filtered, and concentrated to give a residue. The residue was purified by column chromatography (4 g SiO₂, Petroleum ether/Ethyl acetate=30% to 100%) to give (S)-methyl 5-(2-(4-(4-((4-chloro-2-fluorobenzyl)oxy)pyrimidin-2-yl)phenyl)acetamido)-6-((oxetan-2-ylmethyl)amino)picolinate (273 mg, crude) as a brown gum. LCMS: m/z 591.1[M+H]⁺.

### Step F: Preparation of (S)-2-(4-(4-((4-chloro-2-fluorobenzyl)oxy)pyrimidin-2-yl)benzyl)-3-(oxetan-2-ylmethyl)-3H-imidazo[4,5-b]pyridine-5-carboxylic acid

To a solution of (S)-methyl 5-(2-(4-(4-((4-chloro-2-fluorobenzyl)oxy)pyrimidin-2-yl)phenyl)acetamido)-6-((oxetan-2-ylmethyl)amino)picolinate (273 mg, 461.14 umol) in i-PrOH (3 mL) was added t-BuOK (103.49 mg, 922.27 umol). The mixture was stirred at 80° C. for 30 min. LC-MS indicated that one main peak with desired mass was detected. The reaction solution was concentrated under reduced pressure to give a residue. The residue was purified by Prep-HPLC to give (S)-2-(4-(4-((4-chloro-2-fluorobenzyl)oxy)pyrimidin-2-yl)benzyl)-3-(oxetan-2-ylmethyl)-3H-imidazo[4,5-b]pyridine-5-carboxylic acid (2.49 mg, 4.45 umol, 1.0% yield) as white solid. LCMS: m/z 560.3[M+H]⁺.

¹H NMR (400 MHz, CD₃OD) δ ppm 8.53 (d, J=5.87 Hz, 1 H) 8.38 (d, J=8.31 Hz, 2 H) 8.07 - 8.17 (m, 2 H) 7.55 (t, J=8.07 Hz, 1 H) 7.45 (d, J=8.31 Hz, 2 H) 7.21 - 7.29 (m, 2 H) 6.82 (d, J=5.75 Hz, 1 H) 5.63 (s, 2 H) 5.24 (m, 1 H) 4.68 - 4.76 (m, 1 H) 4.57 - 4.67 (m, 4 H) 4.41 - 4.49 (m, 1 H) 2.68 - 2.85 (m, 1 H) 2.39 - 2.55 (m, 1 H).

Example 11W: 2-[[4-(4-benzyloxypyrimidin-2-yl)phenyl]methyl]-3-[[(2S)-oxetan-2-yl]methyl]imidazo[4,5-b]pyridine-5-carboxylic acid (Compound 102aw)

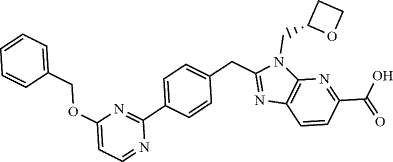 [SMILES: O=C(O)c1ccc2nc(Cc3ccc(-c4nccc(OCc5ccccc5)n4)cc3)n(C[C@@H]3CCO3)c2n1]

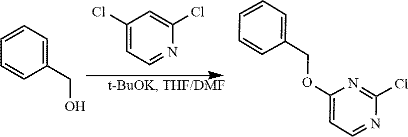

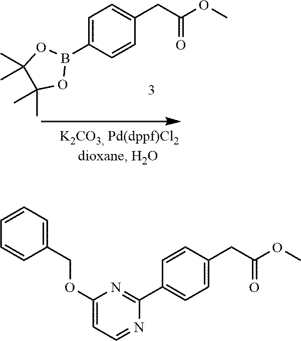

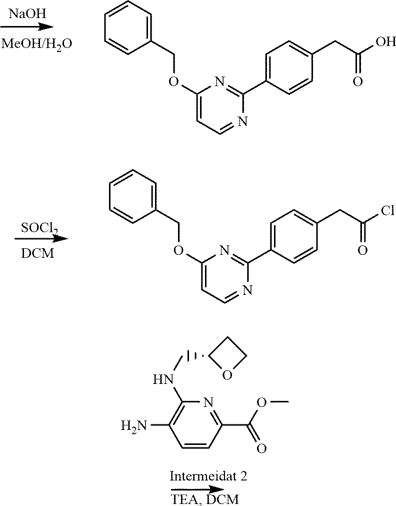

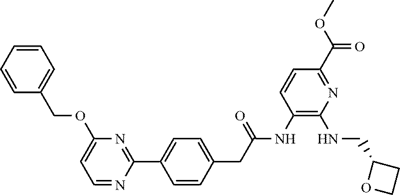 [SMILES: COC(=O)c1ccc(NC(=O)Cc2ccc(-c3nccc(OCc4ccccc4)n3)cc2)c(NC[C@@H]2CCO2)n1]

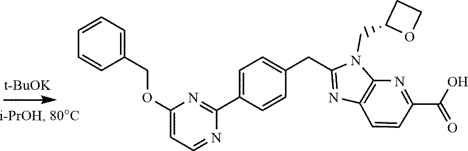

### Step A: Preparation of 4-benzvloxv-2-chloro-pyrimidine

To a solution of phenylmethanol (2.00 g, 18.49 mmol, 1.92 mL) in THF (20 mL) was added t-BuOK (2.49 g, 22.19 mmol) at 80° C. for 0.5 h. Then a mixture of 2, 4-dichloropyrimidine (2.76 g, 18.49 mmol) in DMF (10 mL) at -70° C. was added, and the resulting mixture was stirred at -70° C. for 2 h. The mixture was diluted with water (40 mL), and the resulting solution was extracted with EtOAc (20 mL*3). The combined organic layer was dried over anhydrous Na₂SO₄, filtered and the filtrate was concentrated in vacuo to give a crude residue (3.68 g) as white solid. The residue was purified by combi flash (40 g silica gel column, EtOAc in PE from 0% to 50%). 4-benzyloxy-2-chloro-pyrimidine (2.70 g, 12.24 mmol) was obtained as white solid. ¹H NMR (400 MHz, CDCl₃) δ 8.23 (d, J=4.0 Hz, 1 H), 7.25-7.43 (m, 5 H), 6.63 (d, J=8.0 Hz, 1 H), 5.36 (s, 2 H).

### Step B: Preparation of Methyl 2-[4-(4-benzyloxypyrimidin-2-yl)phenyl]acetate

To a solution of 4-benzyloxy-2-chloro-pyrimidine (1.0 g, 4.53 mmol) and methyl 2-[4-(4,4,5,5-tetramethyl-1,3,2-dioxaborolan-2-yl)phenyl]acetate (1.25 g, 4.53 mmol) in dioxane (10 mL) and H₂O (4 mL) was added Pd(dppf)Cl₂ (331.6 mg, 0.45 mmol) and K₂CO₃ (1.88 g, 13.60 mmol). The reaction was maintained at 80° C. under N₂ for 16 hr. The mixture was washed with water (30 mL) anddiluted with EtOAc (30 mL*2). Then the combined organic layers were dried over anhydrous Na₂SO₄, filtered, and concentrated to give a residue. The residue was purified by column chromatography (25 g SiO₂, Petroleum ether/Ethyl acetate=0% to 50%). Methyl 2-[4-(4-benzyloxypyrimidin-2-yl)phenyl]acetate (517.3 mg, 1.55 mmol, 34.1% yield) was obtained as a yellow oil. LCMS: m/z 335.1 [M+H]⁺.

### Step C: Preparation of 2-[4-(4-benzyloxypyrimidin-2-yl)phenyl]acetic Acid

To a solution of methyl 2-[4-(4-benzyloxypyrimidin-2-yl) phenyl] acetate (500 mg, 1.50 mmol) in EtOH (5 mL) was added NaOH (179.9 mg, 4.50 mmol) in H₂O (1 mL) at 25° C. The mixture was stirred at 25° C. for 1 hr. The mixture was washed with water (30 mL) after dilution with EtOAc (30 mL*2). The combined organic layers were dried over anhydrous Na₂SO₄, filtered, and concentrated to give a residue (304.5 mg). 2-[4-(4-benzyloxypyrimidin-2-yl) phenyl] acetic acid (304.5 mg, crude) was obtained as a yellow solid.

¹H NMR (400 MHz, CD₃OD) δ ppm 8.52 (d, J=5.87 Hz, 1 H)8.34 (d, J=8.31 Hz, 2 H) 7.49 - 7.53 (m, 2 H) 7.42 (d, J=8.19 Hz, 2 H) 7.35 - 7.41 (m, 2 H) 7.33 (d, J=6.97 Hz, 1 H) 6.80 (d, J=5.75 Hz, 1 H) 5.59 (s, 2 H) 3.70 (s, 2 H)

### Step D: Preparation of 2-[4-[4-[(4-chloro-2-fluoro-phenyl)methoxylpyrimidin-2-yl]phenyl]acetyl chloride

To a solution of 2-[4-(4-benzyloxypyrimidin-2-yl) phenyl] acetic acid (300 mg, 0.94 mmol) in DCM (3 mL) was added SOC1₂ (222.8 mg, 1.87 mmol, 0.13 mL) at 0° C. Then the mixture was stirred at 25° C. for 1 hr. TLC (Petroleum ether: Ethyl acetate/2: 1, UV) showed the starting material was consumed, and a new spot was observed. The reaction was concentrated to provide 2-[4-[4-[(4-chloro-2-fluoro-phenyl)methoxy]pyrimidin-2-yl]phenyl]acetyl chloride (315.6 mg, crude) as a yellow solid.

### Step E: Preparation of Methyl 5-[[2-[4-(4-benzyloxypyrimidin-2-yl)phenyl]acetyl]amino] -6-[[(2S)-oxetan-2-yl]methylamino]pyridine-2-carboxylate

To a solution of methyl 5-amino-6-[[(2S)-oxetan-2-yl]methylamino]pyridine-2-carboxylate (147.1 mg, 0.62 mmol) in DCM (4 mL) was added Et₃ N (268.8 mg, 2.66 mmol, 0.37 mL) at 0° C. To the mixture was added the solution of 2-[4-(4-benzyloxypyrimidin-2-yl) phenyl]acetyl chloride (300 mg, crude) in DCM (4 mL). The resulting mixture was stirred at 25° C. for 16 hr, after which it was concentrated to give a residue. The residue was purified by column chromatography (4 g SiO₂, Petroleum ether/Ethyl acetate=0% to 50%). Methyl 5-[[2-[4-(4-benzyloxypyrimidin-2-yl)phenyl]acetyl]amino]-6-[[(2S)-oxetan-2-yl]methylamino]pyridine-2-carboxylate (208 mg, 0.38 mmol, 43.5% yield) was obtained as a yellow solid. LCMS: m/z540.1 [M+H]⁺. ¹H NMR (400 MHz, CDCl₃) δ ppm 8.53 (d, J=5.75 Hz, 1 H8.46 (br d, J=8.13 Hz, 2 H) 7.91-7.97 (m, 1 H) 7.68 (br s, 1 H) 7.45 - 7.55 (m, 5 H) 7.32 - 7.43 (m, 3 H) 6.70 (d, J=5.75 Hz, 1 H) 5.57 (s, 2 H) 4.95 - 5.05 (m, 1 H) 4.82 - 4.92 (m, 1 H) 4.59 - 4.68 (m, 1 H) 4.44 - 4.52 (m, 1 H) 3.92 (s, 3 H) 3.80 - 3.87 (m, 2 H) 3.65 - 3.77 (m, 2 H) 2.57 - 2.68 (m, 1 H) 2.40 - 2.52 (m, 1 H)

### Step F: Preparation of 2-[[4-(4-benzyloxypyrimidin-2-yl)phenyl]methyl]-3-[[(2S)-oxetan-2-yl]methyl]imidazo[4,5-b]pyridine-5-carboxylic acid

To a solution of methyl 5-[[2-[4-(4-benzyloxypyrimidin-2-yl)phenyl]acetyl]amino]-6-[[(2S)-oxetan-2-yl]methylamino] pyridine-2-carboxylate (200 mg, 0.37 mmol) in i-PrOH (3 mL) was added t-BuOK (83.2 mg, 0.74 mmol). The mixture was stirred at 80° C. for 30 min. The reaction mixture was filtered. The mixture was further purified by prep-HPLC (column: YMC-Actus Triart C18 150*30 mm*5 um; mobile phase: [water (0.225% FA)-ACN]; B%: 50%-75%, 11 min). The fraction was dried by lyophilized. 2-[[4-(4-benzyloxypyrimidin-2-yl)phenyl]methyl]-3-[[(2S)-oxetan-2-yl]methyl]imidazo[4,5-b]pyridine-5-carboxylic acid (28.9 mg, 0.05 mmol, 15.1% yield, 98.4% purity) was obtained as a white solid. LCMS: m/z508.2[M+H]⁺. ¹H NMR (400 MHz, DMSO-d₆) δ ppm 8.62 (d, J=5.62 Hz, 1 H) 8.36 (d, J=8.19 Hz, 2 H) 8.10 (d, J=8.19 Hz, 1 H) 7.99 (d, J=8.31 Hz, 1 H) 7.45 - 7.53 (m, 4 H) 7.37 - 7.43 (m, 2 H) 7.30 - 7.36 (m, 1 H) 6.91 (d, J=5.75 Hz, 1 H) 5.56 (s, 2 H) 5.05 - 5.13 (m,1 H) 4.61 - 4.71 (m, 1 H) 4.44 - 4.59 (m, 4 H) 4.32 - 4.40 (m, 1 H) 2.61-2.71 (m,1 H) 2.35-2.46 (m, 1 H).

Example 12W: 2-[[4-[6-[(4-chloro-2-fluoro-phenyl)methoxy]-2-pyridyl]phenyl] methyl]-3-[[(2S)-oxetan-2-yl]methyl]imidazo[4,5-b]pyridine-5-carboxylic acid (Compound 120aw)

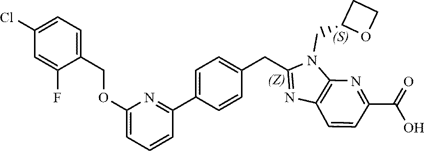

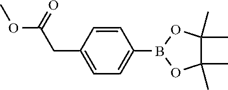

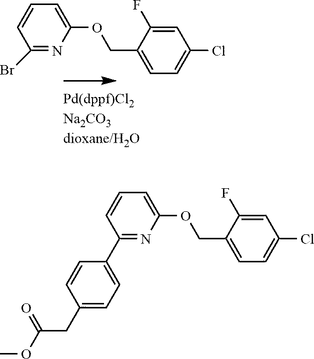

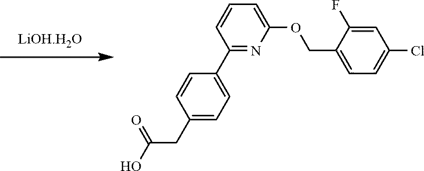 [SMILES: "LiOH.H2O" ; O=C(O)Cc1ccc(-c2cccc(OCc3ccc(Cl)cc3F)n2)cc1]

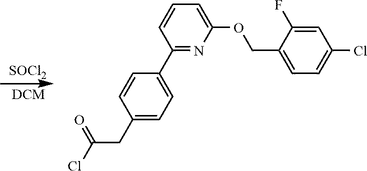

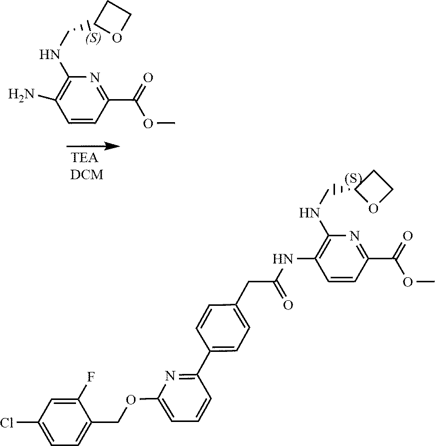

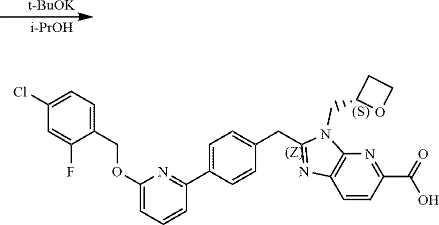

### Step A: Preparation of Methyl 2-(4-(6-((4-chloro-2-fluorobenzyl)oxy)pyridin-2-yl) phenyl)acetate

A mixture of methyl 2-[4-(4,4,5,5-tetramethyl-1,3,2-dioxaborolan-2-yl)phenyl]acetate (500 mg, 1.81 mmol), 2-bromo-6-[(4-chloro-2-fluoro-phenyl)methoxy]pyridine (630 mg, 1.99 mmol), Pd(dppf)Cl₂ (132 mg, 181.07 umol) and Na₂COₛ (384 mg, 3.62 mmol) in H₂O (6 mL) and dioxane (18 mL) was stirred at 80° C. for 16 h under N₂. The reaction mixture was concentrated. The residue was purified by silica gel chromatography (PE: EA = 3:1). Methyl 2-[4-[6-[(4-chloro-2-fluoro-phenyl)methoxy]-2-pyridyl]phenyl]acetate (360 mg, yield: 51.2%) was obtained as yellow oil. LCMS: m/z386.3[M+H]⁺.

### Step B: Preparation of 2-(4-(6-((4-chloro-2-fluorobenzyl)oxy)pyridin-2-yl)phenyl) Acetic acid

A mixture of methyl 2-[4-[6-[(4-chloro-2-fluoro-phenyl)methoxy]-2-pyridyl]phenyl]acetate (360 mg, 933.09 umol) and LiOH.H₂O (196 mg, 4.67 mmol) in MeOH (3 mL), THF (3 mL) and H₂O (3 mL) was stirred at 25° C. for 20 min. The PH of the resulting mixture was adjusted to 7 with HCl (1N). The aqueous phase was extracted with ethyl acetate (10 mL x 3). The combined organic phase was dried with anhydrous Na₂SO₄, filtered and concentrated in vacuo. 2-[4-[6-[(4-chloro-2-fluorophenyl)methoxy]-2-pyridyl]phenyl] acetic acid (280 mg, yield: 80.7%) was obtained as white solid.

### Step C: Preparation of 2-(4-(6-((4-chloro-2-fluorobenzyl)oxy)pyridin-2-yl)phenyl) Acetyl chloride

To a solution of 2-[4-[6-[(4-chloro-2-fluoro-phenyl)methoxy]-2-pyridyl]phenyl]acetic acid (280 mg, 753.12 umol) in DCM (10 mL) was added SOCl₂ (896 mg, 7.53 mmol). Then the reaction mixture was stirred at 25° C. for 30 min. The reaction mixture was concentrated. 2-[4-[6-[(4-chloro-2-fluoro-phenyl)methoxy]-2-pyridyl]phenyl]acetyl chloride (310 mg, crude) was obtained as yellow oil.

### Step D: Preparation of (S)-methyl 5-(2-(4-(6-((4-chloro-2-fluorobenzyl)oxy)pyridin-2-yl) phenyl)acetamido)-6-((oxetan-2-ylmethyl)amino)picolinate

To a solution of methyl 5-amino-6-[[(2S)-oxetan-2-yl]methylamino]pyridine-2-carboxylate (170 mg, 716.53 umol) and TEA (0.30 mL, 2.15 mmol) in DCM (5 mL) was added 2-[4-[6-[(4-chloro-2-fluoro-phenyl)methoxy]-2-pyridyl]phenyl]acetyl chloride (279 mg, 716.53 umol), and the reaction mixture was stirred at 25° C. for 1 h. The reaction mixture was poured into water (5 mL). The aqueous phase was extracted with ethyl acetate (20 mL x 3). The combined organic phase was washed with brine (3 mL x 3), dried with anhydrous Na₂SO₄, filtered, and concentrated in vacuo. The residue was purified by silica gel chromatography (PE: EA = 0:1). Methyl 5-[[2-[4-[6-[(4-chloro-2-fluoro-phenyl)methoxy]-2-pyridyl]phenyl]acetyl]amino]-6-[[(2S)-oxetan-2-yl]methylamino]pyridine-2-carboxylate (140 mg, yield: 33.1%) was obtained as white solid. LCMS: m/z613.0[M+Na]⁺.

### Step E: Preparation of 2-[[4-[6-[(4-chloro-2-fluoro-phenyl)methoxyl)-2-pyridyl]phenyl] methyl]-3-[[(2S)-oxetan-2-yl]methyl]imidazo[4,5-b]pyridine-5-carboxylic acid

To a solution of methyl 5-[[2-[4-[6-[(4-chloro-2-fluoro-phenyl)methoxy]-2-pyridyl]phenyl]acetyl]amino]-6-[[(2S)-oxetan-2-yl]methylamino]pyridine-2-carboxylate (90 mg, 152.28 umol) in i-PrOH (9 mL) was added t-BuOK (34 mg, 304.55 umol). The mixture was stirred at 85° C. for 30 min. The mixture was adjusted to pH = 6 with 1N HCl. The solvent was removed in vacuo. The residue was washed with H₂O (10 mL), extracted with DCM (20 mL x 4). The organics were collected and concentrated. The residue was purified by prep-HPLC (FA) (column: Welch Xtimate C18 100 x 40 mm x 3um; mobile phase: [water (0.225% FA) - ACN]; B%: 62% - 72%, 8 min). 2-[[4-[6-[(4-chloro-2-fluoro-phenyl)methoxy]-2-pyridyl]phenyl] methyl]-3-[[(2S)-oxetan-2-yl]methyl]imidazo[4,5-b]pyridine-5-carboxylic acid (20.1 mg, yield: 21.8%) was obtained as white solid. LCMS: m/z 559.1 [M+H]⁺.

¹H NMR (400 MHz, CD₃OD) δ 8.20 - 8.08 (m, 2 H), 8.07 - 7.98 (m, 2 H), 7.76 - 7.68 (m, 1 H), 7.56 - 7.50 (m, 1 H), 7.49 - 7.44 (m, 1 H), 7.42 - 7.34 (m, 2 H), 7.27 - 7.15 (m, 2 H), 6.81 - 6.72 (m, 1 H), 5.51 (s, 2 H), 5.24 - 5.16 (m, 1 H), 4.75 - 4.54 (m, 4 H), 4.49 - 4.40 (m, 1 H), 2.81 - 2.70 (m, 1 H), 2.52 - 2.41 (m, 1 H).

### Example A: CAMP Assays

Activation of GLP-1 receptor is known to stimulate cyclic AMP (cAMP) production in cells which indicates primary coupling to the Gₐₛ subunit of the G protein heterotrimeric complex. Evidence suggests signaling through Gₐₛ induced cAMP stimulation elicits the desired pharmacological response regarding insulin release from pancreatic β-cells.

Method 1: To optimize functional activity directed toward Gₐₛ coupling, a CHO—K1 cell line developed by DiscoverX stably expressing the GLP-1 Receptor was used. Cells expressing GLP-1 receptor were plated in a 384-well microtiter plates and incubated overnight at 37° C. with 5% CO₂ to allow the cells to attach and grow. Media was then aspirated from the cells and replaced with 15 uL 2:1 Hanks Balanced Salt Solution (HBSS)/10mM Hepes : cAMP XS+ Ab reagent. Five microliters (5 uL) of previously generated compound sample stocks at 4x final concentration in assay buffer were then added to the cells and allowed to incubate at 37° C. for 30 or 60 minutes.

After incubation the assay signal was generated using enzyme fragment complementation (EFC). In EFC, the enzyme B-galactosidase is split into two complementary portions (EA and ED). The fragment ED is fused to cAMP and in the assay format competes with endogenous cAMP for binding to a cAMP specific antibody. Activated β—Gal is formed when exogenous EA fragment binds to free ED-cAMP (not bound to cAMP specific antibody). Activated enzyme levels are detected through conversion of β-gal chemiluminescent substrate which generates a detectable luminescence signal and read on standard microtiter plate.

The methodology for detection of cAMP using EFC requires incubation with 20uL of cAMP XS+ ED/CL lysis cocktail for one hour followed by incubation with 20 uL cAMP XS+ EA reagent for three hours at room temperature. Microplates were read following signal generation with a PerkinElmer Envision instrument utilizing chemiluminescent signal detection. Compound activity was analyzed using CBIS data analysis suite (ChemInnovation, CA). Percentage activity was calculated using the following formula:

%Activity = 100% x (mean RLU of test sample - mean RLU of vehicle control) / (mean RLU of MAX control - mean RLU of vehicle control))

Method 2: Activation of GLP-1 receptor is known to stimulate cyclic AMP (cAMP) production in cells which indicates primary coupling to the Gₐₛ subunit of the G protein heterotrimeric complex. Evidence suggests signaling through Gₐₛ induced cAMP stimulation elicits the desired pharmacological response regarding insulin release from pancreatic β-cells.

To optimize functional activity directed toward Gₐₛ coupling, a HEK293/CRE-Luc cell line developed by HDB stably expressing the GLP-1 Receptor was used. 200× concentration of compound working solutions were prepared (Agilent Technologies Bravo) with ½log serial dilution in 384-well Echo LDV plate (Labcyte, Cat# LP-0200). 50 nL/well 200× concentration of compound working solutions were moved to 384-well white low volume plate (Greiner,Cat#784075) using Labcyte ECHO550. 1×10⁵ cells/mL HEK293/GLP1R/CRE-LUC(HD Biosciences) cell suspensions prepared with assay buffer[DPBS containing 0.5 mM IBMX(Sigma,Cat# 15879) and 0.1% BSA(GENVIEW, Cat# FA016-100g)], 10 uL cell suspensions were added to each well of previous generated assay plate which already contains 50nl compound at 200×concentration using ThermoFisher Multidrop Combi(1000 cells/well). Seal the plate and incubate at 37° C. with 5% CO2 for 30 min.

After incubation the cAMP assay signal was generated using cAMP dynamic 2 Kit (Cisbio). 5 µL cAMP-d2 working solution was added to each well, followed with 5 µL Anti-cAMP antibody-cryptate working solution added to each well using ThermoFisher Multidrop Combi. Incubate at room temperature for 1 hour protected from light. Read the fluorescence at 665 and 615 nm with Reader PerkinElmer EnVision.

%Activity = 100% x (mean RLU of test sample - mean RLU of vehicle control) / (mean RLU of MAX control - mean RLU of vehicle control))

Reported EC₅₀ values with n>= 2 are represented as geometric means of individual measurements of EC₅₀. This is done to account for the lognormal distribution of multiple estimates of EC₅₀ values. In practice geometric mean is calculated by first generating log values of EC₅₀, averaging replicates and then calculating the antilog of the average.

Table 1 shows the biological activity of compounds in GLP-1R agonist cAMP stimulation assay (EC₅₀)

| Compound Number | GLP1R cAMP Stimulation DR: EC₅₀ (nM) Method 1 | GLP1R cAMP Stimulation DR: EC₅₀ (nM) Method 2 (HDB) |
|---|---|---|
| 101a | <0.051 | 0.019 |
| 101b | | 0.0088 |
| 101c | | 0.083 |
| 102a | 0.016 | 0.0096 |
| 102b | | 0.007 |
| 102c | | 0.044 |
| 103a | 0.073 | 0.095 |
| 104a | 0.114 | 0.12 |
| 105a | 0.12 | 0.089 |
| 110a | <0.051 | 0.0074 |
| 111a | 0.18 | 0.073 |
| 112a | 0.26 | 0.17 |
| 113a | | 0.015 |
| 114a | | 0.02 |
| 114b | | 0.012 |
| 114c | | 0.13 |
| 115a | | 0.018 |
| 116a | | 0.73 |
| 117a | | 0.37 |
| 118a | | 0.02 |
| 119a | | 0.17 |
| 120a | | 0.12 |
| 121a | | 0.027 |
| 122 | | 0.43 |
| 123 | | 0.067 |
| 124 | | 0.39 |
| 125 | | 0.045 |
| 126 | | 0.37 |
| 127 | | 0.83 |
| 128 | | 59 |

Table 1W shows the biological activity of compounds in GLP-1R agonist cAMP stimulation assay (EC₅₀)

| Compound Number | GLP1R cAMP Stimulation DR: EC₅₀ (nM) Method 1 | GLP1R cAMP Stimulation DR: EC₅₀ (nM) Method 2 (HDB) |
|---|---|---|
| 101aw | 0.069 | 0.097 |
| 102aw | 2.2 | 1.9 |
| 103aw | 0.38 | 0.89 |
| 104aw | 0.17 | 0.13 |
| 105aw | 11 | 7.1 |
| 106aw | 1.3 | 4.1 |
| 107aw | 0.28 | 0.19 |
| 108aw | 5.9 | 5.7 |
| 109aw | 4.8 | 10 |
| 110aw | 43 | 88 |
| 111aw | 0.77 | 0.54 |
| 120aw | < 0.051 | 0.017 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A compound of Formula I: 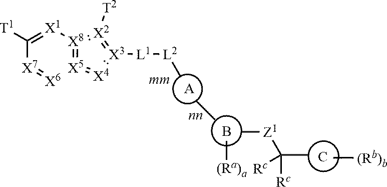
or a pharmaceutically acceptable salt or solvate thereof, wherein: [symbol]
indicates an optional single or double bond, as allowed by valence;
each of X¹, X², X³, X⁴, X⁵, X⁶, X⁷, and X⁸ is independently selected from the group consisting of C, CH, and N, provided that at least two and no more than four of X¹, X², X3, X⁴, X⁵, X⁶, X⁷, and X⁸ are N;
T¹ is C(═O)OH or a carboxylic acid bioisostere;
T² is a (C₁-C₆)alkyl optionally substituted with (C₃-C₆)cycloalkyl, 3- to 6-membered heterocycloalkyl, phenyl, 5- to 6-membered heteroaryl, (C₁-C₆)alkoxy, CN, or (C₂-C₄)alkynyl, wherein each of the (C₃-C₆)cycloalkyl, 3- to 6-membered heterocycloalkyl, phenyl, or 5- to 6-membered heteroaryl is optionally substituted with 1-4 R^{x};
each R^{x} is independently selected from the group consisting of OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino;
L¹ is (C₁-C₃)alkylene, which is optionally substituted with 1-3 R^{L};
L² is a bond, —O—, -S(O)₀₋₂-, or —NH—;
each R^{L} is independently selected from the group consisting of: halogen, (C₁-C₃)alkyl, and (C₁-C₃)haloalkyl; or
a pair of R^{L} on the same or on adjacent carbon atoms, taken together with the atom(s) to which each is attached, forms a (C₃-C₆)cycloalkyl ring;
Ring A is selected from the group consisting of:
partially unsaturated monocyclic (C₅-C₈)cycloalkylene optionally substituted with 1-4 substituents each independently selected from the group consisting of: halogen, (C₁-C₃)alkyl, (C₁-C₃)haloalkyl, (C₁-C₃)alkoxy, and (C₁-C₃)haloalkoxy; and
partially unsaturated monocyclic 5- to 8-membered heterocycloalkylene optionally substituted with 1-4 substituents each independently selected from the group consisting of: halogen, (C₁-C₃)alkyl, (C₁-C₃)haloalkyl, (C₁-C₃)alkoxy, and (C₁-C₃)haloalkoxy;
wherein mm represents the point of attachment to L², and nn represents the point of attachment to Ring B;
Ring B is selected from the group consisting of: 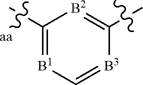 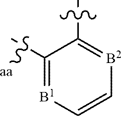 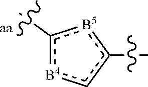 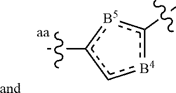
wherein aa represents the point of attachment to Ring A;
each of B¹, B², and B³ is independently selected from the group consisting of CR¹ and N;
each of B⁴ and B⁵ is independently selected from the group consisting of N, NR¹, C, CR¹, O, and S, provided that the ring containing B⁴ and B⁵ is heteroaryl;
R¹ is selected from the group consisting of H, halogen, and (C₁-C₆)alkyl;
each R^{a} is independently selected from the group consisting of (C₁-C₆)alkyl, (C₁-C₃)alkyl(C₃-C₆)cycloalkyl, (C₁-C₃)alkyl(3- to 5-membered heterocycloalkyl), —C(O)NR²R³, and (C₁-C₆)fluoroalkyl;
each R² and R³ is independently selected from the group consisting of H and (C₁-C₆)alkyl;
a is an integer selected from 0-3; 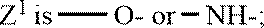
each R^{c} is independently selected from the group consisting of H, (C₁-C₆)alkyl, and (C₁-C₃)haloalkyl;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)alkoxy, halogen, (C₃-C₆)cycloalkyl, and CN; and
b is an integer selected from 0-3.

2. A compound of Formula II: 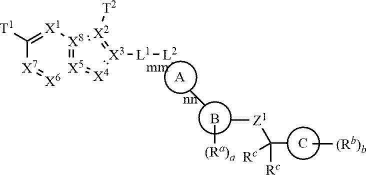
or a pharmaceutically acceptable salt or solvate thereof, wherein: [symbol]
indicates an optional single or double bond, as allowed by valence;
each of X¹, X², X³, X⁴, X⁵, X⁶, X⁷, and X⁸ is independently selected from the group consisting of C, CH, and N, provided that at least two and no more than four of X¹, X², X³, X⁴, X⁵, X⁶, X⁷, and X⁸ are N;
T¹ is C(═O)OH or a carboxylic acid bioisostere;
T² is a (C₁-C₆)alkyl optionally substituted with (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{x};
each R^{x} is independently selected from the group consisting of OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino;
L¹ is (C₁-C₃)alkylene, which is optionally substituted with 1-3 R^{L};
L² is a bond, —O—, -S(O)₀₋₂-, or —NH—;
each R^{L} is independently selected from the group consisting of: halogen, (C₁-C₃)alkyl, and (C₁-C₃)haloalkyl; or
a pair of R^{L} on the same or on adjacent carbon atoms, taken together with the atom(s) to which each is attached, forms a (C₃-C₆)cycloalkyl ring;
Ring A is selected from the group consisting of:
phenylene optionally substituted with 1-4 R^{Y};
5- to 6-membered heteroarylene optionally substituted with 1-3 R^{Y};
wherein mm represents the point of attachment to L², and nn represents the point of attachment to Ring B; and
each R^{Y} is independently selected from the group consisting of halogen, cyano, —OH, oxo, (C₁-C₃)alkyl, (C₁-C₃)haloalkyl, (C₁-C₃)alkoxy, and (C₁-C₃)haloalkoxy;
Ring B is selected from the group consisting of: 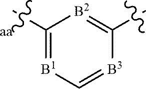 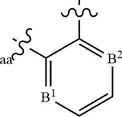 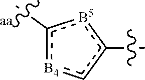 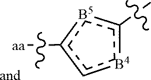
wherein aa represents the point of attachment to Ring A;
each of B¹, B², and B³ is independently selected from the group consisting of CR¹ and N;
each of B⁴ and B⁵ is independently selected from the group consisting of N, NR¹, C, CR¹, O, and S, provided that the ring containing B⁴ and B⁵ is heteroaryl;
R¹ is selected from the group consisting of H, halogen, and (C₁-C₆)alkyl;
each R^{a} is independently selected from the group consisting of (C₁-C₆)alkyl, (C₁-C₃)alkyl(C₃-C₆)cycloalkyl, (C₁-C₃)alkyl(3- to 5-membered heterocycloalkyl), —C(O)NR²R³, and (C₁-C₆)fluoroalkyl;
each R² and R³ is independently selected from the group consisting of H and (C₁-C₆)alkyl;
a is an integer selected from 0-3;
Z¹ is —O— or -NH—;
each R^{c} is independently selected from the group consisting of H, (C₁-C₆)alkyl, and (C₁-C₃)haloalkyl;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)alkoxy, halogen, (C₃-C₆)cycloalkyl, and CN; and
b is an integer selected from 0-3.

3. The compound of claims 1 or 2, wherein X⁸ is C; and X⁵ is C.

4. The compound of any one of claims 1-3, wherein X³ is C.

5. The compound of any one of claims 1-4, wherein X² is N.

6. The compound of any one of claims 1–5, wherein X⁴ is N.

7. The compound of any one of claims 1–6, wherein X⁷ is CH.

8. The compound of any one of claims 1–7, wherein each X⁸, X⁵, and X³ are C; X² and X⁴ are N; X⁷ is CH; and X¹ and X⁶ are independently CH or N.

9. The compound of claim 8, wherein X¹ and X⁶ are CH.

10. The compound of claim 8, wherein X¹ is N; and X⁶ is CH.

11. The compound of claim 8, wherein X¹ is CH; and X⁶ is N.

12. The compound of any one of claims 1–11, wherein T¹ is C(═O)OH.

13. The compound of any one of claims 1–12, wherein T² is (C₁-C₃)alkyl which is substituted with (C₃-C₆)cycloalkyl, 3- to 6-membered heterocycloalkyl, phenyl, or 5- to 6-membered heteroaryl.

14. The compound of any one of claims 1–13, wherein T² is (C₁-C₃)alkyl which is substituted with (C₃-C₆)cycloalkyl or 3- to 6-membered heterocycloalkyl.

15. The compound of any one of claims 1–14, wherein T² is (C₁-C₃)alkyl which is substituted with 3- to 6-membered heterocycloalkyl.

16. The compound of any one of claims 1–15, wherein T² is (C₁-C₃)alkyl which is substituted with 4- to 6-membered heterocycloalkyl.

17. The compound of any one of claims 1–16, wherein T² is (C₁-C₃)alkyl which is substituted with oxetanyl.

18. The compound of any one of claims 1–17, wherein T² is 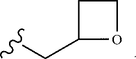
.

19. The compound of any one of claims 1–18, wherein L² is a bond.

20. The compound of any one of claims 1–18, wherein L² is —O—.

21. The compound of any one of claims 1–20, wherein L¹ is C₁₋₂ alkylene, which is optionally substituted with 1-3 R^{L}.

22. The compound of any one of claims 1–21, wherein L¹ is CH₂.

23. The compound of any one of claims 1–21, wherein L¹ is CH₂CH₂.

24. The compound of any one of claims 1–21, wherein L¹ is CH₂CH₂, which is substituted with 1-3 R^{L}.

25. The compound of any one of claims 1–21, wherein L¹ is CH₂CH₂, which is substituted with two R^{L}, wherein the pair of R^{L} on adjacent carbon atoms, taken together with the atoms to which each is attached, forms a C₃-C₅ cycloalkyl ring.

26. The compound of any one of claims 1–18, wherein L² is a bond; and L¹ is CH₂.

27. The compound of any one of claims 1–18, wherein L² is a bond; and L¹ is CH₂CH₂, or 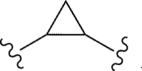
.

28. The compound of any one of claims 1–18, wherein L² is —O—; and L¹ is C₁₋₂ alkylene, which is optionally substituted with 1-3 R^{L}.

29. The compound of claim 28, wherein L¹ is CH₂.

30. The compound of any one of claims 1–29, wherein:
(i) mm is para to nn;
(ii) mm is meta to nn;
(iii) L² is a bond; L¹ is CH₂; and mm is para to nn;
(iv) L² is a bond; L¹ is CH₂CH₂ or 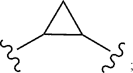
and mm is meta to nn; or
(v) L² is —O—; L¹ is CH₂; and mm is meta to nn.

31. The compound of any one of claims 1 or 3–30 , wherein Ring A is partially unsaturated monocylic (C₅-C₈)cycloalkylene optionally substituted with 1-4 substituents each independently selected from the group consisting of: halogen, (C₁-C₃)alkyl, (C₁-C₃)haloalkyl, (C₁-C₃)alkoxy, and (C₁-C₃)haloalkoxy.

32. The compound of any one of claims 1 or 3–31, wherein Ring A is partially unsaturated monocylic C₆ cycloalkylene optionally substituted with from 1-4 substituents each independently selected from the group consisting of: halogen, (C₁-C₃)alkyl, (C₁-C₃)haloalkyl, (C₁-C₃)alkoxy, and (C₁-C₃)haloalkoxy.

33. The compound of any one of claims 1 or 3–32, wherein Ring A is cyclohexenylene optionally substituted with from 1-4 substituents each independently selected from the group consisting of: halogen, (C₁-C₃)alkyl, (C₁-C₃)haloalkyl, (C₁-C₃)alkoxy, and (C₁-C₃)haloalkoxy.

34. The compound of any one of claims 1 or 3–33, wherein Ring A is unsubstituted cyclohexenylene.

35. The compound of any one of claims 1 or 3–34, wherein Ring A is 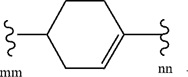
.

36. The compound of any one of claims 1 or 3–30, wherein Ring A is partially unsaturated monocyclic 5- to 8-membered heterocycloalkylene optionally substituted with from 1-4 substituents each independently selected from the group consisting of: halogen, (C₁-C₃)alkyl, (C₁-C₃)haloalkyl, (C₁-C₃)alkoxy, and (C₁-C₃)haloalkoxy.

37. The compound of any one of claims 1, 3–30 or 36, wherein Ring A is partially unsaturated monocyclic 5- to 6-membered heterocycloalkylene optionally substituted with from 1-4 substituents each independently selected from the group consisting of: halogen, (C₁-C₃)alkyl, (C₁-C₃)haloalkyl, (C₁-C₃)alkoxy, and (C₁-C₃)haloalkoxy.

38. The compound of any one of claims 1, 3–30 or 36–37, wherein Ring A is tetrahydropyridinylene which is optionally substituted with from 1-4 substituents each independently selected from the group consisting of: halogen, (C₁-C₃)alkyl, (C₁-C₃)haloalkyl, (C₁-C₃)alkoxy, and (C₁-C₃)haloalkoxy.

39. The compound of any one of claims 1, 3–30 or 36–38, wherein Ring A is unsubstituted tetrahydropyridinylene.

40. The compound of any one of claims 1, 3–30 or 36–39, wherein Ring A is 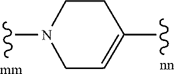
.

41. The compound of any one of claims 2–30, wherein Ring A is phenylene optionally substituted with 1-4 R^{Y}.

42. The compound of any one of claims 2–30, wherein Ring A is 1,4-phenylene or 1,3-phenylene optionally substituted with 1-2 R^{Y}.

43. The compound of any one of claims 2–30 or 42, wherein Ring A is 1,4-phenylene optionally substituted with 1-2 R^{Y}.

44. The compound of any one of claims 2–30 or 42–43, wherein Ring A is 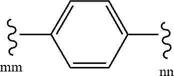
.

45. The compound of any one of claims 2–30, wherein Ring A is 5- to 6-membered heteroarylene optionally substituted with 1-3 R^{Y}.

46. The compound of any one of claims 2–30 or 45, wherein Ring A is 6-membered heteroarylene optionally substituted with 1-3 R^{Y}.

47. The compound of any one of claims 2–30 or 45–46, wherein Ring A is 2,4-pyridinylene or 3,5-pyridinylene optionally substituted with 1-2 R^{Y}.

48. The compound of any one of claims 2–30 or 45–47, wherein Ring A is 2,4-pyridinylene optionally substituted with 1-2 R^{Y}.

49. The compound of any one of claims 2–30 or 45–48, wherein Ring A is selected from the group consisting of: 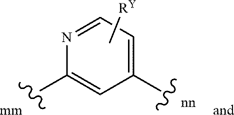 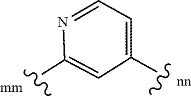
.

50. The compound of any one of claims 2–30 or 45–46, wherein Ring A is 6-membered heteroarylene substituted with 1-3 R^{Y}, provided that at least one R^{Y} is oxo.

51. The compound of any one of claims 2–30 or 45–46 or 50, wherein Ring A is pyridonylene which is further optionally substituted with 1-2 R^{Y}.

52. The compound of any one of claims 2–30 or 45–46 or 50–51, wherein Ring A is 1,4-pyridonylene which is further optionally substituted with 1-2 R^{Y}.

53. The compound of any one of claims 2–30 or 45–46 or 50–52, wherein Ring A is selected from the group consisting of: 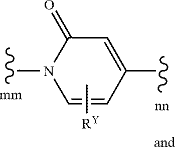 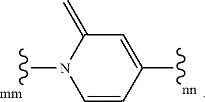
.

54. The compound of any one of claims 2–30 or 45–46, wherein Ring A is 5-membered heteroarylene optionally substituted with 1-2 R^{Y}.

55. The compound of any one of claims 2–30 or 45–46 or 54, wherein Ring A is pyrazolylene optionally substituted with 1-2 R^{Y}.

56. The compound of any one of claims 2–30 or 45–46 or 54–55, wherein Ring A is selected from the group consisting of: 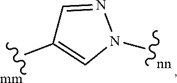 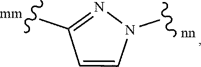 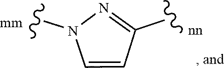 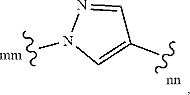
each of which is optionally substituted with one R^{Y}.

57. The compound of any one of claims 1–56, wherein each R^{Y} is independently selected from the group consisting of: halogen and C₁-C₃alkl.

58. The compound of any one of claims 1–57, wherein Ring B is 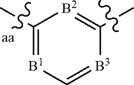
.

59. The compound of claim 58, wherein B² is N.

60. The compound of claim 58 or 59, wherein B¹ and B³ are independently CR¹.

61. The compound of claim 58 or 59, wherein one of B¹ and B³ is N; and the other one of B¹ and B³ is CR¹.

62. The compound of claim 58 or 59, wherein B¹ is N; and B³ is CR¹.

63. The compound of claim 58 or 59, wherein B¹ is CR¹; and B³ is N.

64. The compound of claim 58, wherein B² is CR¹.

65. The compound of claim 58 or 64, wherein B¹ and B³ are independently CR¹.

66. The compound of any one of claims 1-58, wherein Ring B is 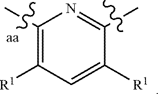
.

67. The compound of any one of claims 1-58, wherein Ring B is 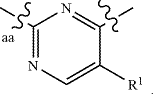
.

68. The compound of any one of claims 1-58, wherein Ring B is 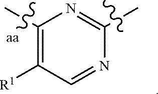
.

69. The compound of any one of claims 1-58, wherein Ring B is 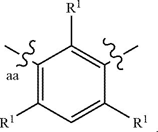
.

70. The compound of any one of claims 1-57, wherein Ring B is 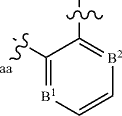
.

71. The compound of claim 70, wherein B² is N; or B² is CR¹.

72. The compound of claim 70 or 71, wherein B¹ is CR¹.

73. The compound of claim 70 or 71, wherein B¹ is N.

74. The compound of any one of claims 1–57 or 70, wherein Ring B is 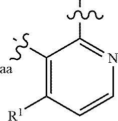
.

75. The compound of any one of claims 1–57 or 70, wherein Ring B is 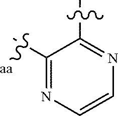
.

76. The compound of any one of claims 1–57, wherein Ring B is 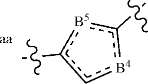
.

77. The compound of claim 76, wherein B⁵ is N.

78. The compound of any one of claims 76–77, wherein B⁴ is selected from the group consisting of NR¹, S, and O.

79. The compound of claim 78, wherein B⁴ is S.

80. The compound of any one of claims 1–57 or 76, wherein Ring B is 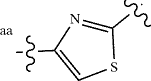
.

81. The compound of any one of , wherein each R¹ is independently H or halogen.

82. The compound of any one of claims 1–81, wherein each R¹ is H.

83. The compound of any one of claims 1–82, wherein a is 0.

84. The compound of any one of claims 1–83, wherein Z¹ is —O—.

85. The compound of any one of claims 1–84, wherein each R^{c} is H.

86. The compound of any one of claims 1–85, wherein Ring C is selected from the group consisting of: phenyl, 5- to 6-membered heteroaryl, and 5- to 10-membered bicycloheteroaryl.

87. The compound of any one of claims 1–86, wherein Ring C is phenyl.

88. The compound of any one of claims 1–87, wherein b is 1-3.

89. The compound of any one of claims 1–88, wherein b is 2.

90. The compound of any one of claims 1–85, wherein Ring C is phenyl; and b is 2.

91. The compound of claim 90, wherein 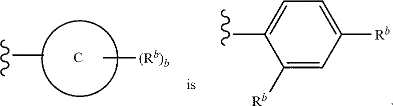
.

92. The compound of any one of claims 1–91, wherein each occurrence of R^{b} is independently selected from the group consisting of: (C₁-C₆)alkyl, (C₁-C₆)alkoxy, halogen, and CN.

93. The compound of any one of claims 1–92, wherein each occurrence of R^{b} is independently selected from the group consisting of —F, —Cl, and CN.

94. The compound of claim 1, wherein the compound of Formula I is a compound of Formula IA: 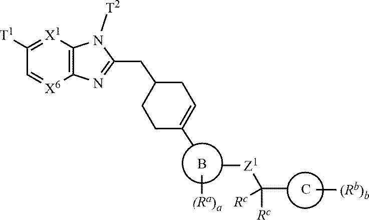
or a pharmaceutically acceptable salt or solvate thereof.

95. The compound of claim 1, wherein the compound of Formula I is a compound of Formula IB: 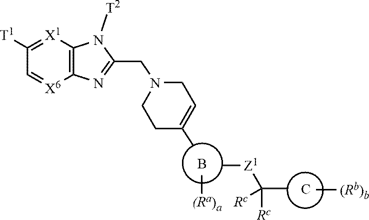
or a pharmaceutically acceptable salt or solvate thereof.

96. The compound of claim 94 or 95, wherein X¹ is N.

97. The compound of any one of claims 94–96, wherein X⁶ is CH.

98. The compound of claim 94 or 95, wherein X is N; and X⁶ is CH.

99. The compound of any one of claims 94–98, wherein T¹ is C(═O)OH.

100. The compound of any one of claims 94–99, wherein T² is (C₁-C₃)alkyl which is substituted with 3- to 6-membered heterocycloalkyl.

101. The compound of any one of claims 94–100, wherein T² is (C₁-C₃)alkyl which is substituted with oxetanyl.

102. The compound of any one of claims 74–101, wherein T² is is 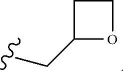
.

103. The compound of any one of claims 94–102, wherein Ring B is 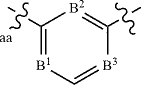
.

104. The compound of any one of claims 94–103, wherein Ring B is 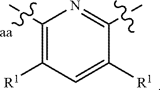
.

105. The compound of any one of claims 94–103, wherein Ring B is selected from the group consisting of: 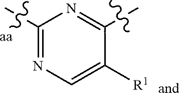 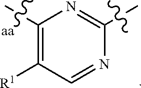
.

106. The compound of any one of claims 94–102, wherein Ring B is 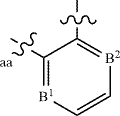
.

107. The compound of any one of claims 94–102 or 106, wherein Ring B is 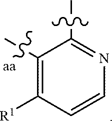
.

108. The compound of any one of claims 94–102 or 106, wherein Ring B is 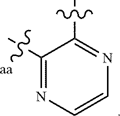
.

109. The compound of any one of claims 94–102, wherein Ring B is 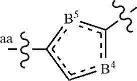
.

110. The compound of any one of claims 94–102 or 109, wherein Ring B is 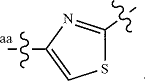
.

111. The compound of any one of claims 94–110, wherein each R¹ is independently H or halogen.

112. The compound of any one of claims 94–111, wherein each R¹ is H.

113. The compound of any one of claims 94–112, wherein a is 0.

114. The compound of any one of claims 94–113, wherein Z¹ is —O—.

115. The compound of any one of claims 94–114, wherein each R^{c} is H.

116. The compound of any one of claims 94–115, wherein Ring C is phenyl.

117. The compound of any one of claims 94–116, wherein b is 1-3.

118. The compound of any one of claims 94–117, wherein b is 2.

119. The compound of any one of claims 94–118, wherein Ring C is phenyl; and b is 2.

120. The compound of any one of claims 94–119, wherein 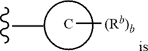 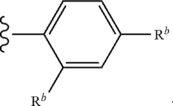
.

121. The compound of any one of claims 94–120, wherein each occurrence of R^{b} is independently selected from the group consisting of: (C₁-C₆)alkyl, (C₁-C₆)alkoxy, halogen, and CN.

122. The compound of any one of claims 94–121, wherein each occurrence of R^{b} is independently selected from the group consisting of —F, —Cl, and CN.

123. The compound of any one of claims 1 or 3-122, wherein the compound of Formula I is selected from the group consisting of the compounds in Table C1, or a pharmaceutically acceptable salt or solvate thereof.

124. The compound of any one of claims 1 or 3-123, wherein the compound of Formula I is selected from the group consisting of the compounds in Table C2, or a pharmaceutically acceptable salt or solvate thereof.

125. A pharmaceutical composition comprising a compound of any one of claims 1-124, or a pharmaceutically acceptable salt or solvate thereof, and a pharmaceutically acceptable excipient.

126. The compound of claim 2, wherein the compound of Formula II is a compound of Formula IIA: 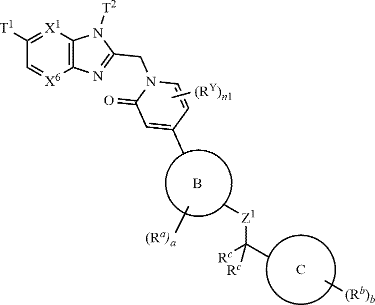
or a pharmaceutically acceptable salt or solvate thereof, wherein n1 is 0 or 1.

127. The compound of claim 2, wherein the compound of Formula II is a compound of Formula IIB: 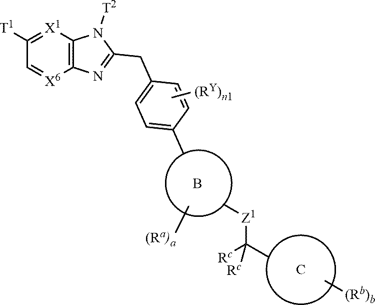
or a pharmaceutically acceptable salt or solvate thereof, wherein n1 is 0 or 1.

128. The compound of claim 2, wherein the compound of Formula II is a compound of Formula IIC: 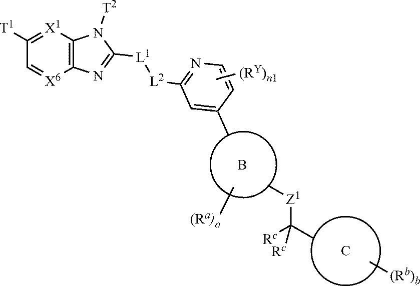
or a pharmaceutically acceptable salt or solvate thereof, wherein n1 is 0 or 1.

129. The compound of claim 128, wherein L¹ is CH₂; and L² is —O—.

130. The compound of claim 128, wherein L¹ is CH₂CH₂; and L² is a bond.

131. The compound of claim 128, wherein L¹ is 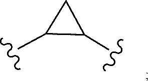
and L² is a bond.

132. The compound of any one of claims 126–128, wherein X¹ is N.

133. The compound of any one of claims 126–128, wherein X¹ is CH.

134. The compound of any one of claims 126–133, wherein X⁶ is CH.

135. The compound of any one of claims 126–128, wherein X¹ is N; and X⁶ is CH.

136. The compound of any one of claims 126–128, wherein X¹ and X⁶ are each CH.

137. The compound of any one of claims 126–136, wherein T¹ is C(═O)OH.

138. The compound of any one of claims 126–137, wherein T² is (C₁-C₃)alkyl which is substituted with 3- to 6-membered heterocycloalkyl.

139. The compound of any one of claims 126-138, wherein T² is (C₁-C₃)alkyl which is substituted with oxetanyl.

140. The compound of any one of claims 126-139, wherein T² is 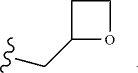
.

141. The compound of any one of claims 126–140, wherein n1 is 0.

142. The compound of any one of claims 126–140, wherein n1 is 1.

143. The compound of claim 142, wherein R^{Y} is independently selected from the group consisting of: halogen and (C₁-C₃)alkyl.

144. The compound of claim 143, wherein R^{Y} is selected from the group consisting of: —F and methyl.

145. The compound of any one of claims 126–144, wherein Ring B is 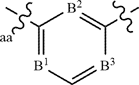
.

146. The compound of any one of claims 126–145, wherein Ring B is 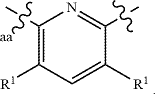
.

147. The compound of any one of claims 126–145, wherein Ring B is selected from the group consisting of: 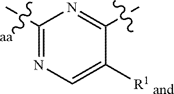 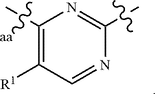
.

148. The compound of any one of claims 126–145, wherein Ring B is 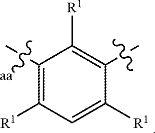
.

149. The compound of any one of claims 126–144, wherein Ring B is 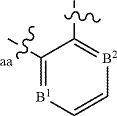
.

150. The compound of any one of claims 126–145 or 149, wherein Ring B is 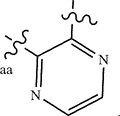
.

151. The compound of any one of claims 126–150, wherein each R¹ is independently H or halogen.

152. The compound of any one of claims 126–151, wherein each R¹ is H.

153. The compound of any one of claims 126–152, wherein a is 0.

154. The compound of any one of claims 126–153, wherein Z¹ is —O—.

155. The compound of any one of claims 126–154, wherein each R^{c} is H.

156. The compound of any one of claims 126–155, wherein Ring C is phenyl.

157. The compound of any one of claims 126–156, wherein b is 1-3.

158. The compound of any one of claims 126–157, wherein b is 2.

159. The compound of any one of claims 126–156, wherein b is 0.

160. The compound of any one of claims 126–158, wherein Ring C is phenyl; and b is 2.

161. The compound of claim 160, wherein 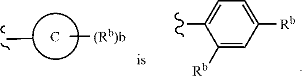
.

162. The compound of any one of claims 126–155, wherein Ring C is phenyl; and b is 0.

163. The compound of any one of claims 126–161, wherein each occurrence of R^{b} is independently selected from the group consisting of: (C₁-C₆)alkyl, (C₁-C₆)alkoxy, halogen, and CN.

164. The compound of any one of claims 126–156 or 163, wherein each occurrence of R^{b} is independently selected from the group consisting of —F, —Cl, and CN.

165. The compound of any one of claims 2-93 or 126-164, wherein the compound of Formula II is selected from the group consisting of the compounds in Table C1-W and Table C2-W, or a pharmaceutically acceptable salt or solvate thereof.

166. A pharmaceutical composition comprising a compound of any one of claims 2-93 or 126-165, or a pharmaceutically acceptable salt or solvate thereof, and a pharmaceutically acceptable excipient.

167. A method of treating type 2 diabetes mellitus in a patient in need thereof, the method comprising administering to the patient a therapeutically effective amount of a compound of any one of claims 1-165, or a pharmaceutically acceptable salt or solvate thereof, or a pharmaceutical composition according to claims 125 or 166.

168. A method for treating type 2 diabetes mellitus in a patient, the method comprising administering to a patient identified or diagnosed as having type 2 diabetes mellitus a therapeutically effective amount of a compound of any one of claims 1–124 or 126–165, or a pharmaceutically acceptable salt or solvate thereof, or a pharmaceutical composition according to claims 125 or 166.

169. A method of treating diabetes mellitus in a patient, the method comprising:
a) determining that the patient has type 2 diabetes mellitus; and
b)administering to the patient a therapeutically effective amount of a compound of any one of claims 1–124 or 126–165, or a pharmaceutically acceptable salt or solvate thereof, or a pharmaceutical composition according to claims 125 or 166.

170. The method of any one of claims 167–169, wherein the step of determining that the patient has type 2 diabetes mellitus includes performing an assay to determine the level of an analyte in a sample from the patient, wherein the analyte is selected from the group consisting of hemoglobin A1c (HbA1c), fasting plasma glucose, non-fasting plasma glucose, or any combination thereof.

171. The method of claim 170, wherein the level of HbA1c is greater than or about 6.5%.

172. The method of any one of claims 170–171, wherein the level of fasting plasma glucose is greater than or about 126 mg/dL.

173. The method of any one of claims 170–171, wherein the level of non-fasting plasma glucose is greater than or about 200 mg/dL.

174. The method of any one of claims 167–173, further comprising obtaining a sample from the patient.

175. The method of claim 174, wherein the sample is a body fluid sample.

176. The method of any one of claims 167–175, wherein the patient is about 40 to about 70 years old and is overweight or obese.

177. The method of any one of claims 167–176, wherein the patient has a body mass index (BMI) greater than or about 22 kg/m².

178. The method of any one of claims 167-177, wherein the patient has a BMI greater than or about 30 kg/m².

179. The method of any one of claims 167-178, wherein the treatment of type 2 diabetes mellitus comprises a reduction in fasting plasma glucose levels.

180. The method of claim 179, wherein the fasting plasma glucose levels are reduced to about or below 100 mg/dL.

181. The method of any one of claims 167–180, wherein the treatment of type 2 diabetes mellitus comprises a reduction in HbA1c levels.

182. The method of claim 181, wherein the HbA1c levels are reduced to about or below 5.7 %.

183. The method of any one of claims 167–182, wherein the treatment of type 2 diabetes mellitus comprises a reduction in glucagon levels.

184. The method of any one of claims 167–182, wherein the treatment of type 2 diabetes mellitus comprises an increase in insulin levels.

185. The method of any one of claims 167–182, wherein the treatment of type 2 diabetes mellitus comprises a decrease in BMI.

186. The method of claim 185, wherein the BMI is decreased to about or below 25 kg/m².

187. The method of any of one of claims 167–186, wherein the compound of any one of claims 1–165, or a pharmaceutically acceptable salt or solvate thereof, or a pharmaceutical composition according to claims 125 or 166, is administered orally.

188. The method of any one of claims 167–187, further comprising administering an additional therapy or therapeutic agent to the patient.

189. The method of claim 188, wherein the additional therapy or therapeutic agent is selected from the group consisting of an anti-diabetic agent, an anti-obesity agent, a GLP-1 receptor agonist, an agent to treat non-alcoholic steatohepatitis (NASH), gastric electrical stimulation, dietary monitoring, physical activity, or any combinations thereof.

190. The method of claim 189, wherein the antidiabetic agent is selected from the group consisting of a biguanide, a sulfonylurea, a glitazar, a thiazolidinedione, a dipeptidyl peptidase 4 (DPP-4) inhibitor, a meglitinide, a sodium-glucose linked transporter 2 (SGLT2) inhibitor, a glitazone, a GRP40 agonist, a glucose-dependent insulinotropic peptide (GIP), an insulin or insulin analogue, an alpha glucosidase inhibitor, a sodium-glucose linked transporter 1 (SGLT1) inhibitor, or any combinations thereof.

191. The method of claim 190, wherein the biguanide is metformin.

192. The method of claim 189, wherein the anti-obesity agent is selected from the group consisting of neuropeptide Y receptor type 2 (NPYR2) agonist, a NPYR1 or NPYR5 antagonist, a human proislet peptide (HIP), a cannabinoid receptor type 1 (CB1R) antagonist, a lipase inhibitor, a melanocortin receptor 4 agonist, a farnesoid X receptor (FXR) agonist, phentermine, zonisamide, a norepinephrine/dopamine reuptake inhibitor, a GDF-15 analog, an opioid receptor antagonist, a cholecystokinin agonist, a serotonergic agent, a methionine aminopeptidase 2 (MetAP2) inhibitor, diethylpropion, phendimetrazine, benzphetamine, a fibroblast growth factor receptor (FGFR) modulator, an AMP-activated protein kinase (AMPK) activator, a sodium-glucose cotransporter 1 (SGLT-1) inhibitor, or any combinations thereof.

193. The method of claim 189, wherein the GLP-1 receptor agonist is selected from the group consisting of liraglutide, exenatide, dulaglutide, albiglutide, taspoglutide, lixisenatide, semaglutide, or any combinations thereof.

194. The method of claim 189, wherein the agent to treat NASH is selected from the group consisting of an FXR agonist, PF-05221304, a synthetic fatty acid-bile conjugate, an anti-lysyl oxidase homologue 2 (LOXL2) monoclonal antibody, a caspase inhibitor, a MAPK5 inhibitor, a galectin 3 inhibitor, a fibroblast growth factor 21 (FGF21) agonist, a niacin analogue, a leukotriene D4 (LTD4) receptor antagonist, an acetyl-CoA carboxylase (ACC) inhibitor, a ketohexokinase (KHK) inhibitor, an ileal bile acid transporter (IBAT) inhibitor, an apoptosis signal-regulating kinase 1 (ASK1) inhibitor, a peroxisome proliferator-activated receptor (PPAR) agonist, a diacylglyceryl acyltransferase 2 (DGAT2) inhibitor, or any combinations thereof.

195. The method of any one of claims 188–194, wherein the compound of any one of claims 1–124 or 126–165 or a pharmaceutically acceptable salt or solvate thereof, or a pharmaceutical composition according to claims 125 or 166, and the additional therapeutic agent are administered as separate dosages sequentially in any order.

196. A method for modulating insulin levels in a patient in need of such modulating, the method comprising administering to the patient an effective amount of a compound as claimed in any one of claims 1–124 or 126–165, or a pharmaceutically acceptable salt or solvate thereof, or a pharmaceutical composition according to claims 125 or 166.

197. The method of claim 196, wherein the modulation results in an increase of insulin levels.

198. A method for modulating glucose levels in a patient in need of such modulating, the method comprising administering to the patient an effective amount of a compound as claimed in any one of claims 1–124 or 126–165, or a pharmaceutically acceptable salt or solvate thereof, or a pharmaceutical composition according to claims 125 or 166 .

199. The method of claim 198, wherein the modulation results in a decrease of glucose levels.

200. A method for treating a GLP-1 associated disease, disorder, or condition, the method comprising administering to a patient in need thereof an effective amount of a compound as claimed in any one of claims 1–124 or 126–165, or a pharmaceutically acceptable salt or solvate thereof, or a pharmaceutical composition according to claims 125 or 166 .

201. The method of claim 200, wherein the disease, disorder, or condition is selected from the group consisting of type 1 diabetes mellitus, type 2 diabetes mellitus, early onset type 2 diabetes mellitus, idiopathic type 1 diabetes mellitus (Type 1b), youthonset atypical diabetes (YOAD), maturity onset diabetes of the young (MODY), latent autoimmune diabetes in adults (LADA), obesity, weight gain from use of other agents, idiopathic intracranial hypertension, Wolfram syndrome, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn’s disease, short bowel syndrome, Parkinson’s, Alzheimer’s disease, impaired cognition, schizophrenia, Polycystic Ovary Syndrome (PCOS), or any combination thereof.

202. The method of claim 201, wherein the disease, disorder, or condition is selected from the group consisting of type 2 diabetes mellitus, early onset type 2 diabetes mellitus, obesity, weight gain from use of other agents, gout, excessive sugar craving, hypertriglyceridemia, dyslipidemia, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson’s disease, Polycystic Ovary Syndrome (PCOS), idiopathic intracranial hypertension, Wolfram syndrome, or any combination thereof.

203. The method of claim 202, wherein the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, idiopathic intracranial hypertension, Wolfram syndrome, or any combination thereof.
